(19) United States
(12) Patent Application Publication
(10) Pub. No.: US 2021/0033619 A1
(43) Pub. Date: Feb. 4, 2021

(54) METHODS OF PREDICTING PRE TERM BIRTH FROM PREECLAMPSIA USING METABOLIC AND PROTEIN BIOMARKERS

(71) Applicant: METABOLOMIC DIAGNOSTICS LIMITED, Co. Cork (IE)

(72) Inventors: Robin TUYTTEN, Co. Cork (IE); Gregoire THOMAS, Lokeren (BE); Louise KENNY, Wilton Cork (IE); Leslie BROWN, Cork (IE)

(73) Assignee: METABOLOMIC DIAGNOSTICS LIMITED, Co. Cork (IE)

(21) Appl. No.: 16/968,292

(22) PCT Filed: Feb. 11, 2019
(86) PCT No.: PCT/EP2019/053349

§ 371 Date: Aug. 7, 2020

(30) Foreign Application Priority Data
Feb. 9, 2018 (GB) 1802207.9
May 16, 2018 (EP) 18172711.6

(51) Int. Cl. G01N 33/68; G16B 5/00

(52) U.S. Cl. CPC G01N 33/689; G01N 2800/368; G16B 5/00

(57) ABSTRACT

A computer implemented method of early prediction of risk of a pregnancy outcome in a pregnant woman, comprising the steps of: inputting into a computational model values for a panel of a plurality of preeclampsia specific biomarkers comprising at least one metabolite, and optionally at least one protein or clinical risk factor, selected from Table 1, in which the values are obtained from the pregnant woman early in pregnancy; selecting a subset of inputted values comprising a value for at least one metabolite and optionally at least one protein or clinical risk factor value, based on a selected pregnancy outcome selected from pre-term preeclampsia, term preeclampsia and all preeclampsia; calculating a predicted risk of the selected pregnancy outcome based on the subset of inputted values; and outputting the predicted risk of the pregnancy outcome for the pregnant woman.

### FIELD OF THE INVENTION

The present invention relates to a method of predicting preeclampsia in a pregnant woman. Also contemplated are methods of predicting pre-term preeclampsia, and term preeclampsia, in a pregnant woman at an early stage of pregnancy.

### BACKGROUND TO THE INVENTION

Preeclampsia (PE) is a disorder specific to pregnancy which occurs in 2-8% of all pregnancies[1]. PE originates in the placenta and manifests as new-onset hypertension and proteinuria after 20 weeks' gestation[2]. PE remains a leading cause of maternal and perinatal morbidity and mortality. Each year 70,000 mothers and 500,000 infants die from the direct consequences of PE[3]. Maternal complications of PE include cerebrovascular accidents, liver rupture, pulmonary oedema or acute renal failure. For the fetus, placental insufficiency causes fetal growth restriction, which is associated with increased neonatal morbidity and mortality. To date, the only cure for PE is delivery of the placenta, and hence the baby. Consequently, iatrogenic prematurity adds to the burden of neonatal morbidity and mortality. The impact of PE on the health of patients is not restricted to the perinatal period. Affected mothers have a lifelong increased risk of cardiovascular disease, stroke and type 2 diabetes mellitus. Children born prematurely as a result of PE may have neurocognitive development issues ranging from mild learning difficulties to severe disabilities. In the longer term young children and adolescents of pregnancies complicated by PE exhibit increased blood pressure and BMI compared to their peers, with increased incidences of diabetes, obesity, hypertension and cardiac disease[4-6].

Whilst there are currently no ready available treatments to cure preeclampsia when it manifests, there are some drug treatments, i.e, aspirin and metformin (and others), which have the potential to prevent some of the preeclampsia cases developing [7,8]. However, for these prophylactic interventions with therapeutics to impact on the incidence of preeclampsia at the population level, health care providers need to have a risk stratification tool, or test, which combines the following two attributes: identification of those pregnancies at increased risk of the disease early in pregnancy and then triage these pregnancies to the appropriate treatment. These requirements follow the precautionary principle that one should not do harm to the pregnant woman and her unborn child. A blanket administration of drugs to all pregnancies in order to prevent preeclampsia in some, might incur unnecessary health risks (e.g., due to treatment side effects) in these who are not at risk in the first place.

Contemporary hypotheses regarding the etiological causes of preeclampsia suggest that PE is syndromic in nature, and that preeclampsia is possibly more than one disease [9]. In this hypothesis framework, the ability for accurately assessing early in pregnancy (several months prior to the manifestation of any clinical symptoms) which pregnant women are at increased (or high) risk of developing preeclampsia, and which women are a decreased (or low) risk of developing preeclampsia, will hinge on this assumption of preeclampsia being a multi-disease. However, currently there are no means to unambiguously delineate the different sub-types of preeclampsia or/and to delineate specific sub-populations at risk for preeclampsia (or any of its subtypes).

Interestingly, recent research suggests that prophylactic treatment to prevent preeclampsia might associate with a specific form of Preeclampsia or a specific pregnancy sub-population, thus validating the concept that different subtypes of preeclampsia exist. In this context, Aspirin has been recently confirmed to prevent a form of preeclampsia which is characterised by placental compromise and which is associated with an early manifestation of preeclampsia, i.e., preterm preeclampsia [10]. In the case of identification of pregnancies at increased risk of preterm preeclampsia, a multivariable prognostic model has been proposed which stratifies up to 10% of the pregnancy population into a high risk group at the end of 1^{st} trimester/beginning of the 2^{nd} trimester, and then to prophylactically treat this at-risk population with aspirin with the aim to prevent some of the early onset and preterm preeclampsia cases [11]. In this case, the risk algorithm combines maternal history (fi, history of preeclampsia) and characteristics (fi, race, Body Mass Index (BMI)), biophysical findings (fi, blood pressure readings and Ultrasound measurements indicative for compromised placental perfusion) and biochemical factors (pregnancy-associated plasma protein A (gene: PAPPA) and Placental Growth Factor (Gene: PIGF)). Whereas the referenced test has some utility, its worldwide adoption in clinical practice has been hampered by the fact that it's performance is highly dependent on the ultrasound measurements, which requires a highly skilled and specifically certified sonographer to perform a specialist ultrasound measurement using advanced ultrasound technology [See also EP2245180B1]. In addition, this referenced prognostic model also derives a significant fraction of its performance from the availability of medical history and prior pregnancy information; the latter compromising its utility to accurately predict risk in first time pregnant women, a sub-population at increased risk compared to the multiparous women.

It can be easily appreciated by the reader that prognostic models for the risk of (preterm) preeclampsia which do not rely on uterine artery pulsatility index (PI) or pregnancy history information, but solely use easily accessible biometric variables like blood pressure, bmi, age etc together with a set of biochemical measurements as present in a biospecimen obtained from a pregnant woman and which can determined within clinical laboratories worldwide, will facilitate the world-wide deployment of such prognostic tests. Moreover, the Applicants realise that the health impact of the prognostic combinations of variables for (preterm/term) preeclampsia, as disclosed in this application, will impact on the (future) health of pregnant women and their children, when these prognostic tests are combined with a suitable prophylactic treatment regimen, like the aforementioned aspirin intervention for preterm preeclampsia.

The availability of a test to identify those at risk for (preterm/term) preeclampsia within the pregnancy population will also allow for testing the effectiveness of other interventions to prevent preeclampsia. Next to enabling aspirin, the treatment effectiveness of metformin; Low Molecular Weight Heparin [12], glycemic index lowering probiotics [13]; citrulline [14] or antioxidants, inclusive but not limited to, antioxidant vitamins (e.g., ascorbic acid, alpha-tocopherol, beta-carotene) [15], inorganic antioxidants (e.g., selenium), and a plant-derived polyphenols, and/or antioxidants to mitochondria [25]inclusive but not limited to, Mito VitE and ergothioneine [16,17]; statins, inclusive but not limited to, Pravastin [18]; anti-hypertensive treatments (using inter alia beta-blockers; vasodilators, inclusive but not limited to H2S [19] or NO-donors like Sildenafil or others [20]; DOPA decarboxylase inhibitors) or anti-inflammatory therapeutics, inclusive but not limited to Digibind [21]; or actors against oxidative stress damage, inclusive but not limited to, (a1-microglobulin) [22] can also be considered. In addition, one can easily envision preferred therapeutic combinations like, but not limited to, aspirin and and/or antioxidants to mitochondria.

It is an object of the invention to overcome at least one of the above-referenced problems.

### SUMMARY OF THE INVENTION

The present invention addresses the need for a predictive test for preeclampsia (PE) that can be employed with a pregnant woman at an early stage of pregnancy prior to the appearance of clinical symptoms of PE to stratify the pregnant woman according to pregnancy outcome (PE, pre-term PE or term PE), and optionally according to risk category (elevated risk or reduced risk). The methods employ patient-specific variables generally selected from PE-specific metabolites and optionally proteins and clinical risk factors such as blood pressure, weight, smoking status, number of pregnancies, etc. which are employed singly and in combination to classify the risk of a selected pregnancy outcome and optionally risk category (Table 1). In a related aspect, the inventors have also identified rule-in biomarkers that may be employed to generate rule-in prognostic signatures (a signature that is indicative of increased risk of preeclampsia) and/or rule-out biomarkers that are employed to generate rule-out signatures (a signature indicative of reduced risk of preeclampsia). Use of at least one rule-in signature or at least one rule-out signature, optionally both and optionally in sequence, and optionally a series of rule-in or rule-out prognostic signatures, allows the patient to be stratified into an increased risk category or a reduced risk category with greater accuracy that known methods.

The prognostic signature (rule-in or rule-out) may be univariable (i.e. be composed of a single variable such as a protein or a metabolite) or multivariable (i.e. be composed of one or more protein(s) and/or one or more metabolite(s)). When the prognostic signature is univariable, detection of the presence of the prognostic signature in the subject (in the case of a clinical risk factor variable) or in the biological sample (in the case of a protein or metabolite variable) generally involves comparing the level with a defined threshold level for the variable and determining whether the test level is above or below the threshold level. The defined threshold level is predetermined (for example based on a study population and a predefined rule-in (or rule-out) test requirement) and may therefore vary from test to test depending on the type of preeclampsia and the desired stringency of the predictive test. When the prognostic signature is a multivariable signature comprising two or more variables (for example a metabolite and a protein, or two metabolites, or a clinical risk factor and a metabolite), determining the presence of the prognostic signature in the subject generally involves inputting the levels into a statistical model configured to provide an output in the form of a score, and comparing the score with a defined threshold score (or a range of reference scores) for the multivariable prognostic signature and determining whether the test score is above or below the threshold score. The defined threshold score may be predetermined based on a study population and a predefined rule-in or rule-out test performance requirement and may therefore vary from test to test depending on the type of preeclampsia and the desired stringency of the predictive test.

In a first aspect, the invention provides a system, and a computer implemented method, of early prediction of risk of a pregnancy outcome in a pregnant woman (i.e. at 8-22 weeks of pregnancy)

The method generally comprises the steps of:

inputting into a computational model,
values for a panel of preeclampsia specific biomarkers comprising at least one metabolite, and optionally at least one protein or clinical risk factor, generally selected from Table 1, in which the values are obtained from the pregnant woman early in pregnancy,
   in which the computational model is configured to:
select a subset of inputted values comprising a value for at least one metabolite and optionally at least one protein or clinical risk factor value, based on a pregnancy outcome typically selected from pre-term preeclampsia, term preeclampsia and all preeclampsia;
calculate a predicted risk of the selected pregnancy outcome based on the subset of inputted values; and
output the predicted risk of the pregnancy outcome for the pregnant woman.

TABLE 1

| | | | All PE | | | | Preterm PE | | | | |
|---|---|---|---|---|---|---|---|---|---|---|---|
| | | Patent | AUC | | Rule-in | Rule-out | AUC | | Rule-in | Rule-out | segregation |
| type | Variable | codes | Univ. | Multiv. | Multiv. | Multiv. | Univ. | Multiv. | Multiv. | Multiv. | Multiv. |
| clinical | blood pressure measurement | bp | 2 | 1 | 1 | 1 | | | | | |
| | bmi related | WRV | 2 | 1 | | | 2 | | | | |
| | fh_pet | fh_pet | | | 1 | | | | | | |
| Metabolite | Dilinoleoyl-glycerol (DLG) | DLG | 2 | 1 | 1 | | 2 | 1 | 1 | 1 | 2 |
| | 1-heptadecanoyl-2-hydroxy-sn- | 1-HD | 2 | 1 | | 1 | | 1 | | 1 | 1 |
| | glycero-3-phosphocholine (1-HD) | | | | | | | | | | |
| | 25-Hydroxyvitamin D3 | HVD3 | | 1 | 1 | 1 | | | | | |
| | Isoleucine (ILE) | L-ISO | | 1 | | | | | | | 1 |
| | Leucine (LEU) | L-LEU | | 1 | | | | | | | 1 |
| | NG-Monomethyl-L-arginine | NGM | | | | | 2 | 1 | | 1 | |
| | Stearoylcarnitine | SC | | | | | | 1 | 1 | 1 | |
| | Ergothioneine (ERG) | L-ERG | | | | | | | 1 | 1 | 1 |
| | 2-Hydroxybutanoic acid | 2-HBA | | | | | 2 | 1 | | | |
| | Decanoylcarnitine | DC | | | | | | | | | |
| | Etiocholanolone glucuronide | ECG | | | | | | 1 | 1 | | |
| | 20-Carboxy-leukotriene B4 | 20-CL | | | | | | 1 | 1 | | |
| | Citrulline | CR | | | 1 | | | | | 1 | |
| | Choline | CL | | | | | 2 | | | | |
| | Eicosapentaenoic acid | EPA | | 1 | | | | | | | |
| | Homo-L-arginine | H-L-ARG | | | | | | | | | |
| | methionine | L-MET | | | 1 | | | | | | |
| | Asymmetric dimethylarginine | ADMA | | | 1 | | | | | | |
| | Taurine | TR | | | | | | | | | |
| Protein | Placental Growth Factor (PIGF) | PIGF | 2 | 1 | 1 | | 2 | 1 | 1 | | 2 |
| | soluble-Endoglin (sENG) | s-ENG | | 1 | | 1 | 2 | 1 | 1 | 1 | 1 |
| | | | | | | | Term PE | | | | |
| | | | | | | | AUC | | Rule-in | Rule-out | |
| | | | | | | type | Univ. | Multiv. | Multiv. | Multiv. | sum |
| | | | | | | clinical | 2 | 1 | 1 | 1 | 10 |
| | | | | | | | 2 | | | | 7 |
| | | | | | | | | | | | 1 |
| | | | | | | | | | | | 11 |
| | | | | | | Metabolite | 2 | 1 | 1 | 1 | 12 |
| | | | | | | | | 1 | 1 | 1 | 6 |
| | | | | | | | 2 | 1 | 1 | | 6 |
| | | | | | | | | 1 | 1 | | 4 |
| | | | | | | | | | | | 4 |
| | | | | | | | | | | | 3 |
| | | | | | | | | | | | 3 |
| | | | | | | | | | | | 3 |
| | | | | | | | 2 | | | | 2 |
| | | | | | | | | | | | 2 |
| | | | | | | | | | | | 2 |
| | | | | | | | | | 1 | | 3 |
| | | | | | | | | | | | 2 |
| | | | | | | | | | | | 1 |
| | | | | | | | | 1 | | | 1 |
| | | | | | | | | | | | 1 |
| | | | | | | | | | | | 1 |
| | | | | | | | | | 1 | | 1 |
| | | | | | | Protein | | | | | 10 |
| | | | | | | | | | | | 8 |

In one embodiment, the pregnancy outcome is selected from pre-term preeclampsia and term preeclampsia.

In one embodiment, the computational model is configured to:
select a second subset of the inputted values comprising a value for at least one metabolite and optionally at least one protein or clinical risk factor value, based on a second pregnancy outcome selected from pre-term preeclampsia, term preeclampsia and all preeclampsia;
calculate a predicted risk of the second pregnancy outcome based on the second subset of inputted values; and
output the predicted risk of the second pregnancy outcome for the pregnant woman.

In one embodiment, the method includes a step of inputting into the computational model a chosen pregnancy outcome, in which case the computational model is configured to select a subset of inputted values based on the inputted pregnancy outcome. In embodiments, where the computational model is configured to select two subsets of inputted values corresponding to different pregnancy outcomes, the method may include a step of inputting two different selected outcomes into the computational model (i.e. term preeclampsia and pre-term preeclampsia). In one embodiment, if the predicted risk calculated based on the first subset of inputted values is inconclusive (for example, neither elevated nor reduced risk of pre-term preeclampsia), the computational model may be configured to select a second subset of inputted values based on a second pregnancy outcome, and calculate a predicted risk of the second pregnancy outcome (for example, detect elevated or reduced risk of term preeclampsia).

For metabolite and protein values, the values are abundance values obtained from a biological sample such as blood obtained from the pregnant woman early in pregnancy.

In one embodiment, the panel of preeclampsia specific biomarkers comprises at least 2, 3, 4, 5, 6, 7, 8, 9, 10, 11 or substantially all of the biomarkers of Table 1.

In one embodiment, the panel of preeclampsia specific biomarkers comprises PIGF and DLG In one embodiment, the panel of preeclampsia specific biomarkers comprises PIGF and DLG and one or more metabolite biomarkers (for example 1, 2, 3, 4, 5, or 6) selected from 1-HD, L-ISO, NGM, 2HBA, DC, and CL. In one embodiment, the panel of preeclampsia specific biomarkers comprises PIGF, DLG and 1-HD, and optionally one or more metabolite biomarkers (for example 1, 2, 3, 4, 5, or 6) selected from 1-HD, L-ISO, NGM, 2HBA, DC, and CL. In one embodiment, the panel of preeclampsia specific biomarkers comprises substantially all of PIGF, DLG, 1-HD, L-ISO, NGM, 2HBA, DC, and CL.

In one embodiment, the or each selected subset of values consist of a value for single metabolite biomarker, and in which the calculation step comprises comparing the abundance value of the single metabolite biomarker with a reference abundance value for the same metabolite biomarker.

In one embodiment, the single metabolite biomarker is selected from DLG, 1-HD, L-ISO, NGM, 2HBA, DC, and CL.

In one embodiment, the selected pregnancy outcome is pre-term PE and the single biomarker is selected from DLG, NGM, 2HBA, and CL.

In one embodiment, the selected pregnancy outcome is term PE and the single biomarker is selected from 1-HD, L-ISO and DC.

In one embodiment, the selected pregnancy outcome is all PE and the single biomarker is selected DLG and 1-HD.

In one embodiment, the or each selected subset of values comprises values for a plurality of biomarkers selected from Table 1. Typically, the calculation step comprises the steps of:
inputting the or each selected subset of values into a risk score calculation specific to the selected pregnancy outcome to calculate a risk score of the pregnancy outcome; and
compare the calculated risk score with at least one reference risk score to provide a predicted risk of the pregnancy outcome for the pregnant woman.

In one embodiment, the selected pregnancy outcome is pre-term PE and in which the selected subset of values comprises values for a plurality of biomarkers selected from DLG, 1-HD, L-ISO, L-LEU, NGM, SC, L-ERG, 2-HBA, ECG, 20-CL, CR, PIGF and s-ENG. In one embodiment, the selected subset of values comprises values for a plurality of biomarkers selected from DLG, 1-HD, NGM, SC, 2-HBA, ECG, 20-CL, PIGF and s-ENG.

In one embodiment, the selected pregnancy outcome is term PE and in which the selected subset of values comprises values for a plurality of biomarkers selected from bp, 1-HD, HVD3, L-ISO, L-LEU, CR, H-L-ARG and TR. In one embodiment, the selected subset of values comprises values for a plurality of biomarkers selected from bp, 1-HD, HVD3, L-ISO, L-LEU, and H-L-ARG.

In one embodiment, the selected pregnancy outcome is all PE and in which the selected subset of values comprises values for a plurality of biomarkers selected from bp, WRV, fh_pet, DLG, 1-HD, HVD3, L-ISO, L-LEU, EPA, L-MET, ADMA, PIGF and s-ENG. In one embodiment, the selected subset of values comprises values for a plurality of biomarkers selected from bp, WRV, 1-HD, HVD3, L-ISO, L-LEU, CR, EPA, PIGF and s-ENG.

In one embodiment, the method includes a step of inputting into a computational model values for a panel of preeclampsia specific biomarkers comprising one or more rule-in and/or rule-out biomarkers as described herein, and typically selected from Table 1.

In one embodiment, the or each subset of inputted values selected by the computational model comprises at least one rule-in biomarker (i.e. one or more rule-in biomarkers of Table 1), wherein the computational model is configured to detect elevated risk of the selected pregnancy outcome based on the subset of inputted values.

In one embodiment, the or each subset of inputted values selected by the computational model comprises at least one rule-out biomarker (i.e. one or more rule-out biomarkers of Table 1), wherein the computational model is configured to detect reduced risk of the selected pregnancy outcome based on the subset of inputted values.

In one embodiment, the method includes the additional step of inputting a risk category selected from elevated risk and reduced risk into the computational model, and in which the or each subset of inputted values selected by the computational model comprises (a) a rule-in subset of inputted values comprising a value for one or more rule-in biomarkers and/or (b) a rule-out subset of inputted values comprising a value for one or more rule-out biomarkers, based on the selected pregnancy outcome and selected rick category.

In one embodiment, the risk category inputted into the computational model is elevated risk, and in which the computational model is configured to:
select a rule-out subset of inputted values comprising a value for one or more rule-out biomarkers, based on the selected pregnancy outcome;
determine if there is a reduced risk of the selected pregnancy outcome based on the rule-out subset of inputted values;
where a reduced risk of the selected pregnancy outcome is not determined, select a rule-in subset of inputted values comprising a value for one or more rule-in biomarkers, based on the selected pregnancy outcome;
determine if there is an elevated risk of the selected pregnancy outcome based on the rule-in subset of inputted values;
output the predicted risk of the pregnancy outcome for the pregnant woman.

In one embodiment, the risk category inputted into the computational model is reduced risk, and in which the computational model is configured to:
select a rule-in subset of inputted values comprising a value for one or more rule-in biomarkers, based on the selected pregnancy outcome;
calculating the predicted risk by determining if there is an elevated risk of the selected pregnancy outcome based on the rule-in subset of inputted values;
where an elevated risk of the selected pregnancy outcome is not determined, select a rule-out subset of inputted values comprising a value for one or more rule-out biomarkers, based on the selected pregnancy outcome;
calculating the predicted risk by determining if there is a reduced risk of the selected pregnancy outcome based on the rule-out subset of inputted values; and
output the predicted risk of the pregnancy outcome for the pregnant woman.

In one embodiment, the one or more rule-in biomarkers comprises DLG and in which the one or more rule-out biomarkers comprises PIGF.

In one embodiment, the selected pregnancy outcome is pre-term preeclampsia, and in which the one or more rule-in biomarkers is selected from DLG, SC, L-ERG, ECG, 20-CL, PIGF and s-ENG.

In one embodiment, the selected pregnancy outcome is term preeclampsia, and in which the one or more rule-in biomarkers is selected from bp, 1-HD, HVD3, L-ISO, L-LEU, CR and TR.

In one embodiment, the selected pregnancy outcome is all preeclampsia, and in which the one or more rule-in biomarkers is selected from bp, fh_pet, DLG, HVD3, CR, L-MET, ADMA and PIGF.

In one embodiment, the selected pregnancy outcome is pre-term preeclampsia, and in which the one or more rule-out biomarkers is selected from DLG, 1-HD, NGM, SC, L-ERG, CR and s-ENG.

In one embodiment, the selected pregnancy outcome is term preeclampsia, and in which the one or more rule-out biomarkers is selected from bp, 1-HD, and HVD3.

In one embodiment, the selected pregnancy outcome is all preeclampsia, and in which the one or more rule-out biomarkers is selected from bp, 1-HD, HVD3 and s-ENG.

In one embodiment, if an elevated risk of the selected pregnancy outcome is not determined based on the rule-in subset of inputted values, the computational model is configured to select a second rule-in or rule-out subset of inputted values, based on the selected pregnancy outcome.

In one embodiment, if a reduced risk of the selected pregnancy outcome is not determined based on the rule-out subset of inputted values, the computational model is configured to select a second rule-in or rule-out subset of inputted values, based on the selected pregnancy outcome.

In another aspect, the invention provides a method of predicting risk of pre-term preeclampsia in a pregnant woman comprising the steps of:

(a) determining a level of a panel of metabolite and protein variables including DLG and a protein selected from PIGF and s-ENG, in which the metabolite and protein levels are measured in a biological sample obtained from the pregnant woman early in pregnancy;
(b) providing a score based on the level of a first subset of the panel of variables comprising PIGF protein and comparing the score with a threshold score to detect the presence of a first rule-in prognostic signature; or
(c) providing a score based on the level of a second subset of the panel of variables comprising DLG or s-ENG and comparing the score with a threshold score to detect the presence of a first rule-out prognostic signature; and
(d) calculating predicted risk of pre-term preeclampsia based on the presence or absence of the rule-in and rule-out prognostic signatures.

In one embodiment, the method includes step (b) and step (c).

In one embodiment, when the first rule-in prognostic signature is detected and the first rule-out prognostic signature is not detected, the pregnant woman is determined to have an elevated risk of developing pre-term preeclampsia, or when the first rule-out prognostic signature is detected, and the first rule-in prognostic signature is not detected, the pregnant woman is determined to have a reduced risk of developing pre-term preeclampsia.

In one embodiment, the panel of variables includes DLG, PIGF, s-ENG, L-ERG, and 1-HD.

In one embodiment, the panel of variables additionally includes at least five of L-LEU, L-ISO, 2-HBA, ECG, SC, DC, CL and NGM.

In one embodiment, the first subset of variables comprises PIGF and the second subset of variables comprises DLG.

In one embodiment, the first subset of variables comprises PIGF and the second subset of variables comprises DLG and any two from s-ENG, L-ERG, L-LEU, L-ISO, or (L-ISO+L-LEU)

In one embodiment, the first subset of variables and/or the second subset of variables each comprise a plurality of variables including at least one metabolite and at least one protein.

In one embodiment, the first subset of variables comprises DLG and PIGF, or DLG and s-ENG.

In one embodiment, the first subset of variables comprises:

PIGF, s-ENG, DLG and 2-HBA;

### PIGF, and DLG;

DLG and s-ENG; or
PIGF, s-ENG, DLG and any one or two from ECG, L-ERG, SC, 20-CL.

In one embodiment, the second subset of variables comprises DLG and s-ENG, or s-ENG and 1-HD.

In one embodiment, the second subset of variables comprises:

DLG and s-ENG;
s-ENG and 1-HD;
s-ENG, DLG, and 1-HD; or
s-ENG, DLG, and any one, two or three from 1-HD, CL, L-ERG, SC, NGM.

In one embodiment, when the presence of the first rule-in prognostic signature is not detected, and the presence of the first rule-out prognostic signature is not detected, the method includes an additional step of providing a score based on the level of a third or fourth subset of the panel of variables and comparing the score with a threshold score to detect the presence of a second rule-in or second rule-out prognostic signature, and calculating predicted risk of pre-term preeclampsia based on the presence or absence of the second rule-in and rule-out prognostic signatures.

In one embodiment, when the presence of the second rule-out prognostic signature is detected, the pregnant woman is determined to have a reduced risk of developing pre-term preeclampsia; or when the presence of the second rule-in prognostic signature is detected, the pregnant woman is determined to have an elevated risk of developing pre-term preeclampsia

In one embodiment, when the presence of the second rule-out prognostic signature is not detected, the method includes an additional step of providing a score based on the level of a fifth or sixth subset of the variables and comparing the score with a threshold score to detect the presence of a third rule-in or third rule-out prognostic signature.

In one embodiment, when the presence of the third rule-out prognostic signature is detected, the pregnant woman is determined to have a reduced risk of developing pre-term preeclampsia, or wherein when the presence of the third rule-out prognostic signature is not detected, the pregnant woman is determined to have an increased risk of developing pre-term preeclampsia.

In another aspect, the invention provides a method of predicting risk of term preeclampsia in a pregnant woman comprising the steps of:

(a) determining a level of a panel of metabolite variables including HVD3 or 1-HD, in which the metabolite levels are measured in a biological sample obtained from the pregnant woman early in pregnancy;
(b) providing a score based on (i) the level of a first subset of the panel of variables comprising HVD3 or 1-HD and (ii) a blood pressure measurement obtained from the pregnant woman early in pregnancy, and comparing the score with a threshold score to detect the presence of a first rule-in prognostic signature; or
(c) providing a score based on (i) the level of a second subset of the panel of variables comprising HVD3 or 1-HD and (ii) a blood pressure measurement obtained from the pregnant woman early in pregnancy, and comparing the score with a threshold score to detect the presence of a first rule-out prognostic signature; and
(d) calculating predicted risk of term preeclampsia based on the presence or absence of the rule-in and rule-out prognostic signatures.

In one embodiment, the method includes step (b) and step (c).

In one embodiment, when the first rule-in prognostic signature is detected and the first rule-out prognostic signature is not detected, the pregnant woman is determined to have an elevated risk of developing term preeclampsia, or

when the first rule-out prognostic signature is detected, and the first rule-in prognostic signature is not detected, the pregnant woman is determined to have a reduced risk of developing term preeclampsia.

In one embodiment, the panel of metabolite variables includes 1-HD and HVD3.

In one embodiment, the panel of variables additionally includes at least one or more of TR, L-LEU, L-ISO, CR, DHA, NGM and BV.

In one embodiment, the first subset of variables and/or the second subset of variables each comprise a plurality of metabolite variables.

In one embodiment, the first subset of variables comprises:

### HVD3 and (TR, 1-HD or L-ISO or L-LEU);

### HVD3 and (L-ISO or L-LEU) and (TR or CR);

### 1-HD and (TR, L-ISO or DHA);

### 1-HD, NGM and H-L-ARG or

### HVD3, 1-HD and (NGM, TR or BV).

In one embodiment, the second subset of variables comprises

### HVD3 and (TR, 1-HD, WRV or H-L-ARG);

### 1-HD and (CR or 20-CL); or

### HVD3, H-L-ARG and (CR or TR).

In one embodiment, when the presence of the first rule-in prognostic signature is not detected, and the presence of the first rule-out prognostic signature is not detected, the method includes an additional step of providing a score based on the level of a third or fourth subset of the panel of variables and optionally a clinical risk factor measurement, and comparing the score with a threshold score to detect the presence of a second rule-in or second rule-out prognostic signature, and calculating predicted risk of term preeclampsia based on the presence or absence of the second rule-in and rule-out prognostic signatures.

In one embodiment, when the presence of the second rule-out prognostic signature is detected, the pregnant woman is determined to have a reduced risk of developing term preeclampsia; or when the presence of the second rule-in prognostic signature is detected, the pregnant woman is determined to have an elevated risk of developing term preeclampsia

In one embodiment, when the presence of the second rule-out prognostic signature is not detected, the method includes an additional step of providing a score based on the level of a fifth or sixth subset of the variables and comparing the score with a threshold score to detect the presence of a third rule-in or third rule-out prognostic signature.

In one embodiment, when the presence of the third rule-out prognostic signature is detected, the pregnant woman is determined to have a reduced risk of developing term preeclampsia, or wherein when the presence of the third rule-out prognostic signature is not detected, the pregnant woman is determined to have an increased risk of developing term preeclampsia.

The methods of the invention may be fully or partly implemented by a computer.

The invention also provides a computer program comprising programme instructions for causing a computer to perform any method of the invention. The computer program may be embodied on a record medium, a carrier signal, or a read-only memory.

In another aspect, the invention provides a method of predicting the risk of preeclampsia in a pregnant woman comprising the steps of:

(a) determining a level of a panel of variables selected from one or more metabolites, proteins and clinical risk factors, in which the metabolite and protein levels are measured in a blood sample obtained from the pregnant woman;
(b) providing a score based on the level of one or more of the variables and comparing the score with a threshold score to detect the presence of a first rule-in or first rule-out prognostic signature;
(c1) when the presence of the first rule-in prognostic signature is not detected, comparing the level(s) of another variable or variables with a threshold established to detect the presence of a first rule-out prognostic signature; or
(c2) when the presence of a first rule-out signature is not detected, comparing the level(s) of another variable or variables with a threshold established to detect the presence of a first rule-in prognostic signature; and
(d) correlating the presence or absence of the rule-in and rule-out prognostic signatures with risk of preeclampsia.

When the rule-in or rule-out signature is a univariable signature, step (b) generally comprises comparing the determined level of the variable with a defined threshold level for the variable and determining whether the test level is above or below the threshold level.

When the rule-in or rule-out prognostic signature is a multivariable signature, step (b) generally comprises inputting the determined levels of the variables into a statistical model configured to provide an output in the form of a score and comparing the score with a threshold score for the multivariable prognostic signature and determining whether the test score is above or below the threshold score.

In one embodiment, when the presence of the first rule-in prognostic signature is detected, the pregnant woman is determined to have an elevated risk of developing preeclampsia or when the presence of the first rule-out prognostic signature is detected, the pregnant woman is determined to have a reduced risk of developing preeclampsia

In one embodiment, when the presence of the first rule-in prognostic signature is not detected, and the presence of the first rule-out prognostic signature is detected, the pregnant woman is determined to have a reduced risk of developing preeclampsia.

In one embodiment, when the presence of the first rule-in prognostic signature is not detected, and the presence of the first rule-out prognostic signature is not detected, the method includes an additional step of providing a score based on the level of one or more of the variables and comparing the score with a threshold score to detect the presence of a second rule-out prognostic signature or a second rule-in prognostic signature. The additional step generally employs a different variable, or different combination of variables, compared with the first and second steps.

In one embodiment, when the presence of the second rule-out prognostic signature mentioned above is detected, the pregnant woman is determined to have a reduced risk of developing preeclampsia.

In one embodiment, when the presence of the second rule-out prognostic signature mentioned above is not detected, the method includes an additional step of providing a score based on the level of one or more of the variables and comparing the score with a threshold score to detect the presence of a third rule-out prognostic signature. The additional step generally employs a different variable, or different combination of variables, compared with the previous comparison steps.

In one embodiment, when the presence of the third rule-out prognostic signature is detected, the pregnant woman is determined to have a reduced risk of developing preeclampsia, or wherein when the presence of the third rule-out prognostic signature is not detected, the pregnant woman is determined to have an increased risk of developing preeclampsia.

In one embodiment, when the presence of the first rule-out prognostic signature is not detected, and the presence of the first rule-in prognostic signature is detected, the pregnant woman is determined to have an increased risk of developing preeclampsia.

In one embodiment, when the presence of the second rule-in prognostic signature is detected, the pregnant woman is determined to have an increased risk of developing preeclampsia.

In one embodiment, when the presence of the first rule-out prognostic signature is not detected, and the presence of the first rule-in prognostic signature is not detected, the method includes an additional step of providing a score based on the level of one or more of the variables and comparing the score with a threshold score to detect the presence of a second rule-in prognostic signature or a second rule-out prognostic signature. The additional step generally employs a different variable, or different combination of variables, compared with the previous comparison steps.

In one embodiment, when the presence of the second rule-in prognostic signature is detected, the pregnant woman is determined to have an increased risk of developing preeclampsia.

In one embodiment, when the presence of the first rule-out prognostic signature is not detected, and the presence of the first rule-in prognostic signature is not detected and the presence of the second rule-in prognostic signature is not detected, the method includes an additional step of providing a score based on the level of one or more of the variables and comparing the score with a threshold score to detect the presence of a third rule-in prognostic signature. The additional step generally employs a different variable, or different combination of variables, compared with the previous comparison steps.

In one embodiment, when the presence of the third rule-in prognostic signature is detected, the pregnant woman is determined to have an increased risk of developing preeclampsia, or wherein when the presence of the third rule-in prognostic signature is not detected, the pregnant woman is determined to have a reduced risk of developing preeclampsia.

In one embodiment, when the presence of the second rule-out prognostic signature is detected, the pregnant woman is determined to have a reduced risk of developing preeclampsia.

In one embodiment, the preeclampsia is pre-term preeclampsia.

In one embodiment, the first rule-in prognostic signature comprises PIGF.

In one embodiment, the first rule-out prognostic signature comprises-DLG.

In one embodiment, the second rule-out prognostic signature comprises L-ERG, s-ENG, L-LEU, L-ISO or (L-ISO and L-LEU).

In one embodiment, the first, second and third rule-out prognostic signature combinations are selected from the group comprising:

First—DLG, Second—L-ERG and Third—s-ENG;
First—DLG, Second—s-ENG and Third—1-HD;
First—DLG, Second—L-LEU and Third—s-ENG;
First—DLG, Second—s-ENG and Third—L-LEU;
First—DLG, Second—L-ISO and Third—s-ENG;
First—DLG, Second—(L-ISO+L-LEU) and Third—s-ENG;

### First—DLG, Second—L-ERG and Third—L-LEU;

### First—DLG, Second—L-ERG and Third—L-ISO; and

### First—DLG, Second—L-ERG and Third—(L-ISO+L-LEU).

In one embodiment, the rule-out prognostic signature is a multivariable signature comprising s-ENG and DLG.

In one embodiment, the rule-out prognostic signature is a multivariable signature comprising s-ENG and 1-HD

In one embodiment, the rule-out prognostic signature is a multivariable signature comprising s-ENG DLG, and 1-HD

In one embodiment, the rule-out prognostic signature is a multivariable signature comprising s-ENG DLG, and any one, two or three from 1-HD, CL, L-ERG, SC, NGM

In one embodiment, the rule-in prognostic signature is a multivariable signature comprising PIGF, s-ENG, DLG and 2-HBA.

In one embodiment, the first rule-in prognostic signature is a multivariable signature comprising PIGF, and DLG.

In one embodiment, the first rule-in prognostic signature is a multivariable signature comprising DLG, and s-ENG.

In one embodiment, the first rule-in prognostic signature is a multivariable signature comprising PIGF, s-ENG, DLG and any one or two from ECG, L-ERG, SC, 2-HBA.

In one embodiment, when the presence of the multivariable rule-out prognostic signature is not detected, and the presence of the multivariable rule-in prognostic signature is detected, the pregnant woman is determined to have an increased risk of developing preeclampsia.

In one embodiment, the preeclampsia is term preeclampsia.

In one embodiment, the first rule-in prognostic signature comprises BP.

In one embodiment, the first rule-in prognostic signature is a multivariable signature comprising a combination of variables selected from: BP and (HVD3 or 1-HD); BP, HVD3 and (TR, 1-HD or L-ISO or L-LEU); BP, HVD3 and (L-ISO or L-LEU) and (TR or CR); BP, 1-HD and (TR, L-ISO or DHA); and BP, HVD3, 1-HD and (NGM, TR or BV)

In one embodiment, the first rule-out prognostic signature comprises BP.

In one embodiment, the first rule-out prognostic signature is a multivariable signature comprising a combination of variables selected from the combinations: BP and (HVD3 or 1-HD); BP, HVD3 and (TR, 1-HD or L-ISO); BP, 1-HD and (TR, L-ISO or DHA); and BP, HVD3, 1-HD and (NGM, TR or BV).

In one embodiment, the rule-out prognostic signature is a multivariable signature comprising BP and 1-HD, and/or the rule-in prognostic signature is a multivariable signature comprising BP and 1-HD and NGM.

In one embodiment, when the presence of the multivariable rule-out prognostic signature is not detected, and the presence of the multivariable rule-in prognostic signature is detected, the pregnant woman is determined to have an increased risk of developing preeclampsia.

In one embodiment, the preeclampsia is term preeclampsia and pre-term preeclampsia (all preeclampsia).

In one embodiment, the first rule-in prognostic signature comprises BP.

In one embodiment, the first rule-in prognostic signature is a multivariable signature comprising BP and one or more variables selected from the group comprising: PIGF, 1-HD, HVD3, DLG, S-1-P, 2-HBA, CR, ADMA, L-ERG, s-ENG, fh_pet, L-LEU, L-ISO, r_glucose, H-L-ARG and gest.

In one embodiment, the first rule-in prognostic signature is a multivariable signature comprising a combination of variables selected from the combinations: BP and (PIGF or 1-HD or HVD3); BP, PIGF and (1-HD or DLG); BP, 1-HD and S-1-P; BP, HVD3 and 2-HBA; BP, HVD3, CR and ADMA; BP, HVD3, DLG, 1-HD and L-ERG; BP, DLG and s-ENG; BP, DLG, PIGF and (fh_pet or L-ERG); BP, DLG, s-ENG and L-ERG; BP, HVD3, 1-HD and L-LEU; (PLGF or BP), 1-HD, s-ENG and L-ISO; BP, HVD3, 2-HBA and (r_glucose or H-L-ARG; and BP, HVD3, fh_pet and gest.

In one embodiment, the first rule-out prognostic signature comprises BP.

In one embodiment, the first rule-out prognostic signature is a multivariable signature comprising BP and one or more variables selected from the group comprising: PIGF, 1-HD, HVD3, DLG, S-1-P, ADMA, s-ENG, fh_pet, L-ARG, NGM, GG, 20-CL, and WRV.

In one embodiment, the first rule-out prognostic signature is a multivariable signature comprising a combination of variables selected from the combinations: BP and 1-HD; BP and (1-HD or HVD3 or DLG); BP and 1-HD and (HVD3 or DLG or s-ENG; BP and 1-HD and (ADMA, GG or sFlt1); BP and HVD3 and WRV; BP and S-1-P and fh_pet; BP and (WRV, L-ARG, sFlt1, NGM, PIGF, GG or 20-CL); and BP and 1-HD and HVD3 and s-ENG.

In one embodiment, the rule-out prognostic signature is a multivariable signature comprising BP and s-ENG and 1-HD.

In one embodiment, the rule-in prognostic signature is a multivariable signature comprising BP and PIGF and DC.

In one embodiment, when the presence of the multivariable rule-out prognostic signature is not detected, and the presence of the multivariable rule-in prognostic signature is detected, the pregnant woman is determined to have an increased risk of developing preeclampsia.

In another aspect, the invention provides a method of predicting risk of preeclampsia in a pregnant woman comprising the steps of:

(i) determining a level of a panel of variables selected from one or more metabolites, proteins and clinical risk factors, in which the metabolite and protein levels are measured in a blood sample obtained from the pregnant woman;
(ii) using a statistical model to provide a score based on the level of one or more of the variables and comparing the score with a threshold score to detect the presence of a rule-in or rule-out prognostic signature (the comparison step); and
(iii) correlating the presence or absence of the rule-in and rule-out prognostic signatures with risk of preeclampsia.

In one embodiment, the comparison step comprises inputting the level of the variables into a statistical model configured to output a score for the combination of variables, and comparing the score with a threshold score to detect the presence of a first rule-in or first rule-out prognostic signature.

In one embodiment, the prognostic selection of the panel of variables includes at least one variable from at least two variables classes selected from metabolites, proteins and clinical risk factors. In one embodiment, the selection of the rule-in or rule-out panel of prognostic variables includes at least one, and preferably a plurality (i.e. 2, 3, 4 or 5) of metabolites. In one embodiment, the selection of the rule-in or rule-out panel of prognostic variables includes at least one metabolite and at least one protein.

In one embodiment, the preeclampsia is pre-term preeclampsia, and the comparison step (ii) is configured to detect the presence of a rule-in prognostic signature, wherein the rule-in prognostic signature comprises a prognostic variable combination selected from the group consisting of: PLGF+s-ENG;

### PLGF+s-ENG+(DLG or ECG or L-ERG or 20-CL); PLGF+s-ENG+DLG; PLGF+s-ENG+ECG PLGF+s-ENG+DLG+20-CL; PLGF+s-ENG+ECG+20-CL; and PLGF+s-ENG+DLG+(L-ERG or SC).

In one embodiment, the preeclampsia is pre-term preeclampsia, and the comparison step (ii) is configured to detect the presence of a rule-out prognostic signature, wherein the rule-out prognostic signature comprises a prognostic variable combination selected from the group consisting of: s-ENG+(DLG or 1-HD); s-ENG+DLG; s-ENG+DLG+one or two of (CL, 1-HD, L-ERG, SC and NGM); s-ENG+DLG+1-HD; and s-ENG+DLG+random glucose.

In one embodiment, the preeclampsia is term preeclampsia, and the comparison step (ii) is configured to detect the presence of a rule-in prognostic signature, wherein the rule-in prognostic signature comprises a prognostic variable combination selected from the group consisting of: BP and (HVD3 or 1-HD); BP and HVD3 and (TR, 1-HD or L-ISO); BP and 1-HD and (TR, L-ISO or DHA); and BP and HVD3 and 1-HD and (NGM, TR or BV).

In one embodiment, the preeclampsia is term preeclampsia, and the comparison step (ii) is configured to detect the presence of a rule-out prognostic signature, wherein the rule-out prognostic signature comprises a prognostic variable combination selected from the group consisting of: BP and (HVD3 or 1-HD); and BP and HVD3 and 1-HD

In one embodiment, the preeclampsia is term preeclampsia and pre-term preeclampsia (all preeclampsia), and the comparison step (ii) is configured to detect the presence of a rule-in prognostic signature, wherein the rule-in prognostic signature comprises a prognostic variable selection comprising BP and one or more variables selected from the group comprising: PIGF, 1-HD, HVD3, DLG, S-1-P, 2-HBA, CR, ADMA, L-ERG, s-ENG, fh_pet, L-LEU, L-ISO, r_glucose, H-L-ARG and gest.

In one embodiment, the preeclampsia is term preeclampsia and pre-term preeclampsia (all preeclampsia), and the comparison step (ii) is configured to detect the presence of a rule-in prognostic signature, wherein the rule-in prognostic signature comprises a prognostic variable combination selected from the group consisting of: BP and (PIGF or 1-HD or HVD3); BP, PIGF and (1-HD or DLG); BP, 1-HD and S-1-P; BP, HVD3 and 2-HBA; BP, HVD3, CR and ADMA; BP, HVD3, DLG, 1-HD and L-ERG; BP, DLG and s-ENG; BP, DLG, PIGF and (fh_pet or L-ERG); BP, DLG, s-ENG and L-ERG; BP, HVD3, 1-HD and L-LEU; (PLGF or BP), 1-HD, s-ENG and L-ISO; BP, HVD3, 2-HBA and (r_glucose or H-L-ARG; and BP, HVD3, fh_pet and gest.

In one embodiment, the preeclampsia is term preeclampsia and pre-term preeclampsia (all preeclampsia), and the comparison step (ii) is configured to detect the presence of a rule-out prognostic signature, wherein the rule-out prognostic signature comprises a prognostic variable selection comprising BP and one or more variables selected from the group comprising: PIGF, 1-HD, HVD3, DLG, S-1-P, ADMA, s-ENG, fh_pet, L-ARG, NGM, GG, 20-CL, and WRV.

In one embodiment, the preeclampsia is term preeclampsia and pre-term preeclampsia (all preeclampsia), and the comparison step (ii) is configured to detect the presence of a rule-out prognostic signature, wherein the rule-out prognostic signature comprises a prognostic variable combination selected from the group consisting of: BP and 1-HD; BP and (1-HD or HVD3 or DLG); BP and 1-HD and (HVD3 or DLG or s-ENG; BP and 1-HD and (ADMA, GG or sFlt1); BP and HVD3 and WRV; BP and S-1-P and fh_pet; BP and (WRV, L-ARG, sFlt1, NGM, PIGF, GG or 20-CL); and BP and 1-HD and HVD3 and s-ENG.

### General and Analytical

In one embodiment, the pre-term preeclampsia, term preeclampsia, or pre-term and term preeclampsia (all preeclampsia), is predicted at a rate of at least 50% with a false positive rate of at most 20%.

In one embodiment, the pre-term preeclampsia, term preeclampsia, or pre-term and term preeclampsia, is predicted at a rate of at least 60% with a false positive rate of at most 20%.

In one embodiment, the pre-term preeclampsia, term preeclampsia, or pre-term and term preeclampsia, is predicted at a rate of at least 60% with a false positive rate of at most 20%.

In one embodiment, the biological sample is obtained from the pregnant woman prior to the appearance of preeclampsia, for example at 8-22 or 11-18 weeks gestation.

In one embodiment, the method includes a step of profiling of metabolites in a biological sample from the pregnant woman. In one embodiment, the method includes a step of profiling of all, or substantially all, of the metabolites of Table 2 in a biological sample from the pregnant woman.

In one embodiment, the rule-in prognostic signature or rule-out prognostic signature is determined by detecting a level of one or more variables, comparing the levels with a threshold level for the or each variable, and determining whether the subject exhibits the rule-in or rule-out prognostic signature based on the comparison.

In one embodiment, the comparison step comprises inputting the level of the variables into a statistical model configured to output a score for the combination of variables, and the score is compared with a threshold level.

In one embodiment, the at least one of the assaying steps comprises quantitative determination of a metabolite in the biological sample by means of mass spectrometry, more preferably liquid chromatography mass spectrometry (LC-MS).

In one embodiment, the mass spectroscopy comprises ionization of metabolites, preferably electrospray ionization, and electrospray-derived ionisation methods. Other LC-MS compatible methods of ionization may also be employed, e.g., continuous flow fast atom bombardment ionization, atmospheric pressure chemical ionization, atmospheric pressure photoionization.

When the methods of the invention are used in such a way that the LC-eluent is fractionated, deposited in discrete droplets on a surface, or traced on a surface, to preserve the spatial resolution as achieved by the chromatography for later analysis, the tandem mass spectrometry can be performed using other ionization techniques also. Among them, for instance, electron ionization, chemical ionization, field desorption ionisation, matrix-assisted laser desorption ionization, surface enhanced laser desorption ionization.

In one embodiment, the mass spectroscopy is carried out under both positive and negative electrospray ionization.

In one embodiment, the mass spectrometry employs selective ion monitoring.

In one embodiment, the mass spectrometry is tandem mass spectrometry (MS/MS).

In one embodiment, the tandem mass spectrometry comprises a step of fragmenting the ionised metabolites.

In one embodiment, the tandem mass spectrometry employs multiple reaction monitoring.

In one embodiment, the method of the invention includes a mass spectrometric analysis comprising one or more of the steps of:

subjecting the biological sample to ionization under conditions suitable to produce positively or negatively charged metabolite ions, or metabolite-adduct ions, derived from the metabolite. These ions are so-called “precursor ions”;
Using a first mass analyser (filter), to differentiate the precursor ions, based on their mass/charge ratio (m/z) from a background of other ions;
Fragmenting the pre-selected “precursor ions” by colliding them with a collision gas, like Nitrogen or Argon, into specific fragment ions, so-called “product ions”;
Using a second mass analyser (filter) to select one, typically 2, or more, specific product ions based on their mass, and determine the amount of one or more charged product ions in the mass spectrometer's ion detector;
Using the amount of the determined product ions to determine the amounts of the corresponding metabolite in the sample;
Whereby the mass spectrometer is configured to ionise multiple different metabolites; creating multiple different precursor ions; select any of the multiple different precursor ions using a first mass analyser; fragment any of the multiple precursor ions into product ions from the any of the multiple precursor ions; select one, typically 2, or more, specific product ions as obtained from the any of the multiple precursor ions; determine the amount of the one or more charged product ions as obtained from the any of the multiple precursor ions in the mass spectrometer's ion detector; using the amount of the determined product ions from the any of the multiple precursor ions to determine the amounts of the corresponding multiple different metabolites in the sample

In one embodiment, the method includes a step of pre-treating the biological sample with a metabolite extraction solvent to provide a pre-treated sample.

In one embodiment, the extraction solvent comprising methanol, isopropanol and an acetate buffer.

In one embodiment, the extraction solvent comprising methanol, isopropanol and an acetate buffer in a ratio of about 10:9:1 (v/v/v).

In one embodiment, the extraction solvent comprises 0.01 to 0.1% BHT (m/v).

In one embodiment, the mixture of biological sample and extraction solvent is incubated at a temperature of less than 5° C. for a period of time to assist protein precipitation, prior to separation of precipitated protein.

In one embodiment, the biological sample is a liquid sample and is collected and stored on an absorptive sampling device, preferably a volume controlling sampling device.

In one embodiment, the method includes the steps of:

separating a first aliquot of the sample by a first form of liquid chromatography, for example reverse phase liquid chromatography (RPLC), to provide a first eluent containing resolved metabolites of a first class (for example hydrophobic metabolites); and
separating a second aliquot of the sample by a second form of liquid chromatography, for example HILIC, to provide a second eluent containing resolved metabolites of a second class, for example hydrophilic metabolites; and
optionally, assaying the first and second eluents using targeted electrospray tandem mass spectroscopy operated in multiple reaction monitoring mode.

In one embodiment, the RPLC employs a varying mixture of a first mobile phase A comprising water, methanol and an acetate buffer and a second mobile phase B comprising methanol, acetonitrile, isopropanol and an acetate buffer.

In one embodiment, the RPLC mobile phases are mixed according to a varying volumetric gradient of about 1-20% (preferably about 10%) to 80-100% (preferably about 100%) mobile phase B over a suitable period, for example 1-20 minutes or about 8-12 minutes, preferably about 10 minutes. The varying volumetric gradient may be a linear gradient, or a stepwise gradient.

In one embodiment, the HILIC employs a varying mixture of a first mobile A phase comprising ammonium formate and a second mobile phase B comprising acetonitrile.

In one embodiment, the HILIC mobile phases are mixed according to a varying volumetric gradient of about 80-100% (preferably about 10%) to 40-60% (preferably about 50%) mobile phase B over a period of about 8-12 minutes, preferably about 10 minutes. The varying volumetric gradient may be a linear gradient, or a stepwise gradient.

In one embodiment, the biological sample comprises at least one stable isotope-labelled internal standard (SIL-IS) corresponding to a metabolite.

A list of mass spectrometry compatible buffers can be found at https://www.nestgrp.com/protocols/trng/buffer.shtml

In one embodiment, the biological sample comprises stable isotope-labelled internal standards (SIL-IS) corresponding to a plurality of metabolites.

In another aspect, the invention relates to a method of detecting or predicting risk of pre-term preeclampsia in a pregnant woman, the method comprising the steps of

(a) assaying a biological sample obtained from the pregnant woman to measure a level of a panel of variables including DLG;
(b) comparing the measured level of the panel of variables with a reference level for the or each variable in the panel; and
(c) detecting or predicting risk of pre-term preeclampsia based on comparison step (b).

In one embodiment, the panel of variables include more than one variable.

In one embodiment, the panel of variables includes one or more proteins, or one or more metabolites.

In one embodiment, the comparison step (b) comprises inputting the level of a combination of variables into a statistical model configured to provide an output score, and then comparing the output score with a reference score for the combination of variables.

In one embodiment, the biological sample is obtained from the pregnant woman prior to the appearance of any clinical symptoms of pre-term preeclampsia, for example at 11-18 weeks gestation.

The invention also relates to a method of treating a pregnant woman identified as having an elevated risk of developing pre-term PE, term-PE, or all-PE, the method comprising a step of applying a prophylactic therapy to the pregnant woman. In one embodiment, the prophylactic therapy is applied prior to the appearance of clinical symptoms of PE.

Thus, the invention also relates to a method of treating a pregnant woman predicted as being at risk of developing pre-term PE, term-PE, or all-PE according to a method of the invention, the method comprising a step of applying a prophylactic therapy to the pregnant woman.

In one embodiment, the prophylactic therapy is applied prior to the appearance of clinical symptoms of PE, and optionally continued during the pregnancy

In one embodiment, the prophylactic therapy comprises administration of agent selected from the group consisting of: aspirin; metformin; Low Molecular Weight Heparin; glycemic index lowering probiotics; citrulline or antioxidants; antioxidants to mitochondria; statins; anti-hypertensive treatment; anti-inflammatory therapeutics; and oxidative stress damage inhibitors.

In one embodiment, the preeclampsia is pre-term preeclampsia, and in which the prophylactic therapy comprises administration of agent of aspirin, metformin or aspirin with metformin.

There is also provided a computer program comprising program instructions for causing a computer program to carry out a method of the invention which may be embodied on a record medium, carrier signal or read-only memory. There is also provided a computer implemented system configured for carrying out a method of the invention. The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. CD ROM, or magnetic recording medium, e.g. a floppy disk or hard disk. The carrier may be an electrical or optical signal which may be transmitted via an electrical or an optical cable or by radio or other means.

There is also provided a computer implemented system configured for carrying out a method of the invention.

In one embodiment, there is provided a computer implemented system for predicting risk of preeclampsia in a pregnant woman, comprising:

(a) means for determining a level of a panel of variables selected from metabolites, proteins and clinical risk factors, in which the metabolite and protein levels are measured in a biological sample obtained from the pregnant woman;
(b) means for providing a score based on the level of one or more of the variables and comparing the score with a threshold score to detect the presence of a first rule-in or first rule-out prognostic signature;
(c1) means for comparing the level of another of the panel of variables with a threshold level for the variable to detect the presence of a first rule-out prognostic signature; or
(c2) means for comparing the level of another of the panel of variables with a threshold level for the variable to detect the presence of a first rule-in prognostic signature; and
(e) means for correlating the presence or absence of the rule-in and rule-out prognostic signatures with risk of preeclampsia.

In one embodiment, there is provided a computer implemented system for detecting or predicting risk of pre-term preeclampsia in a pregnant woman, comprising:

(a) means for assaying a biological sample obtained from the pregnant woman to determine a level of a panel of variables including DLG;
(b) means for comparing the measured level of the panel of variables with a reference level for the or each variable in the panel; and
(c) means for detecting or predicting risk of pre-term preeclampsia based on comparison step (b).

Other aspects and preferred embodiments of the invention are defined and described in the other claims set out below.

### BRIEF DESCRIPTION OF THE FIGURES

FIG. 1 Example 7A; Sequential application of a Rule-out classifier followed by a rule-in classifier to achieve a preset PPV cut-off prognostic performance for predicting “All PE. Panel A: the PPV=0.133 cut-off for a pretest prevalence of future PE of p=0.05 plotted in the ROC space. Panel B: Step 1; ROC curve corresponding a selected rule-out classifier (bp+s-ENG+1-HD) with the statistical model M1: 0.292700587596098 log 10[S-ENG (MoM)]+0.0103090246336299 [2nd_sbp]-0.335817558146904 log 10 [1-HD]; classification of the full test-population (P1) is done at a 10% FNR threshold. This corresponds to a rule-out threshold score of the statistical model M1 being less than (<) 0.66643052785405. This results in 38.3% of the true negatives (future non-cases) being classified at low risk, together with 10% of the future PE cases (false Negatives). These individuals are removed from the test population. Panel C: Step 2; ROC curve corresponding an exemplary rule-in classifier (bp+PIGF+DC) with the statistical model M2: −0.195394942337404 log 10 [PIGF (MoM)]+0.00590836118884227 [map_1^{st}]+0.143670336856774 log 10 [DC] and Model M2 rule-in threshold score of larger than (>) 0.581930006682247, as applied within the remainder of the test population (P2), as a rule-in classifier achieving the preset PPV performance for the prediction of “All PE”. Following the step 1 rule-out classification, the prevalence of future “All PE” is enriched to p=0.071, resulting in a change to the slope of the PPV line thus increasing the sensitivity (detection rate) for the preset PPV criterion.

FIG. 2 Example 7B; Sequential application of a Rule-out classifier followed by a rule-in classifier to achieve a preset PPV cut-off prognostic performance for predicting “Preterm PE”. Panel A: the PPV=0.071 cut-off for a pretest prevalence of future preterm PE of p=0.014, plotted in the ROC space. Panel B: Step 1; ROC curve corresponding a selected rule-out classifier (s-ENG+DLGDLG) with the statistical model M1: 0.22139876465602 log 10 [s-ENG]+0.0162829949120052 log 10 [DLG]; classification of the full test-population (P1) is done at a 10% FNR threshold. This corresponds to a rule-out threshold score of the statistical model M1 being less than (<) 0.710765699780132. This results in 43.7% of the true negatives (future non-cases) being classified at low risk, together with 10% of the future preterm PE cases (false Negatives); these individuals removed from the test population. Panel C: Step 2; ROC curve corresponding an exemplary rule-in classifier (PIGF+s-ENG+DLG+2-HBA) with the statistical model M2; 0.20043337818718 log 10 [s-ENG (MoM)]-0.212088369466248 log 10 [PIGF (MoM)]+0.112046727485729 log 10[2-HBA]+0.227265325783904 log 10 [DLG] and Model M2 rule-in threshold score of larger than (>) 0.668333882056883 as applied within the remainder of the test population (P2), as a rule-in classifier achieving the preset PPV performance for the prediction of “Preterm PE”. Following the step 1 rule-out classification, the prevalence of future “Preterm PE” is enriched to p=0.023, resulting in a change to the slope of the PPV line thus increasing the sensitivity (detection rate) for the preset PPV criterion.

FIG. 3 Example 7C; Sequential application of a Rule-out classifier followed by a rule-in classifier to achieve a preset PPV cut-off prognostic performance for predicting of “Term PE”. Panel A: the PPV=0.154 cut-off for a pretest prevalence of future preterm PE of p=0.037, plotted in the ROC space. Panel B: Step 1; ROC curve corresponding a selected rule-out classifier (bp+1-HD) with the statistical model M1: 0.0115467461789923 [map_1^{st}]-0.324977743714534 log 10 [1-HD]; classification of the full test-population (P1) is done at a 10% FNR threshold. This corresponds to a rule-out threshold score of the statistical model M1 being less than (<) 0.680257687736226. This results in 38.2% of the true negatives (future non-cases) being classified at low risk, together with 10% of the future Term PE cases (false Negatives); these individuals removed from the test population. Panel C: Step 2; ROC curve corresponding an exemplary rule-in classifier (bp+1-HD+NGM) with the statistical model M2; 0.0093936118486756 [2nd_sbp]+0.560572544580583 log 10 [NGM]−0.302082838614281 log 10 [1-HD] and Model M2 rule-in threshold score of larger than (>) 0.581599411310977. as applied within the remainder of the test population (P2), as a rule-in classifier achieving the preset PPV performance for predicting “Term PE”. Following the step 1 rule-out classification, the prevalence of future Preterm PE is enriched to p=0.053, resulting in a change to the slope of the PPV line thus increasing the sensitivity (detection rate) for the preset PPV criterion.

FIG. 4 Example 8A Determination of the minimal prognostic criteria for predicting preterm PE. For any classifier, risk scores which agree with a point in Area “A” would meet the minimum rule-in criterion, risk scores which agree with a point in Area “B” would meet the minimum rule-out criterion. For a classifier to meet both the rule-in and rule-out criteria at the same time, it's associated ROC curve (or paired Sensitivity-specificity value(s)) will have a point in the intersect area (A∩B).

FIG. 5 Example 8B Scatter plot showing PIGF levels at time of sampling vs time of delivery. Star symbol: Preterm PE. “bar” symbol: Term PE. “circle” symbol: no PE. Area “A” contains future preterm PE cases which will be missed by the application of a stand-alone PIGF threshold as indicated. Subjects with PIGF levels below a target threshold classified as “high risk” wherefore PPV>=0.071. Subjects with PIGF levels above the target threshold will be considered for further classification (cf. text).

FIG. 6 Example 8C Scatter plot displaying biomarker values at time of sampling for the variables PIGF and DLG for the study subjects. Study samples labelled according to (future) pregnancy outcome; i.e., no PE or “preterm” PE. Area “A” indicates a large zone in the scatter plot without (future) preterm-PE cases.

FIG. 7 Example 8D: Segmentation of the Study-Pop1 using a PIGF level as a Rule-in classifier.

FIG. 8 Example 8E “Total Classification” as achieved by applying a 1 step PIGF based (rule-in) classification

FIG. 9 Example 8F Segmentation of the Study-Pop2 using a DLG level as a Rule-out classifier. Subjects with DLG levels below a target threshold classified as “low risk” Subjects with DLG levels above the target threshold will be considered for further classification (cf. text),

FIG. 10 Example 8G “Total Classification” as achieved by applying a 2 step classification involving PIGF (rule-in) and DLG (rule-out), whereby the rule-in and the rule-out classifier are considered separately. The negative classification (not-rule-in, not ruled-out) is also plotted.

FIG. 11 Example 8H Further Segmentation of the Study-Pop3 using a L-ERG level as a Rule-out classifier. Subjects with L-ERG levels below a target threshold classified as “low risk” Subjects with L-ERG levels above the target threshold will be considered for further classification (cf. text)

FIG. 12 Example 8I “Total Classification” as achieved by applying a 3 step classification involving PIGF (rule-in), DLG (rule-out), and L-ERG (rule-out), whereby the rule-in and the rule-out classifiers are considered separately. The negative classification (not-rule-in, not ruled-out×2) is also plotted.

FIG. 13 Example 8J Further Segmentation of the Study-Pop4 using a s-ENG as a Rule-out classifier, creating a 3rd ruled-out population (Pop-LR3) as well as a Residual population. Subjects with s-ENG levels below a target threshold classified as “low risk” Subjects with s-ENG levels above the target threshold will be considered for further classification (cf. text)

FIG. 14 Example 8K: Total Classification” as achieved by applying a 3 step classification involving PIGF (rule-in), DLG (rule-out), I-ERG (rule-out), and s-ENG (rule-out). A The rule-in and the rule-out classifiers are considered separately. The negative classification (not-rule-in, not ruled-out×3) is also plotted. B. The total classifier is given as a single classifier classifying any pregnancy population or pregnant woman into a high risk group for developing preterm PE (with a PPV>=0.071) or into a low-risk group for developing preterm PE (with a NPV>=0.9975)

FIG. 15 Example 8L: Alternate Total Classifiers (as identified in the text) also exhibiting exceptional prognostic performance for preterm PE

FIG. 16 Example 9A: Illustration showing that by means of applying chiral LC (lower trace), the dilinoleoyl-glycerol signal as obtained by LC-MS/MS methodology similar to the one elaborated within this application can be resolved in 3 sub-species. Based on comparison with reference materials, it was found that the 1st two signals agreed with the enantiomers 1,2-/2,3-dilinoleoyl-glycerol enantiomers and the 3rd signal with the 1,3-dilinoleoyl-glycerol.

FIG. 17 Example 9B Correlations between the different total DLG and the different DLG-isoforms, as well as between different isoforms.

FIG. 18 Example 9C: Box plots summarizing the levels of the “total dilinoleoyl-glycerol” and the different dilinoleoyl-glycerol isoforms at time of sampling early in pregnancy in function of the pregnancy outcome experienced by the women at the end of pregnancy; i.e., “preterm PE”: n=17; “term PE”: n=42 and “no PE”; n=574. Fold changes for the differences in median values between the groups are also given.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

As used herein, the term “preeclampsia” or “PE” is defined as elevated blood pressure after 20 weeks of gestation (≥140 mm Hg systolic or ≥90 mm Hg diastolic) plus proteinuria (>0.3 g/24 hours). The term includes different types of PE including term PE, pre-term PE and early onset PE. The term “pre-term preeclampsia” refers to the occurrence of preeclampsia which results to the delivery of the infant before 37 weeks of gestation. The term “all preeclampsia” refers to term preeclampsia and pre-term preeclampsia. The methods of the invention relate to the early prediction of preeclampsia in pregnant women. However, the methods of the invention are also applicable for the early prediction of risk of hypertensive disorders in pregnant women, including for example eclampsia, mild preeclampsia, chronic hypertension, EPH gestosis, gestational hypertension, superimposed preeclampsia, HELLP syndrome, or nephropathy. Further, while the invention is described with reference to pregnant humans, it is also applicable to pregnant higher mammals.

As used herein, the term “biological sample” (or the test sample or the control) may be any biological fluid obtained from the subject pregnant woman or the foetus, including blood, serum, plasma, saliva, amniotic fluid, cerebrospinal fluid, nipple aspirate. Ideally, the biological sample is serum. The subject may be fasting or non-fasting when the biological sample is obtained. In a preferred embodiment, the biological sample is, or is derived from, blood obtained from the test pregnant woman.

As used herein, the term “variable” refers to a blood-borne metabolite or protein, or a clinical risk factor. The term “panel of variables selected from metabolites, proteins and clinical risk factors” means at least one and generally more than one variable selected from metabolites, proteins and clinical risk factors. Generally, the panel includes two variable classes, for example metabolite and protein, metabolite and clinical risk factor, or protein and clinical risk factor. In one embodiment, the panel; includes at least one metabolite, protein and clinical risk factor. Generally, the panel comprises a plurality of metabolites.

As used herein, the term “protein” refers to blood borne protein whose levels can be employed, optionally in combination with other variables, to predict preeclampsia. Examples of proteins useful in the prediction of preeclampsia include placental growth factor (PIGF), soluble fms-like tyrosine kinase 1 (sFlt1) and soluble endoglin (s-ENG).

As used herein, the term “clinical risk factor” refers to a clinical measurement other than a protein or metabolite measurement whose levels can be employed, optionally in combination with other variables, to predict preeclampsia. The term includes a blood pressure measurement (systolic, diastolic or mean arterial pressure (MAP), age of subject, family history of preeclampsia (fh_pet)—i.e. subjects mother or sister has PE, weight, body mass index (BMI), waist circumference, number of cigarettes per day in 1^{st} trimester (cig_1^{st}_trim_gp), gestational stage when biological sample is taken (gest), and random glucose measured by glucometer when biological sample is taken.

As used herein, the term “weight related variable” or “WRV” refers to weight, BMI or waist circumference of the subject.

As used herein, the term “BP” refers to a blood-pressure parameter, selected from 1^{st} and 2^{nd} systolic BP, 1^{st} and 2^{nd} diastolic BP, 1^{st} and 2^{nd} mean arterial pressure (MAP). In one embodiment, a composite BP value may be employed, comprising the mean of two measurements taken.

As used herein, the term “metabolite” or “metabolites” refers to intermediates and products of metabolism, and in particular mammalian metabolism. Typically, the metabolite is a metabolite relevant to preeclampsia (PE-relevant metabolite), examples of which are provided in Table 2. Metabolites may be classified according to metabolite class. Examples of metabolite classes include acetyls, acyclic alkanes, acyl carnitines, aldehydes, amino acids, amino ketones, aralkylamines, azacyclic compounds, benzene and substituted derivatives, tetrapyrolles and derivatives, biphenyls and derivatives, carnitines, cholines, corticosteroids and derivatives, coumarins and derivatives, diacylglycerols, dicarboxylic acids, dipeptides, Eicosanoids, fatty acids (hydroperoxyl fatty acids, keto- or hydroxy-fatty acids, saturated fatty acids, unsaturated fatty acids, epoxy fatty acids), glycerophospholipids, hydroxy acids and derivatives, monosaccharide phosphates, N-acyl-alpha amino acids, phenylpropanoic acids, phosphosphingolipids, azacyclic compounds (pryidines), sphingolipids, sugar alcohols, androgens and steroids (testosterones), Vitamin D and derivatives. In one embodiment, the metabolite is selected from the group consisting of Table 2. In one embodiment, the metabolite is a PE-relevant metabolite selected from the group consisting of: 25-Hydroxyvitamin D3 (HVD3); 2-hydroxybutanoid acid (2-HBA); L-leucine (L-LEU); Citrulline (CR); Docosahexaenoic acid (DHA); Dilinoleoyl-glycerol: 1,3Dilinoleoyl-glycerol: 1,2-Dilinoleoyl-glycerol (isomer mixture) (DLG); choline (CL); L-isoleucine (L-ISO); L-methionine (L-MET); NG-Monomethyl-L-arginine (NGM); Asymmetric dimethylarginine (ADMA); Taurine (TR); Stearoylcarnitine (SC); 1-heptadecanoyl-2-hydroxy-sn-glycero-3-phosphocholine (1-HD); Biliverdin (BV); Sphingosine 1-phosphate (S-1-P); and eicosapentaenoic acid (EPA). In one embodiment, the method comprises measuring a level of all, or substantially all, of the PE-relevant metabolites.

Unless stated otherwise, the metabolite and protein markers referenced herein refer to the total level of the metabolite or protein, including any isoforms of the metabolite or protein. However, it will be appreciated that the methods of the invention may be employed using specific isoforms of a given metabolite or protein. In the case of the metabolite DLG (Dilinoleoyl-glycerol), the term “DLG” refers to a total DLG including the sn-1,3-Dilinoleoyl-glycerol, and the racemic mixture of sn-1,2-Dilinoleoyl-glycerol and sn-2,3-Dilinoleoyl-glycerol (the latter 2 sometimes abbreviated to sn-1,2-rac-Dilinoleoyl-glycerol). However, the methods of the invention may be employed using any one or two or all three sterioisomers making up total DLG.

As used herein, the formula notation [variable] relates to the (relative) concentration in blood of this variable as determined with the assay as exemplified in this specification.

As used herein, the formula notation log 10[variable] relates to the logarithm to the base 10 of the (relative) concentration in blood of this variable, whereby the variable is determined with the assay as exemplified in this specification.

As used herein, the formulation [variable (MoM)] relates to multiple-of-median (MoM) normalized concentration of the variable. The variable is determined with the assay as exemplified in this specification.

As used herein, the term “rule-in prognostic signature” refers to a signature of a variable, or combination of variables, whose level or levels are above or below a defined threshold level for the variable or variables, which when detected in a subject is indicative of an increased risk of the subject developing preeclampsia. The defined threshold level for each variable is typically predetermined based on a nested case-control study of a study population in combination with a predefined rule-in test requirement and may therefore vary from test to test depending on the type of preeclampsia and the positive predictive value (PPV) required of the test. The rule-in prognostic signature may be univariable (i.e. be composed of a single variable such as a protein, metabolite or clinical risk factor) or multivariable (i.e. be composed of two or more variables selected from proteins, metabolites or clinical risk factors). When the prognostic signature is univariable, detection of the presence of the prognostic signature in the subject (in the case of a clinical risk factor variable) or in the biological sample (in the case of a protein or metabolite variable) generally involves measuring the level of the variable and comparing the level with a defined threshold level for the variable and determining whether the test level is above or below the threshold level. For example, in the case of prediction of risk of pre-term preeclampsia, detection of the protein PIGF in blood obtained from the subject at a level below the threshold level for PIGF constitutes a rule-in prognostic signature of pre-term preeclampsia. The threshold level in this case is the 7.56% centile as determined for a control population of woman at the same gestational age who did not develop preeclampsia. When the prognostic signature is multivariable signature comprising two or more variables (for example a metabolite and a protein, or two metabolites, or a clinical risk factor and a metabolite), determining the presence of the prognostic signature in the subject generally involves measuring the level of the variables and inputting the levels into a statistical model configured to provide an output in the form of a score, and comparing the score with a defined threshold score for the multivariable prognostic signature and determining whether the test score is above or below the threshold score. The defined threshold score is predetermined based on a study population and a predefined rule-in test requirement and may therefore vary from test to test depending on the type of preeclampsia and the desired stringency of the predictive test. The following are examples of rule-in multivariable prognostic signatures for preeclampsia:

### Pre-Term Preeclampsia:

A rule-in multivariable prognostic signature comprises the levels of the proteins-ENG and PIGF, and metabolites DLG and L-ERG, and the statistical model: 0.0942921407169182 log 10[s-ENG]−0.127933447595162 log 10[PIGF]+0.177562360580178 log 10[DLG]−0.0840930458415515 log 10[L-ERG], wherein when the output score of the statistical model is <0.478254130106926, the rule-in prognostic signature is considered to be present, indicating an increased risk of the subject developing pre-term preeclampsia.

### Term Preeclampsia

A rule-in prognostic signature comprises the levels of the following variables: BP, HVD3, L-ISO and 1-HD, and the statistical model: 0.00853293587443292[bp]+0.096620376132676 log 10 [HVD3]+0.24599289739986 log 10[L-ISO]−0.300891766915803 log 10[1-HD] wherein when the output score of the statistical model is <1.09653388177747, the rule-in prognostic signature is considered to be present, indicating an increased risk of the subject developing term preeclampsia.

### All Preeclampsia

A rule-in prognostic signature for “all” preeclampsia employs the levels of the following variables: BP, HVD3, PIGF and DLG, and the statistical model: −0.176060524601929 log 10[PIGF]+0.0143316978786453 [bp]+0.0149559619104756 log 10[HVD3]+0.116776043392906 log 10[DLG] wherein when the output score of the statistical model is <1.33083720900868, the rule-in prognostic signature is considered to be present, indicating an increased risk of the subject developing “all” preeclampsia.

Other examples of univariable and multivariable rule-in prognostic signatures are provided below in Examples 3 to 8.

As used herein, the term “rule-out prognostic signature” refers to a signature of a variable, or combination of variables, whose level or levels are above or below a defined threshold level for the variable or variables, which when detected in a subject is indicative of a reduced risk of the subject developing preeclampsia. The defined threshold level for each variable is typically predetermined based on a nested case-control study of a study population in combination with a predefined rule-in test requirement, and may therefore vary from test to test depending on the type of preeclampsia and the negative predictive value (NPV) required of the test. The rule-out prognostic signature may be univariable (i.e. be composed of a single variable such as a protein, metabolite or clinical risk factor) or multivariable (i.e. be composed of two or more variables selected from a protein, metabolite or clinical risk factor). When the prognostic signature is univariable, detection of the presence of the prognostic signature in the subject (in the case of a clinical risk factor variable) or in the biological sample (in the case of a protein or metabolite variable) generally involves measuring the level of the variable and comparing the level with a defined threshold level for the variable and determining whether the test level is above or below the threshold level. For example, in the case of prediction of risk of pre-term preeclampsia, detection of the metabolite DLG in blood obtained from the subject at a level below the threshold level constitutes a rule-out prognostic signature of pre-term preeclampsia. The threshold level in this case is the 61.1% centile as determined for a control population of woman at the same gestational age who did not develop preeclampsia. As another example, in the case of prediction of risk of pre-term preeclampsia, detection of the metabolite L-ERG in blood obtained from the subject at a level below the threshold level constitutes a rule-out prognostic signature of pre-term preeclampsia. The threshold level in this case is the 44.1% centile as determined for a control population of woman at the same gestational age who did not develop preeclampsia. When the prognostic signature is multivariable signature comprising two or more variables (for example a metabolite and a protein, or two metabolites, or a clinical risk factor and a metabolite), determining the presence of the prognostic signature in the subject generally involves measuring the level of the variables and inputting the levels into a statistical model configured to provide an output in the form of a score, and comparing the score with a defined threshold score for the multivariable prognostic signature and determining whether the test score is above or below the threshold score. The defined threshold score is predetermined based on a study population and a predefined rule-in test requirement, and may therefore vary from test to test depending on the type of preeclampsia and the desired stringency of the predictive test. The following are examples of rule-out multivariable prognostic signatures for preeclampsia:

### Pre-Term Preeclampsia:

A rule-out prognostic signature comprises the levels of s-ENG, DLG, NGM and 1-HD, and the statistical model: 0.253406507128582 log 10[s-ENG]+0.187688881253026 log 10[DLG]+0.168200074854411 log 10[NGM]−0.213566303707572 log 10[1-HD] wherein when the output score of the statistical model is <0.14882970770599, the rule-out prognostic signature is considered to be present, indicating a reduced risk of the subject developing pre-term preeclampsia.

### Term Preeclampsia

A rule-out prognostic signature comprises the levels of the following variables: BP and 1-HD, and the statistical model: 0.0115467461789923 [bp]−0.324977743714534 log 10[1-HD] wherein when the output score of the statistical model is <1.19965110779133, the rule-out prognostic signature is considered to be present, indicating an increased risk of the subject developing term preeclampsia.

### All Preeclampsia

A rule-out prognostic signature for “all” preeclampsia employs the levels of the following variables: s-ENG, BP, HVD3 and 1-HD, and the statistical model: 0.166860970853811 log 10[s-ENG]+0.0126847727730485 [bp]+0.115675583588397 log 10[HVD3]−0.154060908375255 log 10[1-HD] wherein when the output score of the statistical model is <1.36693873307782, the rule-out prognostic signature is considered to be present, indicating an increased risk of the subject developing “all” preeclampsia.

Other examples of univariable and multivariable rule-in prognostic signatures are provided below in Examples 3 to 8.

It will be appreciated that the same biomarker or variable may be employed in a rule-in prognostic signature and a rule-out prognostic signature. An example is BP in the case of prediction of term preeclampsia.

As used herein, the term “predicting risk of preeclampsia” should be understood to mean predicting increased risk or decreased risk of preeclampsia. In the case of detecting increased risk, the post-test probability is generally higher than the pre-test probability, for example 1.5, 2, 2.5, 3, 3.5, 4, 4.5 5, 5.5, 6, 6.5 7, 7.5 8, 8.5 9, 9.5 or 10 times the pre-test probability. in one embodiment, the method of the invention is configured to detect 40-60% of cases of preeclampsia (i.e. 40%-50% or 50-60%) with a false positive rate (FPR) of 5-25%, and preferably about 10-20% FPR. In the case of detecting decreased risk, the post-test probability is generally lower than the pre-test probability, for example 1.5, 2, 2.5, 3, 3.5, 4, 4.5 5, 5.5, 6, 6.5 7, 7.5 8, 8.5 9, 9.5 or 10 times lower than the pre-test probability. in one embodiment, the method of the invention is configured to detect 40-60% of non-cases of preeclampsia (i.e. 40%-50% or 50-60%) with a false negative rate (FNR) of 5-25%, and preferably about 10-20% FNR.

As used herein, the term “multiple metabolites” as applied to a biological sample refers to sample that contains at least 5 or 10 different metabolites, and in generally contains at least 40, 50, 70, 90 or 100 different metabolites. The methods of the invention may be employed to profile multiple metabolites in a biological sample, and in particular provide a qualitative and quantitative profile of multiple metabolites in a biological sample.

As used herein, the term “metabolic profiling” refers to the determination of a metabolite (or preferably metabolites) in a biological sample by mass spectroscopy, preferably LC-MS, dual LC-MS, and ideally dual LC-MS/MS. The determination of metabolites in the sample may be a determination of all metabolites, or selected metabolites. Preferably, the determination is a determination of metabolites relevant to hypertensive disorders of pregnancy, especially preeclampsia. The determination of metabolites may be qualitative, quantitative, or a combination of qualitative and quantitative. In one embodiment, quantitative determination is relative quantitative determination, i.e. determination of abundance of a specific metabolite in the sample relative to a known quantity of a stable isotope labelled internal standard (i.e. SIL-IS) corresponding to the metabolite of interest. In another embodiment, quantitative determination is determined in absolute terms. Metabolic profiling of a samples can be employed in case control studies (especially nested case control studies) to identify metabolites and combinations of metabolites that can function as prognostic and diagnostic variables of disease. In one embodiment, the metabolic profiling is targeted profiling, for the determination of specific metabolites, that typically employs tuned MS settings, and generally employs electrospray ionisation-triple quadrupole (QqQ) MS/MS analysis.

As used herein, the term “metabolite extraction solvent” refers to a solvent employed to extract metabolites from other components in the sample, especially protein. Generally, the solvent is an extraction/protein precipitation solvent that precipitates protein in the sample which can be separated using conventional separation technology (i.e. centrifugation or filtration), leaving a supernatant enriched in metabolites. The supernatant may then be applied to a chromatography column to resolve the metabolites in the sample and the eluent from the column may then be assayed by on-line mass spectrometry. In one embodiment, the metabolite extraction solvent comprises methanol, isopropanol and buffer. In one embodiment, the buffer is an acetate buffer. In one embodiment, the acetate buffer is an ammonium acetate buffer. Other volatile buffers or/and buffer salts may be employed, such as ammonia: acetic acid, ammonium formate, trimethylamine; acetic acid. In one embodiment, the acetate buffer has a concentration of about 150-250 mM, preferably about 200 mM. In one embodiment, the buffer is configured to buffer the pH of the extraction solvent to about 4-5, preferably about 4.5. In one embodiment, the extraction solvent comprises methanol and isopropanol in a volumetric ratio of about 5-15:5-15, or 8-12:8-12. In one embodiment, the extraction solvent comprises methanol, isopropanol and buffer in a ratio of about 10-30:10-30:1-5 (v/v/v). In one embodiment, the extraction solvent comprises methanol, isopropanol and ammonium acetate buffer in a ratio of about 10:9:1 (v/v/v).

As used herein, the term “chromatography” refers to a process in which a chemical mixture is separated into components as a result of differential distribution and or adsorption due to the differential physico-chemical properties of the components between two phases of different physical state, of which one is stationary and one is mobile.

As used herein, the term “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Examples of “liquid chromatography” include normal phase liquid chromatography (NPLC), reverse phase liquid chromatography (RPLC), high performance liquid chromatography (HPLC), ultra-high performance liquid chromatography (UHPLC), and turbulent flow liquid chromatography (TFLC) (sometimes known as high turbulence liquid chromatography (HTLC) or high throughput liquid chromatography).

As used herein, the term “high performance liquid chromatography” or “HPLC” (sometimes known as “high pressure liquid chromatography”) refers to liquid chromatography in which the degree of separation is increased by forcing the mobile phase under pressure through a stationary phase, typically a densely packed column.

As used herein, the term “ultra-high performance liquid chromatography” or “UHPLC” (sometimes known as “ultra high pressure liquid chromatography”) refers to liquid chromatography in which the degree of separation is increased by forcing the mobile phase under high pressure through a stationary phase, typically a densely packed column with a stationary phase comprising packing particles that have an average diameter of less than 2 μm.

As used herein, the term “turbulent flow liquid chromatography” or “TFLC” (sometimes known as high turbulence liquid chromatography or high throughput liquid chromatography) refers to a form of chromatography that utilizes turbulent flow of the material being assayed through the column packing as the basis for performing the separation. TFLC has been applied in the preparation of samples containing two unnamed drugs prior to analysis by mass spectrometry. See, e.g., Zimmer et al., J Chromatogr A 854: 23-35 (1999); see also, U.S. Pat. Nos. 5,968,367, 5,919,368, 5,795,469, and 5,772,874, which further explain TFLC. Persons of ordinary skill in the art understand “turbulent flow”. When fluid flows slowly and smoothly, the flow is called “laminar flow”. For example, fluid moving through an HPLC column at low flow rates is laminar. In laminar flow the motion of the particles of fluid is orderly with particles moving generally in straight lines. At faster velocities, the inertia of the water overcomes fluid frictional forces and turbulent flow results. Fluid not in contact with the irregular boundary “outruns” that which is slowed by friction or deflected by an uneven surface. When a fluid is flowing turbulently, it flows in eddies and whirls (or vortices), with more “drag” than when the flow is laminar. Many references are available for assisting in determining when fluid flow is laminar or turbulent (e.g., Turbulent Flow Analysis Measurement and Prediction, P. S. Bernard & J. M. Wallace, John Wiley & Sons, Inc., (2000); An Introduction to Turbulent Flow, Jean Mathieu & Julian Scott, Cambridge University Press (2001)).

As used herein, the term “dual liquid chromatography” or “dual LC” as applied to a biological sample refers to separation step in which a first aliquot of the sample is subjected to a first type of LC (i.e. C18 RPLC) and a second aliquot of the sample is subjected to a second type of LC (i.e. HILIC). This is especially suitable for methods of the invention in which multiple metabolites are profiled, as the dual LC separation of the sample provides for improved resolution of the metabolites, and therefore improved analytical determination. In one embodiment, the dual LC step comprises three or more chromatography steps which are performed on separate aliquots of the same sample, for example two RPLC steps which are configured to separate (different) sets of hydrophobic metabolites, and two HILIC steps which are configured to separate (different) sets of hydrophilic metabolites. This may be employed when the set of metabolites in the sample is too expansive to be adequately assayed by in-line mass spectrometry in a single dual RPLC-MS—HILIC-MS analysis.

As used herein, the term “solid phase extraction” or “SPE” refers to a process in which a chemical mixture is separated into components as a result of the affinity of components dissolved or suspended in a solution (i.e., mobile phase) for a solid through or around which the solution is passed (i.e., solid phase). In some instances, as the mobile phase passes through or around the solid phase, undesired components of the mobile phase may be retained by the solid phase resulting in a purification of the analyte in the mobile phase. In other instances, the analyte may be retained by the solid phase, allowing undesired components of the mobile phase to pass through or around the solid phase. In these instances, a second mobile phase is then used to elute the retained analyte off the solid phase for further processing or analysis. SPE, including TFLC, may operate via a unitary or mixed mode mechanism. Mixed mode mechanisms utilize ion exchange and hydrophobic retention in the same column; for example, the solid phase of a mixed-mode SPE column may exhibit strong anion exchange and hydrophobic retention; or may exhibit column exhibit strong cation exchange and hydrophobic retention.

As used herein, the term “in-line” or “on-line” as applied to mass spectrometry refers to mass spectrometry equipped with any ionisation source which enables the real-time ionisation of analytes present in an LC eluent which is directly and continuously led to a mass spectrometer.

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 1999, 2: 264-76; and Merchant and Weinberger, Electrophoresis 2000, 21: 1164-67.

As used herein, the term “tandem mass spectrometry” refers to a method involving at least two stages of mass analysis, either in conjunction with a dissociation process or a chemical reaction that causes a change in the mass or charge of an ion. The main advantage of using MS/MS is the discrimination against the chemical noise, which can originate from different sources (e.g. matrix compounds, column bleed, contamination from an ion source).

There are two different approaches in MS/MS: in space by coupling of two or more physically distinct parts of an instrument (e.g. triple quadrupole (QqQ), or Quadrupole-Time of Flight, Qq-TOF, Triple TOF, quadrupole orbitrap); or in time by performing a sequence of events in an ion storage device (e.g. ion trap, IT) or hybrids thereof (e.g., quadrupole-ion trap-orbitrap). The main tandem MS/MS scan modes are product ion, precursor ion, neutral loss, selected reaction monitoring, multiple reaction monitoring, and MS' scans.

Generally, quantitative tandem MS is performed with a triple quadrupole (QqQ) MS analyser.

MS/MS methods generally involve activation of selected ions, typically by collision with an inert gas, sufficient to induce fragmentation (collision induced dissociation, CID) and generate product ions. The product ion scan involves selection of the precursor ion of interest (using the first mass filter (Q1), its activation (q2) and a mass analysis scan (Q3) to determine its product ions. The product ion scan represents opposite process compared to the precursor ion scan; the 2nd mass filter (Q3) is set to analyse a single a product ion, whereas the first mass filter (Q1) is used to scan for precursor ions which will dissociate (in q2) into said product ion. The neutral loss scan involves scanning for a fragmentation (neutral loss of fixed, predetermined mass); Q1 and Q3 will be scanning a set m/z range in parallel, but with their filters off-set in accordance with predetermined neutral mass. It is useful for rapid screening in metabolic studies. MS' is commonly applied on ion-trap analysers. A precursor ion is selected and isolated by ejecting all other masses from the mass spectrometer. CID of the precursor ion yields ions that may have different masses (MS/MS). A product mass of an analyte is selected and other fragment ions are ejected from the cell. This product ion can be, again, subjected to CID, generating more product ions that are mass analysed (MS/MS/MS). This process can be repeated several times. However, as already mentioned, for small molecules like metabolites only MS/MS or MS/MS/MS is mainly used in practice. Selected reaction monitoring (SRM) is a special case of Selected Ion Monitoring (SIM) in which a tandem instrument is used to enhance the selectivity of SIM, by selecting both the precursor ion and the product ion. The term multiple reaction monitoring (MRM) is used if several different reactions are monitored in parallel.

As used herein, the term “selective ion monitoring” is a detection mode for a mass spectrometric instrument in which only ions within a relatively narrow mass range, typically about one mass unit, are detected.

As used herein, “multiple reaction mode,” sometimes known as “selected reaction monitoring,” is a detection mode for a mass spectrometric instrument in which a precursor ion and one or more fragment ions are selectively detected. In one embodiment, the mass spectrometry of the invention employs multiple reaction mode detection.

As used herein, the term “operating in negative ion mode” refers to those mass spectrometry methods where negative ions are generated and detected. The term “operating in positive ion mode” as used herein, refers to those mass spectrometry methods where positive ions are generated and detected.

As used herein, the term “ionization” or “ionizing” refers to the process of generating an analyte ion having a net electrical charge equal to one or more electron units. Negative ions are those having a net negative charge of one or more electron units, while positive ions are those having a net positive charge of one or more electron units.

As used herein, the term “electron ionization” or “EI” refers to methods in which an analyte of interest in a gaseous or vapor phase interacts with a flow of electrons. Impact of the electrons with the analyte produces analyte ions, which may then be subjected to a mass spectrometry technique.

As used herein, the term “chemical ionization” or “C1” refers to methods in which a reagent gas (e.g. ammonia) is subjected to electron impact, and analyte ions are formed by the interaction of reagent gas ions and analyte molecules.

As used herein, the term “fast atom bombardment” or “FAB” refers to methods in which a beam of high energy atoms (often Xe or Ar) impacts a non-volatile sample, desorbing and ionizing molecules contained in the sample. Test samples are dissolved in a viscous liquid matrix such as glycerol, thioglycerol, m-nitrobenzyl alcohol, 18-crown-6 crown ether, 2-nitrophenyloctyl ether, sulfolane, diethanolamine, and triethanolamine. The choice of an appropriate matrix for a compound or sample is an empirical process.

As used herein, the term “matrix-assisted laser desorption ionization” or “MALDI” refers to methods in which a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules.

As used herein, the term “surface enhanced laser desorption ionization” or “SELDI” refers to another method in which a non-volatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

As used herein, the term “electrospray ionization” or “ESI,” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber. As the droplets get smaller the electrical surface charge density increases until such time that the natural repulsion between like charges causes ions as well as neutral molecules to be released. Heated ESI is similar but includes a heat source for heating the sample while in the capillary tube. As used herein, the Agilent Jet Stream ionisation source refers to an ESI-variant using thermal gradient focusing technology to generate optimized ESI conditions.

As used herein, the term “atmospheric pressure chemical ionization” or “APCI,” refers to mass spectrometry methods that are similar to ESI; however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated N₂ gas may be used to improve removal of solvent. The gas-phase ionization in APCI can be more effective than ESI for analyzing less-polar species.

The term “atmospheric pressure photoionization” or “APPI” as used herein refers to the form of mass spectrometry where the mechanism for the photoionization of molecule M is photon absorption and electron ejection to form the molecular ion M. Because the photon energy typically is just above the ionization potential, the molecular ion is less susceptible to dissociation. In many cases it may be possible to analyse samples without the need for chromatography, thus saving significant time and expense. In the presence of water vapor or protic solvents, the molecular ion can extract H to form MH⁺. This tends to occur if M has a high proton affinity. This does not affect quantitation accuracy because the sum of M⁺ and MH⁺ is constant. Drug compounds in protic solvents are usually observed as whereas nonpolar compounds such as naphthalene or testosterone usually form M. See, e.g., Robb et al., Anal. Chem. 2000, 72(15): 3653-3659.

As used herein, the term “field desorption” refers to methods in which a non-volatile test sample is placed on an ionization surface, and an intense electric field is used to generate analyte ions.

As used herein, the term “desorption” refers to the removal of an analyte from a surface and/or the entry of an analyte into a gaseous phase. Laser desorption thermal desorption is a technique wherein a sample containing the analyte is thermally desorbed into the gas phase by a laser pulse. The laser hits the back of a specially made 96-well plate with a metal base. The laser pulse heats the base and the heat causes the sample to transfer into the gas phase. The gas phase sample is then drawn into the mass spectrometer.

As used herein, an “amount” of an analyte in a body fluid sample refers generally to an absolute value reflecting the mass of the analyte detectable in volume of sample. However, an amount also contemplates a relative amount in comparison to another analyte amount. For example, an amount of an analyte in a sample can be an amount which is greater than a control or normal level of the analyte normally present in the sample.

As used herein, the term “absorptive sampling device” refers to a liquid sampling device for biological material such as blood that employ an absorption medium that rapidly wicks biological fluid on to the absorption medium where the fluid is stored in a dried format. In one embodiment, the absorptive sampling device is a “volume-controlling absorptive sampling device” which is an absorptive sampling device configured to sample fluid in a volumetric, or volume controlled, fashion. Volumetric sampling is achieved by using a fixed reproducible internal volume for the absorption medium (controlling the capacity of the medium), or by controlling the volume deposited onto the absorption medium, the latter often employing microfluidic technology. One example is a “volumetric absorptive microsampling device” or “VAM device” which refers to blood sampling devices that employ a hydrophilic porous material with predefined internal volumes. They are described in EP2785859 and EP16753193 (Neoteryx LLC). Examples include the Neoteryx MITRA microsampler, available from Neoteryx of Torrence Calif., US. Other types of volume controlling sampling devices include DBS Systems HEMAXIS device (control of volume deposition), and HEMASPOT from SpotON Sciences (control of medium capacity).

Samples collected in this way are also known as “dried liquid” or “dried blood” samples As used herein, the term “chromatography” refers to a process in which a chemical mixture carried by a liquid or gas is separated into components as a result of differential distribution of the chemical entities as they flow around or over a stationary liquid or solid phase.

As used herein, the term “prophylactic therapy” refers to a therapeutic intervention for pregnant women to prevent development of preeclampsia typically during the second or third trimester of pregnancy. Examples of therapeutic intervention include aspirin [7], metformin [8]; Low Molecular Weight Heparin [12], glycemic index lowering probiotics [13]; citrulline [14] or antioxidants, inclusive but not limited to, antioxidant vitamins (e.g., ascorbic acid, alpha-tocopherol, beta-carotene) [15], inorganic antioxidants (e.g., selenium), and a plant-derived polyphenols, and/or antioxidants to mitochondria [25] inclusive but not limited to, Mito VitE and ergothioneine [16,17]; statins, inclusive but not limited to, Pravastin [18]; anti-hypertensive treatments (using inter alia beta-blockers; vasodilators, inclusive but not limited to H2S [19] or NO-donors like Sildenafil or others [20]; DOPA decarboxylase inhibitors) or anti-inflammatory therapeutics, inclusive but not limited to Digibind [21]; or actors against oxidative stress damage, inclusive but not limited to, (a1-microglobulin) [22] can also be considered. In addition, one can easily envision preferred therapeutic combinations like, but not limited to, aspirin and and/or antioxidants to mitochondria,

or a combination therapy comprising Metformin and an addition drug, for example aspirin, thiazolidinediones, DPP-4 inhibitors, sulfonylureas, and meglitinide.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

### Participants and Specimens:

Prospective clinical samples were collected from pregnant women with a singleton pregnancy at 15+/−1 and 20+/−1 weeks' gestation and which were either diagnosed with preeclampsia (cases) or not diagnosed with preeclampsia (controls) in the further course of their pregnancy. All samples were obtained from participants in the SCOPE (Screening fOr Pregnancy Endpoints) prospective screening study of nulliparous women [23,24].

Written consent was obtained from each participant. The inclusion criteria applied for the study were nulliparity, singleton pregnancy, gestation age between 14 weeks 0 days and 16 weeks 6 days gestation and informed consent to participate. The exclusion criteria applied were: Unsure of last menstrual period (LMP) and unwilling to have ultrasound scan at <=20 weeks, >=3 miscarriages, >=3 terminations, major fetal anomaly/abnormal karyotype, essential hypertension treated pre-pregnancy, moderate-severe hypertension at booking >=160/100 mmHg, diabetes, renal disease, systemic lupus erythematosus, anti-phospholipid syndrome, sickle cell disease, HIV positive, major uterine anomaly, cervical suture, knife cone biopsy, ruptured membranes now, long term steroids, treatment low-dose aspirin, treatment calcium (>1 g/24 h), treatment eicosopentanoic acid (fish oil), treatment vitaminC>=1000 mg & VitE>=400iu, treatment heparin/low molecular weight heparin.

Preeclampsia defined as gestational hypertension (systolic blood pressure (BP)>=140 mmHg and/or diastolic BP>=90 mmHg (Korotkoff V) on at least 2 occasions 4 hours apart after 20 weeks gestation but before the onset of labour) or postpartum systolic BP>=140 mmHg and/or diastolic BP>=90 mmHg postpartum on at least 2 occasions 4 hours apart with proteinuria >=300 mg/24 h or spot urine protein: creatinine ratio >=30 mg/mmol creatinine or urine dipstick protein >=2 or any multisystem complication of preeclampsia. Multisystem complications include any of the following: 1) Acute renal insufficiency defined as a new increase in serum creatinine >=100 umol/L antepartum or >130 umol/L postpartum 2) Liver disease defined as raised aspartate transaminase and/or alanine transaminase >45 IU/L and/or severe right upper quadrant or epigastric pain or liver rupture 3) Neurological problems defined as eclampsia or imminent eclampsia (severe headache with hyperreflexia and persistent visual disturbance) or cerebral haemorrhage 4) Haematological including thrombocytopenia (platelets <100×109/L), disseminated intravascular coagulation or haemolysis, diagnosed by features on blood film (e.g., fragmented cells, helmet cells) and reduced haptoglobin. Preeclampsia could be diagnosed at any stage during pregnancy after recruitment until delivery or in the first 2 weeks after delivery.

Clinical data on known risk factors for preeclampsia (Zhong et al, Prenatal Diagnosis, 30, p. 293-308, 2010; Sibai et al, 365, p. 785-799, 2005) was collected at 15+/−1 and 20+/−1 weeks' gestation by interview and examination of the women. Ultrasound data were obtained at 20 weeks on fetal measurements, anatomy, uterine and umbilical artery Doppler and cervical length. Fetal growth, uterine and umbilical Dopplers are measured at 24 weeks. Pregnancy outcome was tracked and the woman seen within 48 hours of delivery. Baby measurements are obtained within 48 hours of delivery.

### Sample Set Used:

A nested case-control study was conducted within the European branch of SCOPE, using blood samples taken at 15+/−1 weeks of gestation; the cohort constituted a case:control ratio of ˜1:3.5. Cases are defined as these pregnant women who develop preeclampsia (as defined earlier) in the course of their pregnancy: within the study 97 cases were considered, this corresponds all cases within the European branch of SCOPE for which samples were available. Controls were randomly selected amongst all other pregnancies. To avoid artefacts due to selection bias, the demographic and clinical characteristics of the control population selected to the study was compared and the lack of bias was verified using the appropriate statistical tests; the Chi-squared, Spearman Correlation, Mann Whitney U and Kruskal-Wallis tests were used as appropriate. Samples of 335 controls pregnancies were selected to the study.

In Table 1A, the baseline characteristics of this study cohort are presented.

TABLE 1A
Characteristics of the study population

| Characteristics | Controls | | PE | | Preterm PE | | Non-Preterm PE | |
|---|---|---|---|---|---|---|---|---|
| Number of samples | 335 | | 97 | | 23 | | 74 | |
| Gestation at sampling, wk | 15.6 | (0.726) | 15.6 | (0.698) | 15.6 | (0.491) | 15.6 | (0.754) |
| Population characteristics at | | | | | | | | |
| time of sampling (15 weeks) | | | | | | | | |
| Maternal age, y | 30 | (27-33) | 30 | (27-34) | 31 | (28.0-33.5) | 29.5 | (26.0-33.8) |
| White ethnicity | 315 | (94%) | 92 | (94.8%) | 22 | (95.7%) | 70 | (94.6%) |
| Body mass index at 15 wk, kg/m2 | 23.5 | (21.9-26.9) | 26.1 | (23.1-29.4) ‡ | 25.8 | (24.0-28.3) † | 26.2 | (22.9-29.7) ‡ |
| Waist circumference at 15 wk, cm | 80 | (74-86) | 84 | (78-91) ‡ | 84 | (78.0-89.5) | 84 | (77.2-93.0) † |
| Smoker at 15 wk | 117 | (34.9%) | 28 | (28.9%) | 6 | (26.1%) | 22 | (29.7%) |
| Smoker during first trimester | 106 | (31.6%) | 28 | (28.9%) | 6 | (26.1%) | 22 | (29.7%) |
| Blood pressure at 15 wk, mm | 64.8 | (7.15) | 68.1 | (7.98) ‡ | 66.1 | (8.05) | 68.7 | (7.92) ‡ |
| Hg-Diastolic, 1st | | | | | | | | |
| Blood pressure at 15 wk, mm | 77.8 | (7.68) | 82.1 | (8.51) ‡ | 79.5 | (8.34) | 82.9 | (8.44) ‡ |
| Hg-Mean arterial pressure, 1st | | | | | | | | |
| Blood pressure at 15 wk, mm | 78.6 | (7.45) | 83.1 | (7.84) ‡ | 80.7 | (7.52) | 83.9 | (7.84) ‡ |
| Hg-Mean arterial pressure, 2nd | | | | | | | | |
| Blood pressure at 15 wk, mm | 104 | (10.7) | 110 | (11.2) ‡ | 106 | (9.7) | 111 | (11.5) ‡ |
| Hg-Systolic, 1st | | | | | | | | |
| Random glucose measured by | 5.1 | (4.6-5.6) | 5.2 | (4.5-5.7) | 5.3 | (4.65-6.15) | 5.1 | (4.45-5.70) |
| glucometer at 15 wk, mmol/L | | | | | | | | |
| Population characteristics | | | | | | | | |
| at time of delivery | | | | | | | | |
| Maximum blood pressure- | 80 | (74-85) | 100 | (95-107) ‡ | 107 | (100-116) ‡ | 98 | (95-105) ‡ |
| Diastolic, mm Hg | | | | | | | | |
| Maximum blood pressure- | 125 | (118-134) | 154 | (147-170) ‡ | 167 | (154-180) ‡ | 151 | (145-162) ‡ |
| Systolic, mm Hg | | | | | | | | |
| Proteinuria* | 7 | (2.09%) | 91 | (93.8%) ‡ | 21 | (91.3%) ‡ | 70 | (94.6%) ‡ |
| Multiorgan complications | 0 | (0%) | 23 | (23.7%) ‡ | 9 | (39.1%) ‡ | 14 | (18.9%) † |
| Pregnancy outcome-Gestation | 40.4 | (39.6-41.3) | 38.9 | (37.1-40.3) ‡ | 34.3 | (33.0-35.8) ‡ | 39.5 | (38.6-40.4) ‡ |
| age at delivery, wk | | | | | | | | |

Results are expressed as mean (SD), median (interquartile range), or n (%).
*Urine dipstick ≥2+ or 24-h urine protein excretion ≥300 mg or spot urine protein:creatinine ratio ≥30 mg/mmol.
† P < 0.05;
‡ P < 0.001 cases vs controls, Chi-squared, T or Mann Whitney U test.

### Metabolites of Interest:

TABLE 2
tabulates a non-limiting list of metabolites of interest which are considered in this application. These metabolites, and or
metabolite classes, are deemed relevant by the inventors in view of identifying non-obvious prognostic combinations of
metabolites, to predict risk of preeclampsia in a pregnant woman prior to appearance of clinical symptoms of
preeclampsia in the woman preeclampsia. Where possible the metabolites of interest are identified by their CAS number,
or/and their HMDB identifier; the molecular weights are also given (na: not available).
Metabolites of interest

| Metabolite | Code | Metabolite Class | HMDB | CAS | MW |
|---|---|---|---|---|---|
| 25-Hydroxyvitamin D₃ | HVD3 | Vitamin D and derivatives | 0003550 | CAS 63283-36-3 | 400.6371 |
| 2-Hydroxybutanoic acid | 2-HBA | Keto- or Hydroxy fatty acids | 0000008 | CAS 5094-24-6 | 104.1045 |
| 2-methylglutaric acid | 2-MGA | Dicarboxylic acids | 0000422 | CAS 617-62-9 | 146.1412 |
| 3-Hydroxybutanoic acid | 3-HBA | Keto- or Hydroxy fatty acids | 0000357 | CAS 300-85-6 | 104.1045 |
| adipic acid | ADA | Dicarboxylic acids | 0000448 | CAS 124-04-9 | 146.1412 |
| L-alanine | L-ALA | Amino acids | 0000161 | CAS 56-41-7 | 89.0932 |
| Arachidonic acid | ARA | Fatty acids | 0001043 | CAS 506-32-1 | 304.4669 |
| L-arginine | L-ARG | Amino acids | 0000517 | CAS 74-79-3 | 174.201 |
| L-leucine | L-LEU | Amino acids | 0000687 | CAS 61-90-5 | 131.1729 |
| 8,11,14 Eicosatrienoic acid | DGLA | Eicosanoids | 0002925 | CAS 1783-84-2 | 306.4828 |
| Citrulline | CR | Amino acids | 0000904 | CAS 372-75-8 | 175.1857 |
| Decanoylcarnitine | DC | Carnitines | 0000651 | CAS 1492-27-9 | 315.4482 |
| Dodecanoyl-l-carnitine (c12) | 12CAR | Carnitines | 0002250 | CAS 25518-54-1 | 343.5014 |
| Docosahexaenoic acid | DHA | Fatty acids | 0002183 | CAS 6217-54-5 | 328.4883 |
| Dilinoleoyl-glycerol: | DLG | Diacylglycerols | 0007248 | CAS 15818-46-9 | 616.9542 |
| 1,3-Dilinoleoyl-glycerol | | | | CAS 30606-27-0 | |
| 1,2-rac-Dilinoleoyl- | | | | | |
| glycerol (isomer mixture) | | | | | |
| Choline | CL | Cholines | 0000097 | CAS 62-49-7 | 104.1708 |
| Glycyl-glycine | GG | Dipeptides | 0011733 | CAS 556-50-3 | 132.1179 |
| Homo-L-arginine | H-L-ARG | Amino acids | 0000670 | CAS 156-86-5 | 188.2275 |
| Hexadecanoic acid (palmitic acid) | PALMA | Fatty acids | 0000220 | CAS 57-10-3 | 256.4241 |
| L-Isoleucine | L-ISO | Amino acids | 0000172 | CAS 73-32-5 | 131.1729 |
| Linoleic acid | LINA | Fatty acids | 0000673 | CAS 60-33-3 | 280.4455 |
| L-methionine | L-MET | Amino acids | 0000696 | CAS 63-68-3 | 149.211 |
| NG-Monomethyl-L-arginine | NGM | Amino acids | 0029416 | CAS 17035-90-4 | 188.2275 |
| Oleic acid | OLA | Fatty acids | 0000207 | CAS 112-80-1 | 282.4614 |
| L-Palmitoylcarnitine | 16CAR | Acyl carnitines | 0000222 | CAS 6865-14-1 | 399.6077 |
| Asymmetric dimethylarginine | ADMA | Amino acids | 0001539 | CAS 30315-93-6 | 202.2541 |
| Sphingosine 1-phosphate | S-1-P | Phosphosphingolipids | 0000277 | CAS 26993-30-6 | 379.4718 |
| Sphinganine-1-phosphate (C18 base) | Sa-1-P | Phosphosphingolipids | 0001383 | CAS 19794-97-9 | 381.4877 |
| Symmetric dimethylarginine | sDMA | Amino acids | 0003334 | CAS 30344-00-4 | 202.2541 |
| Taurine | TR | Amino acids | 0000251 | CAS 107-35-7 | 125.147 |
| Isobutyrylglycine | IBG | N-acyl-alpha amino acids | 0000730 | CAS 15926-18-8 | 145.1564 |
| Urea | UR | Amino ketones | 0000294 | CAS 57-13-6 | 60.0553 |
| Stearoylcarnitine | SC | Acyl carnitines | 0000848 | CAS 1976-27-8 | 427.6609 |
| Eicosapentaenoic acid | EPA | Eicosanoids and/or retinoids | 0001999 | CAS 10417-94-4 | 302.451 |
| Ricinoleic acid | RIA | Fatty acids | 0034297 | CAS 141-22-0 | 298.4608 |
| 13-Oxooctadecanoic acid | O-STERA | Fatty acids | na | Not available | 298.4608 |
| 3-Hydroxytetradecanoic acid | 3H-MYRA | Fatty acids | 0061656 | CAS 3422-31-9 | 244.3703 |
| 1-heptadecanoyl-2-hydroxy- | 1-HD | Glycerophospholipids | 0012108 | CAS 50930-23-9 | 509.6566 |
| sn-glycero-3-phosphocholine | | | | | |
| Bilirubin | BR | Bilirubins | 0000054 | CAS 635-65-4 | 584.6621 |
| Biliverdin | BV | Bilirubins | 0001008 | CAS 114-25-0 | 582.6463 |
| Etiocholanolone glucuronide | ECG | Testosterones | 0004484 | CAS 3602-09-3 | 466.5644 |
| Cotinine | COT | Pyridines | 0001046 | CAS 486-56-6 | |
| Myristic acid | MYRA | Fatty acids | 0000806 | CAS 544-63-8 | 228.3709 |
| Stearic acid | STERA | Fatty acids | 0000827 | CAS 57-11-4 | 284.4772 |
| 1-oleoyl-2-hydroxy-sn- | OL-GPS | glycerophospholipids | Na | CAS 326589-90-6 | 522.596 |
| glycero-3-phospho-L-serine | | | | | |
| L-(+)-Ergothioneine | L-ERG | Amino acids | 0003045 | CAS 497-30-3 | 229.299 |
| 20-Carboxy-leukotriene B4 | 20-CL | Fatty acids | 0006059 | CAS 80434-82-8 | 366.4486 |
| 2-Hydroxytetradecanoic acid | 2H-MYRA | Fatty acids | 0002261 | CAS 2507-55-3 | 244.3703 |
| 1-Palmitoyl-2-hydroxy- | PA-GPC | Glycerophospholipids | 0010382 | CAS 17364-16-8 | 495.6301 |
| sn-glycero-3-phosphocholine | | | | | |
| (LysoPC(16:0)) | | | | | |
| L-Acetylcarnitine | AcCAR | Carnitines | 0000201 | CAS 3040-38-8 | 203.2356 |
| 6-Hydroxysphingosine | 6H-Sa | Sphingolipids | Na | Not available | 315.498 |
| L-Lysine | L-LYS | Amino acids | 0000182 | CAS 56-87-1 | 146.1876 |
| L-Glutamine | L_GLU | Amino acids | 0000641 | CAS 56-85-9 | 146.1445 |
| Sphinganine-1-phosphate | Sa-1- | Phosphosphingolipids | Na | CAS 474923-29-0 | |
| (C17 base) | P(17) | | | | |

In order to develop the collection of analytical methods as disclosed herein, reference materials for the above metabolites were purchased from: Fluka (Arklow, Ireland), Fischer scientific (Blanchardstown, Ireland), IsoSciences (King of Prussia, Pa., USA), Sigma-Aldrich (Wicklow, Ireland), Avanti Lipids (Alabaster, Ala., USA), QMX Laboratories (Thaxted, UK), LGC (Teddington, U.K), Alfa Chemistry (Holtsville, N.Y., USA), Generon (Maidenhead, UK), Larodan (Solna, Sweden) and R&D Systems (Abingdon, UK). Depending on physicochemical characteristics of the metabolite of interest, sometimes a salt form of the metabolite of interest was procured.

### Exemplary Prognostic Targets for Preeclampsia Risk Stratification Tests:

As elaborated elsewhere in this application, the methods as disclosed herein enable for the discovery of combinations of variables for total preeclampsia, but also for clinically relevant subtypes of preeclampsia or/and for different patient populations with different risk profiles.

Within this application, the focus is on establishing prognostic combinations for different sub-types of preeclampsia within a specific patient population, i.e., 1^{st} time pregnant women without overt clinical risk factors. Other patient populations, wherefore the inventors applied the collection of methods as disclosed here-in, are the preeclampsia risk within the obese pregnant population, as well as in the non-obese population.

The preeclampsia sub-types targeted here are
total preeclampsia (“all PE”)
preterm preeclampsia (PT-PE): this is defined as preeclampsia which results in a (iatrogenic) delivery before 37 weeks of gestation, or preterm.
term preeclampsia (T-PE): this is defined as preeclampsia which is associated with at delivery at or later than 37 weeks of gestation, or term.

### AUROC Targets:

Royston et al. noted that the AUROC of clinical prognostic models is typically between 0.6 and 0.85.[25]

Therefore, the inventors put a minimal performance of AUC>=0.65, as a minimum AUROC for any combination of variables to be considered a prognostic model/core.

### Rule-in Targets, Based on a False Positive Rate (Specificity) Threshold:

Within this application, the discovery of prognostic models/cores which maximize the detection rate (Sensitivity) of future cases of preeclampsia, for a given False Positive Rate (FPR or (1-Specificity)) of future non-cases, is also considered. These prognostic models focus on identifying individuals who will develop preeclampsia. The following FPRs are considered: 20% FPR (Specificity=0.8) and 10% FPR (Specificity=0.9). In view of delivering a clinical meaningful rule-in test, the following minimal detection rates are put:
20% FPR: >=50% detection rate of future PE cases (Sensitivity >=0.5)
10% FPR: >=40% detection rate of future PE cases (Sensitivity >=0.4)

### Rule-Out Targets, Based on a False Negative Rate (Sensitivity) Threshold:

Within this application, the discovery of prognostic models/cores which maximize the detection rate (Specificity) of future non-cases of preeclampsia, for a given False Negative Rate (FNR or (1-Sensitivity)) of future cases, is also considered. These prognostic models focus on identifying individuals who will not develop preeclampsia. The following FNRs are considered: 20% FNR (Sensitivity=0.8) and 10% FNR (Sensitivity=0.9). In view of delivering a clinical meaningful rule-out test, the following minimal detection rates are put:
20% FNR: >=40% detection rate of future non-PE cases (Specificity >=0.40)
10% FPR: >=30% detection rate of future non-PE cases (Specificity >=0.30)

### Positive- and Negative Predictive Value Thresholds

All PE: First time pregnant women have a risk of ˜ 1/20 to develop preeclampsia,[24] or a relative risk of approximately 2, compared to non-nulliparous.[26]

In our efforts to develop a clinically meaningful screening test, the inventors recently published the following rationale.[27] The prenatal management of a multiparous woman with regards to preeclampsia is largely guided by her previous pregnancy history. Epidemiological studies have shown that previous preeclampsia is associated with an increased risk of recurrence. For a second pregnancy, recurrence risks of about 1 in 8.6 to 1 in 6.8 (or PPV of 0.116 to 0.147) are reported,[28][29] whereas a woman without prior preeclampsia, will have a lower risk of 1 in 77 to 1 in 100 (or NPV of 0.987 to 0.99). [28][29] In line with this, if a woman has experienced preeclampsia in a previous pregnancy, she will be managed more vigilantly in most healthcare systems in high resource settings, with more prenatal visits compared to a woman who did not develop preeclampsia in any earlier pregnancy.

Based on the above, we proposed that a preeclampsia risk stratification test for nulliparous should ideally mimic the preeclampsia risk information as available for a second-time pregnant woman. Therefore, the test should either stratify nulliparous women to a high-risk group with a post-test preeclampsia probability of at least 1 in 7.5 (equivalent to a PPV=0.133; rule-in) or stratify them to a low-risk group with a post-test probability of at least 1 in 90 (equivalent to a NPV=0.988; rule-out) and ideally both. Based on this rationale, and taking into account the prevalence of PE as reported in the SCOPE cohort, the “All PE” PPV and NPV thresholds were established; cf. Table. 3.

Preterm PE: For preterm PE, the PPV and NPV thresholds were adopted from a benchmark preterm PE test, which has been deployed already; as discussed elsewhere in this application. [10][30] Cf. Table 3.
Term PE: For term PE the thresholds were determined in association with clinicians, and grossly correspond with a 5 fold enrichment compared to the pre-test prevalence in either direction; i.e., the high risk threshold corresponds a ˜5× pre-test probability for being a future PE case; the low risk threshold corresponds a ˜5× pre-test probability for being a future non-PE case. Within this application only prognostic models for term PE are elaborated on. Cf. Table 3

Whereas the above paragraphs set the minimum PPV- (rule-in) for each PE sub-type and/or patient sub-population, the following minimum (future) PE case detection rate is pursued for any of the given PPV-thresholds, i.e., the (future) case detection rate of at least 40% (Sensitivity >=0.4). Similarly, for any of the pre-set NPV- (rule-out) criterions, the following minimum future non-PE cases (or “controls”) detection rate is pursued for any of the given NPV-thresholds, i.e., the future non-case detection rate of at least 30% (Specificity >=0.3).

TABLE 3
PPV-and NPV- based performance targets for prognostic tests for predicting the risk
of Preeclampsia in pregnant women prior to appearance of clinical symptoms of PE.

| Outcome sub-type | Prevalence | Rule-in tests | | Rule-out tests | |
|---|---|---|---|---|---|
| outcome in sub-group | in SCOPE | PPV cut-off | Sensitivity target | NPV cut-off | Specificity target |
| All PE [27] | 0.05 [24] | 1./7.5 = 0.133 | Sₙ >= 0.4 | 1-1/90 = 0.988 | Sₚ >= 0.3 |
| Preterm PE (<37 wks) [30] | 0.014 [24] | 1/14 = 0.0714 | Sₙ >= 0.4 | 1-1/400 = 0.9975 | Sₚ >= 0.3 |
| Term PE (>=37) | 0.037 [24] | 1/6.5 = 0.154 | Sₙ >= 0.4 | 1-1/160 = 0.99375 | Sₚ >= 0.3 |

For the avoidance of doubt, albeit the above performance targets are based on clinical relevance, they are not limiting; different healthcare settings may require for different targets. Furthermore, when novel prophylactic treatment options become available, different targets may be required based on e.g., cost of treatment or/and side-effects.

For the avoidance of doubt, the prognostic combinations of variables, as disclosed in this application, will also be relevant to the prognosis of preeclampsia in women in their 2nd or higher pregnancy. As these multiparous women will have a “pregnancy history”, which will impact on their risk for preeclampsia, it is easily understood that this information, when combined with the findings as disclosed within this application, will enhance the prognostic performances for predicting the risk of preeclampsia occurring in their pregnancies.

### Example 2

### Collection of the Analytical Methods and Statistical Models Applied

### A) the Analytical Methods are Based on the Following

1. The use of an extraction solvent/protein precipitation solvent that enables the extraction of the different types (classes) of metabolites. This extraction solvent composition, being a mixture of Methanol, Isopropanol and 200 mM Ammonium Acetate (aqueous) in a 10:9:1 ratio, which in turn is fortified with 0.05% 3,5-Di-tert-4-butyl-hydroxytoluene; in the remainder of this example this solvent is referred to as the “crash”.
2. The use of a dual (High Pressure) Liquid Chromatography (LC) system to enable the identification and quantification of the different classes of metabolites in a short analytical run. The chromatographic systems were developed so that these could be directly hyphenated to a mass spectrometric detection system. This dual chromatography system allows the separation of different metabolite types/classes and at the same time generate a detectable signal at the level of the mass spectrometer. A single chromatographic system, with short turn-around time, is not effective in robustly generating a detectable signal across all classes. The ability to 1) comprehensively analyse metabolites across different classes of metabolites, as relevant to a prognostic question, in 2) a short turn-around time, is important to generate data on sufficiently large sample sets (necessary to enable statistically robust multivariable models) in economically viable time- and cost-frames.

Typical, but non-limiting examples of LC methods, are detailed below:

### Materials and Reagents Used in the Dual Separations.

LC-MS grade ammonium acetate (NH₄OAc) and ammonium formate (NH₄HCOO) were purchased from Fluka (Arklow, Ireland). LC-MS optima grade acetic acid, acetonitrile (ACN), methanol (MeOH) and 2-Propanol (IPA) were purchased from Fischer scientific (Blanchardstown, Ireland).

For the RPLC, the column-type used was a Zorbax Eclipse Plus C18 Rapid Resolution HD 2.1×50 mm, 1.8-Micron column (P.N. 959757-902; Agilent Technologies, Little Island, Ireland). For the HILIC-MS/MS, the column type was an Ascentis Express HILIC 15 cm×2.1 mm, 2.7 Micron (P.N. 53946-U: Sigma-Aldrich, Arklow, Ireland)

Instrument: The LC-MS/MS platform used consisted of a 1260 Infinity LC system (Agilent Technologies, Waldbronn, Germany). The latter was coupled to an Agilent Triple Quadrupole 6460 mass spectrometer (QqQ-MS) equipped with an JetStream Electrospray Ionisation source (Agilent Technologies, Santa Clara, Calif., USA).

### RPLC:

The RPLC method is defined by the following settings/parameters:
Injection volume: 7 μL
Column oven temperature: 60° C.
Gradient RPLC was performed to resolve the hydrophobic metabolites using a binary solvent system:
   mobile phase A: Water:MeOH:NH₄OAc buffer 200 mM at pH 4.5, (92:3:5)
   mobile phase B: MeOH:Acetonitrile:IPA: NH₄OAc 200 mM at pH 4.5 (35:35:25:5)
A linear gradient program was applied: from 10% mobile phase B to 100% mobile phase B in 10 minutes. using the following gradient—flow rate program:

TABLE 3A

| Time (min) | % Mobile phase A | % Mobile phase B | Flow rate (ml/min) |
|---|---|---|---|
| 0.00 | 100% | 0% | 0.350 |
| 6.00 | 0% | 100% | 0.5 |
| 8.00 | 0% | 100% | 0.5 |
| 8.10 | 100% | 0% | 0.5 |
| 9.00 | 100% | 0% | 0.5 |
| 10.00 | 100% | 0% | 0.350 |

The efflux of the RPLC column was led directly to the QqQ-MS for mass spectrometric determination of the hydrophobic compounds of interest (see below)).

### HILIC:

The HILIC method is defined by the following settings/parameters:
Injection volume: 3 μL, whereby the injection plug was bracketed by 3 μL ACN solvent plugs; a specific injector program was devised for this.
Column oven temperature: 30° C.
Gradient HILIC was performed to resolve the hydrophobic metabolites using a binary solvent system:
   mobile phase A: 50 mM Ammonium formate (aqueous)
   mobile phase B: ACN
A linear step gradient program was applied: from 10% mobile phase B to 100% mobile phase B in 10 minutes. using the following gradient-flow rate program:

TABLE 3B

| Time (min) | % Mobile phase A | % Mobile phase B | Flow rate |
|---|---|---|---|
| 0.00 | 12% | 88% | 0.45 mL/min |
| 1.10 | 20% | 80% | 0.45 mL/min |
| 2.0 | 20% | 80% | 0.45 mL/min |
| 2.10 | 30% | 70% | 0.45 mL/min |
| 3.00 | 30% | 70% | 0.45 mL/min |
| 3.10 | 40% | 60% | 0.45 mL/min |
| 4.00 | 40% | 60% | 0.45 mL/min |
| 6.00 | 50% | 50% | 0.45 mL/min |
| 7.20 | 50% | 50% | 0.45 mL/min |
| 7.21 | 12% | 88% | 0.45 mL/min |
| 10.00 | 12% | 88% | 0.45 mL/min |

The efflux of the RPLC column was led directly to the QqQ-MS for mass spectrometric determination of the hydrophobic compounds of interest (see below)).

3. The use of a form of quantitative mass spectrometry, i.e., a tandem mass spectrometry system (MS/MS) operated in the Multiple Reaction Monitoring modus to allow for sensitive and specific analysis of metabolites. Hereto the samples are subjected to ionization under conditions to produce ionized forms of the metabolites of interest. Then the ionized metabolites are fragmented into metabolite derived fragment ions. The amounts of two specific fragments per metabolite are determined to identify and quantify the amounts of the originator metabolites in the sample (for further detail see below). Tandem mass spectroscopy was carried out under both positive and negative electrospray ionization and multiple reaction monitoring (MRM) mode. For each metabolite of interest, as relevant to preeclampsia, the following parameters were specifically established and optimized for each and every metabolite of interest and each SIL-IS available:
appropriate precursor ion m/z, inclusive its preferred ionization mode (positive or negative),
Product ion spectra under various collision voltage conditions (cf. induction of ion-molecule collisions under different energy regimens, leading to specific product ions) and selection of the most appropriate Quantifier and Qualifier product ions to be used for mass spectrometric identification and quantifications.
Establishment of the reference Quantifier ion/Qualification ion ratios which to serve for specificity assessment.
In addition, a number of assay specific instrument parameters were also optimized per compound of interest: quadrupole resolutions, dwell time, Fragmentor Voltage, Collision Energy and Cell Accelerator Voltage.

At the same time, instrument-specific parameters were optimised to maximally maintain compound integrity in the electrospray source and achieve sensitive and specific metabolite analysis; source temperature, sheath gas flow, drying gas flow and capillary voltage. The mass spectrometer used was an Agilent Triple Quadrupole 6460 mass spectrometer (QqQ-MS) equipped with an JetStream Electrospray Ionisation source (Agilent Technologies, Santa Clara, Calif., USA).

### RPLC-ESI-MS/MS

For the mass spectrometric method used for analyzing the hydrophobic metabolites of interest, the optimized electrospray ionization source parameters were as follows:

TABLE 3C

| Source Parameters | Positive mode | Negative mode |
|---|---|---|
| Gas Temperature, °C. | 200 | 200 |
| Gas flow, l/min | 13 | 13 |
| Nebuliser, psi | 40 | 40 |
| Sheath Gas Heater | 400 | 400 |
| Sheath Gas Flow | 11 | 11 |
| Capillary, V | 5000 | 3000 |
| V Charging | 300 | 300 |

### HILIC-MS/MS:

For the mass spectrometric method used for analyzing the hydrophilic metabolites of interest, the optimized electrospray ionization source parameters were as follows:

TABLE 3D

| | Positive | Negative |
|---|---|---|
| Parameters | mode | mode |
| Gas Temperature, ° C. | 200 | 200 |
| Gas flow, l/min | 13 | 13 |
| Nebuliser, psi | 40 | 40 |
| Sheath Gas Heater | 400 | 400 |
| Sheath Gas Flow | 12 | 12 |
| Capillary, V | 2500 | 3000 |
| V Charging | 300 | 300 |

4. For each metabolite a specific LC-MS/MS assay was developed for each of the targets of interest as well as for each of the SIL-IS; a particular LC-MS/MS assay entails a combination of above points 2&3.
5. To unambiguously identify a metabolite/SIL-IS of interest, each of the assays will constitute a specific set of experimental parameters which will unequivocally identify the compound of interest. It is of note that the values of these experimental parameters are specific to and optimized for the used LC-MS/MS technology. In the case of the LC-MS/MS assays under consideration, this set of specific parameters are the following:
a) Retention time (Rt): The time between the injection and the appearance of the peak maximum (at the detector). The specific retention time is established for each metabolite.
b) Precursor ion m/z: Mass/charge ratio of the ion that is directly derived from the target compound by a charging process occurring in the ionisation source of the mass spectrometer. In this work the precursor ion is most often a protonated [M+H]+ or deprotonated form [M−H]− of the target compound. In some instances, the precursor ion considered has undergone an additional loss of a neutral entity (f.i., a water molecule (H₂O)) in the ionisation source. In some other instances, the ionisation of the compound of interest follows the formation of an adduct between the neutral compound and another ion (f.i, a sodium adduct) available. The appropriate precursor ion is established for each metabolite.
c) Precursor ion charge: The charge of the ion that is directly derived from the target compound by a charging process occurring in the ionisation source of the mass spectrometer, the precursor ion can be either positively charged or negatively charged. The appropriate charge state is established for each metabolite.
d) Quantifier Product ion: Ion formed as the product of a reaction involving a particular precursor ion. The reaction can be of different types including unimolecular dissociation to form fragment ions, an ion-molecule collision, an ion-molecule reaction [31], or simply involve a change in the number of charges. In general, the quantifier product ion is the most intense fragment and/or specific to the compound of interest. The quantifier product ion data is used to quantify the compound of interest. The appropriate quantifier product ion is established for each metabolite and SIL-IS.
e) Qualifier Product ion: Ion formed as the product of a reaction involving a particular precursor ion. The reaction can be of different types including unimolecular dissociation to form fragment ions, an ion-molecule collision, an ion-molecule reaction [31], or simply involve a change in the number of charges. In general, the qualifier product ion is a less intense fragment to the compound of interest. The qualifier product ion data is used as an additional confirmation the LC-MS/MS is specific to the compound of interest. In specific cases, the use of more than one qualifier ions is considered. The appropriate qualifier product ion is established for each metabolite and SIL-IS.
f) Quantifier ion/Qualifier ion ratio (or vice versa): under well-defined tandem mass spectrometric conditions, a precursor ion produced from a compound of interest will dissociate in controlled fashion and generate quantifier product ions and qualifier product ions in predictable proportions. By monitoring the quantifier/Qualifier ratio, one gets additional assurance that the LC-MS/MS is specifically quantifying the compound of interest. The chance that an interference will elute at the same retention time, create the same precursor ion, and dissociate in the same quantifier and qualifier ions in the same proportion as the target of interest is deemed very low. In specific cases, the use of more than one quantifier/Qualifier ratio can be considered. The appropriate Quantifier ion/qualifier ion ratio (or vice versa) is established for each metabolite and SIL-IS.

Availability of the above 6 parameters will define with great certainty a highly specific assay to a compound of interest. In some instances, not all 6 parameters will be available, f.i., when the precursor ion will not dissociate in meaningful product ions.

For these metabolite targets wherefore a structurally identical SIL-IS standard is co-analyzed, one has an additional specificity metric: the metabolite target and the SIL-IS are, apart from their mass, chemically identical, and hence they would have the same retention time. In rare instances, perfect co-elution is not achieved due to a so-called deuterium effect[32].

The specific parameter sets established for exemplary metabolites and associated SIL-ISs across the metabolite classes of interest to the prediction of preeclampsia, together with some instrument specific (but non-limiting) ionization source settings are presented in below Tables 4&5.

TABLE 4
LC-MRM parameters for the hydrophobic metabolites of interest and associated SIL-IS

| | | Quant/ | MS1 m/z | MS2 m/z | Quant/ | Dwell | Frag | CE | CAV | |
|---|---|---|---|---|---|---|---|---|---|---|
| Metabolite | Rt (min) | Qual | (Res) | (Res) | Qual ratio | (ms) | (V) | (V) | (V) | Polarity |
| 25-Hydroxyvitamin D₃ | 6.6 | Quant | 401.3 (Wide) | 383.3 (Wide) | 23.6 | 20 | 104 | 4 | 2 | Positive |
| | | Qual | 401.3 (Wide) | 365.3 (Wide) | | 20 | 104 | 4 | 2 | Positive |
| Arachidonic acid | 6.8 | Quant | 303.1 (Unit) | 259.1 (Unit) | 34.7 | 3 | 135 | 3 | 2 | Negative |
| | | Qual | 303.1 (Unit) | 59.1 (Unit) | | 3 | 135 | 15 | 2 | Negative |
| 8,11,14 | 7.0 | Quant | 305.1 (Unit) | 305.0 (Unit) | 152.7 | 3 | 80 | 1 | 2 | Negative |
| Eicosatrienoic acid | | Qual | 305.1 (Unit) | 304.9 (Unit) | | 3 | 80 | 0 | 2 | Negative |
| Decanoylcarnitine | 4.7 | Quant | 316.1 (Unit) | 60.1 (Unit) | 59.9 | 3 | 190 | 24 | 2 | Positive |
| | | Qual | 316.1 (Unit) | 257.1 (Unit) | | 3 | 190 | 12 | 2 | Positive |
| Dodecanoyl-l- | 5.4 | Quant | 344.1 (Unit) | 85.1 (Unit) | 37.8 | 3 | 140 | 21 | 3 | Positive |
| carnitine (C12) | | Qual | 344.1 (Unit) | 85.0 (Unit) | | 3 | 140 | 51 | 3 | Positive |
| Docosahexaenoic acid | 6.7 | Quant | 327.1 (Unit) | 283.1 (Unit) | 11.8 | 3 | 80 | 5 | 2 | Negative |
| | | Qual | 327.1 (Unit) | 229.1 (Unit) | | 3 | 80 | 5 | 2 | Negative |
| Dilinoleoyl- | 8.8 | Quant | 634.4 (Unit) | 337.5 (Unit) | 185.4 | 3 | 84 | 28 | 2 | Positive |
| glycerol⁺ | | Qual | 634.4 (Unit) | 599.2 (Unit) | | 3 | 84 | 16 | 2 | Positive |
| Hexadecanoic | 7.0 | Quant | 255.1 (Unit) | 255.1 (Unit) | 21.5 | 3 | 130 | 15 | 3 | Negative |
| acid | | Qual | 255.1 (Unit) | 255.0 (Unit) | | 3 | 130 | 20 | 3 | Negative |
| Linoleic acid | 6.9 | Quant | 279.1 (Unit) | 279.1 (Unit) | 13.8 | 3 | 104 | 10 | 2 | Negative |
| | | Qual | 279.1 (Unit) | 279.0 (Unit) | | 3 | 104 | 20 | 2 | Negative |
| Oleic acid | 7.1 | Quant | 281.1 (Unit) | 281.1 (Unit) | 22.3 | 3 | 128 | 10 | 3 | Negative |
| | | Qual | 281.1 (Unit) | 281.0 (Unit) | | 3 | 128 | 20 | 3 | Negative |
| L-Palmitoylcarnitine | 6.3 | Quant | 400.2 (Unit) | 60.2 (Unit) | 34.8 | 3 | 110 | 26 | 2 | Positive |
| | | Qual | 400.2 (Unit) | 341.2 (Unit) | | 3 | 110 | 17 | 2 | Positive |
| Sphingosine-1- | 6.1 | Quant | 380.1 (Unit) | 264.2 (Unit) | 5.9 | 3 | 100 | 11 | 3 | Positive |
| phosphate | | Qual | 380.1 (Unit) | 362.2 (Unit) | | 3 | 100 | 11 | 3 | Positive |
| Sphinganine-1- | 6.2 | Quant | 382.0 (Unit) | 284.0 (Unit) | 43.3 | 3 | 100 | 8 | 3 | Positive |
| phosphate (C18 base) | | Qual | 382.0 (Unit) | 266.0 (Unit) | | 3 | 100 | 12 | 3 | Positive |
| Stearoylcarnitine | 6.7 | Quant | 428.3 (Unit) | 85.0 (Unit) | 2.7 | 3 | 130 | 25 | 5 | Positive |
| | | Qual | 428.3 (Unit) | 369.3 (Unit) | | 3 | 130 | 15 | 5 | Positive |
| Eicosapentaenoic acid | 6.6 | Quant | 301.1 (Unit) | 257.0 (Unit) | 15.1 | 3 | 120 | 5 | 5 | Negative |
| | | Qual | 301.1 (Unit) | 59.2 (Unit) | | 3 | 120 | 15 | 5 | Negative |
| Ricinoleic acid | 6.1 | Quant | 297.2 (Unit) | 183.1 (Unit) | 24.7 | 3 | 120 | 15 | 7 | Negative |
| | | Qual | 297.2 (Unit) | 279.0 (Unit) | | 3 | 120 | 10 | 7 | Negative |
| 13-Oxooctadecanoic | 6.3 | Quant | 299.4 (Unit) | 281.2 (Unit) | 12.5 | 3 | 100 | 5 | 4 | Positive |
| acid | | Qual | 299.4 (Unit) | 111.2 (Unit) | | 3 | 100 | 10 | 4 | Positive |
| 3-Hydroxytetradecanoic | 5.8 | Quant | 243.1 (Unit) | 59.1 (Unit) | 2.5 | 3 | 120 | 2 | 2 | Negative |
| acid | | Qual | 243.1 (Unit) | 41.1 (Unit) | | 3 | 120 | 45 | 2 | Negative |
| Bilirubin | 6.5 | Quant | 585.2 (Unit) | 299.1 (Unit) | 0.7 | 3 | 125 | 20 | 5 | Positive |
| | | Qual | 585.2 (Unit) | 213.1 (Unit) | | 3 | 125 | 45 | 3 | Positive |
| Biliverdin | 5.1 | Quant | 583.2 (Unit) | 297.1 (Unit) | 200.7 | 3 | 135 | 35 | 5 | Positive |
| | | Qual | 583.2 (Unit) | 583.1 (Unit) | | 3 | 135 | 0 | 5 | Positive |
| Etiocholanolone | 7.1 | Quant | 465.2 (Unit) | 465.1 (Unit) | Na | 3 | 135 | 0 | 6 | Negative |
| glucuronide | | Qual | 465.2 (Unit) | 113.0 (Unit) | | 3 | 135 | 35 | 6 | Negative |
| Myristic acid | 6.7 | Quant | 227.2 (Unit) | 227.1 (Unit) | 0.5 | 3 | 145 | 0 | 4 | Negative |
| | | Qual | 227.2 (Unit) | 53.8 (Unit) | | 3 | 145 | 45 | 4 | Negative |
| Stearic acid | 7.4 | Quant | 283.2 (Unit) | 265.0 (Unit) | 6.8 | 3 | 145 | 19 | 2 | Negative |
| | | Qual | 283.2 (Unit) | 45.1 (Unit) | | 3 | 145 | 20 | 2 | Negative |
| 1-oleoyl-2-hydroxy-sn- | 6.4 | Quant | 524.4 (Unit) | 339.1 (Unit) | 23.6 | 3 | 120 | 20 | 4 | Positive |
| glycero-3-phospho-L- | | Qual | 524.4 (Unit) | 506.2 (Unit) | | 3 | 120 | 10 | 4 | Positive |
| serine | | | | | | | | | | |
| 20-Carboxy-leukotriene | 7.4 | Quant | 365.0 (Unit) | 364.9 (Unit) | 0.5 | 3 | 120 | 0 | 5 | Negative |
| B4 | | Qual | 365.0 (Unit) | 195.0 (Unit) | | 3 | 120 | 15 | 5 | Negative |
| 2-Hydroxytetradecanoic | 5.8 | Quant | 243.1 (Unit) | 197.2 (Unit) | 374.6 | 3 | 120 | 15 | 2 | Negative |
| acid | | Qual | 243.1 (Unit) | 243.0 (Unit) | | 3 | 120 | 0 | 2 | Negative |
| 1-Palmitoyl-2-hydroxy- | 6.5 | Quant | 496.2 (Unit) | 104.1 (Unit) | 34.3 | 3 | 120 | 10 | 2 | Positive |
| sn-glycero-3- | | Qual | 496.2 (Unit) | 184.0 (Unit) | | 3 | 120 | 5 | 2 | Positive |
| phosphocholine | | | | | | | | | | |
| (LysoPC(16:0)) | | | | | | | | | | |
| 6-Hydroxysphingosine | 5.6 | Quant | 316.2 (Unit) | 60.1 (Unit) | 12.3 | 3 | 100 | 10 | 7 | Positive |
| | | Qual | 316.2 (Unit) | 280.1 (Unit) | | 3 | 100 | 10 | 7 | Positive |
| Sphinganine-1- | 6.0 | Quant | 368.1 (Unit) | 270.0 (Unit) | 16.7 | 3 | 100 | 10 | 4 | Positive |
| phosphate (C17 base) | | Qual | 368.1 (Unit) | 252.0 (Unit) | | 3 | 100 | 25 | 4 | Positive |

SIL-IS

| | | | | | | | | | | |
|---|---|---|---|---|---|---|---|---|---|---|
| 25- | 6.6 | Quant | 404.2 (Wide) | 386.2 (Wide) | 46.6 | 20 | 98 | 10 | 2 | Positive |
| Hydroxyvitamin | | Qual | 404.2 (Wide) | 368.3 (Wide) | | 20 | 98 | 10 | 2 | Positive |
| D₃-[²H₃] | | | | | | | | | | |
| Arachidonic | 6.8 | Quant | 311.1 (Unit) | 267.1 (Unit) | 36.1 | 3 | 135 | 3 | 2 | Negative |
| acid-[²H₈] | | Qual | 311.1 (Unit) | 59.1 (Unit) | | 3 | 135 | 15 | 3 | Negative |
| Decanoylcarnitine- | 4.7 | Quant | 319.1 (Unit) | 63.1 (Unit) | 46.6 | 3 | 190 | 24 | 2 | Positive |
| [²H₃] | | Qual | 319.1 (Unit) | 257.1 (Unit) | | 3 | 190 | 12 | 2 | Positive |
| Dodecanoyl-1- | 5.4 | Quant | 347.1 (Unit) | 85.1 (Unit) | 29.8 | 3 | 140 | 21 | 3 | Positive |
| carnitine-[²H₃] | | Qual | 347.1 (Unit) | 85.0 (Unit) | | 3 | 140 | 51 | 3 | Positive |
| Docosahexaenoic | 6.7 | Quant | 332.1 (Unit) | 288.1 (Unit) | 12.1 | 3 | 80 | 5 | 2 | Negative |
| acid-[²H₅] | | Qual | 332.1 (Unit) | 234.1 (Unit) | | 3 | 80 | 5 | 2 | Negative |
| 1,3-Dilinoleoyl- | 8.8 | Quant | 639.4 (Unit) | 342.5 (Unit) | 141.1 | 3 | 84 | 20 | 2 | Positive |
| rac-glycerol- | | Qual | 639.4 (Unit) | 604.2 (Unit) | | 3 | 84 | 10 | 2 | Positive |
| [²H₅] | | | | | | | | | | |
| Hexadecanoic | 7.0 | Quant | 259.1 (Unit) | 259.1 (Unit) | 24.1 | 3 | 130 | 15 | 3 | Negative |
| acid-[²H₄] | | Qual | 259.1 (Unit) | 259.0 (Unit) | | 3 | 130 | 20 | 3 | Negative |
| Linoleic acid- | 6.9 | Quant | 297.3 (Unit) | 297.3 (Unit) | 21.5 | 3 | 104 | 10 | 3 | Negative |
| [¹³C₁₈] | | Qual | 297.3 (Unit) | 297.2 (Unit) | | 3 | 104 | 20 | 3 | Negative |
| Oleic acid-[¹³C₅] | 7.1 | Quant | 286.3 (Unit) | 286.3 (Unit) | 24.5 | 3 | 128 | 10 | 3 | Negative |
| | | Qual | 286.3 (Unit) | 286.2 (Unit) | | 3 | 128 | 20 | 3 | Negative |
| Palmitoyl | 6.3 | Quant | 403.2 (Unit) | 63.2 (Unit) | 29.7 | 3 | 190 | 5 | 2 | Positive |
| carnitine-[²H₃] | | Qual | 403.2 (Unit) | 341.2 (Unit) | | 3 | 190 | 0 | 2 | Positive |
| | 6.1 | Quant | 384.2 (Unit) | 268.2 (Unit) | 4.3 | 3 | 100 | 11 | 3 | Positive |
| Sphingosine-1- | | Qual | 384.2 (Unit) | 366.2 (Unit) | | 3 | 100 | 11 | 3 | Positive |
| phosphate- | | | | | | | | | | |
| [¹³C₂, ²H₂] | | | | | | | | | | |
| Stearoyl-L- | 6.7 | Quant | 431.3 (Unit) | 85.0 (Unit) | 2.7 | 3 | 130 | 25 | 5 | Positive |
| carnitine [²H₃] | | Qual | 431.3 (Unit) | 369.3 (Unit) | | 3 | 130 | 15 | 5 | Positive |
| Bilirubin [²H₄] | 6.5 | Quant | 590.2 (Unit) | 301.2 (Unit) | 98.8 | 3 | 125 | 15 | 5 | Positive |
| | | Qual | 590.2 (Unit) | 303.2 (Unit) | | 3 | 125 | 15 | 5 | Positive |
| Biliverdin [²H₄] | 5.1 | Quant | 586.0 (Unit) | 299.2 (Unit) | NA | 3 | 130 | 35 | 5 | Positive |

⁺read-out is a combined signal of 1,3-rac-Dilinoleoyl-glycerol and 1,2-rac-Dilinoleoyl-glycerol

TABLE 5
MRM parameters for the hydrophilic metabolites of interest and associated SIL-IS

| | Rt | Quant/ | MS1 m/z | MS2 m/z | Quant/Qual | Dwell | Frag | CE | CAV | |
|---|---|---|---|---|---|---|---|---|---|---|
| Metabolite | (min) | Qual | (Res) | (Res | ratio | (ms) | (V) | (V) | (V) | Polarity |
| 2-Hydroxybutanoic | 2.3 | Quant | 103.0 (Unit) | 57.2 (Unit) | 14.0 | 15 | 84 | 8 | 4 | Negative |
| acid | | Qual | 103.0 (Unit) | 45.2 (Unit) | | 15 | 84 | 5 | 4 | Negative |
| 2-Methylglutaric | 4.8 | Quant | 145.0 (Unit) | 101.2 (Unit) | 9.4 | 15 | 80 | 8 | 4 | Negative |
| | | Qual | 145.0 (Unit) | 83.2 (Unit) | | 15 | 80 | 12 | 4 | Negative |
| | | Quant^{#} | 101.1 (Unit) | 101.2 (Unit) | 179.1 | 3 | 120 | 2 | 5 | Negative |
| | | Quail^{#} | 101.1 (Unit) | 101.0 (Unit) | | 3 | 120 | 0 | 5 | Negative |
| 3-Hydroxybutanoic | 2.7 | Quant | 103.1 (Unit) | 59.1 (Unit) | 344.9 | 15 | 78 | 8 | 4 | Negative |
| acid | | Qual | 103.1 (Unit) | 103.1 (Unit) | | 15 | 78 | 0 | 4 | Negative |
| Adipic acid | 5.1 | Quant | 145.1 (Unit) | 83.2 (Unit) | 221.7 | 3 | 80 | 12 | 4 | Negative |
| | | Qual | 145.1 (Unit) | 101.2 (Unit) | | 3 | 80 | 8 | 4 | Negative |
| L-Alanine | 4.6 | Quant | 90.1 (Unit) | 90.1 (Unit) | 74.7 | 3 | 62 | 0 | 2 | Positive |
| | | Qual | 90.1 (Unit) | 44.1 1 (Unit) | | 3 | 62 | 8 | 2 | Positive |
| L-Arginine | 6.8 | Quant | 175.0 (Unit) | 116.0 (Unit) | 505.9 | 3 | 82 | 15 | 2 | Positive |
| | | Qual | 175.0 (Unit) | 70.1 (Unit) | | 3 | 82 | 20 | 2 | Positive |
| L-Leucine | 3.4 | Quant | 132.0 (Unit) | 86.2 (Unit) | 19.6 | 3 | 104 | 10 | 4 | Positive |
| | | Qual | 132.0 (Unit) | 44.2 (Unit) | | 3 | 104 | 25 | 4 | Positive |
| Citrulline | 5.0 | Quant | 176.0 (Unit) | 113.0 (Unit) | 261.2 | 3 | 68 | 15 | 5 | Positive |
| | | Qual | 176.0 (Unit) | 70.1 (Unit) | | 3 | 68 | 20 | 5 | Positive |
| Choline | 5.7 | Quant | 104.1 (Unit) | 45.3 (Unit) | 171.5 | 3 | 40 | 27 | 2 | Positive |
| | | Qual | 104.1 (Unit) | 60.3 (Unit) | | 3 | 40 | 17 | 2 | Positive |
| Glycyl-glycine | 5.4 | Quant | 133.1 (Unit) | 30.4 (Unit) | 15.7 | 3 | 58 | 20 | 4 | Positive |
| | | Qual | 133.1 (Unit) | 76.2 (Unit) | | 3 | 58 | 5 | 4 | Positive |
| Homo-L-arginine | 6.8 | Quant | 189.0 (Unit) | 144.2 (Unit) | 2.6 | 3 | 88 | 15 | 2 | Positive |
| | | Qual | 189.0 (Unit) | 57.1 (Unit) | | 3 | 88 | 25 | 2 | Positive |
| L-Isoleucine | 3.6 | Quant | 132.0 (Unit) | 69.2 (Unit) | 22.9 | 3 | 104 | 19 | 2 | Positive |
| | | Qual | 132.0 (Unit) | 57.2 (Unit) | | 3 | 104 | 32 | 2 | Positive |
| L-Methionine | 3.5 | Quant | 150.0 (Unit) | 56.2 (Unit) | 37.0 | 3 | 104 | 16 | 2 | Positive |
| | | Qual | 150.0 (Unit) | 104.1 (Unit) | | 3 | 104 | 14 | 2 | Positive |
| NG-Monomethyl- | 7.2 | Quant | 189.0 (Unit) | 116.2 (Unit) | 180.2 | 3 | 88 | 15 | 2 | Positive |
| L-arginine | | Qual | 189.0 (Unit) | 70.2 (Unit) | | 3 | 88 | 15 | 2 | Positive |
| Asymmetric | 7.9 | Quant | 203.0 (Unit) | 46.2 (Unit) | 188.5 | 3 | 100 | 15 | 4 | Positive |
| dimethylarginine | | Qual | 203.0 (Unit) | 70.1 (Unit) | | 3 | 100 | 18 | 4 | Positive |
| Symmetric | 7.7 | Quant | 203.1 (Unit) | 172.2 (Unit) | 49.3 | 3 | 90 | 10 | 4 | Positive |
| dimethylarginine | | Qual | 203.1 (Unit) | 133.0 (Unit) | | 3 | 90 | 6 | 4 | Positive |
| Taurine | 2.9 | Quant | 126.1 (Unit) | 44.2 (Unit) | 35.7 | 3 | 100 | 20 | 2 | Positive |
| | | Qual | 126.1 (Unit) | 108.0 (Unit) | | 3 | 100 | 10 | 2 | Positive |
| Isobutyrylglycine | 3.0 | Quant | 146.0 (Unit) | 76.2 (Unit) | 136.7 | 3 | 60 | 5 | 7 | Positive |
| | | Qual | 146.0 (Unit) | 43.2 (Unit) | | 3 | 60 | 15 | 7 | Positive |
| Urea | 1.3 | Quant | 61.2 (Unit) | 44.3 (Unit) | 167.3 | 3 | 100 | 10 | 2 | Positive |
| | | Qual | 61.2 (Unit) | 61.2 (Unit) | | 3 | 100 | 10 | 2 | Positive |
| Cotinine | 1.3 | Quant | 177.0 (Unit) | 80.0 (Unit) | 19.7 | 3 | 100 | 25 | 5 | Positive |
| | | Qual | 177.0 (Unit) | 98.1 (Unit) | | 3 | 100 | 20 | 5 | Positive |
| L-(+)-Ergothioneine | 4.8 | Quant | 230.1 (Unit) | 127.0 (Unit) | 47.3 | 3 | 100 | 25 | 2 | Positive |
| | | Qual | 230.1 (Unit) | 186.0 (Unit) | | 3 | 100 | 15 | 2 | Positive |
| L-Acetylcarnitine | 6.1 | Quant | 204.2 (Unit) | 60.1 (Unit) | 377.5 | 3 | 100 | 15 | 4 | Positive |
| | | Qual | 204.2 (Unit) | 85.0 (Unit) | | 3 | 100 | 15 | 4 | Positive |
| L-Lysine | 7.4 | Quant | 146.9 (Unit) | 130.2 (Unit) | 613.4 | 1 | 100 | 20 | 2 | Positive |
| | | Qual | 146.9 (Unit) | 84.2 (Unit) | | 1 | 100 | 2 | 2 | Positive |
| L-Glutamine | 4.8 | Quant | 144.9 (Unit) | 127.0 (Unit) | 31.8 | 3 | 100 | 10 | 2 | Negative |
| | | Qual | 144.9 (Unit) | 108.8 (Unit) | | 3 | 100 | 15 | 2 | Negative |

SIL-IS

| | | | | | | | | | | |
|---|---|---|---|---|---|---|---|---|---|---|
| 2-Hydroxybutyrate- | 2.5 | Quant | 106.0 (Unit) | 59.2 (Unit) | 12.9 | 15 | 84 | 8 | 4 | Negative |
| [²H₃] | | Qual | 106.0 (Unit) | 45.2 (Unit) | | 15 | 84 | 5 | 4 | Negative |
| 2-Methylglutaric acid | | Quant | 147.0 (Unit) | 102.0 (Unit) | 24.8 | 15 | 80 | 10 | 4 | Negative |
| [¹³C₂] | | Qual | 147.0 (Unit) | 84.0 (Unit) | | 15 | 80 | 10 | 4 | Negative |
| 3-Hydroxybutanoic | 2.7 | Quant | 107.0 (Unit) | 107.0 (Unit) | 26.7 | 15 | 78 | 0 | 3 | Negative |
| acid [²H₄] | | Qual | 107.0 (Unit) | 59.1 (Unit) | | 15 | 78 | 8 | 3 | Negative |
| Adipic acid [²H₄] | 4.8 | Quant | 149.0 (Unit) | 105.2 (Unit) | 0.25 | 3 | 80 | 10 | 4 | Negative |
| | | Qual | 149.0 (Unit) | 87.2 (Unit) | | 3 | 80 | 10 | 4 | Negative |
| L-Alanine-[¹³C₃] | 4.6 | Quant | 93.1 (Unit) | 93.1 (Unit) | 94.5 | 3 | 62 | 0 | 2 | Positive |
| | | Qual | 93.1 (Unit) | 46.1 (Unit) | | 3 | 62 | 8 | 2 | Positive |
| L-Arginine-[¹³C₆] | 6.8 | Quant | 181.2 (Unit) | 61.3 (Unit) | 55.6 | 3 | 82 | 12 | 2 | Positive |
| | | Qual | 181.2 (Unit) | 121.1 (Unit) | | 3 | 82 | 12 | 2 | Positive |
| Leucine-[¹³C₆] | 3.4 | Quant | 138.0 (Unit) | 46.2 (Unit) | 10.7 | 3 | 104 | 25 | 4 | Positive |
| | | Qual | 138.0 (Unit) | 44.2 (Unit) | | 3 | 104 | 25 | 4 | Positive |
| L-Citrulline [²H₇] | 5.1 | Quant | 183.1 (Unit) | 120.1 (Unit) | 231.6 | 3 | 68 | 16 | 5 | Positive |
| | | Qual | 183.1 (Unit) | 166.1 (Unit) | | 3 | 68 | 4 | 5 | Positive |
| Choline-[²H₉] | 5.7 | Quant | 114.0 (Unit) | 45.2 (Unit) | 141.3 | 3 | 40 | 20 | 2 | Positive |
| | | Qual | 114.0 (Unit) | 69.2 (Unit) | | 3 | 40 | 20 | 2 | Positive |
| Glycyl-glycine | 5.4 | Quant | 138.9 (Unit) | 79.1 (Unit) | 24.5 | 3 | 58 | 5 | 4 | Positive |
| [¹³C₄, ¹⁵N₂] | | Qual | 138.9 (Unit) | 32.2 (Unit) | | 3 | 58 | 20 | 4 | Positive |
| Homo-L-arginine | 6.8 | Quant | 200.0 (Unit) | 153.0 (Unit) | 717.1 | 3 | 88 | 5 | 2 | Positive |
| [¹³C₇, ¹⁵N₄] | | Qual | 200.0 (Unit) | 90.2 (Unit) | | 3 | 88 | 20 | 2 | Positive |
| Isoleucine-[¹³C₆] | 3.6 | Quant | 138.0 (Unit) | 74.2 (Unit) | 24.6 | 3 | 104 | 19 | 3 | Positive |
| | | Qual | 138.0 (Unit) | 60.2 (Unit) | | 3 | 104 | 32 | 3 | Positive |
| L-Methionine-[¹³C₅] | 3.5 | Quant | 155.0 (Unit) | 59.2 (Unit) | 40.0 | 3 | 104 | 16 | 2 | Positive |
| | | Qual | 155.0 (Unit) | 108.2 (Unit) | | 3 | 104 | 14 | 2 | Positive |
| Asymmetric | 7.9 | Quant | 209.2 (Unit) | 52.3 (Unit) | 175.0 | 3 | 100 | 15 | 4 | Positive |
| dimethylarginine [²H₆] | | Qual | 209.2 (Unit) | 70.2 (Unit) | | 3 | 100 | 20 | 4 | Positive |
| Symmetric | 7.7 | Quant | 209.1 (Unit) | 175.1 (Unit) | 41.9 | 3 | 90 | 10 | 4 | Positive |
| Dimethylarginine-[²H₆] | | Qual | 209.1 (Unit) | 164.0 (Unit) | | 3 | 90 | 15 | 4 | Positive |
| Taurine [¹³C₂] | 2.9 | Quant | 128.1 (Unit) | 46.2 (Unit) | 74.2 | 3 | 102 | 16 | 3 | Positive |
| | | Qual | 128.1 (Unit) | 110.2 (Unit) | | 3 | 102 | 8 | 3 | Positive |
| N- | 3.1 | Quant | 149.0 (Unit) | 43.2 (Unit) | 82.9 | 3 | 60 | 15 | 7 | Positive |
| Iscbutyrylglycine | | Qual | 149.0 (Unit) | 79.1 (Unit) | | 3 | 60 | 5 | 7 | Positive |
| [¹³C₂, ¹⁵N] | | | | | | | | | | |
| Urea [¹³C, ¹⁸O] | 1.3 | Quant | 64.2 (Unit) | 47.2 (Unit) | 469.3 | 3 | 100 | 25 | 2 | Positive |
| | | Qual | 64.2 (Unit) | 64.1 (Unit) | | 3 | 100 | 0 | 2 | Positive |
| (±)-Cotinine [²H₃] | 1.4 | Quant | 180.0 (Unit) | 80.0 (Unit) | 21.7 | 3 | 100 | 25 | 5 | Positive |
| | | Qual | 180.0 (Unit) | 101.0 (Unit) | | 3 | 100 | 20 | 5 | Positive |
| L-(+)- | 4.8 | Quant | 239.0 (Unit) | 127.0 (Unit) | 122.3 | 3 | 100 | 25 | 4 | Positive |
| Ergothioneine [²H₉] | | Qual | 239.0 (Unit) | 195.0 (Unit) | | 3 | 100 | 10 | 2 | Positive |
| L-Acetylcarnitine [²H₃] | 6.1 | Quant | 207.2 (Unit) | 60.1 (Unit) | 853.4 | 3 | 100 | 15 | 4 | Positive |
| | | Qual | 207.2 (Unit) | 85.0 (Unit) | | 3 | 100 | 15 | 4 | Positive |
| L-Glutamine [¹³C₅] | 4.7 | Quant | 149.9 (Unit) | 131.9 (Unit) | 24.8 | 3 | 100 | 10 | 2 | Negative |
| | | Qual | 149.9 (Unit) | 113.8 (Unit) | | 3 | 100 | 15 | 2 | Negative |

^{#}in source fragmentation

6. The use of Stable Isotope Labelled Internal Standards (SIL-IS) to enable Stable Isotope dilution mass spectrometry, to achieve accurate and precise and accurate mass spectrometry-bases compound quantifications [33][34]. In brief, Stable Isotope Dilution Mass spectrometry is based on the principle that one fortifies all study samples with the same volume of a well-defined mixture of SIL-ISs at the start of the analytical process. These SIL-IS are typically identical to the endogenous compounds of interest, in this case metabolites, but have a number of specific atoms (typically Hydrogen 1H, Nitrogen 14N or Carbon 12C) within their molecular structure replaced by a stable, heavy isotope of the same element (typically Deuterium 2H, Nitrogen 15N, Carbon 13C). The SIL-IS are therefore chemically identical but have a different “heavier” mass than their endogenous counterparts. Since they are chemically identical they will “experience” all experimental variability alike the endogenous metabolites of interest. For instance, any differential extraction yield between study samples during sample preparation will equally affect the metabolite of interest and its corresponding SIL-IS. Equally, the metabolite of interest and its corresponding SIL-IS will undergo the same chromatography and are typically equally sensitive to variability during mass spectrometric analysis. As a result, the ratio of any target metabolite signal and its according SIL-IS signal are largely invariant to experimental variability, hence the ratio “metabolite signal/corresponding SIL-IS signal” is directly related to the original concentration of the target in the blood sample. So, in the here disclosed methods, the preferred way to precisely quantify the amount of a metabolite of interest in a sample is by means of establishing the ratio of “the amount of the target metabolite quantifier ion/the amount of the quantifier ion of the corresponding SIL-IS”. Whereby the here disclosed methods allow one to quantify a multitude of different target metabolites in a single analysis of the sample. Moreover, as all study samples are fortified with the same volume of a well-defined mixture of SIL-IS, one can readily compare the levels of the metabolites of interest across all study samples. The SIL-IS are exogenous compounds and thus not to be found in the native biological samples, so their spiked levels act as a common reference for all study samples.
7. The use of specific sample processing protocols for the simultaneous processing of large batches of biospecimens with high reproducibility and low technical variability. The details of a non-limiting example of a fit-for-purpose processing protocol is elaborated below.

As part of the methods, a dedicated biospecimen preparation methodology has been established, involving the fortification of the samples with a relevant SIL-IS mixture, and the use of the “crash”, to extract the metabolites of interest. In terms of sample handling, minimizing any potential sources of error is critical to ensure reliable and precise results. The critical source of error in this methodology relates to the control of volumes; with the most critical volumes being the actual specimen volume being available for analysis, and, the volume of the SIL-IS added. Whereas experienced lab analysts will be able to prepare samples precisely, the use robot liquid handlers, is preferred when processing large numbers of biospecimens is warranted to eliminate human induced technical variability.

Here, as a non-limiting example, we elaborate a dedicated blood processing process, as relevant to methods in this application, using a liquid handling robot.

The robot was configured to enable 96 blood specimens in parallel, using the well-established 96 well format; this is also the analytical batch format adopted for the collection of methods herein.

### Instrument:

Agilent Bravo Automated Liquid Handling Platform (BRAVO, Model 16050-102, Agilent Technologies, Santa Clara, Calif., USA), equipped with, a 96 LT disposable Tip Head, an orbital shaker station and a Peltier Thermal Station (Agilent Technologies). The Robot deck has 9 predefined stations, which can be used for 96 well-plates (specimens, reagents, pipette tip boxes) or functional stations (e.g. Peltier Station, etc)

### Experimental Protocol:

In brief the following steps were performed for each batch of 96 40 μl aliquots; partial batches (n<96) are processed identically:
a) A 96-position plate (8×12 positions, PN:W000059X, Wilmut, Barcelona, Spain) with pre-ordered and 40 μl pre-aliquoted specimens (0.65 ml cryovials, PN:W2DST, Wilmut, Barcelona, Spain), constituting an analytical batch, are retrieved from −80° C. storage, and put on BRAVO deck (orbital shaker) and vortexed for 20 minutes to assist thawing. When thawed, the vials are decapped (manually).
b) In the meantime,
   a. a pre-prepared SIL-IS aliquot is retrieved from −20° C. storage for thermal conditioning, the SIL-IS is then vortexed (1 minute) and-sonicated (5 minutes), and the appropriate volumes are then placed in one column (8 wells) of a PolyPropylene (PP) 96 well plate. The SIL-IS plate is then placed on the BRAVO deck (Peltier at 4° C.).
   b. the pre-prepared proprietary [protein precipitation-metabolite extraction] formulation “crash” stock was taken from −20° C. storage, stirred, and a PP 96 well plate filled with the appropriate volumes, the “crash” plate is then put on the robot deck.
c) The Bravo protocol is then initiated, the critical steps of this process are:
d) Draw up 140 μl of SIL-IS from the filled column of the SIL-IS plate and sequentially dispense 10 μl in each of the specimen vials.
e) Fortified specimens will then be vortexed, on deck, for 5 min at 1200 rpm
f) Addition of the “crash” solution; this part of the sample preparation is performed in two separate steps
   a. First step: addition of 200 μl “crash” solution, followed by on deck vortexing for 1 minute at 1200 rpm,
   b. Second step: addition of 140 μl “crash” solution followed by vortexing for 4 minutes at 1000 rpm
g) The specimen plate is then removed from the BRAVO robot and vortexed at 4° C. for 10 min followed by 2 min sonication
h) Transfer of the specimen plate to the freezer, where they are kept at −20° C. for 20 minutes to maximize protein precipitation.
i) After precipitation, the specimen vials are centrifuged at 4° C. for 20 min at a speed of 8000 rpm, then they are returned to the BRAVO robot; the specimen plate is put on the Peltier station at 4° C.
j) Splitting of the supernatant (i.e., the metabolite extract) in two different aliquots to enable the separate analysis of the Hydrophobic and Hydrophilic compounds. Hereto, 240 μl of supernatant is aspirated and 120 μl dispensed is twice, into separate PP 96-well plates (duplicate “specimen extract” plates).
k) The specimen extract plates are then dried by means of vacuum evaporation at 40° C. for 60 minutes. Typically, 1 dried specimen extract plate is transferred to −80° C. until further analysis, the other specimen extract plate is returned to the BRAVO robot for re-constitution, readying the extracted specimens for LC-MS/MS analysis

Whereas the above exemplified method was applied in the analysis of metabolites of interest relevant to preeclampsia; variations of the above methods are also employed as appropriate for the health outcome under consideration, and associated metabolites of interest. Non-limiting variations include
Pre-treatment of the sample and further extraction of metabolites using solid phase extraction instead of precipitation method; robot protocols are in place.
The consecutive addition of different SIL-IS mixtures, e.g., there are SIL-IS which require different dissolution solvents.
   8. The use of specific Quality Assurance procedures to avoid the introduction of experimental bias and to assure the quality of the quantification of the metabolites of interest. These procedures define for instance: Analytical Batch Size and batch composition, Number and type of Quality Controls Samples, Criteria for acceptance of data read-outs, Operator blinding, designing sufficiently powered studies, selection of the appropriate study samples. To avoid experimental bias, specific methods are used to randomize the study samples. The lack of bias in sample order is then confirmed using the appropriate statistical tests. Upon signal processing of the mass spectrometric data, specific post-analysis Quality methods are applied to assess per metabolite of interest, the data missing-ness rate across a clinical study, the presence of any (unwarranted) experimental bias, eventual signal drift, and the appropriateness of the chosen quantitative read-out (i.e., “metabolite quantifier ion/selected SIL-IS quantifier ion ratio”). Where necessary, alternative quantitative read-outs can be selected. Review of the analyte quantitation is routinely performed to quantify the stability and robustness. In the event, there are some inter-day batch drift observed, an appropriate correction can be applied. The appropriate quantification metric is established for each metabolite of interest. Following quality control and the selection of the most robust quantification metric, the imprecision of each metabolite quantification will be gauged, by calculating coefficients of Variance (% CV), using the available QC samples and/or replicate measurements.
   9. The application of a set of selection criteria (“Quality Stage-Gate criteria”) is used to determine which metabolites of interest can be progressed to biomarker performance analysis. Typically, but non-limiting, precision, specificity and missing ness criteria are considered. Alternatively, imputation of missing values can also be considered [35]. Examples of typical precision limits are e.g., % CV<=15%, or <=20% CV or <=25%. The appropriate Quality Stage-Gate criteria are specifically established for each study of biospecimens, and can vary per metabolite of interest. This step will define which metabolites of interest can be progressed to the next steps and be used in multi-component prognostic/diagnostic test discovery; and will vary per study of biospecimens.
   10. The use of methods to pre-process the quantification data of the metabolites of interest in view of performing biomarker analysis. Typically, these methods involve testing for the need for, and when warranted, application of data transformations (e.g., logarithmic transformation to obtain a normal distribution). Additional methods will test for the need for, and when warranted, the application of corrections of metabolite biomarker read-outs for e.g., patient- or sampling characteristics which modulate the metabolite read-outs independent from the prognostic or diagnostic question under investigation. Typically, but not limiting, the methods involve testing for-, and when warranted, establishing Multiples of the Median of the metabolite quantifications. Correction for such factors seeks to reduce the between-sample/-patient variance. In some instances, it might be relevant to dichotomize or categorize metabolite quantifications. The appropriate data transformations and appropriate corrections are specifically established for each study of biospecimens, and can vary per metabolite of interest.

### a) Log-Transformations:

For the selected metabolites of interest (Example 10), the quantitation read-outs were log-transformed before modelling; with exception for the data as presented in Example 8

### b) Multiple of the Means—Transformations:

The dependencies of each analyte quantitation on common patient characteristics such as clinical center, overweight or gestational age at sampling. The analytes that do show a significant dependency (Mann-Whitney U test, Spearman correlation, Benjamini, Hochberg and Yekutieli, p<0.01) on these factors are normalised using a multiple-of-median (MoM) methodology. Multiple of the mean corrections were applied for the following metabolites of interest:

TABLE 5A

| Variable | Clinical variable |
|---|---|
| TR | sample collection center |
| S-1-P | sample collection center |
| Sa-1-P | sample collection center |
| s-ENG | bmi at time of sampling (log) |
| 1-HD | bmi at time of sampling (log) |
| L-GLU | bmi at time of sampling (log) |
| 2-MGA_GLU | bmi at time of sampling (log) |
| L-ERG | maternal age (log) |
| DHA | maternal age (log) |
| PIGF | gestational age at time of |
| | sampling (log) |

All selected metabolites of interest quantified with the mass spectrometry platform were used as predictors for the computation of predictive models for the disease. For MoM-normalised variables both non-normalised and normalized were considered as predictors for the computation of predictive models for the disease.

### c) Dichotomizing of Data: Cotinine

Cotinine (COT) is an alkaloid found in tobacco and is also the predominant metabolite of nicotine. Cotinine has an in vivo half-life of approximately 20 hours, and is typically detectable for several days (up to one week) after the use of tobacco. The level of cotinine in the blood, saliva, and urine is proportionate to the amount of exposure to tobacco smoke, so it is a valuable indicator of tobacco smoke exposure, including secondary (passive) smoke[36]. Whilst smoking is a risk factor of interest for the prediction of preeclampsia [37], it might be prone to under-reporting. In this study cotinine was analyzed to gauge smoking status and to assess whether it would correlate with reported smoking status within SCOPE. Within the data set under consideration the presence of the cotinine indeed associated with smoking status. The missing rate of the readouts for this analyte was indeed associated with the reported “number of cigarettes per day in the 1st trimester (categories)” (Chi square test, p<0.05).

Analytes that are exogenous such as cotinine, are not quantifiable in many patients. This lack of quantitation is usually associated with the lack of exposure. Therefore, the detectability of the molecule may be a better biomarker than the actual concentration of the molecule in blood. This is the case for cotinine whose presence in blood indicates the inhalation of cigarette smoke. The (relative) quantitation for cotinine was therefore binarized, samples without quantifiable cotinine and samples with low cotinine value were given a score of 0. Samples with high cotinine concentration were given a score of 1. The accuracy to predict whether a patient is reporting smoking was used to define an optimal cotinine relative concentration cutoff. This cutoff corresponds to a low density in the cotinine distribution indicating a robustness in the score.

11. The selection of a specific set of measurements which will be considered as input variables (or putative predictors) in multi-component prognostic/diagnostic test discovery. This set of variables will constitute the pre-processed metabolite quantification data as generated in the previous step, and can be augmented with relevant non-metabolite variables as available for the biospecimens under study. For instance, when one aims to create a multi-component risk stratification test (or prognostic test) to establish the probability that an individual will get (or not get) a medical condition, these non-metabolite variable might constitute, for instance, but not limiting, relevant (clinical) risk factors as collected at time of sampling or as available in (medical) records, or the results of relevant, well-established clinical tests (e.g., glucose measurements) or quantification data of other types of relevant putative biomarkers molecules, e.g., proteins, DNA, RNA, etc as available for the same sample/originator individual. The selection of the appropriate set of non-metabolite variables are specifically established per study and per specific aim of the multi-component prognostic/diagnostic test discovery. Since the Applicants specifically set out to find prognostic tests for preeclampsia which can be easily administered by first line care providers and/or in healthcare systems with limited resources and which are robust, only these clinical risk factors which are well established and easy to obtain were selected. For the same reason, the following types of variables were explicitly and deliberately excluded: patient data which is error prone, for instance but not limited to, detailed life-style variables or detailed medical history data, as well as variables which require for specialized staff to collect them, for instance but not limited to: Uteroplacental blood flow assessment by means of Doppler ultrasound (uterine artery doppler) and its derived parameters like pulsatility index or resistance index. Albeit these uteroplacental blood flow metrics associate with preeclampsia risk, they require for an expert sonographer and advanced ultrasound apparatus to be available. Since the metabolites of interest will typically be determined in a clinical laboratory setting, exemplary variables which can also be assessed in a clinical laboratory environment and which are widely reported to associate with preeclampsia risk were also considered and measurements on 3 specific proteins were therefore selected as additional input variables. Below the non-metabolite inputs as applied in the preeclampsia study are given as a typical, but non-limiting example.
a) 1st sbp: 1st systolic BP at blood sampling visit (mm Hg, sphygmomanometer)
b) 1st dbp: 1st diastolic BP at blood sampling visit (mm Hg, sphygmomanometer)
c) map_1^{st}: 1st MAP (mean arterial pressure) BP at blood sampling visit
d) 2nd_sbp: 2nd systolic BP at blood sampling visit (mm Hg, sphygmomanometer)
e) 2nd_dbp: 2nd diastolic BP at blood sampling visit (mm Hg, sphygmomanometer)
f) map_2^{nd}: 2nd MAP (mean arterial pressure) at blood sampling visit
g) Age: Age of participants
h) fh_pet: Family history of pre-eclamspsia (PE), i.e. participant's mother or sister had had PE
i) wgt: at blood sampling visit (kg)
j) bmi: BMI at blood sampling visit
k) waist: Waist circumference at blood sampling visit (cm)
l) cig_1st_trim_gp: number of cigarettes per day in the 1st trimester (categories) 1-5 cigs/6-10 cigs/>10 cigs
m) qest: gestation at blood sampling visit
n) r_glucose: Random (non-fasting) glucose measured by glucometer at blood sampling visit (mmol/L)

Three well-studied blood-borne protein biomarkers implicated in preeclampsia, i.e.,
o) PIGF: Placental Growth Factor (PIGF, PGF (gene)),
p) sFlt1: Soluble fms-Like Tyrosine Kinase 1 (sFlt1, FLT1(gene)), and
q) s-ENG: soluble Endoglin (s-ENG, ENG (gene))[38].

These protein biomarkers were analysed as part of a large scale assessment of putative protein biomarkers within the SCOPE study using ELISA assays.[24] These 3 proteins were also considered as predictors for the computation of predictive models for the disease.

### B) The Statistical Methods are Based on the Following

### 1. Univariable Analyses:

The use of univariable methods to determine the prognostic and/or diagnostic merits for discriminating, f.i., (future) cases from (future) controls is assessed for all the selected input variables. The methods are not limited to 2 categories. Typically, but not limiting, the area under Receiving Operating Curve (AUROC) is applied to quantify the discriminative performance of each of the selected input variables. Input variables that have a lower limit of the 95% confidence interval of AUROC greater or equal to 0.5 are identified as single biomarkers for the outcome of interest. Other statistical tests like, but not limited to, t-test, Mann-Whitney test, chi-squared test, and Fisher exact test are applied to identify single predictors of interest; whereby a p-value <=0.05 is typically considered significant. When deemed relevant, the methods also consider corrections for multiple testing (e.g., Bonferroni, Holm or Hochberg corrections). Where appropriate, univariable logistic regression is performed to determine whether a selected input variable is a risk factor for the outcome under consideration; hereto odds ratios associated with a unit-increase/decrease in input variable is established. Typically, but non-limiting, these units are expressed in standard deviations (e.g., +/−1SD, +/−2SD, etc. . . . ) or Quantiles. Univariable performances for the variables of interest are presented in Example 3.

### 2. Development of Multivariable Models:

The Applicants realised that the relevance of prognostic classifiers to predict the risk (or probability) an individual will develop a future health condition is largely determined by the extent to which the prognostic merits of such classifiers meet the clinical requirements as identified by health care providers and/or healthcare systems.

Yet, different clinical contexts might mandate for different requirements for a classifier. For instance, some clinical contexts will primarily focus on finding individuals at increased risk for a future health outcome. For these individuals with higher risk, care could be escalated, and/or prophylactic treatment could be prescribed. In other contexts, identifying individuals at decreased risk for the future outcome is more appropriate, e.g., to rationalize the use of certain care pathways. In some instances, both classifier questions will be of interest.

In addition, or alternatively, there might exist different sub-types (or grades) of the future health condition, for instance in terms of outcome severity. The requirements for classifiers might vary in function of outcome sub-type. In addition, or alternatively, there might exist sub-groups of individuals, which exhibit a different a-priori risk profile, and/or are more prone to the outcome or any of its subtypes. Once again, the clinical requirements for classifiers might vary for sub-groups of individuals. For this reason, the Applicants adopted, as part of the methods, the best subset regression method to create the space of all possible multivariable prediction models using one or more multivariable modelling techniques as relevant for the set of input of variables and outcomes (e.g., continuous or categorical) under study. By doing so, one has the possibility to address multiple classifier questions and/or classifier requirement questions at the same time, provided that the study being sufficiently large and representative for the populations of interest and provided that the prognostic information as carried by the input variables, and their combinations, do support the discovery of such classifiers. For instance, for a binary classifier for risk, PLS-DA, logistic regression, fractional polynomials could be applied. Depending on the question at hand and the study size available, the number of variables per model allowed can be varied; typically, but not limiting, the model space is construed of all combinations of e.g., 1 to 3, 1 to 4, 1 to 5 or 1 to 6 variables. In addition, methods are applied to ensure that only statistically robust multivariable classifiers are considered, for instance, by applying cross-validation. A description of the methods used to create the comprehensive model space for the preeclampsia study, are elaborated below.

For each possible combination of one to four predictor variables, a model is trained using known cases and controls using either logistic regression or partial least squares discriminant analysis (PLS-DA) to predict the outcome. Three outcomes models were computed, these are preeclampsia, term preeclampsia and preterm preeclampsia. For the outcomes term preeclampsia, the models were trained and tested on patients that did not develop preeclampsia (controls) versus the patients that developed preeclampsia and delivered at gestation age 37 weeks or higher. For the outcomes preterm preeclampsia, the models were trained and tested on patients that did not develop preeclampsia (controls) versus the patients that developed preeclampsia and delivered at gestation age below 37 weeks. This selection of patients was done to take into account the low prevalence of preeclampsia and the strong over-representation of preeclampsia patients in the dataset studied.

For each model, a range of statistics are derived to estimate its discriminative performance and its clinical relevance. These statistics are:
AUC and (95% CI)
sensitivity at PPV (95% CI)
specificity at NPV (95% CI)
sensitivity at 80% specificity (95% CI)
sensitivity at 90% specificity (95% CI)
specificity at 80% sensitivity (95% CI)
specificity at 90% sensitivity (95% CI)
number of controls (full cases)
number of cases (full cases)
   where CI stands for confidence interval.

### 3. Selection of Robust Prognostic Combinations of Variables/Prognostic Cores:

These statistics are computed for the test set over three iterations of a three-fold cross validation. The mean of each statistic over the three iterations was generated and used for model selection using an improvement criterion as elaborated in next paragraph.

Additionally, all the above statistics are also generated as well as for the complete sample sets. In this later case models were trained and evaluated on all controls and cases. To control for over-fitting, only models where the difference of the respective AUC metrics for the “Mean” (cf. 3-fold cross validation) and the “complete” are <=0.1 are retained within the model space.

Furthermore, models with a lower limit of the 95% confidence (ICI) for the AUC statistic lower than <=0.495 in either the “mean” or “complete” were also purged.

To achieve statistically robust results, the selection of prognostic models/prognostic cores is typically based on an assessment of the lower limit of the 95% confidence (ICI) as calculated using the 3-fold cross validation derived “mean” statistic. Further to ensure that sparse models are selected, the improvement as calculated using the 3-fold cross validation derived “mean” statistic is also used as selection criteria.

For reporting purposes, the statistics as calculated for the complete data sets are used. Indeed, due to the conservative modelling and selection methods used, little to no over-fitting is observed.

### Notes:

Whereas the comprehensive model space was established with either logistic regression or partial least squares discriminant analysis (PLS-DA), only the prognostic models/cores following the PLS-DA were considered for this application (for reporting simplicity). Whereas the statistics for individual prognostic models for logistic regression and PLS-DA may differ, both methods lead to approximately the selection of similar predictor combinations and underlying prognostic predictor cores are found to be largely the same.
For the preeclampsia study considered in this application, the limitation to 4 variables/model is driven by 1) the desire to identify sparse prognostic cores 2) the restricted statistical power for preterm PE, 3) the observation that within the preeclampsia data set exemplified here-in little additional “improvement” is achieved when considering more than 4 variables.

To identify relevant prognostic cores within the comprehensive prognostic model space created, the inventors established a logical rule to estimate the relevance of a model. It is important to evaluate whether each of its constituting input variables is contributing to the model discriminative performance. To estimate this, the minimum difference in performance between the model in question and its parent models is computed for each statistic under consideration. Parent models are all models 1) with fewer variables than the model in question and 2) whose variables are all variables of the model in question. The calculated differences are termed “improvement”. For prognostic core selection purposes, only models with “improvement” above a given positive threshold are considered of relevance. For the preeclampsia study reported herein a range of improvements is applied; abbreviated in the remainder as “Imp”.

### 4. Model Space:

For the preeclampsia study considered in this application (Example 1), models were computed for each possible combination of one to four predictor variables, for each of the 3 outcomes under investigation (see higher). Within the generated PLS-DA model space >256.000 models complied with the basic performance requirements as mentioned in Example 15.

The inventors then set out to discover the non-trivial core combinations of variables, with predictive merits for each of the performance targets as outlined in Example 1. To do so, the model space was filtered using the lower limits of the 95% confidence intervals (ICI) as calculated using the 3-fold cross validation derived “mean” of the relevant statistic and the improvement as calculated using the 3-fold cross validation derived “mean” for the same statistic, for each performance target (AUC, Rule-in, Rule-out) for each of the PE-subtypes (All PE, Preterm PE and Term PE). Filtering thresholds were manually adjusted with a view to yielding a limited set (typically between 20 to 60) of core combinations of 2 to 4 variables (models). This was found sufficient to identify these variables which consistently contribute to performant models. These variables and/or specific combinations thereof, constitute prognostic cores with relevance to the prediction of preeclampsia risk. As elaborated elsewhere in this application, prognostic cores of variables may differ depending on the PE-subtype considered and/or whether generic prognostic performance (AUC), prediction of high-risk (“Rule-in”; Sensitivity at FPR- or PPV-thresholds) or prediction of low-risk (“Rule-out”; Specificity at FNR- or NPV-thresholds) are considered. For reporting, the data of the relevant statistic as achieved within the “complete data set”, which from here on is abbreviated as “complete” when considering filtering thresholds.

### 5. Process Applied for the Identification of Clinically Meaningful Models

### a) Generic Prognostic Performance

Typically, but not limiting, the prognostic performance of (multivariable) classifiers is estimated using the apparent Area Under the Receiver Operating Characteristic (AUROC; also known as the c-statistic) curve. The ROC curve follows the calculation of sensitivity and specificity for all the test values obtained for a classifier within a study. In a ROC curve, the true positive rate (Sensitivity) is plotted in function of the false positive rate (100-Specificity) for different cut-off points of a classifier. Each point on the ROC curve represents a [sensitivity-Specificity] pair corresponding to a particular decision threshold. The area under the ROC curve (AUC) is a measure of how well a parameter can distinguish between two diagnostic groups ((future) cases/(future) non-cases). Sensitivity (Sₙ) is equal to the true positive rate, specificity (Sₚ) is equal to the true negative rate. The AUROC is considered a measure of the performance of a prognostic test, ranging from an area of 0.5 (non-discriminative test, the diagonal) up to 1 (a perfect test with perfect discrimination of future cases and controls). The higher the AUROC, the better a classifier. Within the framework of the methods applied here, the model space will be searched for models which, firstly lead to a robust AUROC equal to or above a pre-set AUROC threshold and secondly maximize the AUROC. In addition, sparse models (constituting a minimal number of variables) are preferred over non-sparse models. This is translated in an additional criterion which determines that a model with (n+1) variables shall have an improved performance, as defined by a specific “improvement” quantum, as compared to any of its parent models with n variables.

Using this process, one can also find “prognostic cores”, i.e., specific combinations of variables with exceptional prognostic merits for the health outcome under consideration: By comprehensively evaluating the constituting variables of the models which are selected by the here-outlined process, one will be able to discern recurrent combinations of variables. Such recurrent combination is considered a “prognostic core”.

Whereas the above collection of methods for the identification of prognostic models and/or prognostic cores involves the study of population(s) of many individuals and multiple characteristics thereof (i.e., variables), the resulting prognostic test has applicability at the level of the single individual. In any individual which is like the individuals in the study population (for instance, in the case of preeclampsia prognosis: the individual is pregnant and exhibits no clinical symptoms of preeclampsia), one can now determine the levels/values of, for example but not limiting, specific non-obvious combinations of blood-borne metabolites as per the identified prognostic model/core, calculate the individuals risk score using the identified prognostic model/core, and translate this risk score into a probability (risk) of the outcome occurring in a specific future timeframe. Examples of AUROC based prognostic cores are presented in Examples 4.

### b) Prognostic Performance—“Rule-In” and “Rule-Out” Tests

Clinical decisions and access to certain clinical care pathways are mostly governed by weighing the benefits versus the costs at the level of the intended-use population. For a so-called “rule-in” test, the benefit of the early detection of risk in those who will develop the disease (true positives) needs to be balanced against the cost of wrongly identifying individuals as being at high risk (false positives). Vice versa, for a “rule-out” test, the benefits of finding true negatives will be weighed against wrongly identifying false negatives as being at low risk.

### b.1) Methods Based on a Classic Interpretation;

Classically, to identify a population at high risk, it is common to lock the false positive rate (FPR, 1-specificity) to a target value and then, for any given classifier, to observe at which sensitivity (detection rate of future cases) the ROC curve crosses the specificity criterion[39][40]. Conversely, to identify a population at low risk, it is common to lock the false negative rate (FNR, 1-sensitivity) to a target value and then, for any given classifier, to observe at which specificity (detection for future non-cases) the ROC curve crosses the FNR criterion to identify a population at low risk. Typically, one will first develop a prognostic model which maximizes AUROC and then establish its estimated detection rate at the set criterion. However, prognostic models with high AUROC are not always the best models when the intended clinical application is either rule-in or rule-out.

Differently the methods elaborated in this application do allow for the identification of prognostic models and/or prognostic cores with exceptional future case detection rates at a pre-set FPR criterion when the clinical application requires for a rule-in prognostic test. Similarly, the methods will enable the identification of prognostic models and/or prognostic cores with exceptional future non-case detection rates at a pre-set FNR criterion when the clinical application requires for a rule-out prognostic test. Within this space, one will then identify those prognostic models and/or prognostic cores which maximize detection rate at the given pre-set criterion rather than merely focusing on AUROC. Significantly, this also supports the notion, as realised by the Applicants, that a specific combination of variables constituting a prognostic test with exceptional rule-in merits, is not necessarily the same combination of variables constituting a prognostic test with exceptional rule-out merits.

### b.2) Methods Based on the State-of-Art Interpretation

The statistics AUROC, Sₙ, and Sₚ are considered prevalence-independent statistics, [41] yet prevalence (or incidence; depending on the application) is important when assessing the clinical usefulness of a prognostic test.[42] When a prognostic test is assessed/applied in its clinically relevant context, metrics like positive and negative predictive value (PPV and NPV), which take the disease prevalence (or incidence) into account, are more appropriate[43]. Here, PPV corresponds the fraction of patients that will actually develop the condition (TP, True Positives) within the group of all patients that have a positive test result (True Positives+False Positives (FP)). NPV corresponds to the fraction of patients that will actually not develop the conditions (TN, True Negatives) within the group of all patients that have a negative test result (True Negatives+False Negatives (FN)). It is easily understood that predictive values are important determinants of the performance of a classifier, as it allows quantifying the “cost” associated with a change of clinical pathway following a prognostic test result. For illustrative purposes, consider the following hypothetical scenario. If the total monetary cost (and/or health cost as a result of for instance undesired side effects) of an available prophylactic treatment is high, a health care system might determine, based on a cost-benefit analysis, that it can support treatment of a high-risk group with a 1:5 chance of developing the condition (i.e., where minimally 1 in 5 will effectively develop the condition (and warrants treatment) and maximally 4 in 5 are false positives, and hence will be needlessly offered the treatment). This criterion translates to a prognostic test which should select a high-risk group with a PPV=0.2.

In this scenario, a test which classifies 50% of future cases into a high-risk group with a PPV=0.2 is considered better (from the health economics point of view) than a test which classifies 75% of future cases in a high-risk group with a PPV=0.1. The latter would amount to a “cost” of 9 False Positives per True Positive, which would be deemed not fit-for-purpose by the health care system where the cost of the prophylactic treatment is high. This also limits the utility of ROC curve analysis as widely applied for the assessment of prognostic tests.

To overcome this, the Applicants have developed statistical methodology to seamlessly link these two views upon prognostic test performances: the ability to plot PPV or NPV criteria, which account for prevalence, in the Receiver Operating Characteristic (ROC) space [27]. This novel methodology was published in November 2017, and can be considered the state-of-the art; therefore this methodology is considered in its entirety an integral part of this application[27].

By integrating this novel statistical methodology, the methods outlined in this application are specifically suited for the identification of prognostic models and/or prognostic cores with clinical utility. They enable the identification of prognostic models and/or prognostic cores with exceptional future case detection rates at a pre-set PPV criterion, when the clinical application requires for a rule-in prognostic test which controls the proportion of false positives.

Similarly, the methods will enable the identification of prognostic models and/or prognostic cores with exceptional future non-case detection rates at a pre-set NPV criterion when the clinical application requires for a rule-out prognostic test which controls the proportion of false positives. Again, the methods capitalize on the creation of the comprehensive prognostic model space and the application of specific success criteria therein. Within the model space one will then identify those prognostic models and/or prognostic cores which maximize detection rate at the given pre-set predictive value criterion rather than merely focusing on AUROC. Significantly, this also supports the notion, as realised by the inventors, that a specific combination of variables constituting a prognostic test optimised for a given PPV criterion, is not necessarily the same combination of variables constituting a prognostic test optimised for a given NPV criterion.

By extension, prognostic models and/or prognostic cores which are optimised for a given PPV criterion for a rule-in test do not necessarily constitute the same variables as prognostic models and/or prognostic cores for a rule-in test which is optimised for a given FPR criterion. The same holds true for rule-out test (NPV criterion vs. FNR criterion). Preferred rule-in cores for preeclampsia are considered in the Examples 5; preferred rule-out cores elaborated in Examples 6.

Whereas the above collection of methods for the discovery of specific rule-in (or rule-out) prognostic models and/or prognostic cores involves the study of populations of many individuals and multiple characteristics thereof (i.e., variables), the resulting prognostic test has applicability at the level of the single individual. In any individual which is like the individuals in the study population, one can determine the levels/values of specific variables as per the identified prognostic model/core, and calculate the individuals risk score using the identified rule-in (or rule-out) prognostic model/core. Then, one will assess whether this risk score is higher or lower than a pre-specified threshold, whereby this threshold delineates the classification in “test-positive” or “test-negative”, in accordance with the rule-in (or rule-out) classification established using the collection of methods elaborated in this application. As a result, one will be able to determine for any such individual, if he/she will be at increased risk (high-risk) of the outcome occurring in a specific future timeframe (rule-in), or if he/she will be at decreased risk (low-risk) of the outcome occurring in a specific future timeframe (rule-out).

### b.3) Methods Based on “Beyond State of the Art”

The above paragraphs and clearly show that methods which integrate the use of PPV and/or NPVcriterions to find prognostic models, and/or prognostic cores, are particularly suited to identify prognostic tests with clinically meaningful prognostic performance.

When the clinical requirement mandates for a prognostic test requires for a rule-in test with a specific PPV, the paired [Sensitivity and Specificity] requirements for such test to comply with said specific PPV target become progressively more stringent with decreasing outcome prevalence (or incidence); to the point where the existence of such classifier becomes improbable. At the same time, it can be appreciated that in clinical practice prognostic rule-in tests have the most utility for health outcomes with low prevalence (incidence) in the population of interest.

Conversely, when the clinical requirement mandates for a prognostic test requires for a rule-out test with a specific NPV, the paired [Sensitivity and Specificity] requirements for such test to comply with said specific NPV target become progressively more stringent with increasing outcome prevalence (or incidence). At the same time, it can be appreciated that in clinical practice, prognostic rule-out tests have the most utility for outcomes which are quite common in the population of interest.

Confronted with this problem, which essentially impedes the development of clinically meaningful prognostic tests for many health outcomes, the inventors came up with a process which can overcome this impediment.

### Process Applicable to Rule-in Prognostic Tests:

Given the clinical need for a prognostic rule-in test delivering a minimal detection rate for future cases (S_{n,test}>=S_{n,target}), for a pre-set PPV threshold (PPVₜₑₛₜ>=PPV_{threshold}) for an health outcome with low incidence (or prevalence), the process involves the following discrete steps to enable the establishment of such prognostic performance:
1) Creation of a first comprehensive model space (Model-S₁) of possible prognostic models for a given study population (Study-Pop₁) using the methods as described earlier.
2) Definition of a “permissible” rate (FNR_{permissible}) or proportion (NPV_{permissible}) of future cases which can be misclassified as low-risk.
3) Identification of prognostic rule-out models and/or prognostic cores, in Model-S₁ which maximize the specificity Sₚ (or detection rate of future non-cases) compliant with the rule-out criterion ((FNR_{permissible}) or (NPV_{permissible})) as defined in the previous step.
4) Selection of an appropriate rule-out model (classifier_{Rule-out}) as identified in the previous step and apply it to the study population (Study-Pop₁); this will result in the creation of a defined low-risk population (Pop_{LR}), complying with a pre-specified number (FNR_{permissible}) or proportion (NPV_{permissible}) of false negatives and a significant fraction of the true negatives, i.e., future non-cases. Purge this population from the initial study population to generate a novel study population (Study-Pop₂), where (Study-Pop₁)−(Pop_{LR})=(Study-Pop₂). It is of note that, compared to the initial Study-Pop₁, the new study Study-Pop₂ will have a higher incidence (or prevalence) of future cases.
5) Creation of a second comprehensive model space (Model-S₂) of possible prognostic models for the novel study population (Study-Pop₂) using the methods as described earlier.
6) Identification of prognostic rule-in models and/or prognostic cores, in Model-S₂ which maximize the sensitivity Sₙ (or detection rate of future cases) compliant with the threshold PPV criterion (PPV_{threshold}). As the incidence (or prevalence) in Study-Pop₂ is enriched in future cases compared to the Study-Pop₁, it is much easier to find a classifier which meets the paired [Sensitivity and Specificity] requirements required to comply with said specific PPV_{threshold}.
7) Selection of an appropriate rule-in model (classifier_{Rule-in}) as identified in the previous step which delivers a detection rate for future cases which, after correction for the permissible misclassifications (cf. steps 2 and 3), meets the target sensitivity, i.e., S_{n,test}>=S_{n,target}.

The outcome of this process is a specific pair of prognostic models (or prognostic cores), i.e., a specific rule-out model and a specific rule-in model which, when applied jointly and sequentially will deliver exceptional rule-in prognostic performance, in accordance with a clinical requirement for a prognostic rule-in test.

The outlined process to achieve clinically relevant prognostic rule-in performance forms integral part to the methods regarding the identification of non-obvious prognostic combinations as elaborated within this application. Exemplary combinations of rule-out/rule-in cores for preeclampsia are disclosed in the Examples 7 later in this application.

### Process Applicable to Rule-Out Prognostic Tests

Given the clinical need for a prognostic rule-out test delivering a maximum detection rate for future non-cases (S_{p,test}>=S_{p,target}), for a pre-set NPV threshold (NPVₜₑₛₜ>=NPV_{threshold}) for an health outcome with moderate to high incidence (or prevalence), the process involves the following discrete steps to enable the establishment of such prognostic performance:
1) Creation of a first comprehensive model space (Model-S₁) of possible prognostic models for a given study population (Study-Pop₁) using the methods as described earlier.
2) Definition of a “permissible” rate (FPR_{permissible}) or proportion (PPV_{permissible}) of future non-cases which can be misclassified as high-risk.
3) Identification of prognostic rule-in models and/or prognostic cores, in Model-S₁ which maximize the specificity Sₙ (or detection rate of future cases) compliant with the rule-in criterion ((FPR_{permissible}) or (PPV_{permissible})) as defined in the previous step.
4) Selection of an appropriate rule-in model (classifier_{Rule-in}) as identified in the previous step and apply it to the study population (Study-Pop₁); this will result in the creation of a defined high-risk population (Pop_{HR}), constituting a pre-specified number (FPR_{permissible}) or proportion (PPV_{permissible}) of false positives and a significant fraction of the true positives, i.e., future cases. Purge this population from the initial study population to generate a novel study population (Study-Pop₂), where (Study-Pop₁)−(Pop_{HR})=(Study-Pop₂). It is of note that, compared to the initial Study-Pop₁, the new study Study-Pop₂ will have a higher incidence (or prevalence) of future non-cases.
5) Creation of a second comprehensive model space (Model-S₂) of possible prognostic models for the novel study population (Study-Pop₂) using the methods as described earlier.
6) Identification of prognostic rule-out models and/or prognostic cores, in Model-S₂ which maximize the specificity Sₚ (or detection rate of future non-cases) compliant with the threshold NPV criterion (NPV_{threshold}). As the incidence (or prevalence) in Study-Pop₂ is enriched in future non-cases compared to the Study-Pop₁, it is much easier to find a classifier which meets the paired [Sensitivity and Specificity] requirements required to comply with said specific NPV_{threshold}.
7) Selection of an appropriate rule-out model (classifier_{Rule-}out) as identified in the previous step which delivers detection rate for future non-cases which, after correction for the permissible misclassifications (cf. steps 2 and 3), meets the target specificity, i.e., S_{p,test}>=S_{p,target}.

The outcome of this process is a specific pair of prognostic models (or prognostic cores), i.e., a specific rule-in model and a specific rule-out model which, when applied jointly and sequentially will deliver exceptional rule-out prognostic performance.

Significantly, these elaborated stepwise processes capitalize on the notion, as realised by the inventors, that a specific combination of variables constituting a prognostic test with exceptional rule-out merits, is not necessarily the same combination of variables constituting a prognostic test with exceptional rule-out merits. Moreover, the characteristics of a transient population (Study-Pop₂), following removal of specific sets of individuals, will be different compared to the initial test-population (Study-Pop₁). As a result, the prognostic models within the respective comprehensive model spaces, i.e., model-S₁ and model-52) may be different.

Whereas the above collection of methods for the discovery of specific combination of 1) a rule-in prognostic model and/or prognostic core and 2) a rule-out prognostic model and/or prognostic core (or vice versa), involves the study of populations of many individuals and multiple characteristics thereof (i.e., variables), the resulting prognostic test has applicability at the level of the single individual. In any individual which is like the individuals in the study population, one can determine the levels/values of specific variables as per the first identified prognostic model/core, and calculate the individuals risk score using the identified rule-out (or rule-in) prognostic model/core. Then, one will assess whether this risk score is higher or lower than a pre-specified threshold, whereby this threshold delineates the classification in “test-positive” or “test-negative”, in accordance with the rule-in (or rule-out) classification established using the collection of methods elaborated in this application. In the event that the individual is classified as “test-negative”, one can determine the levels/values of specific variables as per the second identified prognostic model/core, and calculate the individuals risk score using the identified rule-in (or rule-out) prognostic model/core. By executing these two consecutive steps, one can determine for any such individual, if he/she will be at increased risk (high-risk) of the outcome occurring in a specific future timeframe (rule-in), or if he/she will be at decreased risk (low-risk) of the outcome occurring in a specific future timeframe (rule-out).

It is of note that the variables relevant to the two independent classifiers can be determined in a single analysis, and their levels/values used for classification when appropriate. Likewise, calculating the consecutive risk scores, “test-positive”/“test negative” delineations, and final risk classification, i.e., being at high-risk (rule-in) or being at low-risk (rule-out) can be executed in a single calculation process.

### b.4) Prognostic Performance—Performance Maximisation by Process of Sequential Classifiers.

In some instances, and in view of achieving targets as pertinent to meeting clinical utility requirements, the inventors found that a further expansion of the above concept can deliver exceptional prognostic performance.

Given the clinical need for a prognostic test delivering a maximum detection rate of future cases (S_{n,test_maximal}), for a pre-set PPV threshold (PPVₜₑₛₜ>=PPV_{threshold}) or (/and), a prognostic test delivering a maximum detection rate of future non-cases (S_{p,test_maximal}), for a pre-set NPV threshold (NPVₜₑₛₜ>=NPV_{threshold}) for an health outcome with low incidence (or prevalence), the process involves, the following discrete steps to enable the establishment of such prognostic performance:
1) The creation of a first comprehensive model space (Model-S₁) of possible prognostic models for a given study population (Study-Pop₁) using the methods as described earlier.
2)
   a. Identify a classifier within Study-Pop₁ which either identifies a sub-population at high-risk (Pop_{HR1}) compliant with the PPV-threshold (progress to 3.a)), or
   b. Identify a classifier within Study-Pop₁ which identifies a sub-population at low-risk (Pop_{LR1}) compliant with the NPV-threshold (progress to 3.b))
3)
   a. Purge this population from the initial study population to generate a novel study population Study-Pop₂, where (Study-Pop₁)−(Pop_{HR1})=(Study-Pop₂). It is of note that, compared to the initial Study-Pop₁, the new study Study-Pop₂ is effectively enriched in future non-cases. In the event the latter Study-Pop₂ population meets the pre-set NPV criterion also, the classification process is halted as the pre-set goals are met. Otherwise, progress to the next step.
   b. Purge this population from the initial study population to generate a novel study population (Study-Pop₂), where (Study-Pop₁)−(Pop_{LR1})=(Study-Pop₂). It is of note that, compared to the initial Study-Pop₁, the new study Study-Pop₂ is effectively enriched in future cases. In the event the latter Study-Pop₂ population meets the pre-set PPV criterion also, the classification process is halted as the pre-set goals are met. Otherwise, progress to the next step.
4) The creation of a second comprehensive model space (Model-S₂) of possible prognostic models for the novel study population (Study-Pop₂) using the methods as described earlier.
5)
   a. Identify a classifier within Study-Pop₂ which either identifies a sub-population at high-risk (Pop_{HR2}) compliant with the PPV-threshold and add this novel sub-population to Pop_{HR1}, to create a Pop_{HR_total} (progress to 6.a)), or
   b. Identify a classifier within Study-Pop₂ which identifies a sub-population at low-risk (Pop_{LR2}) compliant with the NPV-threshold and add this novel sub-population to Pop_{LR1}, to create a Pop_{LR_total} (progress to 6.b))
6)
   a. Purge this population from the (Study-Pop₂) population to generate a novel study population (Study-Pop₃), where (Study-Pop₂)−(Pop_{HR}2)=(Study-Pop₃). It is of note that, compared to the Study-Pop₂, the new study Study-Pop₃ is effectively enriched in future non-cases. In the event the latter Study-Pop₃ population meets the pre-set NPV criterion also, the classification process is halted as the pre-set goals are met. Otherwise, progress to the next step.
   b. Purge this population from the initial study population to generate a novel study population (Study-Pop₃), where (Study-Pop₂)−(Pop_{LR2})=(Study-Pop₃). It is of note that, compared to Study-Pop₂, the new study Study-Pop₃ is effectively enriched in future cases. In the event the latter Study-Pop₃ population meets the pre-set PPV criterion also, the classification process is halted as the pre-set goals are met. Otherwise, progress to the next step.
7) Repeat the steps 4 to 6, till maximum detection rate of future cases (S_{n,test_maximal}), for a pre-set PPV threshold (PPVₜₑₛₜ>=PPV_{threshold}) or (/and), maximum detection rate of future non-cases (S_{p,test_maximal}), for a pre-set NPV threshold (NPVₜₑₛₜ>=NPV_{threshold}) as achievable using the variables available for the individuals under study.
   The outcome of this process is a specific Total Classifier, which is made up of a set of prognostic models (or prognostic cores) which, when applied jointly and sequentially will deliver exceptional rule-in or/and rule-out prognostic performance, in accordance with pre-set clinical requirements for risk classification.
   It is of note that, in application of this process, one can either select for a rule-in classifier or a rule-out classifier at any point; in other words, to achieve the desired prognostic classification performance, one shall always apply the best classifier and hence possibly but not necessary, alternate between rule-in and rule-out classifiers.
   Furthermore, to comply with the pre-set PPV and/or NPV requirements, it is sufficient that the respective final Pop_{HR_total}=Pop_{HR1}+Pop_{HR2}+ . . . +Pop_{HRn} and/or Pop_{LR2_total}=Pop_{LR1}+Pop_{LR2}+ . . . +Pop_{LRn} comply with the pre-set criterions; the interim classifications can deviate from the set thresholds.

The outlined process to achieve clinically relevant prognostic rule-in performance forms an integral part of the methods regarding the identification of non-obvious prognostic combinations as elaborated within this application. Preferred combinations of multiple prognostic classifiers for preterm preeclampsia will be disclosed in example 8 later in this application.

Whereas the above collection of methods for the discovery of a specific combinations of prognostic models involves the study of populations of many individuals and multiple characteristics thereof (i.e., variables), the resulting total prognostic test has applicability at the level of the single individual. In any individual which is like the individuals in the study population, one can determine the levels/values of specific variables as per the first identified prognostic model/core, and calculate the individuals risk score using the identified prognostic model/core. Then, one will assess whether this risk score is higher or lower than a pre-specified threshold, whereby this threshold delineates the classification in “test-positive” or “test-negative”, in accordance with the classification (rule-in or rule-out) established using the collection of methods elaborated in this application. When the individual is classified “test-positive”, the corresponding result will be reported (either the individual is classified as high-risk or low-risk, depending on the classifier applied). In the event, the individual is classified as “test-negative”, one can determine the levels/values of specific variables as per the second identified prognostic model/core, and calculate the individuals risk score using the identified rule-in (or rule-out) prognostic model/core. Then, one will assess whether this risk score is higher or lower than a pre-specified threshold, whereby this threshold delineates the classification in “test-positive” or “test-negative”, in accordance with the classification (rule-in or rule-out) established using the collection of methods elaborated in this application. When the individual is classified “test-positive” in this 2^{nd} step, the corresponding result will be reported (either the individual is classified as high-risk or low-risk, depending on the classifier applied). In the event, the individual is classified as “test-negative”, one can determine the levels/values of specific variables as per the third identified prognostic model/core, and calculate the individuals risk score using the identified rule-in (or rule-out) prognostic model/core, etc. This will be repeated till such time the individual is classified in a “test-positive” group or till one has calculated for the individual a risk scores for each of the classifiers constituting the “total classifier”. At that time, the individual will be either triaged as being high-risk or low-risk, or remain un-classified with regards to the pre-set PPV- or/and NPV-criteria.

In the event that the clinical requirements stipulate that a prognostic test should consider both the PPV and NPV criterions at the same time, individuals who remain unclassified with regards to the pre-set PPV- and NPV-criterions, are considered unclassified.

It is of note that the variables relevant to the n sequential classifiers can be measured in a single analysis, and their levels/values used for classification when appropriate. Likewise, calculating the consecutive risk scores, “test-positive”/“test negative” delineations, and final risk classification, i.e., being at high-risk (rule-in), being at low-risk (rule-out) or, unclassified (in the event of a combined [rule-in-rule-out] criterion) can be executed in a single calculation process.

### Example 3—Univariable Performance

For the pre-eclamspsia example elaborated in this application, non-limiting tables with univariable performance metrics of all variables considered are presented here. It will be clear from the below tables that depending on the pre-eclampsia type targeted different variables have prognostic relevance. This observation supports the approach as put forward by the inventors in this application.

### Single Marker Prognostic Performance for Pre-Eclampsia Based on AUC

For each of the pre-eclampsia types considered herein, i.e. “all pre-eclampsia” (all PE), “preterm PE” and “term PE”, tables summarizing AUC (95% C1) and fold changes (FC; 95% C1) are presented. Only the variables that had a lower limit of the 95% confidence interval of AUROC greater or equal to 0.50 were selected as predictive (single) markers each of the pre-eclampsia outcomes studied.

### All PE:

TABLE 6
All PE: AUG - based univariable prognostic performance assessment.
FC: fold change, ICI and uCI:
lower and upper limit of the 95% confidence interval.

| | All PE | | | | | |
|---|---|---|---|---|---|---|
| | | AUC | AUC | fold changes | FC | |
| predictors | AUC | ICI | uCI | PE vs Ctrls | ICI | FC uCI |
| 2nd_sbp | 0.67 | 0.61 | 0.73 | 1.06 | 1.04 | 1.09 |
| map_2nd | 0.67 | 0.61 | 0.73 | 1.06 | 1.04 | 1.08 |
| 1st_sbp | 0.66 | 0.59 | 0.72 | 1.06 | 1.02 | 1.09 |
| map_1st | 0.64 | 0.58 | 0.71 | 1.05 | 1.03 | 1.08 |
| 2nd_dbp | 0.64 | 0.58 | 0.70 | 1.06 | 1.03 | 1.09 |
| bmi | 0.64 | 0.57 | 0.70 | 1.08 | 1.04 | 1.13 |
| waist | 0.62 | 0.55 | 0.68 | 1.05 | 1.02 | 1.08 |
| 1st_dbp | 0.62 | 0.55 | 0.68 | 1.04 | 1.00 | 1.08 |
| DLG | 0.61 | 0.55 | 0.67 | 1.23 | 1.09 | 1.37 |
| wgt | 0.61 | 0.54 | 0.67 | 1.07 | 1.03 | 1.11 |
| 1-HD | 0.61 | 0.54 | 0.67 | 0.89 | 0.83 | 0.95 |
| PIGF_MoM | 0.61 | 0.54 | 0.68 | 0.71 | 0.57 | 0.86 |
| PIGF | 0.60 | 0.53 | 0.67 | 0.71 | 0.57 | 0.89 |
| ADMA | 0.59 | 0.53 | 0.65 | 1.04 | 1.01 | 1.08 |
| s-ENG_MoM | 0.59 | 0.52 | 0.65 | 1.12 | 1.03 | 1.23 |
| DC | 0.59 | 0.52 | 0.65 | 1.29 | 1.06 | 1.57 |
| 2-HBA | 0.58 | 0.52 | 0.65 | 1.12 | 1.03 | 1.23 |
| L-ISO | 0.58 | 0.51 | 0.64 | 1.08 | 1.01 | 1.15 |
| EPA | 0.57 | 0.51 | 0.64 | 1.23 | 1.03 | 1.46 |
| DGLA | 0.57 | 0.51 | 0.63 | 1.13 | 1.01 | 1.26 |
| fh_pet | 0.55 | 0.51 | 0.58 | na | na | na |
| ECG | 0.57 | 0.51 | 0.63 | 1.10 | 1.01 | 1.20 |
| PALMA | 0.57 | 0.51 | 0.63 | 1.14 | 1.01 | 1.30 |
| 12CAR | 0.57 | 0.51 | 0.64 | 1.20 | 1.02 | 1.40 |
| CL | 0.57 | 0.50 | 0.63 | 1.06 | 1.00 | 1.12 |
| 1-HD_MoM | 0.57 | 0.50 | 0.63 | 0.94 | 0.88 | 1.00 |
| NGM | 0.56 | 0.50 | 0.63 | 1.05 | 1.00 | 1.10 |
| LINA | 0.56 | 0.50 | 0.62 | 1.18 | 0.99 | 1.43 |
| OLA | 0.56 | 0.50 | 0.62 | 1.18 | 0.99 | 1.43 |
| 3-HBA | 0.56 | 0.50 | 0.63 | 1.15 | 0.33 | 1.33 |
| s-ENG | 0.56 | 0.50 | 0.63 | 1.09 | 1.00 | 1.19 |
| 16CAR | 0.56 | 0.50 | 0.62 | 1.06 | 0.99 | 1.13 |

When prognostic performance is solely assessed by AUC, it can be observed that classic clinical factors have favorable single-variable prognostic merits for predicting all PE, yet a significant number of metabolites of interest also show prognostic merits. From a clinical-analytical point of view, the fold changes are of importance. Taking this into account, the following 1^{st} tier metabolites are found as being relevant to the prognosis of “all-PE” risk (as assessed by AUC); in order of relevance: DLG, 1-HD.

### Preterm-PE:

TABLE 7
Preterm PE: AUG - based univariable prognostic
performance assessment

| | Preterm PE | | | | | |
|---|---|---|---|---|---|---|
| | | AUC | AUC | fold changes | FC | FC |
| predictors | AUC | ICI | uCI | PE vs Ctrls | ICI | uCI |
| PIGF_MoM | 0.74 | 0.62 | 0.86 | 0.42 | 0.28 | 0.64 |
| PIGF | 0.73 | 0.61 | 0.85 | 0.43 | 0.29 | 0.65 |
| DLG | 0.70 | 0.59 | 0.82 | 1.45 | 1.18 | 1.78 |
| s-ENG_MoM | 0.68 | 0.57 | 0.79 | 1.26 | 1.08 | 1.49 |
| s-ENG | 0.65 | 0.54 | 0.77 | 1.22 | 1.44 | 1.04 |
| bmi | 0.65 | 0.54 | 0.75 | 1.08 | 1.01 | 1.15 |
| CL | 0.61 | 0.50 | 0.72 | 1.09 | 0.99 | 1.20 |
| 2-HBA | 0.62 | 0.50 | 0.73 | 1.16 | 0.99 | 1.37 |
| NGM | 0.61 | 0.50 | 0.72 | 1.08 | 0.99 | 1.18 |
| fh_pet | 0.58 | 0.50 | 0.67 | na | na | na |

When prognostic performance is assessed by AUC, it can be observed that classic clinical factors have limited single-variable prognostic merits for predicting preterm PE, yet a number of proteins and metabolites of interest show significant prognostic merits. Taking AUC and “fold changes” into account, the following 1^{st} tier metabolites are found as being relevant to the prognosis of “preterm-PE” risk (as assessed by AUC); in order of relevance: DLG, 2-HBA. Based on these results, it is clear that DLG is highly prognostic to preterm PE, akin to the best-known protein marker, PIGF.

### Term-PE:

TABLE 8
Term PE: AUC - based univariable prognostic
performance assessment

| | Term PE | | | | | |
|---|---|---|---|---|---|---|
| | | AUC | AUC | fold changes | FC | FC |
| predictors | AUC | ICI | uCI | PE vs Ctrls | ICI | uCI |
| map_2nd | 0.69 | 0.63 | 0.76 | 1.07 | 1.04 | 1.10 |
| 2nd_sbp | 0.69 | 0.63 | 0.76 | 1.07 | 1.05 | 1.10 |
| 1st_sbp | 0.68 | 0.61 | 0.75 | 1.08 | 1.04 | 1.10 |
| 1st_vst_map_1st | 0.67 | 0.60 | 0.74 | 1.07 | 1.04 | 1.10 |
| 2nd_dbp | 0.66 | 0.59 | 0.73 | 1.07 | 1.03 | 1.10 |
| 1st_dbp | 0.64 | 0.57 | 0.71 | 1.06 | 1.03 | 1.09 |
| bmi | 0.63 | 0.56 | 0.70 | 1.09 | 1.04 | 1.13 |
| waist | 0.62 | 0.55 | 0.69 | 1.05 | 1.02 | 1.08 |
| wgt | 0.62 | 0.54 | 0.69 | 1.07 | 1.03 | 1.12 |
| L-ISO | 0.61 | 0.54 | 0.68 | 1.12 | 1.04 | 1.20 |
| 1-HD | 0.61 | 0.53 | 0.68 | 0.89 | 0.82 | 0.96 |
| DC | 0.60 | 0.53 | 0.67 | 1.32 | 1.08 | 1.63 |
| ADMA | 0.59 | 0.52 | 0.65 | 1.04 | 1.01 | 1.08 |
| DGLA | 0.59 | 0.52 | 0.65 | 1.15 | 1.02 | 1.30 |
| PALMA | 0.58 | 0.52 | 0.65 | 1.18 | 1.02 | 1.35 |
| L-LEU | 0.59 | 0.52 | 0.66 | 1.09 | 1.02 | 1.17 |
| 12CAR | 0.59 | 0.51 | 0.66 | 1.23 | 1.03 | 1.45 |
| DLG | 0.58 | 0.51 | 0.65 | 1.16 | 1.03 | 1.31 |
| OLA | 0.58 | 0.51 | 0.65 | 1.23 | 1.01 | 1.50 |
| ECG | 0.57 | 0.51 | 0.64 | 1.09 | 1.00 | 1.20 |
| LINA | 0.57 | 0.50 | 0.64 | 1.21 | 1.00 | 1.49 |
| 3-HBA | 0.58 | 0.50 | 0.65 | 1.19 | 1.01 | 1.39 |
| 2-HBA | 0.57 | 0.50 | 0.64 | 1.11 | 1.00 | 1.23 |
| 16CAR | 0.57 | 0.50 | 0.64 | 1.07 | 1.00 | 1.15 |
| EPA | 0.58 | 0.50 | 0.65 | 1.24 | 1.01 | 1.51 |
| H-L-ARG | 0.57 | 0.50 | 0.64 | 1.10 | 1.00 | 1.21 |
| L-LYS | 0.57 | 0.50 | 0.64 | 1.03 | 1.00 | 1.07 |
| fh_pet | 0.54 | 0.50 | 0.57 | na | na | na |

When prognostic performance is assessed by AUC, it can be observed that classic clinical factors have favorable single-variable prognostic merits for predicting preterm PE, yet a significant number of metabolites of interest show prognostic merits; it can be noted that none of the proteins have significant prognostic power to predict term PE. Taking AUC and “fold changes” into account, the following 1^{st} tier metabolites are found as being relevant to the prognosis of “term-PE” risk (as assessed by AUC); in order of relevance: L-ISO, 1-HD and DC

### Example 4: Multivariable Generic Prognostic Performance—AUROC

### Example 4A; PE Sub-Type: All PE

Model Space filters applied: Mean AUC ICI>=0.5; mean AUC>=0.65; Mean AUC Imp: >=0.02.
Rank complete AUC from high to low and select models down to 1^{st} single marker.
Based on this filtering, a set of 44 multivariable models was found, cf Table 9

TABLE 9
All PE

| | (Recurrent) Predictors | | | | | | | | | Complete data set | | | 3-fold cross |
|---|---|---|---|---|---|---|---|---|---|---|---|---|---|
| n predictors/ | Blood | | | | | weight | | | | | AUC | AUC | validation |
| model | pressure | HVD3 | DLG | s-ENG | PIGF | rel | 1-HD | L-ISO | other | AUC | ICI | uCI | AUC impr |
| 1 | 2nd sbp | | | | | | | | | 0.67 | 061 | 0.73 | |
| 2 | 2nd_sbp | x | | | | | | | | 0.70 | 0.64 | 0.76 | 0.033 |
| 2 | 2nd_sbp | | x | | | | | | | 0.70 | 0.64 | 0.75 | 0.025 |
| 2 | 2nd_sbp | | | MoM | | | | | | 0.69 | 0.64 | 0.75 | 0.021 |
| 2 | map_2nd | x | | | | | | | | 0.69 | 0.63 | 0.75 | 0.032 |
| 2 | map_2nd | | | MoM | | | | | | 0.69 | 0.63 | 0.75 | 0.025 |
| 2 | map_2nd | | x | | | | | | | 0.69 | 0.63 | 0.75 | 0.026 |
| 2 | 1st_sbp | | x | | | | | | | 0.69 | 0.63 | 0.75 | 0.034 |
| 2 | 1st_sbp | x | | | | | | | | 0.68 | 0.62 | 0.75 | 0.031 |
| 2 | 1st_sbp | | | | MoM | | | | | 0.68 | 0.62 | 0.75 | 0.028 |
| 2 | 1st_sbp | | | | | bmi | | | | 0.68 | 0.62 | 0.74 | 0.023 |
| 2 | 1st_sbp | | | | x | | | | | 0.68 | 0.62 | 0.74 | 0.024 |
| 2 | map_1st | | | MoM | | | | | | 0.68 | 0.62 | 0.74 | 0.031 |
| 2 | map_1st | | x | | | | | | | 0.68 | 0.61 | 0.74 | 0.035 |
| 2 | 2nd_dbp | | | MoM | | | | | | 0.68 | 0.62 | 0.74 | 0.030 |
| 2 | map_1st | | | | | bmi | | | | 0.67 | 0.62 | 0.73 | 0.026 |
| 2 | map_1st | x | | | | | | | | 0.67 | 0.61 | 0.74 | 0.037 |
| 3 | 1st_sbp | x | | | | bmi | | | | 0.71 | 0.66 | 0.77 | 0.023 |
| 3 | 1st_sbp | x | | | | waist | | | | 0.71 | 0.65 | 0.77 | 0.021 |
| 3 | 1st_sbp | x | | | MoM | | | | | 0.70 | 0.64 | 0.77 | 0.022 |
| 3 | 1st_dbp | x | | | | bmi | | | | 0.69 | 0.64 | 0.75 | 0.031 |
| 3 | 1st_dbp | x | | | | waist | | | | 0.69 | 0.63 | 0.75 | 0.024 |
| 3 | | | | MoM | MoM | | x | | | 0.69 | 0.63 | 0.75 | 0.027 |
| 3 | | | | MoM | x | | x | | | 0.68 | 0.62 | 0.74 | 0.030 |
| 3 | 1st_dbp | | | MoM | | | | x | | 0.68 | 0.62 | 0.74 | 0.021 |
| 3 | | | x | | | waist | | | fh_pet | 0.67 | 0.61 | 0.73 | 0.021 |
| 3 | 1st_dbp | x | | | | | x | | | 0.67 | 0.61 | 0.73 | 0.021 |
| 3 | | | x | x | | waist | | | | 0.67 | 0.61 | 0.73 | 0.021 |
| 3 | | | x | MoM | | waist | | | | 0.67 | 0.61 | 0.73 | 0.021 |
| 3 | | | x | MoM | | | x | | | 0.67 | 0.61 | 0.73 | 0.021 |
| 4 | map_1st | | | x | | | x | x | | 0.71 | 0.65 | 0.77 | 0.022 |
| 4 | 1st_dbp | | | MoM | | | x | x | | 0.70 | 0.65 | 0.76 | 0.023 |
| 4 | 1st_dbp | | | x | | | x | x | | 0.70 | 0.64 | 0.76 | 0.027 |
| 4 | | | | MoM | MoM | | MoM | x | | 0.69 | 0.63 | 0.75 | 0.027 |
| 4 | | | | MoM | | wgt | x | x | | 0.69 | 0.63 | 0.75 | 0.021 |
| 4 | | | | MoM | x | | MoM | x | | 0.69 | 0.63 | 0.75 | 0.022 |
| 4 | | | | MoM | | wgt | MoM | x | | 0.69 | 0.63 | 0.75 | 0.025 |
| 4 | | | | x | | wgt | x | x | | 0.69 | 0.63 | 0.74 | 0.025 |
| 4 | | | | x | MoM | | MoM | x | | 0.69 | 0.63 | 0.75 | 0.024 |
| 4 | | | | x | | waist x | x | | | 0.68 | 0.62 | 0.74 | 0.024 |
| 4 | | | | x | | wgt | MoM | x | | 0.68 | 0.62 | 0.74 | 0.025 |
| 4 | | | | x | x | | MoM | x | | 0.68 | 0.62 | 0.74 | 0.026 |
| 4 | | | | MoM | | | x | | L-LEU + | 0.68 | 0.62 | 0.74 | 0.021 |
| | | | | | | | | | EPA | | | | |
| 4 | | | | x | | | x | x | EPA | 0.68 | 0.62 | 0.74 | 0.022 |
| 4 | | | | x | MoM | | MoM | | L-LEU | 0.67 | 0.61 | 0.73 | 0.020 |

With a blood pressure (bp) measurement being the most performant single variable, it is not surprising that combinations with blood pressures feature a lot. The following blood-pressure centric 2 to 4 variable prognostic cores are found (in order of performance):
1. bp+HVD3, possibly augmented with WRV (bmi/weight/waist) and/or PIGF.
2. bp+DLG
3. bp+s-ENG, possibly augmented with 1-HD and/or L-ISO
4. bp+PIGF
5. bp+WRV (bmi/weight/waist)

Alternatively, some cores without blood pressure were also found; i.e., The following 3 variable prognostic core;
1. s-ENG+{PIGF OR DLG}+{WRV (bmi/weight/waist) OR 1-HD OR fh_pet}

The following 4 variable prognostic core;
2. {s-ENG AND 1-HD}+any 2 variables from {PIGF, L-ISO, L-LEU, EPA}

### Summary

When prognostic performance is assessed by AUC, it can be observed that multi-variable prognostic performance for predicting all PE is complying with the pre-set AUC>=0.65 criterions, when combining a blood pressure measure with HVD3, DLG, s-ENG or PIGF.

Further prognostic performance increments can be achieved by further adding any of the following variables: 1-HD, L-ISO, any weight related variable. Other potential additive variables are EPA or L-LEU. Within all these variables, the following variable pairs are associated with additive prognostic performance (s-ENG+1-HD), (s-ENG+DLG) or (s-ENG+PIGF).

### Example 4B; PE Sub-Type: Preterm PE

Model Space Filters applied: Mean AUC ICI>=0.5; mean AUC>=0.70; Mean AUC Imp: >=0.02.
Rank complete AUC from high to low and select models down to 1^{st} single marker.
Based on this filtering, a set of 39 multivariable models was found, cf TABLE 10

TABLE 10
Preterm PE

| | | | | | | | | | | | | | | 3-fold |
|---|---|---|---|---|---|---|---|---|---|---|---|---|---|---|
| | | | | | | | | | | | Complete | | | cross |
| n | (Recurrent) Predictors | | | | | | | | | | data set | | | validation |
| predictors/ | | | | | | | | | 2-HBA | | | AUC | AUC | AUC |
| model | PIGF | DLG | s-END | SC | 1-HD | L-ERG | ECG | 20-CL | OR NGM | other | AUC | ICI | uCI | imp |
| 1 | MoM | | | | | | | | | | 0.74 | 0.62 | 0.86 | |
| 2 | x | x | | | | | | | | | 0.79 | 0.69 | 0.89 | 0.030 |
| 2 | MoM | x | | | | | | | | | 0.79 | 0.69 | 0.89 | 0.022 |
| 2 | MoM | | MoM | | | | | | | | 0.78 | 0.67 | 0.90 | 0.036 |
| 2 | MoM | | x | | | | | | | | 0.78 | 0.66 | 0.89 | 0.030 |
| 2 | x | | MoM | | | | | | | | 0.78 | 0.66 | 0.89 | 0.040 |
| 2 | | x | MoM | | | | | | | | 0.77 | 0.67 | 0.86 | 0.064 |
| 2 | x | | x | | | | | | | | 0.77 | 0.65 | 0.88 | 0.031 |
| 2 | | x | x | | | | | | | | 0.76 | 0.67 | 0.86 | 0.058 |
| 2 | MoM | | | | | | | | | fh_pet | 0.76 | 0.64 | 0.88 | 0.022 |
| 3 | MoM | x | MoM | | | | | | | | 0.82 | 0.72 | 0.92 | 0.035 |
| 3 | x | x | MoM | | | | | | | | 0.82 | 0.72 | 0.92 | 0.036 |
| 3 | MoM | x | x | | | | | | | | 0.82 | 0.72 | 0.92 | 0.038 |
| 3 | x | x | x | | | | | | | | 0.81 | 0.71 | 0.92 | 0.038 |
| 3 | MoM | | | x | | | | | | CL | 0.79 | 0.69 | 0.89 | 0.023 |
| 3 | x | | x | | x | | | | | | 0.78 | 0.67 | 0.90 | 0.021 |
| 3 | | x | x | | x | | | | | | 0.78 | 0.69 | 0.87 | 0.022 |
| 3 | | x | MoM | | MoM | | | | | | 0.78 | 0.69 | 0.87 | 0.020 |
| 3 | x | | | x | | | | | | CL | 0.78 | 0.67 | 0.88 | 0.021 |
| 3 | | x | | x | | MoM | | | | | 0.75 | 0.65 | 0.85 | 0.030 |
| 3 | | | MoM | x | | | | | 2-HBA | | 0.74 | 0.64 | 0.85 | 0.026 |
| 4 | | x | MoM | x | | MoM | | | | | 0.80 | 0.72 | 0.89 | 0.026 |
| 4 | | x | x | x | | MoM | | | | | 0.80 | 0.71 | 0.89 | 0.025 |
| 4 | | x | | x | | MoM | | | | fh_pet | 0.77 | 0.68 | 0.87 | 0.022 |
| 4 | | | MoM | | | | x | x | 2-HBA | | 0.77 | 0.67 | 0.87 | 0.024 |
| 4 | MoM | | | | | | x | x | | OLA | 0.77 | 0 . 66 | 0.88 | 0.025 |
| 4 | | | MoM | | | | x | | 2-HBA | DHA_MoM | 0.77 | 0.67 | 0.87 | 0.020 |
| 4 | | x | | x | | x | | | | fh_pet | 0.77 | 0.67 | 0.86 | 0.024 |
| 4 | | | x | | | | x | x | 2-HBA | | 0.76 | 0.66 | 0.87 | 0.022 |
| 4 | | x | | | | | | | | r_glucose + | 0.76 | 0.66 | 0.86 | 0.022 |
| | | | | | | | | | | fh_pet + | | | | |
| | | | | | | | | | | HVD3 | | | | |
| 4 | | | x | | | | x | x | NGM | | 0.76 | 0.67 | 0.85 | 0.022 |
| 4 | | | MoM | | | | x | | NGM | AcCAR | 0.76 | 0.67 | 0.85 | 0.027 |
| 4 | | | x | x | | | x | | NGM | | 0.76 | 0.67 | 0.84 | 0.023 |
| 4 | | x | | x | | | | | | DHA_MoM + | 0.75 | 0.65 | 0.86 | 0.021 |
| | | | | | | | | | | DGLA | | | | |
| 4 | MoM | | | | | | | x | | LINA + | 0.75 | 0.63 | 0.87 | 0.021 |
| | | | | | | | | | | PALMA | | | | |
| 4 | | | x | | | | x | | 2-HBA | DHA_MoM | 0.75 | 0.65 | 0.86 | 0.022 |
| 4 | x | | | | | | x | x | | LINA | 0.75 | 0.63 | 0.86 | 0.021 |
| 4 | | | x | | | | x | | 2-HBA | DHA | 0.75 | 0.65 | 0.85 | 0.020 |
| 4 | | | x | | | | x | | NGM | AcCAR | 0.75 | 0.66 | 0.84 | 0.030 |
| 4 | | | MoM | | | | x | | NGM | STERA | 0.75 | 0.65 | 0.84 | 0.024 |

From Table 10, it is clear 3 variables have very complementary predictive performance, i.e., PIGF, DLG and s-ENG. with PIGF combined with DLG delivering the strongest performance. As apparent from the improvements, combining these 3 variables in pairs as well as combining all 3 of them results in significant performance gains.

So based on this, one can discern the following high performant prognostic 2-marker cores; i.e.,
1. PIGF+DLG
2. PIGF+s-ENG
3. DLG+s-ENG
4. PIGF+fh_pet

When considering 3 variables, a particularly strong core is found in
1. PIGF+DLG+s-ENG

Alternative 3 variable prognostic cores always feature 1 or 2 of the core predictors PIGF, DLG, s-ENG:
2. PIGF+s-ENG+1-HD
3. PIGF+SC+CL
4. DLG+s-ENG+1-HD
5. DLG+SC+L-ERG
6. s-ENG+SC+2-HBA

Within this data set, there was no further improvement achieved by adding a 4th predictor to the above cores. However, further additive value may become apparent in larger patient cohorts. To find the variables which will likely be part of more expansive cores for predicting Preterm PE with exceptional AUCs, the following alternative 4 variable prognostic cores were also established. Once again, alternative 4 marker cores always feature 1 or 2 of the core predictors PIGF, DLG, s-ENG
1. {DLG AND SC}+L-ERG+{s-ENG OR fh_pet}
2. {DLG AND SC}+DHA+DGLA
3. DLG+HVD3+fh_pet+r_glucose
4. {s-ENG AND ECG}+{2-HBA OR NGM}+any 1 predictor from {DHA, STERA, 20-CL, AcCAR}
5. {PIGF AND 20-CL}+ECG+{MYRA OR LINA}
6. {PIGF AND 20-CL}+PALMA+LINA

### Summary

When prognostic performance for Preterm PE is assessed by AUC, it can be observed that exceptional multi-variable prognostic performance is achieved when combining DLG with PIGF or s-ENG, with all 2 variable combinations outperforming PIGF, the best single variable, significantly. Moreover, a further additive effect is found when combining all of DLG and PIGF and s-ENG. This metabolite—protein combination has truly exceptional prognostic merits for preterm PE. Other potential additive variables relevant to the generic (AUC) prognostic performance for Preterm PE are: SC, 1-HD, ECG, 20-CL, 2-HBA and NGM.

### Example 4C; PE Sub-Type: Term PE

Model Space Filters applied: Mean AUC ICI>=0.5; Mean AUC>=0.65: Mean AUC Imp: >=0.01.
Rank complete AUC from high to low and select models down to 1^{st} single marker.
Based on this filtering, a set of 56 multivariable models was found, et TABLE 11.

TABLE 11
Term PE

| | | | | | | | | | | | 3 fold |
|---|---|---|---|---|---|---|---|---|---|---|---|
| | | | | | | | | Complete | | | cross |
| n | (Recurrent) Predictors | | | | | | | data set | | | validation |
| predictors/ | Blood | | weight | | | | | | AUC | AUC | AUC |
| model | pressure | HVD3 | related | L-ISO | 1-HD | L-LEU | Other | AUC | ICI | uCI | imp |
| 1 | map_2nd | | | | | | | 0.69 | 0.63 | 0.76 | |
| 2 | 2nd_sbp | x | | | | | | 0.72 | 0.66 | 0.79 | 0.029 |
| 2 | map_2nd | | | x | | | | 0.72 | 0.66 | 0.78 | 0.031 |
| 2 | map_2nd | x | | | | | | 0.72 | 0.65 | 0.78 | 0.029 |
| 2 | 2nd_sbp | | | | x | | | 0.71 | 0.65 | 0.77 | 0.018 |
| 2 | map_2nd | | | | | x | | 0.71 | 0.64 | 0.78 | 0.018 |
| 2 | 1st_sbp | x | | | | | | 0.71 | 0.64 | 0.78 | 0.029 |
| 2 | map_2nd | | | | x | | | 0.71 | 0.65 | 0.77 | 0.010 |
| 2 | 2nd_sbp | | bmi | | | | | 0.71 | 0.64 | 0.77 | 0.015 |
| 2 | map_2nd | | | | | | s-ENG_MoM | 0.71 | 0.64 | 0.77 | 0.016 |
| 2 | 2nd_sbp | | | x | | | | 0.71 | 0.64 | 0.77 | 0.017 |
| 2 | map_2nd | | | | | | ADMA | 0.71 | 0.64 | 0.77 | 0.013 |
| 2 | map_2nd | | | | | | s-ENG | 0.70 | 0.64 | 0.77 | 0.011 |
| 2 | 2nd_sbp | | waist | | | | | 0.70 | 0.64 | 0.77 | 0.013 |
| 2 | 2nd_dbp | | | x | | | | 0.70 | 0.64 | 0.77 | 0.042 |
| 2 | map_2nd | | | | | | L-MET | 0.70 | 0.63 | 0.77 | 0.011 |
| 2 | 1st_sbp | | | | | | DLG | 0.70 | 0.63 | 0.77 | 0.012 |
| 2 | map_1st | | | x | | | | 0.70 | 0.63 | 0.77 | 0.033 |
| 2 | map_2nd | | | | | | L-GLU_MoM | 0.70 | 0.63 | 0.76 | 0.011 |
| 2 | 1st_sbp | | | x | | | | 0.70 | 0.63 | 0.77 | 0.020 |
| 2 | 1st_sbp | | | | x | | | 0.70 | 0.64 | 0.76 | 0.011 |
| 2 | map_1st | x | | | | | | 0.70 | 0.63 | 0.77 | 0.032 |
| 2 | 1st_sbp | | waist | | | | | 0.70 | 0.63 | 0.76 | 0.018 |
| 2 | 1st_sbp | | bmi | | | | | 0.70 | 0.63 | 0.76 | 0.015 |
| 2 | map_1st | | | | x | | | 0.70 | 0.63 | 0.76 | 0.023 |
| 2 | map_1st | | | | | x | | 0.69 | 0.62 | 0.76 | 0.028 |
| 3 | 2nd_sbp | x | bmi | | | | | 0.74 | 0.67 | 0.80 | 0.018 |
| 3 | 2nd_sbp | x | waist | | | | | 0.74 | 0.67 | 0.80 | 0.012 |
| 3 | 2nd_sbp | x | | | x | | | 0.74 | 0.67 | 0.80 | 0.013 |
| 3 | map_2nd | x | | x | | | | 0.73 | 0.67 | 0.80 | 0.011 |
| 3 | 2nd_sbp | x | | | | | H-L-ARG | 0.73 | 0.67 | 0.80 | 0.010 |
| 3 | 2nd_sbp | x | | x | | | | 0.73 | 0.67 | 0.80 | 0.010 |
| 3 | 1st_sbp | x | waist | | | | | 0.73 | 0.67 | 0.79 | 0.021 |
| 3 | 1st_sbp | x | bmi | | | | | 0.73 | 0.66 | 0.79 | 0.018 |
| 3 | map_1st | x | | x | | | | 0.72 | 0.66 | 0.79 | 0.022 |
| 3 | 2nd_sbp | | | x | x | | | 0.72 | 0.66 | 0.79 | 0.011 |
| 3 | map_1st | | | x | x | | | 0.72 | 0.66 | 0.78 | 0.017 |
| 3 | map_1st | x | | | | x | | 0.72 | 0.65 | 0.79 | 0.017 |
| 3 | map_1st | x | bmi | | | | | 0.72 | 0.66 | 0.78 | 0.015 |
| 3 | map_1st | x | waist | | | | | 0.72 | 0.65 | 0.78 | 0.018 |
| 3 | 1st_sbp | | | x | x | | | 0.72 | 0.65 | 0.78 | 0.018 |
| 3 | 1st_sbp | | | x | MoM | | | 0.72 | 0.65 | 0.78 | 0.011 |
| 3 | 1st_sbp | | | | x | x | | 0.71 | 0.65 | 0.78 | 0.013 |
| 3 | 1st_sbp | | | | | | DLG + fh_pet | 0.71 | 0.64 | 0.78 | 0.011 |
| 3 | 1st_dbp | | | x | x | | | 0.71 | 0.65 | 0.77 | 0.018 |
| 3 | 1st_dbp | x | | x | | | | 0.70 | 0.63 | 0.77 | 0.013 |
| 3 | 1st_dbp | x | bmi | | | | | 0.70 | 0.64 | 0.76 | 0.020 |
| 3 | 1st_dbp | | | | x | x | | 0.70 | 0.64 | 0.76 | 0.013 |
| 3 | 1st_dbp | x | waist | | | | | 0.70 | 0.63 | 0.77 | 0.019 |
| 3 | 1st_dbp | x | | | | x | | 0.70 | 0.62 | 0.77 | 0.015 |
| 3 | 2nd_dbp | | | | | | s-ENG_MoM + | 0.69 | 0.63 | 0.76 | 0.011 |
| | | | | | | | L-MET | | | | |
| 3 | 2nd_dbp | | | | | | DLG + fh_pet | 0.69 | 0.63 | 0.76 | 0.010 |
| 4 | map_1st | x | | x | x | | | 0.73 | 0.67 | 0.80 | 0.010 |
| 4 | 1st_sbp | x | | x | x | | | 0.73 | 0.67 | 0.80 | 0.014 |
| 4 | 1st_sbp | x | | x | | | H-L-ARG | 0.73 | 0.66 | 0.79 | 0.010 |
| 4 | map_1st | x | | | x | | H-L-ARG | 0.72 | 0.66 | 0.79 | 0.011 |
| 4 | 1st_dbp | x | | | x | | H-L-ARG | 0.71 | 0.64 | 0.77 | 0.011 |

### Prognostic Cores:

From TABLE 11, it is clear that a blood pressure measurement is mandatory to achieve meaningful predictive performance for term preeclampsia. As apparent from the improvements, combining blood pressure with a number of metabolites can add to the predictive performance; the following 1^{st} tier 2 predictor cores are therefore found:
1. bp+HVD3
2. bp+L-ISO
3. bp+1-HD
4. bp+L-LEU

Some 2^{nd} tier 2 predictor cores are also found:
5. bp+any one predictor from {WRV (bmi/weight/waist), s-ENG, ADMA, L-MET, DLG, L-GLU}

Building on this, combinations of 3 variables typically constitute a blood pressure measurement and 1 of the 1^{st} tier metabolites, augmented with a 3rd marker (often another 1^{st} tier or 2^{nd} tier variable as found in the 2 predictor cores). The following 3 variable prognostic cores were found:
1. {bp AND HVD3}+any 1 predictor from {L-LEU, L-ISO, H-L-ARG, 1-HD}
2. {bp AND 1-HD}+{L-LEU OR L-ISO}
3. bp+DLG+fh_pet
4. bp+s-ENG+L-MET

Combinations of 4 variables all constitute a blood pressure measurement as well as HVD3. This predictor pair is then augmented with any 2 out of a set of 3 predictors to yield the following 4 variable prognostic cores:
1. {bp AND HVD3}+any 2 variables from {H-L-ARG, L-ISO, 1-HD}

### Summary

When prognostic performance is assessed by AUC, it can be observed that multi-variable prognostic performance for predicting Term PE is exceeding the pre-set AUC>=0.65 criterion, when combining a blood pressure measure with HVD3, 1-HD, L-ISO or L-LEU. Further prognostic performance increments can be achieved by combining {blood pressure AND HVD3} with 1-HD, L-ISO, L-LEU or H-L-ARG, and/or combinations thereof.

### Example 5: Rule-in Prognostic Performance

In order to identify robust prognostic rule-in cores, models are selected that deliver prognostic performance for the Sensitivity (Sens) at 20% FPR (i.e., specificity=0.8) criterion as well as the Sens at 10% FPR (i.e., specificity=0.9) criterion, whereby the Sens at 20% FPR is considered the more robust criterion, albeit less relevant in a clinical context. Typically, more stringent filters are used for the Sens At Spec 0.80 criterion, both for the lower limits of the 90% confidence interval as well as for the improvement, than for the Sens at Spec 0.90 criterion.

The analysis for the Sens at PPV threshold, which is different for each disease sub-type considered (cf. Example 1), is reported on separately.

### Example 5A; PE Sub-Type—all PE

### FPR Thresholds:

Model Space Filters applied: mean AUC ICI>=0.5; for the statistic mean SensAtSpec 0.80: ICI>=0.2 and improvement >=0.03; for the statistic mean SensAtSpec 0.90: ICI>=0.1 and imp>=0.02.
Rank complete SensAtSpec 0.80 from high to low and select models down to 1^{st} single marker
Based on this filtering, a set of 49 multivariable models was found, cf TABLE 12.

All PE

| | (Recurrent) | | | | | | | | | |
|---|---|---|---|---|---|---|---|---|---|---|
| n | Predictors | | | | | | | | | |
| predictors/ | blood | | | | L- | | s- | | other | |
| model | pressure | PICF | DLG | HVD3 | MET | EPA | ENG | fh_pet | recurrent | other |
| 1 | 2nd_sbp | | | | | | | | | |
| 2 | 1st_sbp | MoM | | | | | | | | |
| 2 | 2nd_sbp | | x | | | | | | | |
| 2 | 1st_sbp | x | | | | | | | | |
| 2 | 2nd_sbp | | | x | | | | | | |
| 2 | map_2nd | | | | x | | | | | |
| 2 | map_2nd | MoM | | | | | | | | |
| 2 | map_2nd | x | | | | | | | | |
| 2 | 1st_dbp | MoM | | | | | | | | |
| 2 | 2nd_sbp | | | | x | | | | | |
| 2 | map_1st | | | | x | | | | | |
| 2 | 2nd_sbp | x | | | | | | | | |
| 2 | 2nd_sbp | MoM | | | | | | | | |
| 2 | map_1st | | x | | | | | | | |
| 2 | 2nd_sbp | | | | | x | | | | |
| 2 | 1st_sbp | | x | | | | | | | |
| 2 | 1st_sbp | | | | | | MoM | | | |
| 2 | map_1st | | | | | | | | L-LEU | |
| 2 | 2nd_sbp | | | | | | | | L-ALA | |
| 2 | 2nd_sbp | | | | | | | | L-LEU | |
| 2 | map_2nd | | x | | | | | | | |
| 2 | map_2nd | | | | | | | | | L-ARG |
| 2 | 1st_sbp | | | | | | | | L-ALA | |
| 2 | 1st_sbp | | | x | | | | | | |
| 2 | map_1st | MoM | | | | | | | | |
| 3 | 1st_sbp | | | | | | | | NGM | |
| 3 | 1st_dbp | x | | | | | | | | |
| 3 | map_2nd | | | | | | | | NGM | |
| 3 | 2nd_dbp | MoM | | | | | | | | |
| 3 | 2nd_sbp | | | x | | | | x | | |
| 3 | map_2nd | x | | x | | | | | | |
| 3 | 1st_sbp | | | | | | MoM | | | ECG |
| 3 | map_2nd | | | | | | MoM | | L-ISO | |
| 3 | 2nd_sbp | MoM | | x | | | | | | |
| 3 | 2nd_sbp | | | | | | | x | CR | |
| 3 | 2nd_sbp | | | | | | | x | | H-L-ARG |
| 3 | 2nd_sbp | | | | | | | x | BV | |
| 3 | | x | | | | | MoM | | 1-HD | |
| 3 | 2nd_dbp | | x | | | x | | | | |
| 3 | 1st_sbp | | | | | | | x | | sDMA |
| 3 | 1st_sbp | | | | | | | x | S-1-P_MoM | |
| 3 | 1st_sbp | | | | | | | x | S-1-P | |
| 3 | map_1st | | | | | | x | | L-ISO | |
| 4 | map_2nd | | | x | | | | | CR | ADMA |
| 4 | | x | | | | | x | | L-LEU + 1-HD | |
| 4 | map_2nd | | x | | | x | | | L-ERG | |
| 4 | map_2nd | | x | | | | MoM | | L-ERG | |
| 4 | map_1st | | x | x | | | | | L-ERG | |
| 4 | map_2nd | | | x | | | | x | gest | |

All PE

| | Complete data set | | | | | | 3 fold cross validation | |
|---|---|---|---|---|---|---|---|---|
| n | Senstivity at | | | Senstivity at | | | Imp | |
| predictors/ | Specificity 0.8 | | | Specificity 0.9 | | | SnAtSp | SnAtSp |
| model | Sn | lCl | uCl | Sn | lCl | uCl | 0.8 | 0.9 |
| 1 | 0.42 | 0.30 | 0.53 | 0.23 | 0.11 | 0.35 | 0.42 | 0.23 |
| 2 | 0.52 | 0.37 | 0.63 | 0.31 | 0.19 | 0.43 | 0.10 | 0.05 |
| 2 | 0.48 | 0.37 | 0.59 | 0.29 | 0.19 | 0.39 | 0.04 | 0.09 |
| 2 | 0.47 | 0.35 | 0.59 | 0.30 | 0.21 | 0.42 | 0.10 | 0.11 |
| 2 | 0.47 | 0.35 | 0.60 | 0.33 | 0.20 | 0.44 | 0.06 | 0.08 |
| 2 | 0.46 | 0.36 | 0.58 | 0.26 | 0.14 | 0.39 | 0.06 | 0.06 |
| 2 | 0.45 | 0.34 | 0.58 | 0.32 | 0.22 | 0.41 | 0.07 | 0.04 |
| 2 | 0.45 | 0.34 | 0.57 | 0.30 | 0.19 | 0.42 | 0.06 | 0.06 |
| 2 | 0.45 | 0.35 | 0.57 | 0.28 | p.19 | 0.40 | 0.10 | 0.02 |
| 2 | 0.45 | 0.33 | 0.56 | 0.28 | 0.18 | 0.40 | 0.04 | 0.06 |
| 2 | 0.45 | 0.32 | 0.59 | 0.26 | 0.15 | 0.40 | 0.09 | 0.03 |
| 2 | 0.44 | 0.33 | 0.55 | 0.33 | 0.23 | 0.43 | 0.04 | 0.10 |
| 2 | 0.44 | 0.33 | 0.55 | 0.31 | 0.21 | 0.44 | 0.06 | 0.06 |
| 2 | 0.44 | 0.30 | 0.56 | 0.30 | 0.21 | 0.40 | 0.07 | 0.06 |
| 2 | 0.44 | 0.32 | 0.58 | 0.30 | p.15 | 0.42 | 0.03 | 0.05 |
| 2 | 0.44 | 0.34 | 0.57 | 0.29 | 0.14 | 0.41 | 0.07 | 0.07 |
| 2 | 0.44 | 0.34 | 0.55 | 0.29 | 0.19 | 0.39 | 0.04 | 0.06 |
| 2 | 0.44 | 0.32 | 0.56 | 0.28 | 0.19 | 0.38 | 0.03 | 0.03 |
| 2 | 0.44 | 0.34 | 0.56 | 0.27 | 0.18 | 0.38 | 0.05 | 0.07 |
| 2 | 0.44 | 0.32 | 0.56 | 0.27 | 0.16 | 0.38 | 0.03 | 0.07 |
| 2 | 0.44 | 0.34 | 0.55 | 0.27 | 0.18 | 0.39 | 0.03 | 0.09 |
| 2 | 0.44 | 0.34 | 0.56 | 0.26 | 0.15 | 0.38 | 0.03 | 0.07 |
| 2 | 0.44 | 0.31 | 0.58 | 0.25 | 0.14 | 0.38 | 0.04 | 0.12 |
| 2 | 0.44 | 0.33 | 0.58 | 0.30 | 0.19 | 0.43 | 0.08 | 0.15 |
| 2 | 0.43 | 0.32 | 0.55 | 0.34 | 0.24 | 0.44 | 0.08 | 0.06 |
| 3 | 0.43 | 0.32 | 0.56 | 0.30 | 0.20 | 0.41 | 0.03 | 0.15 |
| 3 | 0.43 | 0.32 | 0.55 | 0.29 | 0.18 | 0.41 | 0.07 | 0.07 |
| 3 | 0.43 | 0.33 | 0.54 | 0.28 | 0.16 | 0.39 | 0.05 | 0.06 |
| 3 | 0.43 | 0.31 | 0.54 | 0.27 | 0.16 | 0.39 | 0.07 | 0.03 |
| 3 | 0.51 | 0.38 | 0.64 | 0.34 | 0.21 | 0.47 | 0.03 | 0.02 |
| 3 | 0.49 | 0.37 | 0.63 | 0.31 | 0.20 | 0.45 | 0.03 | 0.04 |
| 3 | 0.48 | 0.37 | 0.59 | 0.33 | 0.21 | 0.46 | 0.04 | 0.03 |
| 3 | 0.48 | 0.35 | 0.61 | 0.28 | 0.16 | 0.41 | 0.03 | 0.03 |
| 3 | 0.48 | 0.37 | 0.60 | 0.34 | 0.24 | 0.46 | 0.03 | 0.03 |
| 3 | 0.47 | 0.35 | 0.59 | 0.31 | 0.22 | 0.42 | 0.03 | 0.05 |
| 3 | 0.47 | 0.35 | 0.59 | 0.30 | 0.21 | 0.41 | 0.03 | 0.03 |
| 3 | 0.46 | 0.34 | 0.59 | 0.29 | 0.20 | 0.40 | 0.04 | 0.03 |
| 3 | 0.46 | 0.34 | 0.59 | 0.27 | 0.18 | 0.36 | 0.04 | 0.03 |
| 3 | 0.45 | 0.35 | 0.56 | 0.32 | 0.20 | 0.45 | 0.04 | 0.05 |
| 3 | 0.45 | 0.34 | 0.56 | 0.26 | 0.16 | 0.38 | 0.03 | 0.02 |
| 3 | 0.44 | 0.33 | 0.56 | 0.32 | 0.19 | 0.42 | 0.04 | 0.04 |
| 3 | 0.44 | 0.34 | 0.56 | 0.32 | 0.19 | 0.42 | 0.04 | 0.05 |
| 3 | 0.43 | 0.31 | 0.56 | 0.30 | 0.18 | 0.42 | 0.03 | 0.05 |
| 4 | 0.52 | 0.40 | 0.63 | 0.34 | 0.19 | 0.46 | 0.03 | 0.03 |
| 4 | 0.49 | 0.34 | 0.62 | 0.28 | 0.16 | 0.39 | 0.03 | 0.03 |
| 4 | 0.48 | 0.38 | 0.60 | 0.37 | 0.27 | 0.48 | 0.03 | 0.02 |
| 4 | 0.48 | 0.38 | 0.60 | 0.30 | 0.19 | 0.46 | 0.04 | 0.02 |
| 4 | 0.47 | 0.36 | 0.58 | 0.36 | 0.26 | 0.47 | 0.03 | 0.03 |
| 4 | 0.46 | 0.33 | 0.58 | 0.31 | 0.21 | 0.42 | 0.04 | 0.03 |

### Prognostic Cores:

From the TABLE 12 it is clear that a blood pressure measurement is key variable in achieving meaningful rule-in predictive performance for all preeclampsia. As apparent from the improvements, combining blood pressure with a number of metabolites can add to the predictive performance; the following 1^{st} tier 2 predictor cores are therefore found:
1. bp+PIGF
2. bp+DLG
3. bp+HVD3
4. bp+L-MET

Some 2^{nd} tier 2 variable prognostic cores are also found:
5. bp+any one variable from {L-ALA, L-ARG, L-LEU, NGM, EPA, s-ENG}

The majority of combinations of 3 variables also include a bp measurement. A strong predictive core is found in:
1. bp+HVD3+fh_pet

HVD3 and fh_pet also feature in other 3 variable prognostic core permutations:
2. {bp AND HVD3}+PIGF
3. {bp AND fh_pet}+any one variable from {CR, H-L-ARG, BV, SDMA, S-1-P}

Alternative 3 variable prognostic cores featuring bp are:
4. bp+s-ENG+{ECG OR L-ISO}
5. bp+DLG+EPA

Another 3 variable prognostic core, without a blood pressure measurement was also found as follows;
6. s-ENG+PIGF+1-HD

Only one 4 variable prognostic core improves on the 3 variable combinations, but other compliant 4 variable prognostic cores are reported as further additive value may become apparent in larger patient cohorts.
1. {bp AND HVD3}+{CR+ADMA} OR {fh_pet+gest}
2. {bp AND DLG AND L-ERG}+any one variable from {HVD3, s-ENG, EPA}

A 4 variable prognostic core without a blood pressure measurement was also found. It builds on the earlier mentioned 3 variable combination:
3. s-ENG+PIGF+1-HD+L-LEU

### PPV Threshold:

For the preeclampsia example elaborated here, the PPV threshold for “all PE”, was set to PPV=0.133, and calculated for PE prevalence=0.05 in accordance with the rationale elaborated in Example 1 and the target thresholds as summarized in Table 3.

Model Space Filters applied: mean AUC ICI>=0.5; for the statistic mean SensAtPPV 0.133: ICI>=0.1; and imp>=0.05; Rank complete SensAtPPV from high to low and select models down to 1^{st} single marker
Based on this filtering, a set of 23 multivariable models was found, cf TABLE 13

TABLE 13
All PE

| | | | | | | | | | Complete | | | 3 fold |
|---|---|---|---|---|---|---|---|---|---|---|---|---|
| | | | | | | | | | data set | | | cross |
| n | (Recurrent) predictors | | | | | | | | Sensitivity at | | | validation |
| predictors/ | blood | | | | | | recurrent | | PPV = 0.133 | | | Imp |
| model | pressure | PIGF | 1-HD | HVD3 | DLG | 2-HBA | other | other | Sn | lCl | uCl | SnAtPPV |
| 1 | 2nd_sbp | | | | | | | | 0.19 | 0.02 | 0.52 | |
| 1 | | (x) | | | | | | | 0.26 | 0.00 | 0.43 | |
| 2 | 2nd_sbp | MoM | | | | | | | 0.41 | 0.18 | 0.54 | 0.23 |
| 2 | 2nd_sbp | | x | | | | | | 0.37 | 0.11 | 0.52 | 0.14 |
| 2 | 2nd_sbp | | MoM | | | | | | 0.36 | 0.12 | 0.54 | 0.14 |
| 3 | 2nd_sbp | MoM | | x | | | | | 0.45 | 0.21 | 0.63 | 0.09 |
| 3 | 1st_sbp | x | | x | | | | | 0.43 | 0.18 | 0.67 | 0.05 |
| 3 | map_2nd | x | | | x | | | | 0.42 | 0.19 | 0.59 | 0.14 |
| 3 | map_2nd | MoM | | | x | | | | 0.42 | 0.19 | 0.61 | 0.06 |
| 3 | map_2nd | MoM | | x | | | | | 0.42 | 0.12 | 0.67 | 0.07 |
| 3 | 2nd_sbp | x | | x | | | | | 0.39 | 0.21 | 0.60 | 0.06 |
| 3 | 2nd_sbp | | MoM | | | | | S-1-P | 0.37 | 0.12 | 0.53 | 0.07 |
| 3 | 1st_dbp | | | x | | x | | | 0.30 | 0.01 | 0.52 | 0.06 |
| 4 | map_2nd | | | x | | | | CR + | 0.48 | 0.01 | 0.65 | 0.07 |
| | | | | | | | | ADMA | | | | |
| 4 | map_2nd | | | | x | | s-ENG + | | 0.46 | 0.15 | 0.62 | 0.05 |
| | | | | | | | L-ERG | | | | | |
| 4 | 2nd_dbp | | | x | x | | | EPA | 0.46 | 0.03 | 0.60 | 0.08 |
| 4 | map_2nd | MoM | | | x | | fh_pet | | 0.43 | 0.18 | 0.64 | 0.05 |
| 4 | 2nd_sbp | MoM | | | x | | L-ERG | | 0.41 | 0.25 | 0.54 | 0.06 |
| 4 | map_2nd | | MoM | | | | | L-ARG + | 0.39 | 0.14 | 0.53 | 0.06 |
| | | | | | | | | L-MET | | | | |
| 4 | 2nd_sbp | | MoM | | | | s-ENG + | | 0.39 | 0.25 | 0.60 | 0.06 |
| | | | | | | | L-ISO | | | | | |
| 4 | map_2nd | | x | x | | | | L-LEU | 0.37 | 0.04 | 0.62 | 0.06 |
| 4 | | MoM | x | | | | s-ENG_MoM + | | 0.36 | 0.03 | 0.69 | 0.07 |
| | | | | | | | L-ISO | | | | | |
| 4 | map_2nd | | | x | | | fh_pet | gest | 0.34 | 0.19 | 0.60 | 0.06 |
| 4 | 1st_dbp | | | x | | x | | r_glucose | 0.33 | 0.00 | 0.60 | 0.05 |
| 4 | 1st_dbp | | | x | | x | | H-L-ARG | 0.29 | 0.08 | 0.54 | 0.05 |

### Prognostic Cores:

From the TABLE 13, it is clear that a blood pressure measurement is key variable in achieving meaningful rule-in predictive performance (PPV) for all preeclampsia. As apparent from the improvements, combining blood pressure with a number of metabolites can add to this predictive performance.

The following 1^{st} tier 2 variable prognostic cores are therefore found:
1. bp+PIGF
2. bp+1-HD

The 1^{st} tier 3 variable prognostic cores also feature bp measurements. Strong 3 variable predictive cores built on the 1^{st} tier 2 variable prognostic cores, include:
1. {bp AND PIGF}+HVD3
2. {bp AND PIGF}+DLG

Some 2^{nd} tier 3 variables cores were also found:
3. bp+1-HD+S-1-P
4. bp+HVD3+2-HBA

Whereas several other variables appear when considering combinations of 4 variables, it is also clear that they consistently constitute 2 or more of the variables as found in the 3 variable-cores, i.e., any 2 or more variables from: blood pressure measurement, HVD3, DLG, PIGF, 1-HD. This confirms their relevance to rule-in prognostic cores for all PE, when applying a clinically relevant PPV criterions as the performance threshold.

Some exemplary high performance 4 variable prognostic cores are:
1. bp+HVD3+CR+ADMA
2. bp+HVD3+DLG+EPA
3. bp+DLG+s-ENG+L-ERG
   Other 4 variable prognostic cores found, are:
1. bp+DLG+PIGF+(fh_pet OR L-ERG)
2. bp+DLG+s-ENG+L-ERG
3. bp+HVD3+1-HD+L-LEU
4. {PIGF OR bp}+1-HD+s-ENG+L-ISO
5. bp+HVD3+2-HBA+{r_glucose OR H-L-ARG}
6. bp+HVD3+fh_pet+gest

### Summary—Rule in Prognostic Performance all PE.

When prognostic performance is expressed as Sensitivity (i.e., detection rate of future cases) at set Rule-in thresholds like FPR or PPV, it can be observed that multi-variable prognostic performance for predicting all PE, is not achieved easily.

For the rule-in metrics considered, meeting the clinically relevant PPV threshold with good detection rates is found possible when combining a blood pressure measure AND PIGF with HVD3 or DLG. Further variables of relevance in achieving rule-in performance for “all PE” are fh_pet, L-MET, s-ENG. A particular performant core across the different rule-in criteria is bp+HVD3+CR+ADMA. It is of note that within the examples elaborated herein, the multivariable models are restricted to combinations of 4, and strict improvement criteria are applied. Further prognostic performance increments may follow when considering more variables/model, changing the improvement and/or thresholds.

### Example 5B; PE Sub-Type: Preterm PE

### FPR Thresholds:

Model Space Filters applied: mean AUC ICI>=0.5; for the statistic mean SensAtSpec 0.80: ICI>=0.2, and imp>=0.03; for the statistic mean SensAtSpec 0.90: ICI>=0.1 and imp>=0.01.
Rank complete SensAtSpec 0.80 from high to low and select models down to 1^{st} single marker Based on this filtering, a set of 20 multivariable models was found, cf TABLE 14

TABLE 14
Preterm PE

| | | | | | | | | | | | | | | | | | | 3 fold cross | |
|---|---|---|---|---|---|---|---|---|---|---|---|---|---|---|---|---|---|---|---|
| | | | | | | | | | | | | Complete Data Set | | | | | | validation | |
| n pre- | (Recurrent) Predictors | | | | | | | | | | | Senstivity at | | | Senstivity at | | | Imp | |
| dictors/ | | s- | | | | | 20- | 1- | | | | Specificity 0.8 | | | Specificity 0.9 | | | SnAtSp | SnAtSp |
| model | PIGF | ENG | DLG | ECG | fh_pet | CL | CL | HD | BR | CR | other | Sn | lCl | uCl | Sn | lCl | uCl | 0.8 | 0.9 |
| 1 | MoM | | | | | | | | | | | 0.57 | 0.35 | 0.78 | 0.48 | 0.26 | 0.70 | 0.57 | 0.48 |
| 2 | MoM | MoM | | | | | | | | | | 0.61 | 0.43 | 0.83 | 0.48 | 0.30 | 0.70 | 0.04 | 0.05 |
| 2 | x | MoM | | | | | | | | | | 0.61 | 0.43 | 0.83 | 0.48 | 0.30 | 0.70 | 0.08 | 0.09 |
| 3 | x | x | x | | | | | | | | | 0.78 | 0.57 | 0.91 | 0.52 | 0.30 | 0.74 | 0.11 | 0.07 |
| 3 | MoM | x | x | | | | | | | | | 0.74 | 0.52 | 0.91 | 0.48 | 0.26 | 0.74 | 0.11 | 0.05 |
| 3 | MoM | MoM | | x | | | | | | | | 0.70 | 0.48 | 0.87 | 0.57 | 0.35 | 0.78 | 0.05 | 0.03 |
| 3 | x | MoM | | x | | | | | | | | 0.70 | 0.48 | 0.87 | 0.57 | 0.35 | 0.78 | 0.05 | 0.03 |
| 3 | x | x | | x | | | | | | | | 0.70 | 0.48 | 0.87 | 0.57 | 0.35 | 0.74 | 0.05 | 0.07 |
| 3 | MoM | x | | x | | | | | | | | 0.70 | 0.48 | 0.87 | 0.57 | 0.35 | 0.74 | 0.05 | 0.03 |
| 3 | x | x | | | | x | | | | | | 0.65 | 0.43 | 0.87 | 0.52 | 0.30 | 0.70 | 0.03 | 0.05 |
| 3 | x | MoM | | | | | | | | | L-LEU | 0.65 | 0.43 | 0.87 | 0.52 | 0.30 | 0.74 | 0.03 | 0.05 |
| 4 | MoM | x | | x | | | x | | | | | 0.78 | 0.57 | 0.91 | 0.57 | 0.35 | 0.78 | 0.07 | 0.05 |
| 4 | x | x | | x | | | x | | | | | 0.74 | 0.57 | 0.91 | 0.61 | 0.35 | 0.83 | 0.07 | 0.05 |
| 4 | x | MoM | | x | x | | | | | | | 0.70 | 0.48 | 0.87 | 0.61 | 0.35 | 0.83 | 0.03 | 0.03 |
| 4 | x | x | | x | x | | | | | | | 0.70 | 0.48 | 0.87 | 0.57 | 0.35 | 0.78 | 0.03 | 0.03 |
| 4 | x | x | | | | x | | x | | | | 0.70 | 0.52 | 0.87 | 0.52 | 0.30 | 0.74 | 0.03 | 0.05 |
| 4 | MoM | | | | x | | | | x | | map_1st | 0.65 | 0.43 | 0.83 | 0.52 | 0.30 | 0.74 | 0.06 | 0.01 |
| 4 | x | x | | | | | | x | | x | | 0.65 | 0.43 | 0.83 | 0.48 | 0.26 | 0.70 | 0.03 | 0.01 |
| 4 | MoM | | | | x | | | | x | | 1st_dbp | 0.61 | 0.39 | 0.83 | 0.52 | 0.30 | 0.74 | 0.06 | 0.01 |
| 4 | MoM | | | | x | | | x | | | H-L-ARG | 0.61 | 0.43 | 0.83 | 0.52 | 0.26 | 0.70 | 0.05 | 0.03 |
| 4 | x | x | | | | | | | | x | Sa-1-P_ | 0.61 | 0.39 | 0.83 | 0.43 | 0.26 | 0.65 | 0.03 | 0.03 |
| | | | | | | | | | | | MoM | | | | | | | | |

### Prognostic Cores:

From the Table 14, it is clear that PIGF is common to all performant cores. A second, highly recurrent variable is s-ENG.

Together these variables constitute the only 2-variable prognostic core:
1. PIGF+s-ENG

Building on this, 2 specific metabolites can significantly improve the specific rule-in performance of the PIGF+s-ENG combination, leading to 2 exceptionally strong 3 variable rule-in prognostic cores:
1. PIGF+s-ENG+DLG
2. PIGF+s-ENG+ECG

Alternative 3 variable prognostic cores are:
3. PIGF+s-ENG+CL
4. PIGF+s-ENG+L-LEU

One of the highly performant 3 variable prognostic cores can be improved further by adding a 4th variable to yield this 1^{st} tier, 4 variable prognostic core:
1. {PIGF AND s-ENG AND ECG}+{20-CL OR fh_pet}

Some 2^{nd} tier alternative 4 variable prognostic cores are also found:
2. {PIGF AND s-ENG}+CR+{L-ISO OR Sa-1-P}
3. {PIGF AND s-ENG}+1-HD+CL
4. {PIGF AND fh_pet}+BR+bp
5. {PIGF AND fh_pet}+1-HD+H-L-ARG

### PPV Threshold:

For the preeclampsia example elaborated here, the PPV threshold for “preterm PE”, was set to PPV=0.071; and was calculated for a PE prevalence=0.014 in accordance with Table 3.

Model Space Filters applied: mean AUC ICI>=0.5; for the statistic mean SensAtPPV 0.071: ICI>=0.05, and impt>=0.05.
Rank complete SensAtPPV from high to low and select models down to 1^{st} single marker
Based on this filtering, a set of 19 multivariable models was found, cf TABLE 15

TABLE 15
Preterm PE

| | | | | | | | | | | | | | | Complete data set | | | 3 fold cross |
|---|---|---|---|---|---|---|---|---|---|---|---|---|---|---|---|---|---|
| n | | | | | | | | | | | | | | Sensitivity (Sn) | | | validation |
| predictors/ | (Recurrent) predictors | | | | | | | | | | | | | at PPV = 0.071 | | | imp |
| model | PIGF | s-ENG | fh_pet | ECG | L-ERG | DLG | L-ISO | DHA | BR | SC | CL | L-LEU | other | Sn | lCl | uCl | SnAtPPV |
| 1 | | | | | | | | | | | | | AcCAR | 0.22 | 0.04 | 0.39 | |
| 1 | (MoM) | | | | | | | | | | | | | 0.48 | 0.00 | 0.70 | |
| 3 | MoM | x | x | | | | | | | | | | | 0.61 | 0.13 | 0.83 | 0.07 |
| 3 | x | x | | x | | | | | | | | | | 0.57 | 0.30 | 0.87 | 0.06 |
| 3 | MoM | | x | | MoM | | | | | | | | | 0.57 | 0.17 | 0.83 | 0.08 |
| 3 | x | x | | | | x | | | | | | | | 0.57 | 0.09 | 0.91 | 0.08 |
| 3 | MoM | | | | | | x | x | | | | | | 0.52 | 0.13 | 0.70 | 0.09 |
| 4 | x | MoM | | | x | x | | | | | | | | 0.65 | 0.09 | 0.91 | 0.06 |
| 4 | x | x | | | x | x | | | | | | | | 0.65 | 0.09 | 0.87 | 0.06 |
| 4 | MoM | x | | | | x | | | x | | | | | 0.61 | 0.35 | 0.91 | 0.07 |
| 4 | MoM | x | | | | x | | | | x | | | | 0.61 | 0.26 | 0.91 | 0.11 |
| 4 | MoM | MoM | | | | x | | | | x | | | | 0.61 | 0.26 | 0.91 | 0.06 |
| 4 | x | x | | | | x | | | | x | | | | 0.61 | 0.26 | 0.91 | 0.09 |
| 4 | x | x | x | x | | | | | | | | | | 0.61 | 0.26 | 0.83 | 0.07 |
| 4 | x | x | | x | | | | | | | | | UR | 0.57 | 0.26 | 0.83 | 0.06 |
| 4 | x | MoM | | | | x | | | x | | | | | 0.57 | 0.26 | 0.91 | 0.06 |
| 4 | MoM | x | | | | x | | | | | | | CR | 0.57 | 0.26 | 0.91 | 0.05 |
| 4 | MoM | x | | | | x | | | | | | | ARA | 0.57 | 0.26 | 0.91 | 0.07 |
| 4 | MoM | | | | | | x | MoM | | | x | | | 0.52 | 0.26 | 0.74 | 0.06 |
| 4 | MoM | | | | | | x | MoM | | | | x | | 0.52 | 0.26 | 0.74 | 0.06 |
| 4 | MoM | | | | | | | MoM | | | x | x | | 0.52 | 0.26 | 0.74 | 0.06 |

### Prognostic Cores:

PIGF is the only variable that offers material single variable performance, but it does not, on its own, meet the filter criteria.

From the Table 15, it is clear that combinations of at least 3 variables are required to improve significantly on the rule-in performance of PIGF.

1^{st} tier, 3 variable prognostic cores include the following:
1. PIGF+s-ENG+ECG
2. PIGF+s-ENG+L-ERG
3. PIGF+s-ENG+DLG
   2^{nd} tier, 3 variable prognostic cores include:
4. PIGF+s-ENG+fh_pet.

While this core is very performant, fh_pet is a clinical risk factor which is prone to error as it requires a detailed knowledge of the medical pregnancy history of relatives. Hence its qualification as a 2^{nd} tier prognostic core
5. PIGF+DHA+L-ISO

Interestingly, PIGF, s-ENG and DLG and L-ERG are also part of a very performant 4 marker model, with the most performant core featuring said 4 variables
1. PIGF+s-ENG+DLG+L-ERG

An alternative 1^{st} tier, 4 variable prognostic core was also found, whereby L-ERG is exchanged by SC or BR. i.e.,
2. {PIGF AND s-ENG AND DLG}+{SC OR BR}

The PIGF, s-ENG, DLG combination features also in a further 2^{nd}′ tier 4 variable prognostic core as follows,
3. {PIGF AND s-ENG AND DLG}+{CR OR ARA}

Some alternative 2^{nd} tier, 4 variable prognostic cores, with a 3 variable-base different from the main 3 variable-base (PIGF+s-ENG+DLG) were also found:
4. {PIGF AND s-ENG AND ECG}+{fh_pet OR UR}
5. {PIGF AND DHA} and any 2 variables from {L-LEU, CL, L-ISO}

### Summary—Rule in Prognostic Performance Preterm PE.

When prognostic performance expressed as Sensitivity (i.e., detection rate of future cases) at set Rule-in thresholds like FPR or PPV, it can be observed that exceptional multi-variable prognostic performance for predicting preterm PE, is achieved following the combination of protein and metabolite variables. Each of the pre-set success criteria (cf. Example 1—Exemplary Prognostic targets for preeclampsia risk stratification tests) are met with ease.

For the FPR rule-in metrics considered, achieving exceptional detection rates at the clinically relevant FPR thresholds is met with the following combinations PIGF AND s-ENG AND DLG, as well as with PIGF AND s-ENG AND ECG, possibly augmented with 20-CL.

For the PPV rule-in metrics considered, achieving exceptional detection rates at the clinically relevant PPV thresholds is met with the following combinations PIGF AND s-ENG with any of the following metabolite variables: ECG, L-ERG and DLG. An extra increment in performance can be achieved when the PIGF AND s-ENG AND DLG core is supplemented with L-ERG or SC.

### Example 5C; PE Sub-Type: Term PE

### FPR Thresholds:

Model Space Filters applied: mean AUC ICI>=0.5; for the statistic mean SensAtSpec 0.80: ICI>=0.2; and imp>=0.03; for the statistic mean SensAtSpec 0.90: ICI>=0.1 and imp>=0.02.
Rank complete SensAtSpec 0.80 from high to low and select models down to 1^{st} single marker
Based on this filtering, a set of 34 multivariable models was found, cf TABLE 16

TABLE 16
Term PE

| | (Recurrent) | | | | | | | | | | |
|---|---|---|---|---|---|---|---|---|---|---|---|
| n | predictors | | | | | | | | | | |
| predictors/ | | | | L- | | L- | L- | L- | | other | |
| model | Bp | PIGF | HVD3 | MET | NGM | LEU | ISO | ARG | BV | recurrent | other |
| 1 | 2nd_sbp | | | | | | | | | | |
| 2 | 2nd_sbp | | x | | | | | | | | |
| 2 | 1st_sbp | MoM | | | | | | | | | |
| 2 | 2nd_sbp | | | x | | | | | | | |
| 2 | 1st_sbp | | | | x | | | | | | |
| 2 | 1st_sbp | | | | | x | | | | | |
| 2 | 1st_sbp | | | | | | x | | | | |
| 2 | 1st_sbp | x | | | | | | | | | |
| 2 | map_1st | | | | | x | | | | | |
| 2 | map_1st | | | x | | | | | | | |
| 2 | 2nd_sbp | | | | | x | | | | | |
| 2 | map_2nd | | | | | | | x | | | |
| 2 | 2nd_sbp | | | | | | | | x | | |
| 2 | 1st_sbp | | x | | | | | | | | |
| 2 | 1st_sbp | | | | | | | x | | | |
| 2 | 1st_sbp | | | | | | | | | | L-ERG_ |
| | | | | | | | | | | | MoM |
| 3 | map_2nd | | x | | | | x | | | | |
| 3 | map_2nd | | x | | | | | x | | | |
| 3 | 1st_dbp | | x | | | | x | | | | |
| 3 | 2nd_sbp | | | | x | | | | | | ECG |
| 3 | 2nd_sbp | | | | | | x | | | | |
| 3 | 1st_sbp | | | | | x | | | | TR_ | |
| | | | | | | | | | | MoM | |
| 3 | 2nd_sbp | | | | | | x | | | 1-HD_ | |
| | | | | | | | | | | MoM | |
| 3 | map_1st | | x | | x | | | | | | |
| 3 | map_1st | | | | | x | | | | ADMA | |
| 3 | 2nd_sbp | | | | | | | | | ADMA | 2-HBA |
| 3 | map_2nd | | | | | x | | | | 1-HD | |
| 3 | 1st_sbp | | | | | | | x | x | | |
| 3 | 1st_sbp | | | | | | | | | | r_glucose + |
| | | | | | | | | | | | s-ENG |
| 4 | map_2nd | | x | | | x | | | | | CR |
| 4 | map_2nd | | x | | | x | | | | TR_ | |
| | | | | | | | | | | MoM | |
| 4 | 2nd_dbp | | | | | | | x | x | | Sa-1-P_ |
| | | | | | | | | | | | MoM |
| 4 | 1st_dbp | | x | | | | | | x | | |
| 4 | | MoM | | | | | x | | | 1-HD | DHA_ |
| | | | | | | | | | | | MoM |

Term PE

| | Complete data set | | | | | | 3 fold cross validation | |
|---|---|---|---|---|---|---|---|---|
| n | Senstivity at | | | Senstivity at | | | Imp | |
| predictors/ | Specificity 0.8 | | | Specificity 0.9 | | | SnAtSp | SnAtSp |
| model | Sn | lCl | uCl | Sn | lCl | uCl | 0.8 | 0.9 |
| 1 | 0.46 | 0.31 | 0.59 | 0.23 | 0.14 | 0.38 | | |
| 2 | 0.51 | 0.35 | 0.66 | 0.34 | 0.19 | 0.47 | 0.07 | 0.09 |
| 2 | 0.50 | 0.35 | 0.62 | 0.34 | 0.19 | 0.46 | 0.07 | 0.12 |
| 2 | 0.50 | 0.38 | 0.64 | 0.31 | 0.20 | 0.45 | 0.04 | 0.11 |
| 2 | 0.49 | 0.35 | 0.62 | 0.36 | 0.24 | 0.49 | 0.04 | 0.16 |
| 2 | 0.49 | 0.34 | 0.62 | 0.35 | 0.24 | 0.46 | 0.03 | 0.16 |
| 2 | 0.49 | 0.36 | 0.62 | 0.32 | 0.20 | 0.46 | 0.04 | 0.18 |
| 2 | 0.49 | 0.35 | 0.64 | 0.31 | 0.19 | 0.45 | 0.07 | 0.14 |
| 2 | 0.49 | 0.36 | 0.62 | 0.30 | 0.19 | 0.42 | 0.11 | 0.03 |
| 2 | 0.49 | 0.34 | 0.64 | 0.28 | 0.19 | 0.43 | 0.14 | 0.05 |
| 2 | 0.49 | 0.34 | 0.62 | 0.28 | 0.19 | 0.42 | 0.06 | 0.11 |
| 2 | 0.49 | 0.36 | 0.62 | 0.28 | 0.16 | 0.42 | 0.06 | 0.07 |
| 2 | 0.49 | 0.35 | 0.62 | 0.27 | 0.16 | 0.42 | 0.03 | 0.08 |
| 2 | 0.49 | 0.34 | 0.63 | 0.37 | 0.22 | 0.50 | 0.09 | 0.15 |
| 2 | 0.47 | 0.36 | 0.61 | 0.36 | 0.26 | 0.47 | 0.04 | 0.18 |
| 2 | 0.47 | 0.32 | 0.62 | 0.31 | 0.19 | 0.43 | 0.03 | 0.11 |
| 3 | 0.56 | 0.43 | 0.68 | 0.34 | 0.21 | 0.50 | 0.05 | 0.03 |
| 3 | 0.54 | 0.41 | 0.68 | 0.34 | 0.22 | 0.49 | 0.04 | 0.06 |
| 3 | 0.53 | 0.41 | 0.66 | 0.31 | 0.18 | 0.46 | 0.04 | 0.01 |
| 3 | 0.51 | 0.39 | 0.64 | 0.39 | 0.26 | 0.53 | 0.04 | 0.01 |
| 3 | 0.51 | 0.39 | 0.64 | 0.38 | 0.24 | 0.51 | 0.06 | 0.02 |
| 3 | 0.50 | 0.36 | 0.62 | 0.36 | 0.24 | 0.49 | 0.08 | 0.01 |
| 3 | 0.50 | 0.39 | 0.62 | 0.35 | 0.23 | 0.50 | 0.04 | 0.02 |
| 3 | 0.50 | 0.37 | 0.63 | 0.34 | 0.21 | 0.50 | 0.05 | 0.03 |
| 3 | 0.50 | 0.35 | 0.64 | 0.32 | 0.20 | 0.45 | 0.04 | 0.03 |
| 3 | 0.50 | 0.38 | 0.64 | 0.30 | 0.16 | 0.45 | 0.05 | 0.01 |
| 3 | 0.49 | 0.36 | 0.61 | 0.36 | 0.22 | 0.50 | 0.04 | 0.02 |
| 3 | 0.49 | 0.35 | 0.62 | 0.36 | 0.26 | 0.49 | 0.03 | 0.01 |
| 3 | 0.47 | 0.34 | 0.61 | 0.31 | 0.19 | 0.43 | 0.03 | 0.01 |
| 4 | 0.57 | 0.44 | 0.71 | 0.41 | 0.21 | 0.57 | 0.06 | 0.03 |
| 4 | 0.56 | 0.43 | 0.69 | 0.38 | 0.25 | 0.53 | 0.03 | 0.01 |
| 4 | 0.51 | 0.36 | 0.65 | 0.32 | 0.20 | 0.46 | 0.04 | 0.03 |
| 4 | 0.49 | 0.35 | 0.63 | 0.25 | 0.13 | 0.37 | 0.03 | 0.02 |
| 4 | 0.47 | 0.35 | 0.58 | 0.27 | 0.16 | 0.45 | 0.03 | 0.02 |

### Prognostic Cores:

From the Table 16, it is clear that a blood pressure measurement is common to all performant combinations. HVD3, L-LEU and L-ISO are other recurrent variables.

From the improvement data it can be seen that supplementing blood pressure with another variable can markedly improve the Rule-in performance. A variety of such 2-variables cores were found:
1. Bp+any one variable from {PIGF, HVD3, L-MET, NGM, L-LEU, L-ISO, L-ARG, BV, L-ERG}

Some of these 2 marker cores are repeated in these 1^{st} tier, 3 variable prognostic cores:
2. {bp AND HVD3}+any one variable from {L-ARG, L-ISO, NGM}
3. {bp AND L-LEU}+1-HD
4. {bp AND L-LEU}+any one variable from {ADMA, TR, 1-HD}

Alternative 3 variable rule-in cores are:
5. bp+NGM+ECG
6. bp+2-HBA+ADMA
7. bp+L-ARG+BV
8. bp+r_glucose+s-ENG

The following 4 variable prognostic cores were found:
1. {bp AND HVD3 AND L-LEU}+{CR OR TR}
2. {bp AND HVD3}+BV+ADMA
3. bp+L-ARG+BV+Sa-1-P

Only one 4 variable prognostic core was found which did not use a blood pressure measurement;
4. PIGF+L-ISO+DHA+1-HD

### PPV Threshold:

For the preeclampsia example elaborated here, the PPV threshold for “term PE”, was set to PPV=0.154; and was calculated for a PE prevalence=0.037 in accordance with Table 3.

Model Space Filters applied: mean AUC ICI>=0.5; for the statistic mean SensAtPPV 0.154: ICI>=0.05; and imp>=0.05.
Rank complete SensAtPPV from high to low and select models down to 1^{st} single marker
Based on this filtering, a set of 41 multivariable models was found, cf TABLE 17, yet only 11 of these reach significance when gauged based on the lower limit of 95% CI, i.e. SnAtPPV ICI>0.05

TABLE 17
Term PE

| | | | | | | | | | | | | | Complete | | | |
|---|---|---|---|---|---|---|---|---|---|---|---|---|---|---|---|---|
| | | | | | | | | | | | | | data set | | | 3 fold cross |
| n pre- | (Recurrent) Predictors | | | | | | | | | | | | Sensitivity at | | | validation |
| dictors/ | blood | | weight | | | | | L- | L- | L- | recurrent | | PPV = 0.154 | | | Imp |
| model | pressure | L-LEU | related | TR | 1-HD | BV | HVD3 | ARG | ISO | LYS | other | other | Sn | lCl | uCl | SnAtPPV |
| 1 | (2nd_sbp) | | | | | | | | | | | | 0.19 | 0.00 | 0.31 | |
| 2 | 1st_sbp | x | | | | | | | | | | | 0.32 | 0.00 | 0.46 | 0.26 |
| 2 | 2nd_sbp | | x | | | | | | | | | | 0.30 | 0.00 | 0.42 | 0.06 |
| 2 | 1st_sbp | | | | | | | | | | | DC | 0.27 | 0.00 | 0.42 | 0.09 |
| 2 | 1st_sbp | | | MoM | | | | | | | | | 0.26 | 0.00 | 0.45 | 0.20 |
| 2 | 1st_sbp | | | | | | | | | | | 12CAR | 0.24 | 0.00 | 0.43 | 0.12 |
| 2 | 2nd_sbp | | | | MoM | | | | | | | | 0.22 | 0.00 | 0.47 | 0.09 |
| 2 | 2nd_sbp | | | | | x | | | | | | | 0.22 | 0.00 | 0.36 | 0.07 |
| 2 | 2nd_sbp | | | | x | | | | | | | | 0.20 | 0.00 | 0.51 | 0.12 |
| 2 | map_2nd | | | | | | | x | | | | | 0.20 | 0.00 | 0.38 | 0.20 |
| 2 | map_2nd | | | | | | x | | | | | | 0.19 | 0.00 | 0.46 | 0.14 |
| 3 | 1st_sbp | | | MoM | | | x | | | | | | 0.35 | 0.15 | 0.53 | 0.05 |
| 3 | 2nd_sbp | | x | | | | x | | | | | | 0.32 | 0.00 | 0.50 | 0.06 |
| 3 | 1st_sbp | | | | | | x | | | x | | | 0.32 | 0.00 | 0.54 | 0.06 |
| 3 | 2nd_sbp | | | MoM | | | x | | | | | | 0.29 | 0.16 | 0.49 | 0.05 |
| 3 | 2nd_sbp | | | | x | | x | | | | | | 0.28 | 0.07 | 0.53 | 0.06 |
| 3 | 2nd_sbp | | | | | | x | | x | | | | 0.28 | 0.07 | 0.51 | 0.06 |
| 3 | map_2nd | | | | x | | | | x | | | | 0.27 | 0.00 | 0.50 | 0.06 |
| 3 | 2nd_sbp | | | x | x | | | | | | | | 0.27 | 0.11 | 0.53 | 0.05 |
| 3 | 2nd_sbp | | | | | | | | | | s-ENG | 3-HBA | 0.24 | 0.00 | 0.45 | 0.06 |
| 3 | 2nd_sbp | | | | MoM | | | | | | | DHA_ | 0.22 | 0.05 | 0.49 | 0.06 |
| | | | | | | | | | | | | MoM | | | | |
| 3 | map_2nd | | | | | x | | x | | | | | 0.22 | 0.00 | 0.42 | 0.06 |
| 3 | map_2nd | | | | | | x | | | x | | | 0.19 | 0.00 | 0.51 | 0.07 |
| 4 | 2nd_sbp | | | | MoM | | | | x | | NGM | | 0.36 | 0.13 | 0.57 | 0.05 |
| 4 | 2nd_sbp | | | MoM | x | | | | x | | | | 0.36 | 0.15 | 0.57 | 0.08 |
| 4 | 2nd_sbp | | | | x | | | | x | | NGM | | 0.35 | 0.15 | 0.57 | 0.06 |
| 4 | 2nd_sbp | | | | MoM | x | | | x | | | | 0.32 | 0.09 | 0.54 | 0.05 |
| 4 | map_1st | x | | | | | x | x | | | | | 0.29 | 0.00 | 0.53 | 0.09 |
| 4 | map_2nd | x | | MoM | x | | | | | | | | 0.28 | 0.00 | 0.55 | 0.06 |
| 4 | map_2nd | | | | MoM | | | | x | | s-ENG_ | | 0.28 | 0.00 | 0.49 | 0.05 |
| | | | | | | | | | | | MoM | | | | | |
| 4 | map_2nd | | | MoM | x | | | | | | | L-MET | 0.28 | 0.00 | 0.54 | 0.05 |
| 4 | 2nd_sbp | | | | | | | | | | s-ENG_ | ADMA + | 0.28 | 0.01 | 0.49 | 0.05 |
| | | | | | | | | | | | MoM | 2-HBA | | | | |
| 4 | map_2nd | x | | MoM | MoM | | | | | | | | 0.28 | 0.00 | 0.54 | 0.11 |
| 4 | 1st_dbp | | | | x | | | | | | DLG | gest | 0.27 | 0.00 | 0.43 | 0.08 |
| 4 | map_1st | | | | | | | | x | x | ECG | | 0.27 | 0.00 | 0.46 | 0.05 |
| 4 | map_1st | x | | | | | x | | | x | | | 0.26 | 0.00 | 0.50 | 0.06 |
| 4 | map_1st | x | | | | | x | | | | ADMA | | 0.25 | 0.00 | 0.51 | 0.07 |
| 4 | 1st_dbp | | | | MoM | | | | | | pLG | gest | 0.24 | 0.00 | 0.42 | 0.07 |
| 4 | 2nd_sbp | | | | | x | | | | | ECG | L-ALA | 0.24 | 0.00 | 0.53 | 0.05 |
| 4 | map_1st | x | | | | | x | | | | | CR | 0.24 | 0.00 | 0.49 | 0.06 |
| 4 | 2nd_sbp | | | x | | x | | | | | | STERA | 0.23 | 0.00 | 0.50 | 0.05 |
| 4 | map_2nd | | | | x | | | | | | ADMA | sDMA | 0.20 | 0.00 | 0.41 | 0.06 |

### Prognostic Cores:

From the TABLE 17, it is clear that a blood pressure measurement is common to all performant combinations and that at least 3 variables are needed to reach significance.

The following 2 variable prognostic cores were identified;
1. {bp AND HVD3}
2. {bp AND 1-HD}

These 2 marker cores are repeated in the 3 variable-prognostic cores:
1. {bp AND HVD3}+any one variable from {TR, 1-HD, L-ISO}
2. {bp AND 1-HD}+any one variable from {TR, L-ISO, DHA}

Within the significant 4 variable combinations, only one of the earlier cores is propagated:
1. {bp AND 1-HD AND L-ISO}+any one variable from {NGM, TR, BV}

### Summary—Rule in Prognostic Performance Term PE.

When prognostic performance is expressed as Sensitivity (i.e., detection rate of future cases) at set Rule-in thresholds like FPR or PPV, it can be observed that multi-variable prognostic performance for predicting term PE, is not achieved easily. For the FPR based rule-in metrics considered, meeting clinically relevant FPR threshold with acceptable detection rates is found possible when combining a blood pressure measure AND HVD3 preferentially with L-LEU or L-ISO. When Considering 4 variable combinations, the further addition of CR or TR is found favorable.

For the PPV rule-in metrics considered, only moderate detection rates (below the set success criterion of Sens>=0.4 at PPV) are found. Combinations of at least 3 variables are required, featuring the following preferred combinations of variables, i.e., blood pressure measure AND HVD3, blood pressure measure AND 1-HD, blood pressure measure AND 1-HD AND L-ISO.

It is of note that within the examples elaborated herein, the multivariable models are restricted to combinations of 4, and strict improvement criteria are applied. Further prognostic performance increments may follow when considering more variables/model, changing the improvement target and/or the thresholds.

### Example 6: Rule-Out Prognostic Performance

In order to identify robust prognostic rule-out cores, models are selected that deliver prognostic performance for the Specificity at 20% FNR (i.e., sensitivity=0.8) criterion as well as the Specificity at 10% FNR (i.e., sensitivity=0.9) criterion, whereby the spec at 20% FNR is considered the more robust criterion, albeit less relevant in a clinical context. Typically, more stringent filters are used for the Spec at Sens 0.80 criterion, both for the lower limits of the 90% confidence interval as well as for the improvement, than for the Spec at Sens 0.90 criterion.

The analysis for the spec at NPV threshold, which is different for each disease sub-type considered (cf. Example 1), is reported on separately.

### Example 6A: PE Sub-Type: All PE

### FNR Thresholds:

Model Space Filters applied: mean AUC ICI>=0.5; for the statistic mean SpecAtSens 0.80: ICI>=0.2, and imp>=0.03; for the statistic mean SpecAtSens 0.90: ICI>=0.1 and imp>=0.02.
Rank complete SensAtSpec 0.80 from high to low and select models down to 1^{st} single marker
Based on this filtering, a set of 54 multivariable models was found, cf TABLE 18

TABLE 18
All PE

| | (Recurrent) | | | | | | | | |
|---|---|---|---|---|---|---|---|---|---|
| n | predictors | | | | | | | | |
| predictors/ | Blood | | | weight | | H-L- | | recurrent | |
| model | pressure | 1-HD | s-ENG | related | HVD3 | ARG | PIGF | other | other |
| 1 | map_2nd | | | | | | | | |
| 2 | map_2nd | x | | | | | | | |
| 2 | map_2nd | | MoM | | | | | | |
| 2 | 2nd_sbp | | | bmi | | | | | |
| 2 | 2nd_sbp | | | | | | | GG | |
| 2 | 2nd_sbp | | | | | | | L-GLU_MoM | |
| 2 | 1st_dbp | | | bmi | | | | | |
| 2 | map_1st | | | bmi | | | | | |
| 2 | 2nd_sbp | | | | | | | | DLG |
| 2 | 1st_sbp | x | | | | | | | |
| 2 | 2nd_sbp | | | | x | | | | |
| 2 | 2nd_sbp | x | | | | | | | |
| 2 | 2nd_sbp | | | | | | | | sDMA |
| 2 | 1st_sbp | MoM | | | | | | | |
| 2 | 2nd_sbp | | | | | x | | | |
| 2 | 1st_sbp | | | bmi | | | | | |
| 2 | 2nd_sbp | | | | | | | L-GLU | |
| 3 | map_2nd | | | bmi | x | | | | |
| 3 | map_2nd | x | MoM | | | | | | |
| 3 | 1st_dbp | | | bmi | x | | | | |
| 3 | map_1st | | | bmi | x | | | | |
| 3 | map_2nd | x | x | | | | | | |
| 3 | map_2nd | | | wgt | x | | | | |
| 3 | 2nd_dbp | | | bmi | x | | | | |
| 3 | 2nd_sbp | x | MoM | | | | | | |
| 3 | map_2nd | x | | | x | | | | |
| 3 | 1st_sbp | | | bmi | x | | | | |
| 3 | 2nd_sbp | x | x | | | | | | |
| 3 | map_1st | x | MoM | | | | | | |
| 3 | 2nd_dbp | x | MoM | | | | | | |
| 3 | 2nd_sbp | x | | | x | | | | |
| 3 | map_1st | x | | | x | | | | |
| 3 | 2nd_sbp | MoM | x | | | | | | |
| 3 | map_1st | | | wgt | x | | | | |
| 3 | 1st_sbp | x | MoM | | | | | | |
| 3 | 1st_sbp | | | waist | x | | | | |
| 3 | map_1st | MoM | MoM | | | | | | |
| 3 | | x | MoM | | | | MoM | | |
| 3 | 1st_dbp | | MoM | | | | | NGM | |
| 3 | | x | | | | | MoM | 2-HBA | |
| 3 | map_1st | | MoM | | | | | | L-MET |
| 3 | | x | MoM | | | | x | | |
| 4 | | x | MoM | | | | x | 2-HBA | |
| 4 | 2nd_sbp | x | | | x | x | | | |
| 4 | | x | MoM | | | | MoM | 2-HBA | |
| 4 | 2nd_sbp | MoM | | | x | x | | | |
| 4 | | x | x | | | | MoM | EPA | |
| 4 | 1st_dbp | x | | | x | | | NGM | |
| 4 | | | | bmi | x | | | | fh_pet + gest |
| 4 | | x | | | | | MoM | L-ISO + EPA | |
| 4 | map_2nd | | | | | | | GG | 20-CL + CR |
| 4 | map_1st | | MoM | | x | x | | | |
| 4 | | MoM | | | | | MoM | L-ISO + EPA | |
| 4 | 1st_dbp | x | | | x | x | | | |
| 4 | 1st_dbp | | MoM | | | | | L-ISO | ECG |

All PE

| | Complete data set | | | | | | 3 fold cross validation | |
|---|---|---|---|---|---|---|---|---|
| n | Senstivity at | | | Senstivity at | | | Imp | |
| predictors/ | Specificity = 0.8 | | | Specificity = 0.9 | | | SnAtSp | SnAtSp |
| model | Sn | lCl | uCl | Sn | lCl | uCl | 0.8 | 0.9 |
| 1 | 0.43 | 0.28 | 0.53 | 0.23 | 0.13 | 0.39 | | |
| 2 | 0.50 | 0.33 | 0.59 | 0.33 | 0.19 | 0.43 | 0.05 | 0.09 |
| 2 | 0.48 | 0.36 | 0.58 | 0.28 | 0.18 | 0.44 | 0.05 | 0.12 |
| 2 | 0.45 | 0.36 | 0.59 | 0.30 | 0.18 | 0.44 | 0.09 | 0.04 |
| 2 | 0.45 | 0.34 | 0.52 | 0.29 | 0.19 | 0.42 | 0.06 | 0.04 |
| 2 | 0.44 | 0.33 | 0.53 | 0.27 | 0.14 | 0.41 | 0.05 | 0.02 |
| 2 | 0.44 | 0.30 | 0.62 | 0.27 | 0.16 | 0.41 | 0.09 | 0.09 |
| 2 | 0.44 | 0.35 | 0.61 | 0.29 | 0.19 | 0.42 | 0.07 | 0.07 |
| 2 | 0.44 | 0.33 | 0.54 | 0.30 | 0.23 | 0.42 | 0.12 | 0.07 |
| 2 | 0.44 | 0.33 | 0.52 | 0.29 | 0.21 | 0.41 | 0.15 | 0.10 |
| 2 | 0.44 | 0.36 | 0.51 | 0.33 | 0.22 | 0.46 | 0.08 | 0.09 |
| 2 | 0.44 | 0.34 | 0.54 | 0.35 | 0.20 | 0.41 | 0.09 | 0.04 |
| 2 | 0.43 | 0.32 | 0.51 | 0.27 | 0.18 | 0.40 | 0.05 | 0.02 |
| 2 | 0.43 | 0.31 | 0.51 | 0.29 | 0.19 | 0.39 | 0.12 | 0.09 |
| 2 | 0.43 | 0.35 | 0.50 | 0.28 | 0.19 | 0.42 | 0.08 | 0.04 |
| 2 | 0.43 | 0.34 | 0.59 | 0.31 | 0.21 | 0.41 | 0.05 | 0.09 |
| 2 | 0.43 | 0.30 | 0.51 | 0.27 | 0.14 | 0.39 | 0.04 | 0.02 |
| 3 | 0.55 | 0.46 | 0.63 | 0.37 | 0.20 | 0.54 | 0.05 | 0.05 |
| 3 | 0.55 | 0.45 | 0.62 | 0.41 | 0.28 | 0.53 | 0.05 | 0.03 |
| 3 | 0.54 | 0.38 | 0.66 | 0.33 | 0.20 | 0.49 | 0.12 | 0.09 |
| 3 | 0.53 | 0.39 | 0.62 | 0.37 | 0.21 | 0.50 | 0.08 | 0.07 |
| 3 | 0.53 | 0.44 | 0.60 | 0.40 | 0.29 | 0.52 | 0.03 | 0.03 |
| 3 | 0.53 | 0.44 | 0.60 | 0.37 | 0.17 | 0.52 | 0.04 | 0.03 |
| 3 | 0.53 | 0.40 | 0.64 | 0.35 | 0.23 | 0.50 | 0.04 | 0.02 |
| 3 | 0.51 | 0.42 | 0.60 | 0.38 | 0.23 | 0.49 | 0.08 | 0.05 |
| 3 | 0.51 | 0.36 | 0.61 | 0.34 | 0.26 | 0.45 | 0.03 | 0.06 |
| 3 | 0.51 | 0.37 | 0.66 | 0.34 | 0.25 | 0.46 | 0.09 | 0.05 |
| 3 | 0.50 | 0.40 | 0.58 | 0.37 | 0.23 | 0.49 | 0.04 | 0.03 |
| 3 | 0.50 | 0.40 | 0.57 | 0.39 | 0.25 | 0.49 | 0.04 | 0.06 |
| 3 | 0.49 | 0.39 | 0.60 | 0.37 | 0.26 | 0.47 | 0.04 | 0.08 |
| 3 | 0.48 | 0.38 | 0.59 | 0.37 | 0.27 | 0.46 | 0.05 | 0.04 |
| 3 | 0.48 | 0.38 | 0.56 | 0.36 | 0.19 | 0.47 | 0.03 | 0.08 |
| 3 | 0.48 | 0.40 | 0.57 | 0.35 | 0.19 | 0.48 | 0.04 | 0.02 |
| 3 | 0.48 | 0.37 | 0.58 | 0.34 | 0.18 | 0.46 | 0.06 | 0.05 |
| 3 | 0.47 | 0.40 | 0.54 | 0.36 | 0.26 | 0.48 | 0.06 | 0.09 |
| 3 | 0.47 | 0.36 | 0.63 | 0.35 | 0.19 | 0.44 | 0.10 | 0.02 |
| 3 | 0.47 | 0.36 | 0.59 | 0.34 | 0.24 | 0.43 | 0.04 | 0.04 |
| 3 | 0.46 | 0.27 | 0.56 | 0.21 | 0.11 | 0.38 | 0.07 | 0.07 |
| 3 | 0.45 | 0.32 | 0.57 | 0.29 | 0.12 | 0.40 | 0.04 | 0.04 |
| 3 | 0.45 | 0.36 | 0.57 | 0.34 | 0.24 | 0.44 | 0.06 | 0.08 |
| 3 | 0.44 | 0.31 | 0.57 | 0.28 | 0.15 | 0.40 | 0.03 | 0.02 |
| 3 | 0.44 | 0.26 | 0.56 | 0.21 | 0.14 | 0.37 | 0.09 | 0.06 |
| 4 | 0.51 | 0.33 | 0.59 | 0.31 | 0.23 | 0.44 | 0.05 | 0.05 |
| 4 | 0.50 | 0.41 | 0.60 | 0.37 | 0.29 | 0.49 | 0.03 | 0.03 |
| 4 | 0.48 | 0.36 | 0.61 | 0.32 | 0.20 | 0.44 | 0.05 | 0.05 |
| 4 | 0.48 | 0.38 | 0.56 | 0.36 | 0.28 | 0.49 | 0.03 | 0.02 |
| 4 | 0.47 | 0.32 | 0.59 | 0.23 | 0.15 | 0.44 | 0.06 | 0.02 |
| 4 | 0.46 | 0.39 | 0.55 | 0.37 | 0.16 | 0.47 | 0.06 | 0.04 |
| 4 | 0.46 | 0.36 | 0.55 | 0.37 | 0.16 | 0.45 | 0.03 | 0.05 |
| 4 | 0.46 | 0.33 | 0.61 | 0.28 | 0.17 | 0.40 | 0.05 | 0.06 |
| 4 | 0.45 | 0.31 | 0.56 | 0.30 | 0.18 | 0.39 | 0.03 | 0.02 |
| 4 | 0.45 | 0.36 | 0.61 | 0.20 | 0.13 | 0.45 | 0.03 | 0.02 |
| 4 | 0.44 | 0.27 | 0.60 | 0.23 | 0.12 | 0.40 | 0.04 | 0.05 |
| 4 | 0.44 | 0.31 | 0.56 | 0.28 | 0.18 | 0.41 | 0.04 | 0.05 |
| 4 | 0.43 | 0.35 | 0.59 | 0.30 | 0.12 | 0.41 | 0.05 | 0.04 |

### Prognostic Cores:

From the Table 18, it is very apparent that a combination of 3 variables can deliver very good rule-out performance, e.g., a blood pressure measurement, together with {a weight-related measurement OR 1-HD} and {HVD3 OR s-ENG}.

As apparent from the improvements, combining blood pressure with a number of metabolites can add to the predictive performance. With the mentioned variables, the following 1^{st} tier 2 variable prognostic cores were found:
1. bp+1-HD
2. bp+WRV
3. bp+HVD3
4. bp+s-ENG

Some 2^{nd} tier 2 variable prognostic cores were also found:
5. bp+any one variable from {GG, L-GLU, DLG, H-L-ARG, SDMA}

Two very pertinent cores within the 1^{st} tier 3 variable prognostic cores are:
1. bp AND HVD3 AND {1-HD OR WRV}.
2. bp AND s-ENG AND {1-HD OR WRV}.

A 2^{nd} tier 3-variable prognostic core which does not require 1-HD or WRV, is also present:
3. {bp AND s-ENG}+{L-MET OR NGM}

An alternative 2^{nd} tier 3-variable prognostic core which does not feature blood pressure, is:
4. {1-HD AND PIGF}+{s-ENG OR 2-HBA}

The combinations of 4 variables do not improve any of the 3 variable prognostic cores, but some compliant 4 variable prognostic cores are reported as further additive value may become apparent in larger patient cohorts.
1. bp AND HVD3 AND s-ENG+H-L-ARG
2. 1-HD AND PIGF+s-ENG+{2-HBA OR EPA}
3. 1-HD AND PIGF+L-ISO+EPA

### NPV Threshold:

For the preeclampsia example elaborated here, the NPV threshold for “all PE”, was set to NPV=0.9889, and calculated for PE prevalence=0.05 in accordance with Table 3.

Model Space Filters applied: mean AUC ICI>=0.5; for the statistic mean SpecAtNPV 0.9889: ICI>=0.075; and imp>=0.05.
Rank complete SpecAtNPV from high to low and select models down to 1^{st} single marker.
Based on this filtering, a set of 47 multivariable models was found, cf TABLE 19

TABLE 19
All PE

| | | | | | | | | | | | | Complete | | | |
|---|---|---|---|---|---|---|---|---|---|---|---|---|---|---|---|
| | | | | | | | | | | | | data set | | | 3 fold cross |
| n | (Recurrent) predictors | | | | | | | | | | | Specificity at | | | validation |
| predictors/ | blood | | | | weight | | | | | other | | NPV = 0.9889 | | | Improvement |
| model | pressure | 1-HD | HVD3 | DLG | related | L-ARG | s-Flt1 | PIGF | s-ENG | recurrent | other | Sp | lCl | uCl | SpAtNPV |
| 1 | | | | | | | | | | L-ERG | | 0.15 | 0.09 | 0.20 | |
| 2 | map_2nd | x | | | | | | | | | | 0.29 | 0.04 | 0.49 | 0.12 |
| 2 | 2nd_sbp | | x | | | | | | | | | 0.29 | 0.03 | 0.46 | 0.16 |
| 2 | 2nd_sbp | | | x | | | | | | | | 0.26 | 0.17 | 0.41 | 0.12 |
| 2 | 1st_sbp | x | | | | | | | | | | 0.24 | 0.16 | 0.40 | 0.22 |
| 2 | 1st_sbp | | | | bmi | | | | | | | 0.24 | 0.04 | 0.40 | 0.18 |
| 2 | 2nd_sbp | | | | | x | | | | | | 0.24 | 0.10 | 0.35 | 0.11 |
| 2 | map_2nd | | | | | x | | | | | | 0.22 | 0.13 | 0.28 | 0.09 |
| 2 | map_2nd | | | | | | x | | | | | 0.21 | 0.01 | 0.40 | 0.14 |
| 2 | map_2nd | | | | | | | | | NGM | | 0.21 | 0.06 | 0.31 | 0.12 |
| 2 | map_1st | | | | bmi | | | | | | | 0.20 | 0.03 | 0.41 | 0.20 |
| 2 | map_2nd | | | | | | MoM | | | | | 0.20 | 0.01 | 0.41 | 0.14 |
| 2 | 2nd_sbp | | | | bmi | | | | | | | 0.20 | 0.01 | 0.44 | 0.05 |
| 2 | 2nd_sbp | | | | | | | MoM | | | | 0.19 | 0.10 | 0.34 | 0.05 |
| 2 | map_2nd | | x | | | | | | | | | 0.18 | 0.11 | 0.45 | 0.15 |
| 2 | 1st_sbp | | x | | | | | | | | | 0.17 | 0.06 | 0.36 | 0.19 |
| 2 | 1st_sbp | | | | | | x | | | | | 0.17 | 0.05 | 0.30 | 0.22 |
| 2 | 2nd_sbp | | | | | | | x | | | | 0.17 | 0.11 | 0.35 | 0.07 |
| 2 | 2nd_dbp | | | | | | | | | NGM | | 0.17 | 0.01 | 0.30 | 0.17 |
| 2 | 1st_sbp | | | | | | | | | GG | | 0.17 | 0.12 | 0.34 | 0.20 |
| 2 | map_2nd | | | | | | | | | 20-CL | | 0.16 | 0.01 | 0.36 | 0.07 |
| 3 | 2nd_sbp | x | x | | | | | | | | | 0.36 | 0.03 | 0.48 | 0.06 |
| 3 | map_1st | | x | | bmi | | | | | | | 0.33 | 0.05 | 0.56 | 0.10 |
| 3 | map_2nd | x | x | | | | | | | | | 0.32 | 0.12 | 0.50 | 0.09 |
| 3 | 1st_sbp | | x | | bmi | | | | | | | 0.30 | 0.07 | 0.52 | 0.06 |
| 3 | 2nd_sbp | MoM | | x | | | | | | | | 0.30 | 0.15 | 0.47 | 0.07 |
| 3 | 2nd_sbp | x | | x | | | | | | | | 0.29 | 0.14 | 0.49 | 0.09 |
| 3 | map_1st | x | | | | | | | | ADMA | | 0.29 | 0.02 | 0.45 | 0.05 |
| 3 | map_2nd | x | | | | | | | | GG | | 0.27 | 0.03 | 0.52 | 0.07 |
| 3 | map_1st | MoM | | | | | x | | | | | 0.19 | 0.01 | 0.35 | 0.07 |
| 3 | 1st_sbp | | | | | | | | | fh_pet | S-1- | 0.16 | 0.08 | 0.37 | 0.07 |
| | | | | | | | | | | | P_MoM | | | | |
| 4 | map_1st | x | x | | | | | | MoM | | | 0.38 | 0.04 | 0.52 | 0.07 |
| 4 | | x | | | | x | | MoM | | 2-HBA | | 0.34 | 0.00 | 0.45 | 0.05 |
| 4 | | x | | | | x | | x | | 2-HBA | | 0.31 | 0.00 | 0.47 | 0.05 |
| 4 | | x | | | | | | x | x | 2-HBA | | 0.26 | 0.01 | 0.48 | 0.05 |
| 4 | | | | | wgt | | | | x | ADMA + | | 0.26 | 0.00 | 0.35 | 0.09 |
| | | | | | | | | | | H-L-ARG | | | | | |
| 4 | | x | | | | | | x | MoM | 2-HBA | | 0.26 | 0.00 | 0.48 | 0.06 |
| 4 | map_2nd | | x | | bmi | | | | | | f1_age | 0.26 | 0.04 | 0.59 | 0.07 |
| 4 | | | | | wgt | | | | MoM | ADMA + | | 0.25 | 0.00 | 0.34 | 0.10 |
| | | | | | | | | | | H-L-ARG | | | | | |
| 4 | 1st_sbp | x | x | | | | | | | UR | | 0.25 | 0.11 | 0.50 | 0.05 |
| 4 | map_2nd | MoM | x | | | | | | | BV | | 0.25 | 0.12 | 0.42 | 0.05 |
| 4 | | MoM | | | | | | MoM | MoM | L-ISO | | 0.25 | 0.00 | 0.35 | 0.06 |
| 4 | | x | | | | | | MoM | MoM | 2-HBA | | 0.23 | 0.00 | 0.44 | 0.05 |
| 4 | | x | x | | | | x | | | fh_pet | | 0.19 | 0.07 | 0.36 | 0.07 |
| 4 | | x | | | | | | MoM | | L-ISO | EPA | 0.19 | 0.00 | 0.39 | 0.06 |
| 4 | | x | | | | x | | MoM | x | | | 0.17 | 0.00 | 0.30 | 0.07 |
| 4 | | | | | | | | x | MoM | NGM + | | 0.16 | 0.00 | 0.28 | 0.07 |
| | | | | | | | | | | L-ISO | | | | | |
| 4 | | x | | | | | | x | MoM | H-L-ARG | | 0.16 | 0.00 | 0.37 | 0.06 |

### Prognostic Cores:

From the TABLE 19, it is clear that combining blood pressure with a number of variables can add to the predictive rule-out performance (as per the PPV criterion). 1-HD is found as a strong additive variable with significant rule-out performance throughout.

The following 1^{st} tier 2 variable prognostic cores were found:
1. bp+1-HD
2. bp+HVD3
3. bp+DLG

Some 2^{nd} tier 2 variable prognostic cores were also found:
4. bp+any one variable from {WRV, L-ARG, s-Flt1, NGM, PIGF, GG and 20-CL}

Two very pertinent cores within the combinations of 3 variables constitutes:
1. {bp AND 1-HD}+HVD3
2. {bp AND 1-HD}+DLG

Some 2^{nd} tier 3-variable prognostic cores are also found:
3. {bp AND 1-HD}+any one variable from {ADMA, GG, sFlt1}
4. bp+HVD3+WRV
5. bp+S-1-P+fh_pet

A sole 4 variable prognostic core was found to improve specificity compared to the 3-variable prognostic cores:
1. bp+1-HD+HVD3+s-ENG

The other combinations of 4 variables do not improve any of the 3 variable combinations, but a set of compliant 4 variable combinations are reported as further additive value may become apparent in larger patient cohorts. It is of note that 1-HD remains a recurrent constituent in these 4 variable prognostic cores, whereas blood pressure is not so anymore (no significant improvements over bp models found)
2. bp AND 1-HD AND HVD3+any one variable from {PIGF, UR, BV}
3. bp AND HVD3+WRV+age
4. 1-HD AND PIGF AND s-ENG+any one variable from {2-HBA, L-ISO, L-ARG, H-L-ARG}
5. 1-HD AND PIGF+L-ARG+2-HBA
6. 1-HD AND PIGF+L-ISO+EPA
7. 1-HD AND HVD3+s-Flt1+fh_pet
8. s-ENG+WRV+ADMA+H-L-ARG
9. s-ENG+PIGF+NGM+L-ISO

### Summary—Rule-out Prognostic Performance All PE

When prognostic performance is expressed as specificity (i.e., detection rate of future non-cases) at set Rule-out thresholds like FNR or NPV, it can be observed that exceptional multi-variable prognostic performance for predicting the absence of future PE, is best achieved following the combination of a blood pressure AND 1-HD augmented with s-ENG OR/AND HVD3. By doing so, the pre-set FNR success criteria (cf. Example 1—Exemplary Prognostic targets for preeclampsia risk stratification tests) are met, whereas the NPV success criterion is nearly achieved.

### Example 6B: PE Sub-Type: Preterm PE

### FNR Thresholds:

Model Space Filters applied: mean AUC ICI>=0.5; for the statistic mean SpecAtSens 0.80: ICI>=0.3, and imp>=0.03; for the statistic mean SpecAtSens 0.90: ICI>=0.2, and imp>=0.02. Rank complete SensAtSpec 0.80 from high to low and select models down to 1^{st} single marker. Based on this filtering, a set of 26 multivariable models was found, cf TABLE 20.

TABLE 20
Preterm PE

| | | | | | | | | | | | | | | | | | 3 fold cross | |
|---|---|---|---|---|---|---|---|---|---|---|---|---|---|---|---|---|---|---|
| | | | | | | | | | | | | | | | | | validation | |
| n pre- | (Recurrent) Predictors | | | | | | | | | | Specificity at | | | Specificity at | | | Improvement | |
| dictors/ | s- | | 1- | | L- | 2- | | fh_ | recurrent | | Sensitivity 0.8 | | | Sensitivity 0.9 | | | SnAtSp | SnAtSp |
| model | ENG | DLG | HD | CL | ERG | HBA | HVD3 | pet | other | other | Sp | lCl | uCl | Sp | lCl | uCl | 0.8 | 0.9 |
| 1 | | | | | | | | | | PIGF | 0.47 | 0.2 | 0.74 | 0.28 | 0.03 | 0.63 | | |
| 2 | x | x | | | | | | | | | 0.63 | 0.26 | 0.77 | 0.45 | 0.17 | 0.71 | 0.09 | 0.03 |
| 2 | MoM | x | | | | | | | | | 0.63 | 0.27 | 0.78 | 0.51 | 0.18 | 0.73 | 0.10 | 0.04 |
| 2 | MoM | | x | | | | | | | | 0.55 | 0.17 | 0.69 | 0.48 | 0.13 | 0.62 | 0.14 | 0.02 |
| 2 | x | | x | | | | | | | | 0.54 | 0.14 | 0.68 | 0.49 | 0.11 | 0.62 | 0.14 | 0.03 |
| 3 | x | x | | x | | | | | | | 0.66 | 0.39 | 0.80 | 0.50 | 0.13 | 0.72 | 0.05 | 0.04 |
| 3 | MoM | x | | x | | | | | | | 0.66 | 0.37 | 0.80 | 0.49 | 0.17 | 0.73 | 0.04 | 0.03 |
| 3 | MoM | | x | | | | | | COT | | 0.58 | 0.20 | 0.70 | 0.52 | 0.06 | 0.66 | 0.03 | 0.03 |
| 3 | | | x | x | MoM | | | | | | 0.50 | 0.38 | 0.61 | 0.43 | 0.31 | 0.56 | 0.08 | 0.05 |
| 3 | | | | | | x | x | | | L- | 0.48 | 0.38 | 0.62 | 0.43 | 0.08 | 0.53 | 0.03 | 0.17 |
| | | | | | | | | | | ALA | | | | | | | | |
| 3 | | | | | | | | | ARA + | | 0.47 | 0.25 | 0.62 | 0.40 | 0.04 | 0.56 | 0.08 | 0.13 |
| | | | | | | | | | DHA_ | | | | | | | | | |
| | | | | | | | | | MoM + | | | | | | | | | |
| | | | | | | | | | EPA | | | | | | | | | |
| 4 | x | x | x | | | | | | NGM | | 0.69 | 0.30 | 0.77 | 0.64 | 0.24 | 0.73 | 0.04 | 0.03 |
| 4 | x | x | | | MoM | | | | SC | | 0.67 | 0.38 | 0.79 | 0.57 | 0.18 | 0.74 | 0.04 | 0.04 |
| 4 | MoM | | x | | | x | | | L-ARG | | 0.64 | 0.53 | 0.72 | 0.57 | 0.10 | 0.69 | 0.04 | 0.05 |
| 4 | | | | | MoM | | | x | SC + EPA | | 0.61 | 0.42 | 0.71 | 0.47 | 0.05 | 0.66 | 0.05 | 0.03 |
| 4 | x | | MoM | | | x | x | | | | 0.58 | 0.27 | 0.78 | 0.50 | 0.03 | 0.72 | 0.04 | 0.06 |
| 4 | | | x | x | x | | | x | | | 0.57 | 0.34 | 0.70 | 0.40 | 0.29 | 0.63 | 0.04 | 0.06 |
| 4 | | x | | x | | | | | ADMA | CR | 0.57 | 0.30 | 0.74 | 0.36 | 0.19 | 0.68 | 0.05 | 0.03 |
| 4 | | | MoM | x | MoM | | | | NGM | | 0.56 | 0.37 | 0.65 | 0.47 | 0.18 | 0.62 | 0.06 | 0.03 |
| 4 | | | x | x | MoM | | | | L-ARG | | 0.56 | 0.27 | 0.66 | 0.39 | 0.22 | 0.62 | 0.04 | 0.05 |
| 4 | | | | x | | x | x | | | map_ | 0.55 | 0.12 | 0.73 | 0.29 | 0.09 | 0.65 | 0.05 | 0.03 |
| | | | | | | | | | | 1st | | | | | | | | |
| 4 | | | | | | x | x | | ARA | 20-CL | 0.54 | 0.30 | 0.75 | 0.37 | 0.06 | 0.59 | 0.07 | 0.12 |
| 4 | | | | x | | x | x | | DHA | | 0.53 | 0.27 | 0.69 | 0.42 | 0.22 | 0.61 | 0.03 | 0.05 |
| 4 | | | | x | x | | | x | L-ARG | | 0.53 | 0.36 | 0.65 | 0.44 | 0.12 | 0.60 | 0.06 | 0.03 |
| 4 | | | | x | | x | x | | DHA_ | | 0.50 | 0.27 | 0.71 | 0.43 | 0.17 | 0.61 | 0.06 | 0.08 |
| | | | | | | | | | MoM | | | | | | | | | |
| 4 | | | | x | x | | | x | ADMA | | 0.50 | 0.39 | 0.63 | 0.43 | 0.16 | 0.56 | 0.03 | 0.03 |
| 4 | | | | | | | | | ADMA + | | 0.49 | 0.38 | 0.56 | 0.46 | 0.01 | 0.53 | 0.03 | 0.03 |
| | | | | | | | | | DHA_ | | | | | | | | | |
| | | | | | | | | | MoM + | | | | | | | | | |
| | | | | | | | | | EPA | | | | | | | | | |

### Prognostic Cores:

From the TABLE 20, it is clear that Rule-out models (as assessed by FNR), are governed by a different set of variables than the corresponding preterm PE rule-in models. s-ENG is common to 2 highly prognostic 2-variable rule-out cores:

The two 2 variable prognostic cores identified are:
1. s-ENG+DLG
2. s-ENG+1-HD

Building on this, two highly performant, first tier rule-out 3-variable prognostic cores are found;
1. {s-ENG AND DLG}+CL
2. {s-ENG AND 1-HD}+{CL OR COT}

Alternative, 2^{nd} tier 3 variable rule-out prognostic cores are:
3. 1-HD+CL+L-ERG
4. 2-HBA+HVD3+L-ALA
5. ARA+DHA+EPA

The highest prognostic performances within the 4 variable combinations feature the strong 2 variable prognostic cores:
1. {s-ENG AND DLG AND 1-HD}+NGM
2. {s-ENG AND DLG}+L-ERG+L-ARG
3. {s-ENG AND 1-HD}+2-HBA+{HVD3 OR L-ARG}

Some 2^{nd} tier alternative 4 variable prognostic cores feature CL instead of s-ENG:
4. {CL AND 1-HD AND L-ERG}+any one variable from +{L-ARG, NGM, ADMA, fh_pet}
5. {CL AND 2-HBA AND HVD3}+{bp OR DHA}
6. {CL AND L-ERG}+L-ARG+ADMA

### NPV Threshold:

For the preeclampsia example elaborated here, the NPV threshold for “preterm PE”, was set to NPV=0.9975; and was calculated for a preterm PE prevalence=0.014 in accordance with TABLE 3.

Model Space Filters applied: mean AUC ICI>=0.5; for the statistic mean SpecAtNPV 0.9975: ICI>=0.25 and imp>=0.1.
Rank complete SpecAtNPV from high to low and select models down to 1^{st} single marker.
Based on this filtering, a set of 37 multivariable models was found, cf TABLE 21

TABLE 21
Preterm PE

| | (Recurrent) | | | | | | | | | | | |
|---|---|---|---|---|---|---|---|---|---|---|---|---|
| n | Predictors | | | | | | | | | | | |
| predictors/ | s- | | 1- | weight | | 2- | L- | L- | | | | |
| model | ENG | DLG | HD | related | HVD3 | HBA | ALA | ERG | CL | ARA | DHA | DGLA |
| 1 | | | | | | x | | | | | | |
| 2 | MoM | x | | | | | | | | | | |
| 2 | x | | x | | | | | | | | | |
| 2 | MoM | | x | | | | | | | | | |
| 2 | MoM | | | bmi | | | | | | | | |
| 2 | x | | | bmi | | | | | | | | |
| 2 | x | x | | | | | | | | | | |
| 3 | MoM | x | MoM | | | | | | | | | |
| 3 | MoM | | x | | | | | | | | | |
| 3 | | | | | x | x | x | | | | | |
| 3 | | | | bmi | x | | | MoM | | | | |
| 3 | | | | | | | | x | x | | | |
| 3 | | | | | | | | | | x | MoM | |
| 4 | x | x | | | | | | | | | x | x |
| 4 | MoM | x | | | | | | | | | MoM | |
| 4 | MoM | | | | | | | | x | | | |
| 4 | MoM | x | | | | | | | | x | | |
| 4 | x | x | | | | | | | | | MoM | |
| 4 | x | x | | | | x | | | | x | | |
| 4 | MoM | x | | | | x | | | | | | |
| 4 | x | x | | | | | | | | x | | |
| 4 | x | x | | | | | | MoM | | | | |
| 4 | x | | | | | | | | x | | | |
| 4 | MoM | x | | | | | | MoM | | | | |
| 4 | MoM | | | | | | x | | | | | |
| 4 | x | | | | | | x | | | | | |
| 4 | MoM | | | | | | | | | | | |
| 4 | x | x | | | | | | | | x | | |
| 4 | MoM | | | | | | | | | | | |
| 4 | MoM | | | | | | | | x | | | |
| 4 | MoM | | | | | | | | | x | | |
| 4 | x | | | | | | | | x | | | |
| 4 | | | | | | | | | x | | | |
| 4 | x | | | | | | | | x | | | |
| 4 | | | | | x | x | | | x | | | |
| 4 | | | | | | x | | | | | MoM | |
| 4 | | | | | | | | | x | | x | x |
| 4 | | | | | | x | | | | | | |

| | | | | | Complete data set | | | 3 fold cross |
|---|---|---|---|---|---|---|---|---|
| n | (Recurrent) | | | | Specificity at | | | validation |
| predictors/ | Predictors | | recurrent | | NPV = 0.9975 | | | Imp |
| model | PIGF | SC | other | other | Sp | lCl | uCl | SpAtNPV |
| 1 | | | | | 0.21 | 0.16 | 0.32 | |
| 2 | | | | | 0.51 | 0.16 | 0.75 | 0.30 |
| 2 | | | | | 0.47 | 0.10 | 0.62 | 0.32 |
| 2 | | | | | 0.46 | 0.13 | 0.62 | 0.28 |
| 2 | | | | | 0.43 | 0.36 | 0.65 | 0.36 |
| 2 | | | | | 0.43 | 0.36 | 0.65 | 0.36 |
| 2 | | | | | 0.30 | 0.15 | 0.73 | 0.27 |
| 3 | | | | | 0.59 | 0.16 | 0.77 | 0.14 |
| 3 | | | | r_glucose | 0.56 | 0.04 | 0.63 | 0.14 |
| 3 | | | | | 0.39 | 0.07 | 0.50 | 0.13 |
| 3 | | | | | 0.30 | 0.18 | 0.57 | 0.14 |
| 3 | | | | LINA | 0.27 | 0.16 | 0.54 | 0.11 |
| 3 | | | | EPA | 0.26 | 0.03 | 0.56 | 0.13 |
| 4 | | | | | 0.59 | 0.15 | 0.74 | 0.14 |
| 4 | | | H-L-ARG | | 0.58 | 0.11 | 0.70 | 0.11 |
| 4 | | | ECG + MYRA | | 0.57 | 0.21 | 0.65 | 0.11 |
| 4 | MoM | | | | 0.56 | 0.14 | 0.86 | 0.10 |
| 4 | | | H-L-ARG | | 0.56 | 0.11 | 0.69 | 0.12 |
| 4 | | | | | 0.55 | 0.12 | 0.71 | 0.14 |
| 4 | | | | 3-HBA | 0.55 | 0.19 | 0.75 | 0.10 |
| 4 | MoM | | | | 0.55 | 0.15 | 0.86 | 0.11 |
| 4 | | | cig_1st_trim_gp | | 0.55 | 0.20 | 0.67 | 0.13 |
| 4 | | | ECG + MYRA | | 0.54 | 0.14 | 0.64 | 0.12 |
| 4 | | | cig_1st_trim_gp | | 0.54 | 0.20 | 0.67 | 0.13 |
| 4 | MoM | x | | | 0.54 | 0.11 | 0.79 | 0.17 |
| 4 | MoM | x | | | 0.54 | 0.10 | 0.81 | 0.17 |
| 4 | MoM | x | NGM | | 0.53 | 0.19 | 0.78 | 0.11 |
| 4 | | | | UR | 0.51 | 0.15 | 0.68 | 0.12 |
| 4 | MoM | x | | L-MET | 0.50 | 0.12 | 0.80 | 0.10 |
| 4 | MoM | x | | | 0.50 | 0.12 | 0.82 | 0.13 |
| 4 | MoM | x | | | 0.50 | 0.12 | 0.79 | 0.16 |
| 4 | MoM | x | | | 0.50 | 0.12 | 0.83 | 0.16 |
| 4 | MoM | | L-ISO | TR | 0.50 | 0.16 | 0.79 | 0.17 |
| 4 | PIGF | x | | | 0.39 | 0.12 | 0.82 | 0.12 |
| 4 | | | | 20-CL | 0.35 | 0.25 | 0.67 | 0.14 |
| 4 | | | fh_pet + NGM | | 0.33 | 0.24 | 0.52 | 0.11 |
| 4 | | | fh_pet | | 0.31 | 0.24 | 0.51 | 0.12 |
| 4 | | | ADMA | BV + L-LYS | 0.27 | 0.09 | 0.33 | 0.13 |

### Prognostic Cores:

From Table 21, the following rule-out 2 variable prognostic cores are apparent:
1. s-ENG+DLG
2. s-ENG+1-HD
3. s-ENG+WRV

Building on this, 2 highly prognostic 3-variable rule-out cores can be found:
1. s-ENG AND DLG AND 1-HD
2. {s-ENG AND 1-HD}+r_glucose

A number of other noteworthy 2^{nd} tier 3 variable prognostic cores are also found:
3. 2-HBA+L-ALA+HVD3
4. HVD3+L-ISO+WRV

None of the combinations of 4 variables improve on the best of the 3 variable combinations, but a set of compliant 4 variable combinations are reported as further additive value may become apparent in larger patient cohorts.
1. {s-ENG AND DLG}+DHA+{DGLA OR H-L-ARG}
2. {s-ENG AND DLG}+ARA+any one variable from {2-HBA, UR, PIGF}
3. {s-ENG AND DLG}+2-HBA+3-HBA
4. {s-ENG AND DLG}+L-ERG+cig_1 st_trim_gp
5. s-ENG+CL+ECG+MYRA
6. s-ENG+{PIGF AND SC}+any one variable from {CL, L-ALA, L-MET, NGM}
7. s-ENG+PIGF+L-ISO+TR

### Summary—Rule-out Prognostic Performance Preterm PE.

When prognostic performance is expressed as specificity (i.e., detection rate of future non-cases) at set Rule-out thresholds like FNR or NPV, it can be observed that exceptional multi-variable prognostic performance for predicting the absence of future preterm PE, is achieved through a combination of protein and metabolite variables. Using such combinations, each of the pre-set success criteria (cf. Example 1—Exemplary Prognostic targets for preeclampsia risk stratification tests) are met with ease. For the FNR rule-out metrics considered, exceptional detection rates of future non-cases, is achieved with combinations that feature s-ENG AND DLG, possibly supplemented with one or 2 variables from the list CL, 1-HD, L-ERG, SC and NGM.

For the NPV rule-out metrics considered, exceptional detection of future non-cases is achieved with the following 2 variable prognostic cores, i.e., s-ENG and DLG and s-ENG AND 1-HD, and the 3-marker core s-ENG AND DLG AND 1-HD.

### Example 6C: PE Sub-Type: Term PE

### FNR Thresholds:

Model Space Filters applied: mean AUC ICI>=0.5; for the statistic mean SpecAtSens 0.80: ICI>=0.2, and imp>=0.03; for the statistic mean SpecAtSens 0.90: ICI>=0.1 and imp>=0.02.
Rank complete SensAtSpec 0.80 from high to low and select models down to 1^{st} single marker.
Based on this filtering, a set of 16 multivariable models was found, cf TABLE 22

TABLE 22
Term PE

| | | | | | | | Complete data set | | | | | | | |
|---|---|---|---|---|---|---|---|---|---|---|---|---|---|---|
| n | (Recurrent) Predictors | | | | | | Specificity (Sp) at | | | Specificity (Sp) at | | | 3 fold cross validation | |
| predictors/ | blood | | | weight | recurrent | | Sensitivity = 0.8 | | | Sensitivity = 0.9 | | | Imp | |
| model | pressure | 1-HD | HVD3 | related | other | other | Sp | lCl | uCl | Sp | lCl | uCl | SnAtSp 0.8 | SnAtSp 0.9 |
| 1 | map_2nd | | | | | | 0.47 | 0.29 | 0.59 | 0.26 | 0.15 | 0.47 | | |
| 2 | map_2nd | x | | | | | 0.54 | 0.36 | 0.63 | 0.36 | 0.27 | 0.53 | 0.05 | 0.14 |
| 2 | map_2nd | | | bmi | | | 0.53 | 0.34 | 0.62 | 0.30 | 0.16 | 0.53 | 0.04 | 0.08 |
| 2 | map_2nd | MoM | | | | | 0.50 | 0.36 | 0.63 | 0.33 | 0.25 | 0.49 | 0.06 | 0.11 |
| 2 | 1st_sbp | | | | | DC | 0.50 | 0.38 | 0.61 | 0.35 | 0.23 | 0.51 | 0.15 | 0.11 |
| 2 | map_2nd | | x | | | | 0.50 | 0.34 | 0.65 | 0.31 | 0.18 | 0.48 | 0.03 | 0.14 |
| 2 | map_2nd | | | | | ADMA | 0.48 | 0.34 | 0.59 | 0.34 | 0.16 | 0.45 | 0.04 | 0.08 |
| 2 | map_2nd | | | | | L-ALA | 0.47 | 0.31 | 0.60 | 0.30 | 0.15 | 0.47 | 0.04 | 0.11 |
| 3 | 1st_sbp | x | x | | | | 0.54 | 0.38 | 0.61 | 0.37 | 0.20 | 0.53 | 0.03 | 0.02 |
| 3 | map_1st | | x | bmi | | | 0.53 | 0.38 | 0.70 | 0.37 | 0.17 | 0.52 | 0.06 | 0.08 |
| 3 | 1st_sbp | | x | waist | | | 0.52 | 0.36 | 0.69 | 0.35 | 0.17 | 0.48 | 0.10 | 0.05 |
| 3 | 2nd_dbp | | | wgt | | age | 0.50 | 0.30 | 0.59 | 0.28 | 0.18 | 0.49 | 0.04 | 0.07 |
| 3 | 2nd_sbp | | x | | PIGF_MoM | | 0.50 | 0.32 | 0.62 | 0.31 | 0.23 | 0.50 | 0.04 | 0.03 |
| 3 | 1st_sbp | | x | wgt | | | 0.50 | 0.31 | 0.66 | 0.30 | 0.22 | 0.41 | 0.06 | 0.02 |
| 3 | 2nd_sbp | | x | | PIGF | | 0.49 | 0.33 | 0.63 | 0.32 | 0.23 | 0.48 | 0.04 | 0.04 |
| 4 | 2nd_sbp | | x | | H-L-ARG | TR_MoM | 0.48 | 0.37 | 0.63 | 0.36 | 0.27 | 0.47 | 0.03 | 0.02 |
| 4 | 1st_dbp | | x | | H-L-ARG | CR | 0.48 | 0.37 | 0.57 | 0.24 | 0.05 | 0.48 | 0.03 | 0.03 |

### Prognostic Cores:

From the TABLE 22, it is clear that in Rule-out models (as assessed by FNR), are governed by a blood pressure measure supplemented with minimally one variable.

The best 2 variable performance is achieved with the following 2 variable prognostic core;
1. Bp+1-HD

An additional, 2^{nd} tier 2 variable prognostic core is as follows:
2. Bp+any one variable from {WRV, DC, HVD3, ADMA, L-ALA}

Whereas, none of the combinations of 3 and 4 variables improve on the best 2 variable combination, a set of compliant 3 and 4 variable combinations are reported as further additive value may become apparent in larger patient cohorts.
1. Bp+1-HD+HVD3
2. {Bp AND HVD3}+any one variable from {WRV, PIGf, age}
3. {bp AND HVD3 AND H-L-ARG}+{CR or TR}

### NPV Threshold:

For the preeclampsia example elaborated here, the NPV threshold for “term PE”, was set to NPV=0.99375; and was calculated for a preterm PE prevalence=0.037 in accordance with TABLE 3.

Model Space Filters applied: mean AUC ICI>=0.5; for the statistic mean SpecAtNPV 0.99375: ICI>=0.1 and improvement >=0.05.
Rank complete SpecAtNPV from high to low and select models down to 1^{st} single marker.
Based on this filtering, a set of 39 multivariable models was found, cf TABLE 23

TABLE 23
Term PE

| | | | | | | | | Complete | | | |
|---|---|---|---|---|---|---|---|---|---|---|---|
| | | | | | | | | data set | | | 3 fold cross |
| n | (Recurrent) Predictors | | | | | | | Specificity at | | | validation |
| predictors/ | blood | | | | | recurrent | | NPV = 0.99375 | | | imp |
| model | pressure | 1-HD | HVD3 | DLG | L-ARG | other | other | Sp | lCl | uCl | SpAtNPV |
| 1 | map_2nd | | | | | | | 0.15 | 0.06 | 0.26 | |
| 2 | map_1st | x | | | | | | 0.35 | 0.04 | 0.45 | 0.29 |
| 2 | 2nd_sbp | x | | | | | | 0.34 | 0.03 | 0.41 | 0.19 |
| 2 | map_1st | MoM | | | | | | 0.31 | 0.05 | 0.43 | 0.26 |
| 2 | map_2nd | x | | | | | | 0.30 | 0.03 | 0.50 | 0.17 |
| 2 | map_2nd | MoM | | | | | | 0.27 | 0.02 | 0.44 | 0.15 |
| 2 | 2nd_sbp | | x | | | | | 0.27 | 0.03 | 0.43 | 0.09 |
| 2 | 2nd_sbp | | | x | | | | 0.25 | 0.04 | 0.36 | 0.06 |
| 2 | 1st_sbp | x | | | | | | 0.25 | 0.15 | 0.43 | 0.11 |
| 2 | 2nd_sbp | | | | x | | | 0.22 | 0.09 | 0.37 | 0.13 |
| 2 | 1st_sbp | MoM | | | | | | 0.22 | 0.10 | 0.41 | 0.11 |
| 2 | map_2nd | | x | | | | | 0.20 | 0.10 | 0.45 | 0.18 |
| 2 | map_2nd | | | | x | | | 0.18 | 0.12 | 0.28 | 0.11 |
| 3 | map_2nd | MoM | | | | sFlt1_MoM | | 0.36 | 0.03 | 0.44 | 0.06 |
| 3 | map_2nd | MoM | | | | sFlt1 | | 0.34 | 0.23 | 0.43 | 0.06 |
| 3 | 2nd_sbp | | x | | | TR_MoM | | 0.33 | 0.05 | 0.46 | 0.07 |
| 3 | 2nd_sbp | | x | | | s-ENG_MoM | | 0.31 | 0.03 | 0.43 | 0.05 |
| 3 | 2nd_sbp | | x | | | | H-L-ARG | 0.31 | 0.09 | 0.41 | 0.10 |
| 3 | map_2nd | x | x | | | | | 0.30 | 0.11 | 0.49 | 0.07 |
| 3 | 2nd_sbp | | x | | | sFlt1 | | 0.29 | 0.09 | 0.46 | 0.06 |
| 3 | 2nd_sbp | | | x | | sFlt1_MoM | | 0.29 | 0.09 | 0.37 | 0.05 |
| 3 | 2nd_sbp | | x | | | sFlt1_MoM | | 0.28 | 0.10 | 0.46 | 0.07 |
| 3 | 2nd_sbp | | | x | | sFlt1 | | 0.28 | 0.06 | 0.36 | 0.05 |
| 3 | map_2nd | MoM | | | | | CR | 0.28 | 0.02 | 0.46 | 0.06 |
| 3 | 2nd_sbp | | | | | sFlt1_MoM + | | 0.28 | 0.04 | 0.40 | 0.08 |
| | | | | | | L-ISO | | | | | |
| 3 | 2nd_sbp | | x | | | PIGF | | 0.28 | 0.09 | 0.47 | 0.06 |
| 3 | 2nd_sbp | | x | | | PIGF_MoM | | 0.27 | 0.06 | 0.48 | 0.06 |
| 3 | 1st_sbp | | x | | | wgt | | 0.27 | 0.07 | 0.37 | 0.05 |
| 3 | map_2nd | x | | | | 20-CL | | 0.27 | 0.03 | 0.45 | 0.06 |
| 3 | map_2nd | MoM | | | | 20-CL | | 0.25 | 0.02 | 0.40 | 0.06 |
| 3 | 2nd_sbp | | | | | sFlt1 + L-ISO | | 0.25 | 0.04 | 0.36 | 0.07 |
| 3 | 2nd_sbp | | x | | | bmi | | 0.23 | 0.05 | 0.46 | 0.06 |
| 3 | 2nd_sbp | | x | | | | MYRA | 0.22 | 0.10 | 0.40 | 0.06 |
| 3 | map_1st | | x | | | wgt | | 0.18 | 0.05 | 0.46 | 0.14 |
| 3 | map_1st | | x | | | bmi | | 0.18 | 0.04 | 0.50 | 0.05 |
| 4 | 2nd_sbp | | x | | | 20-CL | PALMA | 0.34 | 0.04 | 0.48 | 0.05 |
| 4 | map_2nd | x | x | | | | ADMA | 0.33 | 0.08 | 0.50 | 0.05 |
| 4 | map_2nd | x | | | | | ADMA + | 0.30 | 0.01 | 0.50 | 0.05 |
| | | | | | | | EPA | | | | |
| 4 | 1st_sbp | x | x | | | | UR | 0.25 | 0.09 | 0.50 | 0.06 |
| 4 | 2nd_sbp | | x | | | TR | S-1- | 0.24 | 0.04 | 0.46 | 0.06 |
| | | | | | | | P_MoM | | | | |

### Prognostic Cores:

From TABLE 23, the following prognostic rule-out 2-variable prognostic cores are apparent:
1. bp+1-HD
2. bp+HVD3
3. bp+DLG
4. bp_L-ARG

A minor improvement can be found in the following 3 variable prognostic core, by expanding on the first 2 variable prognostic core: 1. {bp AND 1-HD}+sFlt1

A number of other noteworthy 2^{nd} tier 3 variable prognostic cores can also be found as follows:
2. {Bp AND HVD3}+any one variable from {TR, s-ENG, H-L-ARG, 1-HD, PIGF, MYRA, WRV}
3. Bp+DLG+sFlt1
4. {Bp AND 1-HD}+{CR or 20-CL}
5. Bp+sFlt1+L-ISO

None of the combinations of 4 variables improve on the best of the 2 variable combinations.

### Summary—Rule-Out Prognostic Performance Term PE.

When prognostic performance is expressed as specificity (i.e., detection rate of future non-cases of term PE) at set Rule-out thresholds like FNR or NPV, it can be observed that moderate multi-variable prognostic performance for predicting the absence of future term PE, is achieved (FNR) or nearly achieved (NPV) by combining a blood pressure measure with 1-HD OR/AND HVD3.

### Example 7: Sequential Application of Rule-Out and Rule-in

### Introduction

As elaborated in Example 2, the inventors conceptualized a process which has the potential to improve the detection rate (Sensitivity) at a pre-set PPV rule-in cut-off, by means of firstly establishing a rule-out model (using the entirety of methods as elaborated elsewhere in this application), secondly applying this statistical model to identify those individuals at (a defined) low probability of developing the preeclampsia, and thirdly establishing a rule-in model (using the entirety of methods as elaborated elsewhere in this application), which maximizes the detection rate for future cases at a pre-set PPV threshold.

Here we demonstrate the validity of this concept using the following inputs:
pre-test preeclampsia prevalence values as per Example 1, i.e.
   prevalence all PE=0.05
   prevalence Preterm PE=0.014
   prevalence Term PE=0.037
post-test PPV cut-offs as per Table 3
   PPV All PE=0.133
   PPV Preterm PE=0.071
   PPV term PE=0.154
Application of a (sparse) rule-out model (as per Examples C1, C2, C3), whereby the specificity (detection rate of future non-cases) at 10% FNR cut-off (10% of true future cases will be lost for rule-in classification) is used to rule-out a fraction of the test population.

### Example 7A: All PE

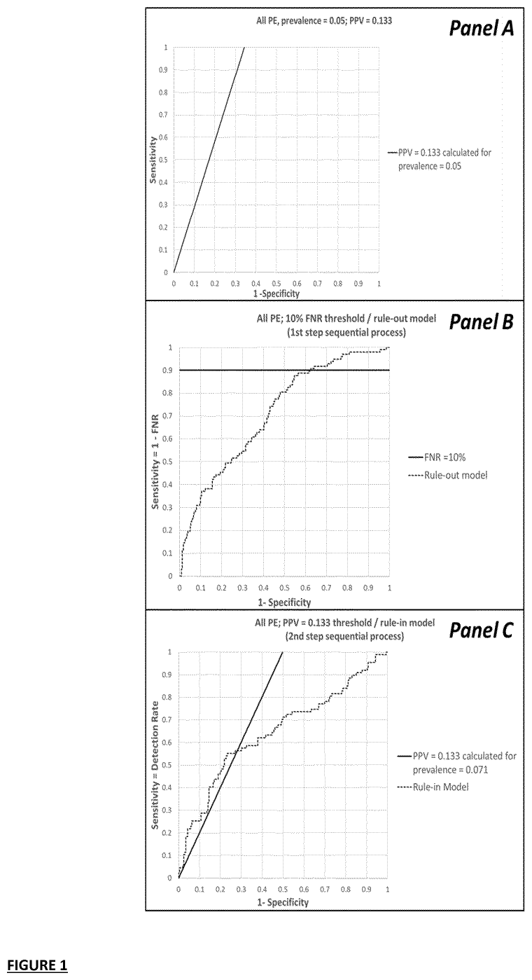

In accordance with Thomas et al [27], the PPV criterion can be plotted in the ROC-space, whereby the criterion is dependent on the pre-test preeclampsia prevalence. This is illustrated in FIG. 1 panel A. Within the exemplary framework used in Example 5A, the most performant (single step) rule-in multivariable model (bp+HVD3+CR+ADMA) delivers a detection rate of 48%.

In view of applying a sequence of a rule-out model followed by a rule-in model, an exemplary (sparse) rule-out model was considered, as per Example 6A, which is exemplified in FIG. 1 panel B.
All PE Rule-out model: bp+s-ENG+1-HD

Following the application of this rule-out model, 38.3% of the future non-cases as well as 10% of future cases (cf. 10% FNR) are removed from the test population. As a result, the prevalence of preeclampsia in the remaining test population (P2) increase to 0.071 (from 0.05). This has the effect of changing the PPV cut-off line as seen in FIG. 1 panel C. Within the residual population (P2), a novel set of predictive models was generated and assessed for their rule-in performance against the PPV criterion. The ROC curve as obtained with an exemplary (sparse) model is plotted in FIG. 1 panel C.
All PE Rule-in model: bp+PIGF+DC

Within test population P2, the latter model will deliver a sensitivity of 0.56 (56% detection rate). When correcting for the 10% future cases which were disregarded during the rule-out step, the overall detection rate is 51% (56%×0.9).

This overall detection rate, following the sequential application of a rule-out model and a rule-in model, is better than the 48% obtained with the application of a single rule-in model as per Example 5A. Therefore, combinations of rule-out prognostic cores as per Example 6A, and rule-in prognostic cores featuring multivariable combinations of any of the variables considered in this application will deliver exceptional rule-in prognostic performance for all preeclampsia.

### Example 7B: Preterm PE

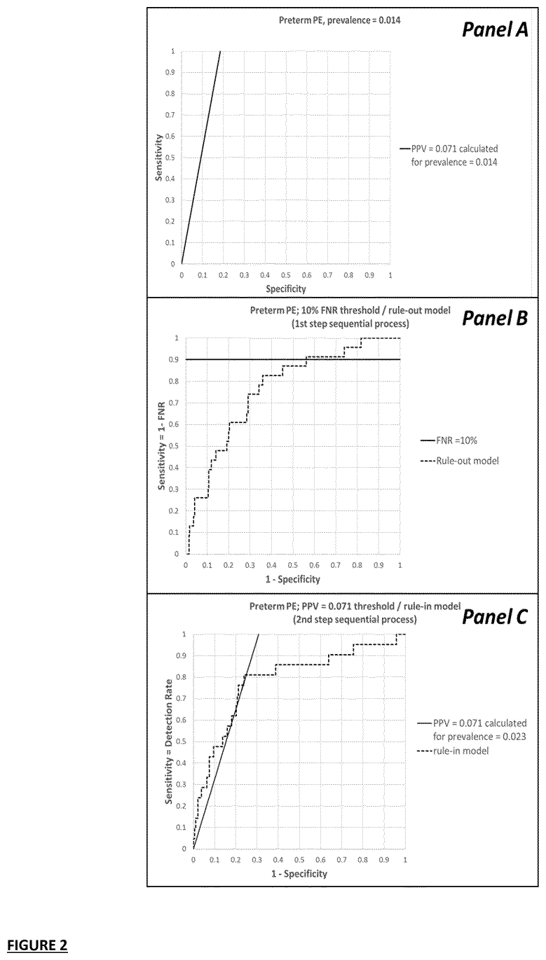

In accordance with Thomas et al [27], the PPV criterion can be plotted in the ROC-space, whereby the criterion is dependent on the pre-test preeclampsia prevalence. This is illustrated in FIG. 2 panel A. Within the exemplary framework used in Example 5B, the most performant (single step) rule-in multivariable model (PIGF+s-ENG+DLG+L-ERG) delivers a detection rate of 65%.

In view of applying a sequence of a rule-out model followed by a rule-in model, an exemplary (sparse) rule-out model was considered, as per Example 6B, which is exemplified in FIG. 2 panel B.
Preterm PE Rule-out model: s-ENG+DLG

Following the application of this rule-out model, 43.7% of the future non-cases as well as 10% of future cases (cf. 10% FNR) are removed from the test population. As a result, the prevalence of preterm preeclampsia in the remaining test population (P2) has gone up to 0.023 (from 0.014). This has the effect of changing the PPV cut-off line as seen in FIG. 2 panel C. Within the population (P2), a novel set of predictive models was generated and assessed for their rule-in performance against the PPV criterion. The ROC curve as obtained with an exemplary model is plotted in FIG. 2 panel C.
Preterm PE Rule-in model: PIGF+s-ENG+DLG+2-HBA

Within test population P2, the latter model will deliver a sensitivity of 0.81 (81% detection rate). When correcting for the 10% future cases which were disregarded during the rule-out step, the overall detection rate is 73% (81%×0.9).

This overall detection rate, following the sequential application of a rule-out model and a rule-in model, is better than the 65% obtained with the application of a single rule-in model as per Example 5B.

Therefore, combinations of rule-out prognostic cores as per Example 6B, and rule-in prognostic cores featuring multivariable combinations of any of the variables considered in this application will deliver exceptional rule-in prognostic performance for preterm preeclampsia.

### Example 7C: Term PE

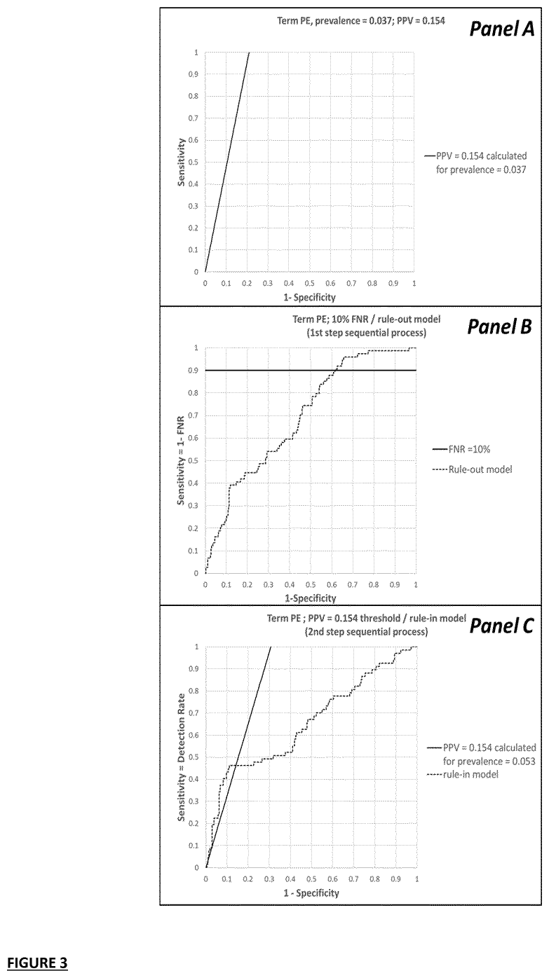

In accordance with Thomas et al [27], the PPV criterion can be plotted in the ROC-space, whereby the criterion is dependent on the pre-test preeclampsia prevalence. This is illustrated in FIG. 3 panel A. Within the exemplary framework used in Example 5C, the most performant (single step) rule-in multivariable model (bp+HVD3+TR) delivers a detection rate of 35%, which is not meeting the pre-set minimum detection rate as set in Table 3.

In view of applying a sequence of a rule-out model followed by a rule-in model, an exemplary (sparse) rule-out model was considered, as per Example 6C, which is exemplified in Figure D panel B.
Term PE Rule-out model: bp+1-HD

Following the application of this rule-out model, 38.2% of the future non-cases as well as 10% of future cases (cf. 10% FNR) are removed from the test population. As a result, the prevalence of term preeclampsia in the remaining test population (P2) has gone up to 0.053 (from 0.037). This has the effect of changing the PPV cut-off line as seen in Figure D panel C. Within the population (P2), a novel set of predictive models was generated and assessed for their rule-in performance against the PPV criterion. The ROC curve as obtained with an exemplary model is plotted in FIG. 3 panel C.
Term PE Rule-in model: bp+1-HD+NGM+H-L-ARG

Within test population P2, the latter model will deliver a sensitivity of 0.465 (46.5% detection rate). When correcting for the 10% future cases which were disregarded during the rule-out step, the overall detection rate is 42% (46.5%×0.9).

This overall detection rate, following the sequential application of a rule-out model and a rule-in model, is better than the 35% obtained with the application of a single rule-in model as per Example 5C, and does meet the preset minimal detection rate as put forward in Table 3.

Therefore, combinations of rule-out prognostic cores as per Example 6C, and rule-in prognostic cores featuring multivariable combinations of any of the variables considered in this application will deliver exceptional rule-in prognostic performance for preterm preeclampsia.

### Example 8: Prognostic Performance Maximisation by a Process of Sequential Classifiers

### Introduction

In Examples 5, 6 and 7 the primary aim for the prognostic test, whether the test being a single step or the result of the novel 2-step process, was the achievement of either exceptional rule-in performance or exceptional rule-out performance.

With specific combinations of variables, as considered throughout this application, consistently showing exceptional prognostic relevance for preterm PE, the inventors explored whether it is conceivable to develop a prognostic test that delivers simultaneously exceptional rule-in and rule out performance as expressed by the clinically relevant metrics PPV (rule-in) and NPV (rule-out).

As per Table 3, the following clinically relevant cut-offs were considered:
Rule-in: the test shall classify a population into a high risk group, wherein the probability of developing preterm preeclampsia is >=1/14 or PPV>=0.071
Rule-out: the test shall classify a population into a low risk group, wherein the probability of developing preterm preeclampsia is =<1/400 or NPV>=0.9975.

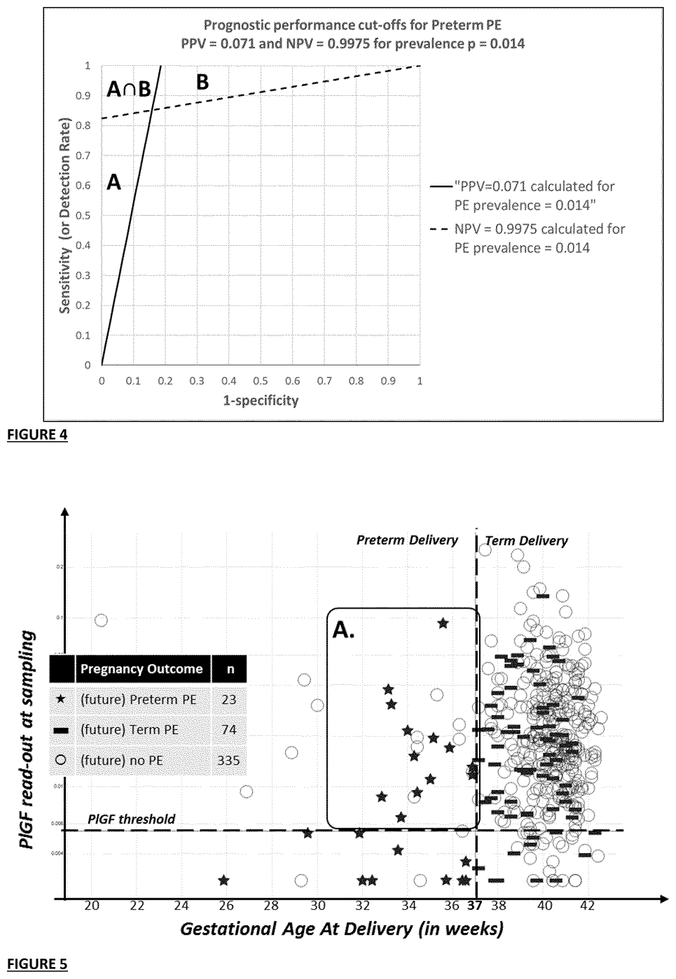

By accounting for the prevalence of (future) preeclampsia, i.e., p=0.014 (cf. Table 3), the prognostic requirements for such test can be represented in the ROC space; The clinically relevant PPV and NPV thresholds as relevant to Preterm PE are illustrated in FIG. 4.

### Preterm PE: Sequential Classifiers

The inventors found that by application of specific sequences of “rule-in” and “rule-out” classifications, as elaborated theoretically in Example 2, using the variables of interest as considered within this application, prognostic tests with exceptional Rule-in AND rule-out performance metrics can be established.

In a first step, the inventors utilized the well-known predictive merits of PIGF to predict preterm preeclampsia, as published for the SCOPE study in Kenny et al [24]. In FIG. 5, the PIGF levels as determined in maternal blood samples at ca. 15 weeks of pregnancy vs. the gestational age at delivery is given for all subjects of the study considered in this application (cf. Example 1). Please note, that at blood sampling all the women are considered healthy, and exhibited no clinical symptoms of preeclampsia, nor any clinical risk factors for preeclampsia. Women who delivered preterm, i.e., before 37 weeks of gestation, due to preeclampsia (“preterm preeclampsia”) are represented by “star” symbols, women who experienced preeclampsia, but delivered at term, i.e., at or later than 37 weeks of gestation, are represented by “bar” symbols, women who delivered without experiencing preeclampsia are represented by “circle” symbols.

As can be seen from Figure Example 5, using PIGF levels at time of sampling will allow to classify the test population in 2 groups. The women with PIGF below the shown threshold will have a PPV>0.071 to develop preterm PE, and are considered high risk. It is also clear that the group with PIGF levels higher (or equal) to the threshold constitutes >50% of the future preterm PE cases (Area “A”).

For the remainder of this exemplification, the (future) term PE cases are not considered any further. This results in the following Study-population Study-pop1, constituting (as per Example 1)
(Future) Preterm PE; n=23
(Future) no PE; n=335

To appreciate the prognostic performance of the classifiers as established within this Example 8, one needs to correct the Study data as the Study is based on a Case-Controls study design and thus has an over-representation of (future) preterm PE cases compared to the natural disease prevalence p=0.014. Hereto any classification data is recalculated for a hypothetical population of 10,000 pregnancies, constituting
(Future) Preterm PE; n=140
(Future) no PE; n=9860

By applying the following scaling factors
(future) Preterm PE: 140/23=6.087
(Future) no PE; 9860/335=29.43

Study results can be interpreted for a population of 10,000 pregnancies, whilst accounting for the natural disease prevalence.

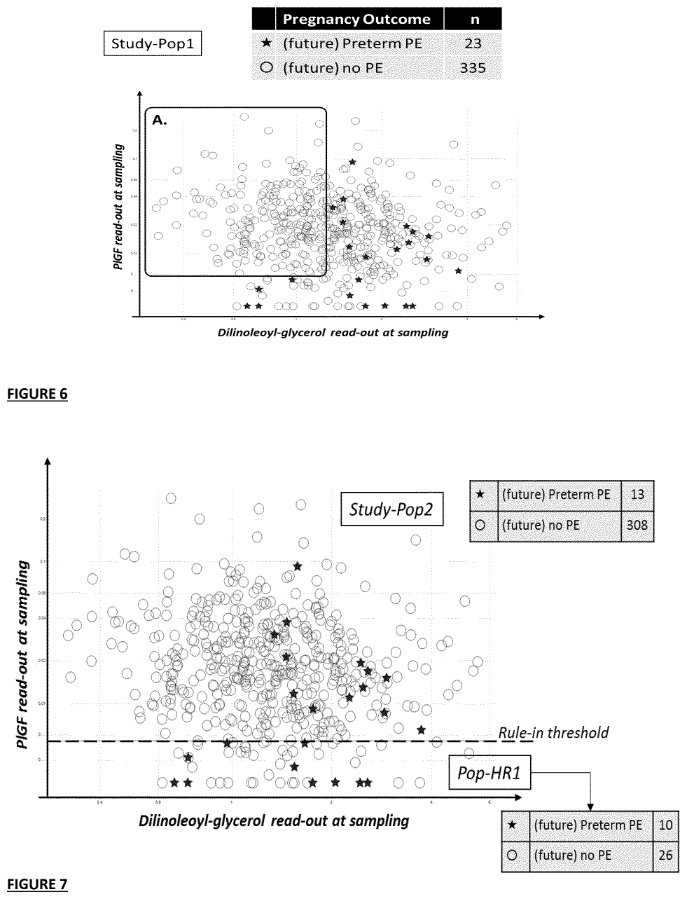

The inventors found that PIGF and DLG exhibit complementary classification potential, which becomes apparent when plotting both, as illustrated in FIG. 6.

In view of the clinical relevant Rule-in/Rule Out classification targets under consideration, this prognostic complementarity can be utilized as follows:

### Step 1

Application of a Rule-in classifier using the PIGF cut-off, as exemplified in FIG. 5. This classifier will segment the Study-Pop₁ into a “Ruled-in” or High Risk population (Pop-HR1), as per Figure Example 7, and a new study population Study-Pop2.

Using the rule PIGF<0.005445, the following classification is achieved:

Study - Pop 1

| Rule-in (Pop-HR1) | | Study - Pop2 | |
|---|---|---|---|
| Cases | controls | cases | controls |
| 10 | 26 | 13 | 309 |

population of 10000

| Rule in (Pop-HR1) | | | Study - Pop2 | | |
|---|---|---|---|---|---|
| Cases | controls | total | cases | controls | total |
| 60.87 | 765.254 | 826.12 | 79.1 | 9094.75 | 9173.9 |

When expressed as part of a total classifier, this 1^{st} step results following:

| | Prognostic performance metrics - Total | | | |
|---|---|---|---|---|
| Classifier | classifier | | | |
| classes | Sn | Sp | PPV | NPV |
| Rule-in | 0.43 | 0.92 | 0.074 | 0.926 |
| (Pop-HR1) | | | | |
| Rule-out | na | na | na | na |
| Residual | 0.57 | 0.08 | 0.009 | 0.991 |
| (Study-Pop2) | | | | |

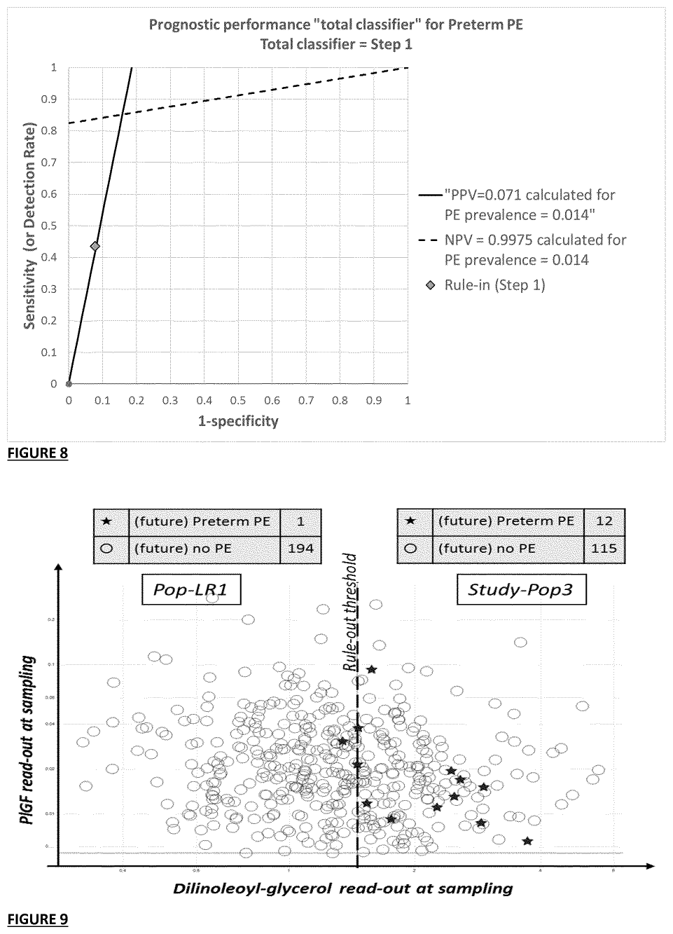

The PPV within the established Rule-in group, Pop-HR1, is compliant with the preset-PPV criterion (PPV>=0.071). The overall detection rate of this single step classifier is 43% (Sₙ=0.43), i.e., one will find 43% of all future preterm PE cases when applying just this PIGF based cut-off. It is of note that the non-ruled-in group (Study-Pop₂) is not compliant with either the PPV or NPV criterion. This single step classifier can also be plotted in the ROC space, as illustrated in FIG. 8; confirming compliance with the PPV-criterion (cf. FIG. 4)

Because population Pop-HR1 is fully compliant with the pre-set PPV criterion, PoP-HR1 is considered fully classified and not considered further (removed from the Study). This means that the next step in classification will only consider Study-Pop2.

### Step 2

Following the removal of Pop-HR1 from the study-Pop1 the additive prognostic value of DLG becomes very apparent. A Rule-out classifier using the DLG cut-off, is exemplified in FIG. 9. This classifier will segment the Study-Pop2 into a “Ruled-out” or Low Risk population (Pop-LR1), and a new study population Study-Pop3.

Using the rule DLG<0.1454243, the following classification is achieved:

Study - Pop 2

| Rule out (Pop-LR1) | | Study-Pop3 | |
|---|---|---|---|
| Cases | controls | cases | controls |
| 1 | 194 | 12 | 115 |

population of (10000 − 826.12)

| Rule out (Pop-LR1) | | | Study-Pop3 | | |
|---|---|---|---|---|---|
| Cases | controls | total | cases | controls | total |
| 6.09 | 5709.97 | 5716.06 | 73 | 3384.78 | 3457.8 |

When added to the total classifier, this 2^{nd} step results in the following:

| | Prognostic performance metrics - Total | | | |
|---|---|---|---|---|
| Classifier | classifier | | | |
| classes | Sn | Sp | PPV | NPV |
| Rule-in | 0.43 | 0.92 | 0.074 | 0.926 |
| (Pop-HR1) | | | | |
| Rule-out | 0.96 | 0.58 | 0.001 | 0.999 |
| (Pop-LR1) | | | | |
| Residual | 0.52 | 0.66 | 0.021 | 0.979 |
| (Study-Pop3) | | | | |

Whereby the NPV within the established Rule-out group, Pop-LR1, is compliant with the preset-NPV criterion (NPV>=0.9975).

The overall detection rate of this dual step classifier is 43% (Sₙ=0.43), i.e., one will find 43% of all future preterm PE cases when applying the PIGF based cut-off (step 1; lower than). For any subject which has a PIGF higher than (or equal to) the PIGF cut-off, one will determine whether the subject has a value lower (ruled-out) than the DLG based cut-off, or higher than the DLG cut-off (becoming part of Study-pop3). 58% (Sp=0.58) of the (future) non-PE cases will be stratified into the Pop-LR1 and will be considered low-risk. It is of note that the composition of Study-Pop3 is not compliant with either the PPV or NPV criterion.

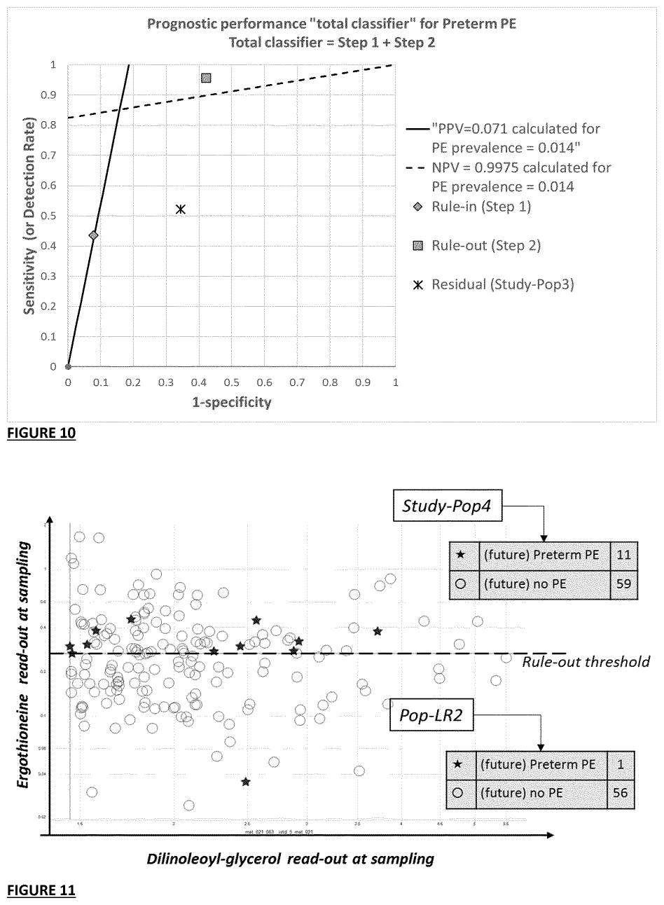

This two step classifier can also be plotted in the ROC space, as illustrated in FIG. 10. As the total classifier considers the Rule-in classification and Rule-out classification separately, the total test classifier corresponds to 2 separate (Sₙ-Sp) pairs. One can see that the resulting Rule-in classification and Rule-out classification are compliant with either the pre-set PPV- or NPV-cut off. These subjects which are not classified to either be at high-risk or at low risk, constitute Study-Pop₃. One can also plot the metrics of Study-pop3 in the ROC space. It is clear that this group (residual) is not compliant.

Because population Pop-LR1 is fully compliant with the pre-set NPV criterion, Pop-LR1 is also considered fully classified and not considered further (removed from the Study). This means that the next step in classification will only consider Study-Pop3.

### Step 3

Following the removal of Pop-LR1 from the study-Pop2, the inventors found that L-ERG can be used to stratify Study-Pop₃ once more, to rule-out an additional group of subjects, and classify them as low-risk as well. Application of a Rule-out classifier using a L-ERG cut-off, is exemplified in FIG. 11. This classifier will segment the Study-Pop3 into a “Ruled-out” or Low Risk population (Pop-LR2), and a new study population Study-Pop4.

Using the rule L-ERG<0.266432, the following classification is achieved:

Study - Pop 3

| Rule-out (Pop-LR2) | | Study-Pop4 | |
|---|---|---|---|
| Cases | controls | cases | controls |
| 1 | 56 | 11 | 59 |

population of (10000 − 826.12 − 5716.06)

| Rule out (Pop-LR2) | | | Study-Pop4 | | |
|---|---|---|---|---|---|
| Cases | controls | total | cases | controls | total |
| 6.09 | 1648.24 | 1654.33 | 67 | 1736.54 | 1803.5 |

Within the new Ruled-out group, i.e., Pop-LR2, the preset NPV criterion is just missed (NPV=0.996), yet when the 3^{rd} step is considered in combination with Pop-LR1, the cumulative rule-out criterion is met.

In combination with the previous steps, the 3^{rd} step gives rise to the following combined Rule-in and Rule-out classification:

| | Prognostic performance metrics - Total | | | |
|---|---|---|---|---|
| Classifier | classifier | | | |
| classes | Sn | Sp | PPV | NPV |
| Rule-in | 0.43 | 0.92 | 0.074 | 0.926 |
| (Pop-HR1) | | | | |
| Rule-out | 0.91 | 0.75 | 0.002 | 0.998 |
| (Pop LR1 + LR2) | | | | |
| Residual | 0.48 | 0.82 | 0.037 | 0.963 |
| (Study-Pop4) | | | | |

The overall detection rate of this dual step classifier is 43% (Sₙ=0.43), i.e., one will find 43% of all future preterm PE cases when applying the PIGF based cut-off (step 1; lower than; Pop-HR1).

For any subject which has a PIGF higher than (or equal to) the PIGF cut-off, it is then determined whether the subject has a value lower than the DLG based cut-off; if yes, these subjects are considered low risk (step 2; Pop-LR1).

For any subject which has a PIGF blood value >=PIGF cut-off AND a DLG blood value >=DLG cut-off, one will determine whether the subject has a value lower than the L-ERG based cut-off (ruled-out; Pop_{LR2}), or higher than the L-ERG cut-off (becoming part of Study-pop4). When combining these 2 consecutive rule-out segmentation steps 75% (Sp=0.75) of the (future) non-preterm PE cases will be stratified into the total Low risk Group. It is of note that the composition of Study-Pop₄ is not compliant with either the PPV or NPV criterion.

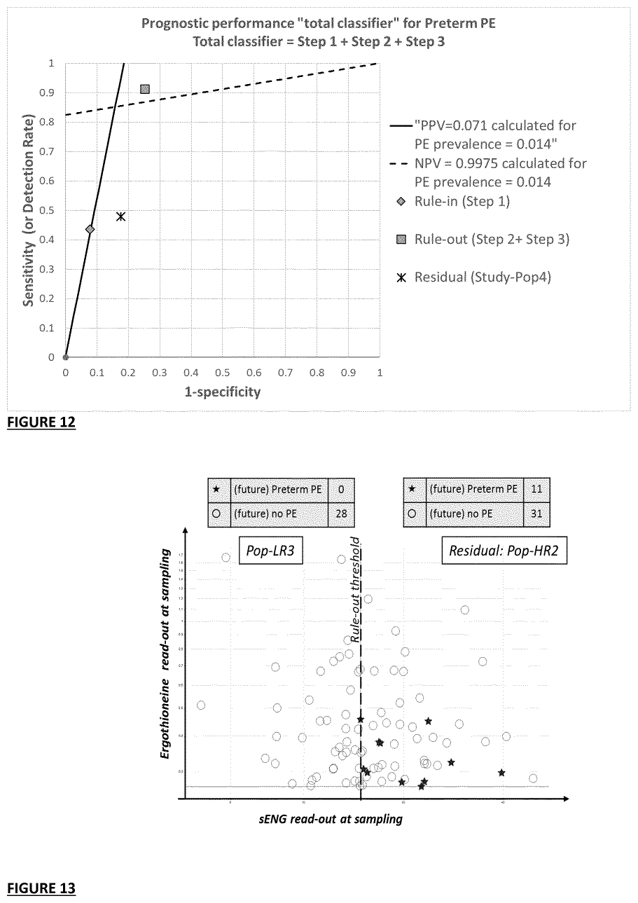

This three step classifier can also be plotted in the ROC space, as illustrated in FIG. 12. As the total classifier considers the Rule-in classification and Rule-out classification separately, the total test classifier corresponds to 2 separate (Sₙ-Sp) pairs. One can see that the resulting Rule-in classification and Rule-out classification are compliant with either the pre-set PPV- or NPV-cut off. These subjects which are not classified to either be at high-risk or at low risk, constitute Study-Pop₄. One can also plot the metrics of this “negative” test, corresponding to Study-pop4 in the ROC space. It is clear that this group (residual) is not compliant.

### Step 4

The inventors found that s-ENG can be used to stratify Study-Pop₄ once more, to rule-out an additional group of subjects, and classify them as low-risk as well. Application of a Rule-out classifier using a s-ENG cut-off, is exemplified in FIG. 13. This classifier will segment the Study-Pop₄ into a “Ruled-out” or Low Risk population (Pop-LR2), and a residual study population.

Using the rule s-ENG<14.8293, the following classification of Study-Pop 4 is achieved:

Study - Pop 3

| Rule-out | | Residual = Rule-in | |
|---|---|---|---|
| Cases | controls | cases | controls |
| 0 | 28 | 11 | 31 |

population of (10000 − 826.12 − 5716.06 − 1648.24)

| Rule out | | | Residual = rule -in | | |
|---|---|---|---|---|---|
| Cases | controls | total | cases | controls | total |
| 0.00 | 824.12 | 824.12 | 67 | 912.42 | 979.37 |

Within the new Ruled-out group, i.e., Pop-LR3, the preset NPV criterion is met once more (NPV=1), which will ensure that when the 4th step is considered as part of the total classifier, the cumulative rule-out criterion is met.

When added to the total classifier, the 4th step gives rise to the following combined Rule-in and Rule-out classification:

| | Prognostic performance metrics - Total | | | |
|---|---|---|---|---|
| Classifier | classifier | | | |
| classes | Sn | Sp | PPV | NPV |
| Rule-in | 0.43 | 0.92 | 0.074 | 0.926 |
| (Pop-HR1) | | | | |
| Rule-out | 0.91 | 0.83 | 0.001 | 0.999 |
| (Pop LR1 + LR2 + LR3) | | | | |
| Residual | 0.48 | 0.91 | 0.068 | 0.932 |

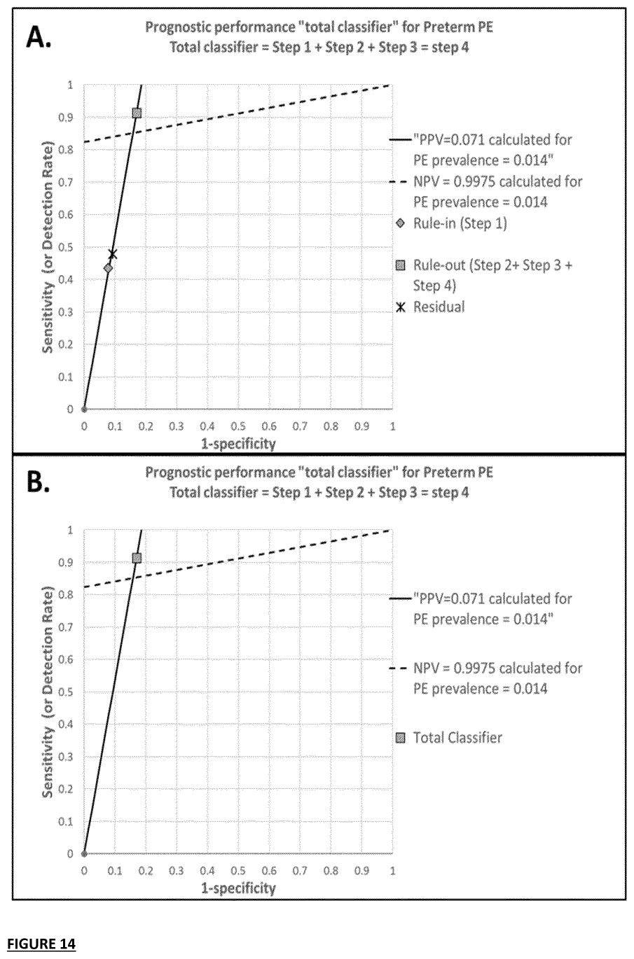

Interestingly, it can be observed that within the Residual population the risk to get preterm PE later in pregnancy is virtually compliant with the PPV threshold. This is also apparent when plotting the Total Classifier in the ROC space as illustrated in FIG. 14 Panel A.

From FIG. 14-panel A, one can appreciate that, following the application of the last Rule-out classification, the totality of “Rule-out” classifications is compliant with the NPV rule-out criterion (as intended), but it is also compliant with the pre-set PPV “Rule-in” criterion. In other words, as a result of this specific sequential application of individual classifiers, any subject which is not classified as Low-risk (as per the previous rule-out classifiers), is High-risk. This is also clear from the “residual” population, which also complies with the PPV criterion (similar to the first Rule-in group Pop-HR1). The iterative removal (or “ruling-out”) of “low risk” subjects lead to a residual population highly enriched in (future) preterm PE cases. Therefore, one can consider a total High Risk group which constitutes: Pop-HR1+Residual (or Pop-HR2).

By doing so, this “total classifier”, as illustrated in FIG. 14-panel B, will segment the original study Study-pop1 population in 2 groups; i.e.
a high risk group which contains 91% of (future) preterm PE cases, and wherein any subject has a risk of >= 1/14 (i.e., PPV>=0.071) of effectively developing the disease later in pregnancy
a low risk group which contains 83% of (future) non-PE cases, and wherein any subject has a risk of =<11400 or (NPV>=0.9975) of effectively developing preeclampsia.

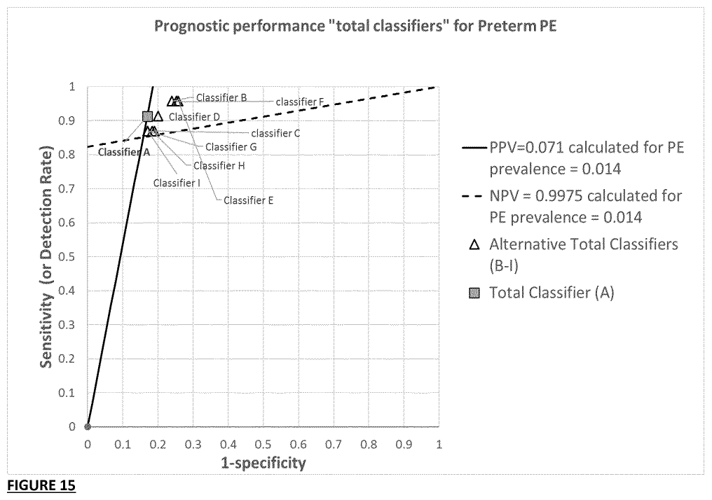

In addition to the fully exemplified Total classifier (Classifier A; in Table 20), additional Total classifiers constituting the ordered application of a set of 4 variables, were also found. Their key prognostic performance statistics are presented in Table 24 and illustrated in the ROC space in FIG. 15:

TABLE 24
Prognostic models for Preterm PE: Variables, in order of application in the Sequential classifier,
and exemplary prognostic performance metrics for the preterm PE example elaborated
in this application.

| | | Prognostic Metrics Total | | | |
|---|---|---|---|---|---|
| | Variables - and order of their | Classifier | | | |
| Classifier | application to achieve | Sn | Sp | PPV | NPV |
| Classifier A | PIGF, DLG, L-ERG, s-ENG | 0.913 | 0.830 | 0.071 | 0.999 |
| Classifier B | PIGF, DLG, s-ENG, 1-HD | 0.957 | 0.76 | 0.054 | 0.999 |
| classifier C | PIGF, DLG, L-LEU, s-ENG | 0.87 | 0.81 | 0.061 | 0.998 |
| Classifier D | PIGF, DLG, s-ENG, L-LEU | 0.913 | 0.799 | 0.061 | 0.998 |
| Classifier E | PIGF, DLG, L-ISO, s-ENG | 0.957 | 0.742 | 0.050 | 0.999 |
| classifier F* | PIGF, DLG, (L-LEU + L-ISO)*, s-ENG | 0.957 | 0.748 | 0.051 | 0.999 |
| Classifier G | PIGF, DLG, L-ERG, L-LEU | 0.87 | 0.829 | 0.067 | 0.998 |
| Classifier H | PIGF, DLG, L-ERG, L-ISO | 0.87 | 0.817 | 0.063 | 0.998 |
| Classifier I* | PIGF, DLG, L-ERG, (L-LEU + L-ISO)* | 0.87 | 0.829 | 0.067 | 0.998 |

*With L-LEU and L-ISO strongly correlating, and being closely related compounds, it was investigated whether the summed signal of L-LEU and L-ISO delivers similar prognostic performance compared to classifiers wherein L-LEU and L-ISO were used individually. The tabulated data confirmed that the summed signal of L-LEU and L-ISO can be considered. The summed quantification data constituted the ratio of the (summed quantifier ion signals of L-LEU and L-ISO) over the quantifier ion signal of either the ISTD_3_L-LEU or ISTD_6_L-ISO SIL standard. The 2 possible read-outs give similar results; the data reported used the ISTD_3_L-LEU.

The exemplary total classifications as achievable with each of these classifiers is also illustrated in FIG. 15.

### Summary Preterm PE: Sequential Classifiers

It will be apparent to reader that the following Total Classifiers constituting the ordered application of any, 2, 3 or 4 variable classifiers will deliver highly prognostic stratification for preterm PE in pregnant women early in pregnancy prior to showing any clinical symptoms of preeclampsia:
PIGF, DLG, L-ERG and s-ENG
PIGF, DLG, s-ENG, 1-HD
PIGF, DLG, L-LEU, s-ENG
PIGF, DLG, s-ENG, L-LEU
PIGF, DLG, L-ISO, s-ENG
PIGF, DLG, (L-LEU+L-ISO), s-ENG
PIGF, DLG, L-ERG, L-LEU
PIGF, DLG, L-ERG, L-ISO
PIGF, DLG, L-ERG, (L-LEU+L-ISO)

### Example 9 Dilinoleoyl-Glycerol Isoforms

### Introduction

From the examples elaborated herein, it is clear that DLG is a key variable in multivariable prognostic models for preeclamspsia, and specifically for preterm preeclampsia.

The Applicants found that diacylglycerols can exist in three stereochemical forms; i.e., sn-1,2-diacylglycerol, sn-2,3-diacylglycerol, and sn-1,3-diacylglycerol, whereby the 1^{st} two are enantiomers. In addition, the literature learns isomerization by means of acyl migrations can occur. To confirm whether the prognostic merits of the analysed signal is indeed the result of a mixture of different dilinoleoyl-glycerol isomers and whether the prognostic merits are differential between the different possible isoforms, the Applicants developed a separate LC-MS method to firstly resolve any possible isomers and analyse a study population with this dedicated method.

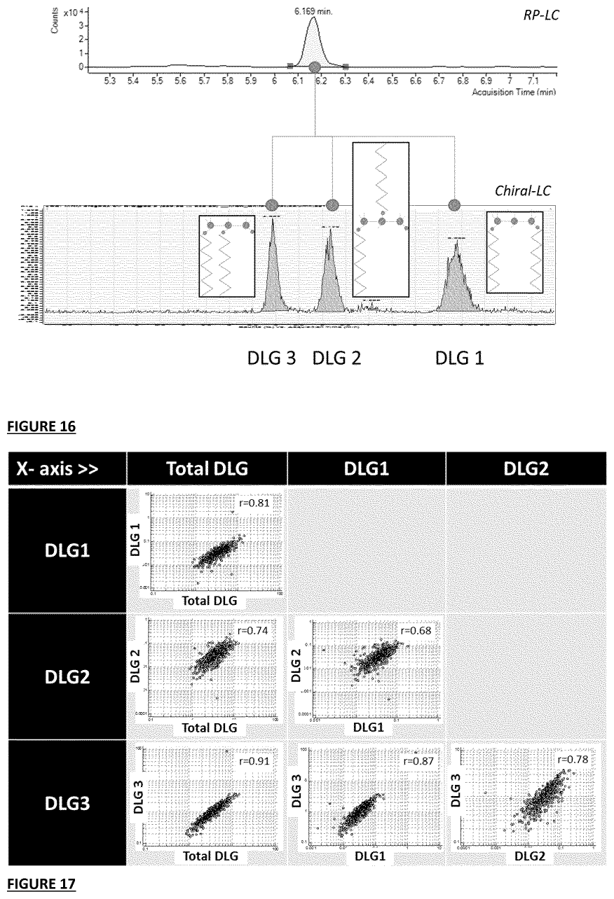

Using chiral chromatography (stationary phase Lux-Amylose-1; Phenomenex, Cheshire, UK) in combination with MS/MS settings as established for the Met 021_063 reference material, it was indeed shown that the observed Dilinoleoyl-glycerol signal was indeed the result of the summed signal of 3 isomers, as can be seen in FIG. 16.

For the purpose of this example, the different isoforms are abbreviated as follows: DLG1 (1,3-). DLG2 (2,3-) and DLG3 (1,2-). The total signal dilinoleoyl-glycerol signal as obtained with LC-MS methodology as elaborated elsewhere in this application is abbreviated as “Total DLG”.

### Experiment

Based on this a second experiment was conducted, whereby a different case:control study within the same SCOPE study as elaborated in example 1 was used; In brief this study constituted:
(future) PE cases: 53, whereof
   (future) preterm PE cases: 17
   (future) term PE cases: 42
(future) non-PE (controls): 574

This sample set was analyzed in duplicate, once with a methodology akin to the one elaborated within this application (an RPLC-only variant; cf, DLG is an hydrophobic metabolite), and secondly with a chiral LC-MSMS method, whereby the mass spectrometric analysis used the metabolite settings as derived for Met_021_063 and a chiral LC method.

In brief, the chiral LC method involved the following:
mobile phase A: H₂O:MeOH:Ammonium acetate buffer pH4.5 92:3:5
mobile phase B MeOH:ACN:IPA: Ammonium acetate buffer pH4.5 35:35:25:5

The chromatography was run under isocratic conditions at 95% B:5% A using a (4×20) mm Lux Amylose-1 guard column and a 100×4.6 mm 5 um Lux Amylose-1 analytical column.

The quantification data was based on the quantifier ion for each dilinoleoyl-glycerol type/quantifier ion of 1,3-Dilinoleoyl-rac-glycerol-[²H5].

### Data:

It was found that one of the isoforms was more abundant than the other 2, this isoform being one of the two enantiomers (i.e., 1,2- or 2,3-dilinoleoyl-glycerol), most likely being sn-1,2-dilinoleoyl-glycerol (DLG3). The other 2 isoforms are estimated to be about 30˜less abundant; due to analytical sensitivity limitations. More importantly however, it became clear that all 3 isoforms correlated strongly with each other as well as with the total signal as obtained with the generic LC-MS method, as illustrated in FIG. 17. With the signals for DLG1 and DLG2 being close to detection limit, the associated imprecision, as established for replicate injections, is higher, i.e., % CV=19% and % CV=27% respectively; whereas in this experiment % CV for total DLG and DLG3 were % CV=16% and % CV=15% respectively. The lower precision for DLG1 and DLG2 underlies the slightly lower correlation coefficients (r) found.

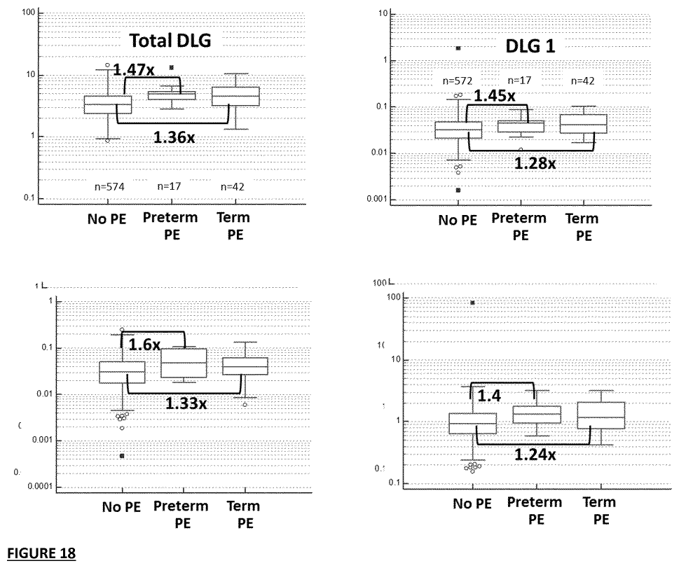

In addition, the prognostic merits for each of the isoforms were shown to be equivalent; as one can observe in FIG. 18; the median fold changes between the “no (future) PE” group versus either the “(future) Preterm PE” group or the “(future) Term PE” group are very similar between the “total dilinoleoyl-glycerol” as well as any of the different dilinoleoyl-glycerol isoforms. It is of note that these fold changes are also in agreement with the fold changes reported in Tables Example 13.2 and Example 13.3. The study samples in this specific study overlapped with the Study population as reported on throughout the application (cf. Example 1) in “future” pre-eclampsia cases, but utilized a different random “no-PE” group (some samples may overlap). This adds further proof to the relevance of dilinoleoyl-glycerol as a relevant prognostic variable for pre-eclampsia and more specifically preterm Pre-eclampsia.

This confirms that it is appropriate to analyse “total Dilinoleoyl-glycerol”, as per the analytical methods elaborated elsewhere in this application, and use its signal as a prognostic variable in the context of pre-eclampsia. This does not preclude the Applicants from appreciating that any combination of 1, 2 or 3 (“total”) dilinoleoyl-glycerol isomers, i.e., sn-1,2-diacylglycerol, sn-2,3-diacylglycerol, and sn-1,3-diacylglycerol, carries prognostic potential for pre-eclampsia.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

### REFERENCES

1 Steegers E a P, von Dadelszen P, Duvekot J J, et al. Pre-eclampsia. Lancet 2010; 376:631-44. doi:10.1016/S0140-6736(10)60279-6
2 Chaiworapongsa T, Chaemsaithong P, Yeo L, et al. Pre-eclampsia part 1: current understanding of its pathophysiology. Nat Rev Nephrol 2014; 10:466-80. doi:nrneph.2014.102 [pii]\n10.1038/nrneph.2014.102
3 Duley L. The global impact of pre-eclampsia and eclampsia. Semin Perinatol 2009; 33:130-7. doi:10.1053/j.semperi.2009.02.010
4 Davis E F, Lazdam M, Lewandowski A J, et al. Cardiovascular risk factors in children and young adults born to preeclamptic pregnancies: a systematic review. Pediatrics 2012; 129:e1552-61. doi:10.1542/peds.2011-3093
5 Washburn L, Nixon P, Russell G, et al. Adiposity in adolescent offspring born prematurely to mothers with preeclampsia. J Pediatr 2013; 162:912-7.e1. doi:10.1016/j.jpeds.2012.10.044
6 Ros H S, Lichtenstein P, Ekbom a, et al. Tall or short? Twenty years after preeclampsia exposure in utero: comparisons of final height, body mass index, waist-to-hip ratio, and age at menarche among women, exposed and unexposed to preeclampsia during fetal life. Pediatr Res 2001; 49:763-9. doi:10.1203/00006450-200106000-00008
7 Bujold E, Roberge S, Lacasse Y, et al. Prevention of preeclampsia and intrauterine growth restriction with aspirin started in early pregnancy: a meta-analysis. Obstet Gynecol 2010; 116:402-14. doi:10.1097/AOG.0b013e3181e9322a
8 Syngelaki A, Nicolaides K H, Balani J, et al. Metformin versus Placebo in Obese Pregnant Women without Diabetes Mellitus. N Engl J Med 2016; 374:434-43. doi:10.1056/NEJMoa1509819
9 Roberts J M, Bell M J. If we know so much about preeclampsia, why haven't we cured the disease? J Reprod Immunol 2013; 99:1-9. doi:10.1016/j.jri.2013.05.003
10 Rolnik D L, Wright D, Poon L C, et al. Aspirin versus Placebo in Pregnancies at High Risk for Preterm Preeclampsia. N Engl J Med 2017; 377:613-22. doi:10.1056/NEJMoa1704559
11 O'Gorman N, Wright D, Rolnik D L, et al. Study protocol for the randomised controlled trial: combined multimarker screening and randomised patient treatment with ASpirin for evidence-based PREeclampsia prevention (ASPRE). BMJ Open 2016; 6:e011801. doi:10.1136/bmjopen-2016-011801
12 McLaughlin K, Baczyk D, Potts A, et al. Low Molecular Weight Heparin Improves Endothelial Function in Pregnant Women at High Risk of Preeclampsia Novelty and Significance. Hypertension 2017; 69:180-8. doi:10.1161/HYPERTENSIONAHA.116.08298
13 Ruan Y, Sun J, He J, et al. Effect of probiotics on glycemic control: A systematic review and meta-analysis of randomized, controlled trials. PLoS One 2015; 10:1-15. doi:10.1371/journal.pone.0132121
14 Powers R, Weissgerber T L, McGonigal S, et al. L-Citrulline administration increases the arginine/ADMA ratio, decreases blood pressure and improves vascular function in obese pregnant women. Pregnancy Hypertens An Int J Women's Cardiovasc Heal 2015; 5:4. doi:10.1016/j.preghy.2014.10.011
15 Roberts J M, Myatt L, Spong C Y, et al. Vitamins C and E to prevent complications of pregnancy-associated hypertension. N Engl J Med 2010; 362:1282-91. doi:10.1056/NEJMoa0908056
16 McCarthy C, Kenny L C. Therapeutically targeting mitochondrial redox signalling alleviates endothelial dysfunction in preeclampsia. Sci Rep 2016; 6:32683. doi:10.1038/srep32683
17 Kerley R N, McCarthy C, Kell D B, et al. The potential therapeutic effects of ergothioneine in pre-eclampsia. Free Radic Biol Med 2018; 117:145-57. doi:10.1016/j.freeradbiomed.2017.12.030
18 Costantine M M, Cleary K. Pravastatin for the Prevention of Preeclampsia in High-Risk Pregnant Women. Obstet Gynecol 2013; 121:349-53. doi:10.1097/AOG.0b013e31827d8ad5
19 Wang K, Ahmad S, Cai M, et al. Dysregulation of hydrogen sulfide producing enzyme cystathionine γ-lyase contributes to maternal hypertension and placental abnormalities in preeclampsia. Circulation 2013; 127:2514-22. doi:10.1161/CIRCULATIONAHA.113.001631
20 Johal T, Lees C C, Everett T R, et al. The nitric oxide pathway and possible therapeutic options in pre-eclampsia. Br J Clin Pharmacol 2014; 78:244-57. doi:10.1111/bcp.12301
21 Wang Y, Lewis D F, Adair C D, et al. Digibind attenuates cytokine TNFalpha-induced endothelial inflammatory response: potential benefit role of digibind in preeclampsia. J Perinatol 2009; 29:195-200. doi:10.1038/jp.2008.222
22 Wester-Rosenlöf L, Gassién V, Axelsson J, et al. A1M/α1-Microglobulin Protects from Herne-Induced Placental and Renal Damage in a Pregnant Sheep Model of Preeclampsia. PLoS One 2014; 9:e86353. doi:10.1371/journal.pone.0086353
23 North R A, Mccowan L M E, Dekker G A, et al. Clinical risk prediction for pre-eclampsia in nulliparous women: development of model in international prospective cohort. BMJ 2011; 342:d1875. doi:10.1136/bmj.d1875\rbmj.d1875 [pii]
24 Kenny L C, Black M A, Poston L, et al. Early Pregnancy Prediction of Preeclampsia in Nulliparous Women, Combining Clinical Risk and Biomarkers: The Screening for Pregnancy Endpoints (SCOPE) International Cohort Study. Hypertension 2014; 64:644-52. doi:10.1161/HYPERTENSIONAHA.114.03578
25 Royston P, Moons K G M, Altman D G, et al. Prognosis and prognostic research: Developing a prognostic model. BMJ 2009; 338:b604. doi:10.1136/bmj.b604
26 Bartsch E, Medcalf K E, Park A L, et al. Clinical risk factors for pre-eclampsia determined in early pregnancy: systematic review and meta-analysis of large cohort studies. Bmj 2016:i1753. doi:10.1136/bmj.i1753
27 Thomas G, Kenny L C, Baker P N, et al. A novel method for interrogating receiver operating characteristic curves for assessing prognostic tests. Diagnostic Progn Res 2017; 1:17. doi:10.1186/s41512-017-0017-y
28 Boghossian N S, Yeung E, Mendola P, et al. Risk factors differ between recurrent and incident preeclampsia: A hospital-based cohort study. Ann Epidemiol 2014; 24:871-7. doi:10.1016/j.annepidem.2014.10.003
29 Hernández-Diaz S, Toh S, Cnattingius S. Risk of pre-eclampsia in first and subsequent pregnancies: prospective cohort study. BMJ 2009; 338:b2255. doi:10.1136/bmj.b2255
30 Rolnik D L, Wright D, Poon L C, et al. ASPRE trial: performance of screening for preterm pre-eclampsia. Ultrasound Obstet Gynecol Published Online First: 25 Jul. 2017. doi:10.1002/uog.18816
31 Tuytten R, Lemière F, Esmans E L, et al. In-Source CID of Guanosine: Gas Phase Ion-Molecule Reactions. J Am Soc Mass Spectrom 2006; 17:1050-62. doi:10.1016/j.jasms.2006.03.012
32 Berg T, Strand D H. 13C labelled internal standards-A solution to minimize ion suppression effects in liquid chromatography-tandem mass spectrometry analyses of drugs in biological samples? J Chromatogr A 2011; 1218:9366-74. doi:10.1016/j.chroma.2011.10.081
33 Pitt J J. Principles and Applications of Liquid Chromatography—Mass Spectrometry in Clinical Biochemistry. Clin Biochem Rev 2009; 30:19-34.
34 CLSI. Liquid Chromatography—Mass Spectromtry Methods; Approved Guideline CLSI document 62-A. October 20. Wayne, Pa.: Clinical and Laboratory Standards Institute 2014.
35 Hrydziuszko O, Viant M R. Missing values in mass spectrometry based metabolomics: an undervalued step in the data processing pipeline. Metabolomics 2012; 8:161-74. doi:10.1007/s11306-011-0366-4
36 Florescu A, Ferrence R, Einarson T, et al. Methods for quantification of exposure to cigarette smoking and environmental tobacco smoke: focus on developmental toxicology. Ther Drug Monit 2009; 31:14-30. doi:10.1097/FTD.0b013e3181957a3b
37 Conde-Agudelo A, Althabe F, Belizan J M, et al. Cigarette smoking during pregnancy and risk of preeclampsia: A systematic review. Am J Obstet Gynecol 1999; 181:1026-35. doi:10.1016/50002-9378(99)70341-8
38 Romero R, Nien J K, Espinoza J, et al. A longitudinal study of angiogenic (placental growth factor) and anti-angiogenic (soluble endoglin and soluble vascular endothelial growth factor receptor-1) factors in normal pregnancy and patients destined to develop preeclampsia and deliver a small for. J Matern Fetal Neonatal Med 2008; 21:9-23. doi:10.1080/14767050701830480
39 Pepe M S, Etzioni R, Feng Z, et al. Phases of biomarker development for early detection of cancer. J Natl Cancer Inst 2001; 93:1054-61. http://www.ncbi.nlm.nih.gov/pubmed/11459866
40 Wald N J, Cuckle H S, Densem J W, et al. Maternal serum screening for Down's syndrome in early pregnancy. Br Med J 1988; 297:883-7. doi:10.1136/bmj.297.6653.883
41 Mandic S, Go C, Aggarwal I, et al. Relationship of Predictive Modeling to Receiver Operating Characteristics. J Cardiopulm Rehabil Prev 2008; 28:415-9. doi:10.1097/HCR.0b013e31818c3c78
42 Zweig M H, Campbell G. Receiver-operating characteristic (ROC) plots: a fundamental evaluation tool in clinical medicine. Clin Chem 1993; 39:561-77. http://www.ncbi.nlm.nih.gov/pubmed/8472349
43 Romero-Brufau S, Huddleston J M, Escobar G J, et al. Why the C-statistic is not informative to evaluate early warning scores and what metrics to use. Crit Care 2015; 19:285. doi:10.1186/s13054-015-0999-1

1.-62. (canceled)

63. A computer implemented method of early prediction of risk of a pregnancy outcome in a pregnant woman, comprising the steps of:
inputting into a computational model:
values for a panel of a plurality of preeclampsia specific biomarkers selected from Table 1 and comprising at least one metabolite, and at least one protein or clinical risk factor, in which the values are obtained from the pregnant woman early in pregnancy;
in which the computational model is configured to:
select a subset of the inputted values comprising a value for at least one metabolite and at least one protein or clinical risk factor value, based on a pregnancy outcome selected from pre-term preeclampsia, term preeclampsia and all preeclampsia;
calculate a predicted risk of the selected pregnancy outcome based on the subset of inputted values; and
output the predicted risk of the pregnancy outcome for the pregnant woman.

64.-66. (canceled)

67. The computer implemented method according to claim 63, in which the subset of the inputted values selected by the computational model comprises a value for at least one metabolite of Table 1 and a value for at least one protein selected from placental growth factor (PlGF) and soluble endoglin (sENG).

68. The computer implemented method according to claim 63, in which the subset of the inputted values selected by the computational model comprises a value for the metabolite dilinoleoyl glycerol (DLG).

69. The computer implemented method according to claim 63, in which the subset of the inputted values selected by the computational model comprises a value for the metabolite dilinoleoyl glycerol (DLG) and a value for the protein placental growth factor (PlGF).

70. The computer implemented method according to claim 63, in which the selected pregnancy outcome is pre-term PE and in which subset of the inputted values selected by the computational model comprises values for a plurality of biomarkers selected from dilinoleoyl glycerol (DLG), 1-heptadecanoyl-2-hydroxy-sn-glycero-3-phosphocholine (1-HD), L-isoleucine (L-ISO), L-leucine (L-LEU), NG-monomethyl-L-arginine (NGM), stearoylcarnitine (SC), ergothioneine (L-ERG), 2-hydroxybutanoic acid (2-HBA), Etiocholanolone glucuronide (ECG), 20-Carboxy-leukotriene B4 (20-CL), citrulline (CR), placental growth factor (PlGF) and soluble endoglin (s-ENG).

71. The computer implemented method according to claim 63, in which the selected pregnancy outcome is pre-term PE and in which subset of the inputted values selected by the computational model comprises values for a plurality of biomarkers including PlGF and one or more selected from dilinoleoyl glycerol (DLG), 1-heptadecanoyl-2-hydroxy-sn-glycero-3-phosphocholine (1-HD), L-isoleucine (L-ISO), L-leucine (L-LEU), NG-monomethyl-L-arginine (NGM), stearoylcarnitine (SC), ergothioneine (L-ERG), 2-hydroxybutanoic acid (2-HBA), Etiocholanolone glucuronide (ECG), 20-Carboxy-leukotriene B4 (20-CL), citrulline (CR) and soluble endoglin (s-ENG).

72. The computer implemented method according to claim 63, in which the selected pregnancy outcome is term PE and in which the selected subset of values comprises values for a plurality of biomarkers selected from blood pressure (bp), 1-heptadecanoyl-2-hydroxy-sn-glycero-3-phosphocholine (1-HD), 25-Hydroxyvitamin D3 (HVD3), L-isoleucine (L-ISO), L-leucine (L-LEU), citrulline (CR), homo-L-arginine (H-L-ARG) and taurine (TR).

73. The computer implemented method according to claim 63, in which the subset of the inputted values selected by the computational model comprises a value for a clinical risk factor selected from (a) a weight related variable selected from weight, BMI or waist circumference of the pregnant woman and/or (b) blood pressure of the pregnant woman.

74. The computer implemented method according to claim 63, in which the computational model is configured to:
select a second subset of the inputted values comprising a value for at least one metabolite and at least one protein or clinical risk factor value, based on a second pregnancy outcome selected from pre-term preeclampsia, term preeclampsia and all preeclampsia;
calculate a predicted risk of the second pregnancy outcome based on the second subset of inputted values; and
output the predicted risk of the second pregnancy outcome for the pregnant woman.

75. The computer implemented method according to claim 63, in which the panel of preeclampsia specific biomarkers comprises at least three biomarkers of Table 1 including placental growth factor (PlGF), dilinoleoyl glycerol (DLG) and a further metabolite biomarker selected from 1-heptadecanoyl-2-hydroxy-sn-glycero-3-phosphocholine (1-HD), L-isoleucine (L-ISO), NG-monomethyl-L-arginine (NGM), 2-hydroxybutanoic acid (2HBA), decanoylcarnitine (DC), and choline (CL).

76. The computer implemented method according to claim 63, in which the panel of preeclampsia specific biomarkers comprises at least four biomarkers of Table 1 including placental growth factor (PlGF), dilinoleoyl glycerol (DLG) and at least two metabolite biomarkers selected from 1-heptadecanoyl-2-hydroxy-sn-glycero-3-phosphocholine (1-HD), L-isoleucine (L-ISO), NG-monomethyl-L-arginine (NGM), 2-hydroxybutanoic acid (2HBA), decanoylcarnitine (DC), and choline (CL).

77. The computer implemented method according to claim 63, in which the selected subset of values comprises values for a plurality of biomarkers selected from Table 1, and in which the or each calculation step comprises the steps of:
inputting the selected subset of values into a risk score calculation specific to the selected pregnancy outcome to calculate a risk score of the pregnancy outcome; and
compare the calculated risk score with at least one reference risk score to provide a predicted risk of the pregnancy outcome for the pregnant woman.

78. The computer implemented method according to claim 63, in which the method includes the additional step of inputting a risk category selected from elevated risk and reduced risk into the computational model, and in which the or each subset of inputted values selected by the computational model comprises (a) a rule-in subset of inputted values comprising a value for one or more rule-in biomarkers and/or (b) a rule-out subset of inputted values comprising a value for one or more rule-out biomarkers, based on the selected pregnancy outcome and selected risk category.

79. The computer implemented method according to claim 63, in which the method includes an additional step of inputting a risk category selected from elevated risk and reduced risk into the computational model, in which when the risk category inputted into the computational model is elevated risk, the computational model is configured to:
select a rule-out subset of inputted values comprising a value for one or more rule-out biomarkers, based on the selected pregnancy outcome;
determine if there is a reduced risk of the selected pregnancy outcome based on the rule-out subset of inputted values;
where a reduced risk of the selected pregnancy outcome is not determined, select a rule-in subset of inputted values comprising a value for one or more rule-in biomarkers, based on the selected pregnancy outcome;
determine if there is an elevated risk of the selected pregnancy outcome based on the rule-in subset of inputted values;
output the predicted risk of the pregnancy outcome for the pregnant woman.

80. The computer implemented method according to claim 79, in which the one or more rule-in biomarkers comprises PlGF and in which the one or more rule-out biomarkers comprises DLG.

81. The computer implemented method according to claim 79, in which the selected pregnancy outcome is pre-term preeclampsia, and in which the one or more rule-in biomarkers is selected from DLG, SC, L-ERG, ECG, 20-CL, PlGF and s-ENG.

82. The computer implemented method according to claim 79, in which the selected pregnancy outcome is term preeclampsia, and in which the one or more rule-in biomarkers is selected from bp, 1-HD, HVD3, L-ISO, L-LEU, CR and TR.

83. The computer implemented method according to claim 63, in which the method includes an additional step of inputting a risk category selected from elevated risk and reduced risk into the computational model, in which when the risk category inputted into the computational model is reduced risk, the computational model is configured to:
select a rule-in subset of inputted values comprising a value for one or more rule-in biomarkers, based on the selected pregnancy outcome;
calculating the predicted risk by determining if there is an elevated risk of the selected pregnancy outcome based on the rule-in subset of inputted values;
where an elevated risk of the selected pregnancy outcome is not determined, select a rule-out subset of inputted values comprising a value for one or more rule-out biomarkers, based on the selected pregnancy outcome;
calculating the predicted risk by determining if there is a reduced risk of the selected pregnancy outcome based on the rule-out subset of inputted values; and
output the predicted risk of the pregnancy outcome for the pregnant woman.

84. The computer implemented method according to claim 83, in which the one or more rule-in biomarkers comprises PlGF and in which the one or more rule-out biomarkers comprises DLG.

85. The computer implemented method according to claim 83, in which the selected pregnancy outcome is pre-term preeclampsia, and in which the one or more rule-in biomarkers is selected from DLG, SC, L-ERG, ECG, 20-CL, PlGF and s-ENG.

86. The computer implemented method according to claim 83, in which the selected pregnancy outcome is term preeclampsia, and in which the one or more rule-in biomarkers is selected from bp, 1-HD, HVD3, L-ISO, L-LEU, CR and TR.
